SEQUENCE LISTING (164 sequences)

SEQ ID NO: 1 - DNA, 62 bp, Artificial Sequence
  5′-UTR of human peroxisomal multifunctional enzyme type 2 lacking the 5′-terminal oligopyrimidine tract (HSD17B4)
gtcccgcagtcggcgtccagcggctctgcttgttcgtgtgtgtgtcgttgcaggccttattc

SEQ ID NO: 2 - RNA, 62 nt, Artificial Sequence
  5′-UTR of human peroxisomal multifunctional enzyme type 2 lacking the 5′-terminal oligopyrimidine tract (HSD17B4) mRNA
gucccgcagucggcguccagcggcucugcuuguucgugugugugucguugcaggccuuauuc

SEQ ID NO: 3 - DNA, 42 bp, Artificial Sequence
  5′-UTR of human ribosomal protein Large 32 lacking the 5′-terminal oligopyrimidine tract (RPL32, 32L4, 32L3)
ggcgctgcctacggaggtggcagccatctccttctcggcatc

SEQ ID NO: 4 - RNA, 42 nt, Artificial Sequence
  5′-UTR of human ribosomal protein Large 32 lacking the 5′-terminal oligopyrimidine tract (RPL32, 32L4, 32L3) mRNA
ggcgcugccuacggagguggcagccaucuccuucucggcauc

SEQ ID NO: 5 - DNA, 31 bp, Artificial Sequence
  5′-UTR of human androgen-induced gene 1 protein lacking the 5′-terminal oligopyrimidine tract (AIG1)
gccgcccagccggtccaggcctctggcgaac

SEQ ID NO: 6 - RNA, 31 nt, Artificial Sequence
  5′-UTR of human androgen-induced gene 1 protein lacking the 5′-terminal oligopyrimidine tract (AIG1) mRNA
gccgcccagccgguccaggccucuggcgaac

SEQ ID NO: 7 - DNA, 42 bp, Artificial Sequence
  5′-UTR of human acid ceramidase lacking the 5′-terminal oligopyrimidine tract (ASAH1)
gcctctgctggagtccggggagtggcgttggctgctagagcg

SEQ ID NO: 8 - RNA, 42 nt, Artificial Sequence
  5′-UTR of human acid ceramidase lacking the 5′-terminal oligopyrimidine tract (ASAH1) mRNA
gccucugcuggaguccggggaguggcguuggcugcuagagcg

SEQ ID NO: 9 - DNA, 75 bp, Artificial Sequence
  5′-UTR of human mitochondrial ATP synthase subunit alpha lacking the 5′-terminal oligopyrimidine tract (ATP5A1)
gcggctcggccattttgtcccagtcagtccggaggctgcggctgcagaagtaccgcctgcggagtaactgcaaag

SEQ ID NO: 10 - RNA, 75 nt, Artificial Sequence
  5′-UTR of human mitochondrial ATP synthase subunit alpha lacking the 5′-terminal oligopyrimidine tract (ATP5A1) mRNA
gcggcucggccauuuugucccagucaguccggaggcugcggcugcagaaguaccgccugcggaguaacugcaaag

SEQ ID NO: 11 - DNA, 56 bp, Artificial Sequence
  5′-UTR of human cytochrome c oxidase subunit 6C lacking the 5′-terminal oligopyrimidine tract (COX6C)
agtcaggaaggacgttggtgttgaggttagcatacgtatcaaggacagtaactacc

SEQ ID NO: 12 - RNA, 56 nt, Artificial Sequence
  5′-UTR of human cytochrome c oxidase subunit 6C lacking the 5′-terminal oligopyrimidine tract (COX6C) mRNA
agucaggaaggacguugguguugagguuagcauacguaucaaggacaguaacuacc

SEQ ID NO: 13 - DNA, 232 bp, Artificial Sequence
  5′-UTR of mouse dihydropyrimidinase-like 2 (Dpysl2)
ctctctccctctttcttttttccgccctagctggatctgtgttggaggagaggaaaagagacagaggattgcatctgttttgaaatctcctaatagcaagaccagttaagggattgtacctttttcctacaaatataaatatatatatattttaaaccaagtctttttttttccggctatctttgctttaaagctgtcctcttgagattacttcctcccgccccccggagag

SEQ ID NO: 14 - RNA, 232 nt, Artificial Sequence
  5′-UTR of mouse dihydropyrimidinase-like 2 (Dpysl2) mRNA
cucucucccucuuucuuuuuuccgcccuagcuggaucuguguuggaggagaggaaaagagacagaggauugcaucuguuuugaaaucuccuaauagcaagaccaguuaagggauuguaccuuuuuccuacaaauauaaauauauauauauuuuaaaccaagucuuuuuuuuuccggcuaucuuugcuuuaaagcuguccucuugagauuacuuccucccgccccccggagag

SEQ ID NO: 15 - DNA, 22 bp, Artificial Sequence
  5′-UTR of human ATP binding cassette subfamily B member 7 lacking the 5′-terminal oligopyrimidine tract (MDR, ABCB7)
ggttcctctttcctcgctcaag

SEQ ID NO: 16 - RNA, 22 nt, Artificial Sequence
  5′-UTR of human ATP binding cassette subfamily B member 7 lacking the 5′-terminal oligopyrimidine tract (MDR, ABCB7) mRNA
gguuccucuuuccucgcucaag

SEQ ID NO: 17 - DNA, 54 bp, Artificial Sequence
  5′-UTR of mouse RIKEN cDNA 2010107E04 gene (Mp68, 2010107E04Rik)
ctttcccattctgtagcagaatttggtgttgcctgtggtcttggtcccgcggag

SEQ ID NO: 18 - RNA, 54 nt, Artificial Sequence
  5′-UTR of mouse RIKEN cDNA 2010107E04 gene (Mp68, 2010107E04Rik) mRNA
cuuucccauucuguagcagaauuugguguugccuguggucuuggucccgcggag

SEQ ID NO: 19 - DNA, 81 bp, Artificial Sequence
  5′-UTR of mouse NADH dehydrogenase (ubiquinone) 1 alpha subcomplex, 4 (Ndufa4)
gtccgctcagccaggttgcagaagcggcttagcgtgtgtcctaatcttctctctgcgtgtaggtaggcctgtgccgcaaac

SEQ ID NO: 20 - RNA, 81 nt, Artificial Sequence
  5′-UTR of mouse NADH dehydrogenase (ubiquinone) 1 alpha subcomplex, 4 (Ndufa4) mRNA
guccgcucagccagguugcagaagcggcuuagcguguguccuaaucuucucucugcguguagguaggccugugccgcaaac

SEQ ID NO: 21 - DNA, 108 bp, Artificial Sequence
  5′-UTR of mouse nitric oxide synthase interacting protein (Nosip)
ctcctgtcgggcggaagtaggaggagtagagtttaaaaacagtactctttttccggttcgggacgtagttgaagcaacgacaagccggataaccgctcttgagacagg

SEQ ID NO: 22 - RNA, 108 nt, Artificial Sequence
  5′-UTR of mouse nitric oxide synthase interacting protein (Nosip) mRNA
cuccugucgggcggaaguaggaggaguagaguuuaaaaacaguacucuuuuuccgguucgggacguaguugaagcaacgacaagccggauaaccgcucuugagacagg

SEQ ID NO: 23 - DNA, 84 bp, Artificial Sequence
  5′-UTR of mouse ribosomal protein L31 (Rpl31)
cccgtgacccggaagttgtacggctacgcgactttccctcccacaaaccctcgcgcccttcctttcctacttgggcccggcaga

SEQ ID NO: 24 - RNA, 84 nt, Artificial Sequence
  5′-UTR of mouse ribosomal protein L31 (Rpl31) mRNA
cccgugacccggaaguuguacggcuacgcgacuuucccucccacaaacccucgcgcccuuccuuuccuacuugggcccggcaga

SEQ ID NO: 25 - DNA, 56 bp, Artificial Sequence
  5′-UTR of human 60S ribosomal protein L35a lacking the 5′-terminal oligopyrimidine tract (RPL35A)
accgccatcttggctcctgtggaggcctgctgggaacgggacttctaaaaggaact

SEQ ID NO: 26 - RNA, 56 nt, Artificial Sequence
  5′-UTR of human 60S ribosomal protein L35a lacking the 5′-terminal oligopyrimidine tract (RPL35A) mRNA
accgccaucuuggcuccuguggaggccugcugggaacgggacuucuaaaaggaacu

SEQ ID NO: 27 - DNA, 159 bp, Artificial Sequence
  5′-UTR of mouse solute carrier family 7 (cationic amino acid transporter, y+ system), member 3 (Slc7a3)
gggcgcttggcttgcaaggaccctgagctgcggcattgaagcacacccaacccaactcgactgaagtcagcctcactgaaccggatctgagaatcttctctctctgggcttgccagggctctccgaacctagctagcatcctcttcaattccaactaga

SEQ ID NO: 28 - RNA, 159 nt, Artificial Sequence
  5′-UTR of mouse solute carrier family 7 (cationic amino acid transporter, y+ system), member 3 (Slc7a3) mRNA
gggcgcuuggcuugcaaggacccugagcugcggcauugaagcacacccaacccaacucgacugaagucagccucacugaaccggaucugagaaucuucucucucugggcuugccagggcucuccgaaccuagcuagcauccucuucaauuccaacuaga

SEQ ID NO: 29 - DNA, 102 bp, Artificial Sequence
  5′-UTR of human tubulin beta 4B class Ivb (TUBB4B)
atataagcgttggcggagcgtcggttgtagcactctgcgcgcccgctcttctgctgctgtttgtctacttcctcctgcttccccgccgccgccgccgccatc

SEQ ID NO: 30 - RNA, 102 nt, Artificial Sequence
  5′-UTR of human tubulin beta 4B class Ivb (TUBB4B) mRNA
auauaagcguuggcggagcgucgguuguagcacucugcgcgcccgcucuucugcugcuguuugucuacuuccuccugcuuccccgccgccgccgccgccauc

SEQ ID NO: 31 - DNA, 222 bp, Artificial Sequence
  5′-UTR of mouse ubiquilin 2 (Ubqln2)
cggagacggcctgcaggacctgctctctcagccctcagccgaggcctacgccgagccgagtgcgcagccgacgaccgggaggagccgcagccttcaactctgaggtactgtgatccgcgctgcccgccgggccgccccagtccgctgctgcggcacctccttccctcgcgccctcttcgctcgccagcgccttccctgtgagcctgcgtcaccgcggccgcc

SEQ ID NO: 32 - RNA, 222 nt, Artificial Sequence
  5′-UTR of mouse ubiquilin 2 (Ubqln2) mRNA
cggagacggccugcaggaccugcucucucagcccucagccgaggccuacgccgagccgagugcgcagccgacgaccgggaggagccgcagccuucaacucugagguacugugauccgcgcugcccgccgggccgccccaguccgcugcugcggcaccuccuucccucgcgcccucuucgcucgccagcgccuucccugugagccugcgucaccgcggccgcc

SEQ ID NO: 33 - DNA, 44 bp, Artificial Sequence
  Center, alpha-complex-binding portion of the 3′-UTR of an alpha-globin gene (muag)
gcccgatgggcctcccaacgggccctcctcccctccttgcaccg

SEQ ID NO: 34 - RNA, 44 nt, Artificial Sequence
  Center, alpha-complex-binding portion of the 3′-UTR of an alpha-globin gene (muag) mRNA
gcccgaugggccucccaacgggcccuccuccccuccuugcaccg

SEQ ID NO: 35 - DNA, 57 bp, Artificial Sequence
  3′-UTR of human proteasome subunit beta 3 (PSMB3)
ccctgttcccagagcccacttttttttctttttttgaaataaaatagcctgtctttc

SEQ ID NO: 36 - RNA, 57 nt, Artificial Sequence
  3′-UTR of human proteasome subunit beta 3 (PSMB3) mRNA
cccuguucccagagcccacuuuuuuuucuuuuuuugaaauaaaauagccugucuuuc

SEQ ID NO: 37 - DNA, 187 bp, Artificial Sequence
  3′-UTR ALB7
gcatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacct

SEQ ID NO: 38 - RNA, 187 nt, Artificial Sequence
  3′-UTR ALB7 mRNA
gcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccu

SEQ ID NO: 39 - DNA, 266 bp, Artificial Sequence
  3′-UTR of human annexin A4 (ANXA4)
aataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaa

SEQ ID NO: 40 - RNA, 266 nt, Artificial Sequence
  3′-UTR of human annexin A4 (ANXA4) mRNA
aauaaaaaucccagaaggacaggaggauucucaacacuuugaauuuuuuuaacuucauuuuucuacacugcuauuaucauuaucucagaaugcuuauuuccaauuaaaacgccuacagcugccuccuagaauauagacugucuguauuauuauucaccuauaauuagucauuaugaugcuuuaaagcuguacuugcauuucaaagcuuauaagauauaaauggagauuuuaaaguagaaauaaauauguauuccauguuuuuaaaa

SEQ ID NO: 41 - DNA, 121 bp, Artificial Sequence
  3′-UTR of human caspase 1 (CASP1)
aataaggaaactgtatgaatgtctgtgggcaggaagtgaagagatccttctgtaaaggtttttggaattatgtctgctgaataataaacttttttgaaataataaatctggtagaaaaatg

SEQ ID NO: 42 - RNA, 121 nt, Artificial Sequence
  3′-UTR of human caspase 1 (CASP1) mRNA
aauaaggaaacuguaugaaugucugugggcaggaagugaagagauccuucuguaaagguuuuuggaauuaugucugcugaauaauaaacuuuuuugaaauaauaaaucugguagaaaaaug

SEQ ID NO: 43 - DNA, 137 bp, Artificial Sequence
  3′-UTR of human cytochrome c oxidase subunit 6B1 (COX6B1)
actggctgcatctccctttcctctgtcctccatccttctcccaggatggtgaagggggacctggtacccagtgatccccaccccaggatcctaaatcatgacttacctgctaataaaaactcattggaaaagtgaga

SEQ ID NO: 44 - RNA, 137 nt, Artificial Sequence
  3′-UTR of human cytochrome c oxidase subunit 6B1 (COX6B1) mRNA
acuggcugcaucucccuuuccucuguccuccauccuucucccaggauggugaagggggaccugguacccagugauccccaccccaggauccuaaaucaugacuuaccugcuaauaaaaacucauuggaaaagugaga

SEQ ID NO: 45 - DNA, 166 bp, Artificial Sequence
  3′-UTR of human FIG4 phosphoinositide 5-phosphatase (FIG4)
aaagagcgcaggtccacctggtggacacgtctgattagcttagaacctgtcttgtctcatcttcaaaaggtaacttattaaaagtcctttgcgtctgaagcctttctccttttctgtcacttgcaaattccaaattatagctaataaagatgactagataatttgc

SEQ ID NO: 46 - RNA, 166 nt, Artificial Sequence
  3′-UTR of human FIG4 phosphoinositide 5-phosphatase (FIG4) mRNA
aaagagcgcagguccaccugguggacacgucugauuagcuuagaaccugucuugucucaucuucaaaagguaacuuauuaaaaguccuuugcgucugaagccuuucuccuuuucugucacuugcaaauuccaaauuauagcuaauaaagaugacuagauaauuugc

SEQ ID NO: 47 - DNA, 353 bp, Artificial Sequence
  3′-UTR of mouse GNAS (guanine nucleotide binding protein, alpha stimulating) complex locus (Gnas)
gaagggaacacccaaatttaattcagccttaagcacaattaattaagagtgaaacgtaattgtacaagcagttggtcacccaccatagggcatgatcaacaccgcaacctttcctttttcccccagtgattctgaaaaacccctcttcccttcagcttgcttagatgttccaaatttagtaagcttaaggcggcctacagaagaaaaagaaaaaaaaggccacaaaagttccctctcactttcagtaaataaaataaaagcagcaacagaaataaagaaataaatgaaattcaaaatgaaataaatattgtgttgtgcagcattaaaaaatcaataaaaattaaaaatgagca

SEQ ID NO: 48 - RNA, 353 nt, Artificial Sequence
  3′-UTR of mouse GNAS (guanine nucleotide binding protein, alpha stimulating) complex locus (Gnas) mRNA
gaagggaacacccaaauuuaauucagccuuaagcacaauuaauuaagagugaaacguaauuguacaagcaguuggucacccaccauagggcaugaucaacaccgcaaccuuuccuuuuucccccagugauucugaaaaaccccucuucccuucagcuugcuuagauguuccaaauuuaguaagcuuaaggcggccuacagaagaaaaagaaaaaaaaggccacaaaaguucccucucacuuucaguaaauaaaauaaaagcagcaacagaaauaaagaaauaaaugaaauucaaaaugaaauaaauauuguguugugcagcauuaaaaaaucaauaaaaauuaaaaaugagca

SEQ ID NO: 49 - DNA, 133 bp, Artificial Sequence
  3′-UTR of mouse NADH dehydrogenase (ubiquinone) 1 alpha subcomplex, 1 (Ndufa1)
ggaagcattttcctggctgattaaaagaaattactcagctatggtcatctgttcctgttagaaggctatgcagcatattatatactatgcgcatgttatgaaatgcataataaaaaattttaaaaaatctaaa

SEQ ID NO: 50 - RNA, 133 nt, Artificial Sequence
  3′-UTR of mouse NADH dehydrogenase (ubiquinone) 1 alpha subcomplex, 1 (Ndufa1) mRNA
ggaagcauuuuccuggcugauuaaaagaaauuacucagcuauggucaucuguuccuguuagaaggcuaugcagcauauuauauacuaugcgcauguuaugaaaugcauaauaaaaaauuuuaaaaaaucuaaa

SEQ ID NO: 51 - DNA, 70 bp, Artificial Sequence
  3′-UTR of human ribosomal protein S9 (RPS9)
gtccacctgtccctcctgggctgctggattgtctcgttttcctgccaaataaacaggatcagcgctttac

SEQ ID NO: 52 - RNA, 70 nt, Artificial Sequence
  3′-UTR of human ribosomal protein S9 (RPS9) mRNA
guccaccugucccuccugggcugcuggauugucucguuuuccugccaaauaaacaggaucagcgcuuuac

SEQ ID NO: 53 - DNA, 240 bp, Artificial Sequence
  3′-UTR of mouse solute carrier family 7 (cationic amino acid transporter, y+ system), member 3 (Slc7a3)
cctaaccatacctaattgttgtcttgttctcctatacaataatggagagtactcttgaccccacgaagagctgggcctctcatgtggtgttagtgacggatatgaaataatgctctgtatttcttgtgaaatgatggctatgtctttgctgtttctcatttgtttttaacttgtatactttgaaatagtctctgtagttgtttcctggctttgaaaaaaatgttattaaaggaaaattgt

SEQ ID NO: 54 - RNA, 240 nt, Artificial Sequence
  3′-UTR of mouse solute carrier family 7 (cationic amino acid transporter, y+ system), member 3 (Slc7a3) mRNA
ccuaaccauaccuaauuguugucuuguucuccuauacaauaauggagaguacucuugaccccacgaagagcugggccucucaugugguguuagugacggauaugaaauaaugcucuguauuucuugugaaaugauggcuaugucuuugcuguuucucauuuguuuuuaacuuguauacuuugaaauagucucuguaguuguuuccuggcuuugaaaaaaauguuauuaaaggaaaauugu

SEQ ID NO: 55 - DNA, 151 bp, Artificial Sequence
  3′-UTR of human tubulin beta 4B class Ivb (TUBB4B)
agccttcagtcactggggaaagcagggaagcagtgtgaactctttattcactcccagcctgtcctgtggcctgtcccactgtgtgcacttgctgttttccctgtccacatccatgctgtacagacaccaccattaaagcattttcatagtg

SEQ ID NO: 56 - RNA, 151 nt, Artificial Sequence
  3′-UTR of human tubulin beta 4B class Ivb (TUBB4B) mRNA
agccuucagucacuggggaaagcagggaagcagugugaacucuuuauucacucccagccuguccuguggccugucccacugugugcacuugcuguuuucccuguccacauccaugcuguacagacaccaccauuaaagcauuuucauagug

SEQ ID NO: 57 - RNA, 6 nt, Artificial Sequence
  mRNA 5′-end
gggaga

SEQ ID NO: 58 - RNA, 6 nt, Artificial Sequence
  mRNA 5′-end
aggaga

SEQ ID NO: 59 - DNA, 13 bp, Artificial Sequence
  Kozak
gccgccaccatgg

SEQ ID NO: 60 - RNA, 13 nt, Artificial Sequence
  Kozak mRNA
gccgccaccaugg

SEQ ID NO: 61 - DNA, 24 bp, Artificial Sequence
  histone stem-loop sequence (histoneSL, hSL)
caaaggctcttttcagagccacca

SEQ ID NO: 62 - RNA, 24 nt, Artificial Sequence
  histone stem-loop sequence (histoneSL, hSL) mRNA
caaaggcucuuuucagagccacca

SEQ ID NO: 63 - RNA, 128 nt, Artificial Sequence
  mRNA 3′-end A64-N5-C30-histoneSL-N5
aaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 64 - RNA, 129 nt, Artificial Sequence
  mRNA 3′-end histoneSL-A100-N5
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 65 - RNA, 93 nt, Artificial Sequence
  mRNA 3′-end histoneSL-A64-N5
caaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 66 - RNA, 105 nt, Artificial Sequence
  mRNA 3′-end A100-N5
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 67 - RNA, 69 nt, Artificial Sequence
  mRNA 3′-end A64-N5
aaaaaaaaaaaaaaaaaaaaaaaaaaaaaaaaaaaaaaaaaaaaaaaaaaaaaaaaaaaaaaaagaauu

SEQ ID NO: 68 - RNA, 124 nt, Artificial Sequence
  mRNA 3′-end histoneSL-A100
caaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 69 - RNA, 88 nt, Artificial Sequence
  mRNA 3′-end histoneSL-A64
caaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 70 - RNA, 100 nt, Artificial Sequence
  mRNA 3′-end A100
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 71 - RNA, 64 nt, Artificial Sequence
  mRNA 3′-end A64
aaaaaaaaaaaaaaaaaaaaaaaaaaaaaaaaaaaaaaaaaaaaaaaaaaaaaaaaaaaaaaaa

SEQ ID NO: 72 - RNA, 93 nt, Artificial Sequence
  mRNA 3′-end A64-histoneSL-N5
aaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 73 - RNA, 134 nt, Artificial Sequence
  mRNA 3′-end N6-A64-N5-C30-histoneSL-N5
auuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 74 - RNA, 134 nt, Artificial Sequence
  mRNA 3′-end N6-A64-N5-C30-histoneSL-N5
agaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 75 - RNA, 135 nt, Artificial Sequence
  mRNA 3′-end N6-histoneSL-A100-N5
auuaaucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 76 - RNA, 135 nt, Artificial Sequence
  mRNA 3′-end N6-histoneSL-A100-N5
agaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 77 - RNA, 99 nt, Artificial Sequence
  mRNA 3′-end N6-histoneSL-A64-N5
auuaaucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 78 - RNA, 99 nt, Artificial Sequence
  mRNA 3′-end N6-histoneSL-A64-N5
agaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 79 - RNA, 111 nt, Artificial Sequence
  mRNA 3′-end N6-A100-N5
auuaauaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 80 - RNA, 111 nt, Artificial Sequence
  mRNA 3′-end N6-A100-N5
agaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 81 - RNA, 75 nt, Artificial Sequence
  mRNA 3′-end N6-A64-N5
auuaauaaaaaaaaaaaaaaaaaaaaaaaaaaaaaaaaaaaaaaaaaaaaaaaaaaaaaaaaaaaaaaaagaauu

SEQ ID NO: 82 - RNA, 75 nt, Artificial Sequence
  mRNA 3′-end N6-A64-N5
agaucuaaaaaaaaaaaaaaaaaaaaaaaaaaaaaaaaaaaaaaaaaaaaaaaaaaaaaaaaaaaaaaaagaauu

SEQ ID NO: 83 - RNA, 130 nt, Artificial Sequence
  mRNA 3′-end N6-histoneSL-A100
auuaaucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 84 - RNA, 130 nt, Artificial Sequence
  mRNA 3′-end N6-histoneSL-A100
agaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 85 - RNA, 94 nt, Artificial Sequence
  mRNA 3′-end N6-histoneSL-A64
auuaaucaaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 86 - RNA, 94 nt, Artificial Sequence
  mRNA 3′-end N6-histoneSL-A64
agaucucaaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 87 - RNA, 106 nt, Artificial Sequence
  mRNA 3′-end N6-A100
auuaauaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 88 - RNA, 106 nt, Artificial Sequence
  mRNA 3′-end N6-A100
agaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 89 - RNA, 70 nt, Artificial Sequence
  mRNA 3′-end N6-A64
auuaauaaaaaaaaaaaaaaaaaaaaaaaaaaaaaaaaaaaaaaaaaaaaaaaaaaaaaaaaaaaaaaaa

SEQ ID NO: 90 - RNA, 70 nt, Artificial Sequence
  mRNA 3′-end N6-A64
agaucuaaaaaaaaaaaaaaaaaaaaaaaaaaaaaaaaaaaaaaaaaaaaaaaaaaaaaaaaaaaaaaaa

SEQ ID NO: 91 - RNA, 99 nt, Artificial Sequence
  mRNA 3′-end N6-A64-histoneSL-N5
auuaauaaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 92 - RNA, 99 nt, Artificial Sequence
  mRNA 3′-end N6-A64-histoneSL-N5
agaucuaaaaaaaaaaaaaaaaaaaaaaaaaaaaaaaaaaaaaaaaaaaaaaaaaaaaaaaaaaaaaaaacaaaggcucuuuucagagccaccagaauu

SEQ ID NO: 93 - protein, 14 aa, Artificial Sequence
  CR12C carrier peptide
CRRRRRRRRRRRRC

SEQ ID NO: 94 - protein, 13 aa, Artificial Sequence
  CR12 carrier peptide
CRRRRRRRRRRRR

SEQ ID NO: 95 - protein, 14 aa, Artificial Sequence
  WR12C carrier peptide
WRRRRRRRRRRRRC

SEQ ID NO: 96 - protein, 18 aa, Artificial Sequence
  CH6R4H6C carrier peptide
CHHHHHHRRRRHHHHHHC

SEQ ID NO: 97 - protein, 18 aa, Artificial Sequence
  CGH5R4H5GC carrier peptide
CGHHHHHRRRRHHHHHGC

SEQ ID NO: 98 - RNA, 1435 nt, Artificial Sequence
  malaria HsALB(1-18)_Pf-CSP(19-397)(GC) mRNA Design a-1 (hSL-A64-N5, EcoRI) R8523
aggagagucccgcagucggcguccagcggcucugcuuguucgugugugugucguugcaggccuuauucaagcuuaccaugaagugggugaccuucaucagccugcucuuccuguucuccagcgccuacucccuguuccaggaguaccagugcuacggcuccagcuccaacacccgggugcucaacgagcugaacuacgacaacgcggggaccaaccuguacaacgagcucgagaugaacuacuacggcaagcaggagaacugguacagccugaagaagaacucccgcagccugggggagaacgacgacggcaacaacgaggacaacgagaagcuccggaagcccaagcacaagaagcugaagcagccggccgacgggaaccccgaccccaacgccaaccccaacguggacccgaacgccaaccccaacgucgaccccaacgccaaccccaacguggacccgaacgcgaaccccaacgccaaccccaacgccaaccccaacgccaacccgaacgcgaaccccaacgccaaccccaacgccaaccccaacgccaacccgaacgccaaccccaacgcgaaccccaacgccaaccccaacgccaacccgaacgccaaccccaacgccaaccccaacgcgaaccccaacgccaacccgaacgccaaccccaacguggaccccaacgccaaccccaacgccaacccgaacgcgaaccccaacgccaaccccaacgccaacccgaacgccaaccccaacgccaaccccaacgcgaaccccaacgccaacccgaacgccaaccccaacgccaaccccaacgcgaaccccaacgccaacccgaacgccaaccccaacgccaaccccaacgccaaccccaacgcgaacccgaacgccaaccccaacaagaacaaccagggcaacggccaggggcacaacaugcccaacgaccccaaccgcaacgucgacgagaacgccaacgccaacuccgccgugaagaacaacaacaacgaggagccgagcgacaagcacaucaaggaguaccugaacaagauccagaacucccucagcacggaguggucccccugcagcgugaccugcggcaacgggauccagguccggaucaagcccggcuccgcgaacaagcccaaggacgagcuggacuacgccaacgacaucgagaagaagaucugcaagauggagaagugcagcuccguguucaacguggucaacagcuccaucgggcugaucauggugcucagcuuccuguuccugaacugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 99 - RNA, 1466 nt, Artificial Sequence
  malaria HsALB(1-18)_Pf-CSP(19-397)(GC) mRNA Design a-1 (hSL-A100, SapI) R8520
aggagagucccgcagucggcguccagcggcucugcuuguucgugugugugucguugcaggccuuauucaagcuuaccaugaagugggugaccuucaucagccuccuguuccuguucuccagcgccuacucccuguuccaggaguaccagugcuacggcuccagcuccaacacccgggugcucaacgagcugaacuacgacaacgcggggaccaaccuguacaacgagcucgagaugaacuacuacggcaagcaggagaacugguacagccugaagaagaacucccgcagccugggggagaacgacgacggcaacaacgaggacaacgagaagcuccggaagcccaagcacaagaagcugaagcagccggccgacgggaaccccgaccccaacgccaaccccaacguggacccgaacgccaaccccaacgucgaccccaacgccaaccccaacguggacccgaacgcgaaccccaacgccaaccccaacgccaaccccaacgccaacccgaacgcgaaccccaacgccaaccccaacgccaaccccaacgccaacccgaacgccaaccccaacgcgaaccccaacgccaaccccaacgccaacccgaacgccaaccccaacgccaaccccaacgcgaaccccaacgccaacccgaacgccaaccccaacguggaccccaacgccaaccccaacgccaacccgaacgcgaaccccaacgccaaccccaacgccaacccgaacgccaaccccaacgccaaccccaacgcgaaccccaacgccaacccgaacgccaaccccaacgccaaccccaacgcgaaccccaacgccaacccgaacgccaaccccaacgccaaccccaacgccaaccccaacgcgaacccgaacgccaaccccaacaagaacaaccagggcaacggccaggggcacaacaugcccaacgaccccaaccgcaacgucgacgagaacgccaacgccaacuccgccgugaagaacaacaacaacgaggagccgagcgacaagcacaucaaggaguaccugaacaagauccagaacucccucagcacggaguggucccccugcagcgugaccugcggcaacgggauccagguccggaucaagcccggcuccgcgaacaagcccaaggacgagcuggacuacgccaacgacaucgagaagaagaucugcaagauggagaagugcagcuccguguucaacguggucaacagcuccaucgggcugaucauggugcucagcuuccuguuccugaacugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 100 - RNA, 1471 nt, Artificial Sequence
  malaria HsALB(1-18)_Pf-CSP(19-397)(GC) mRNA Design a-1 (hSL-A100-N5, EcoRI) R8987
aggagagucccgcagucggcguccagcggcucugcuuguucgugugugugucguugcaggccuuauucaagcuuaccaugaagugggugaccuucaucagccugcucuuccuguucuccagcgccuacucccuguuccaggaguaccagugcuacggcuccagcuccaacacccgggugcucaacgagcugaacuacgacaacgcggggaccaaccuguacaacgagcucgagaugaacuacuacggcaagcaggagaacugguacagccugaagaagaacucccgcagccugggggagaacgacgacggcaacaacgaggacaacgagaagcuccggaagcccaagcacaagaagcugaagcagccggccgacgggaaccccgaccccaacgccaaccccaacguggacccgaacgccaaccccaacgucgaccccaacgccaaccccaacguggacccgaacgcgaaccccaacgccaaccccaacgccaaccccaacgccaacccgaacgcgaaccccaacgccaaccccaacgccaaccccaacgccaacccgaacgccaaccccaacgcgaaccccaacgccaaccccaacgccaacccgaacgccaaccccaacgccaaccccaacgcgaaccccaacgccaacccgaacgccaaccccaacguggaccccaacgccaaccccaacgccaacccgaacgcgaaccccaacgccaaccccaacgccaacccgaacgccaaccccaacgccaaccccaacgcgaaccccaacgccaacccgaacgccaaccccaacgccaaccccaacgcgaaccccaacgccaacccgaacgccaaccccaacgccaaccccaacgccaaccccaacgcgaacccgaacgccaaccccaacaagaacaaccagggcaacggccaggggcacaacaugcccaacgaccccaaccgcaacgucgacgagaacgccaacgccaacuccgccgugaagaacaacaacaacgaggagccgagcgacaagcacaucaaggaguaccugaacaagauccagaacucccucagcacggaguggucccccugcagcgugaccugcggcaacgggauccagguccggaucaagcccggcuccgcgaacaagcccaaggacgagcuggacuacgccaacgacaucgagaagaagaucugcaagauggagaagugcagcuccguguucaacguggucaacagcuccaucgggcugaucauggugcucagcuuccuguuccugaacugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 101 - protein, 28 aa, Artificial Sequence
  malaria [NANP]7 peptide (ELISA)
NANPNANPNANPNANPNANPNANPNANP

SEQ ID NO: 102 - RNA, 1703 nt, Artificial Sequence
  anti-Rabies-mAb-HC(GC) mRNA Design a-1 (hSL-A100, SapI) R8585
aggagagucccgcagucggcguccagcggcucugcuuguucgugugugugucguugcaggccuuauucaagcuuaccauggacuggaccuggcgcuuccuguucguggucgccgccgcgaccggcgugcagagccaggugcagcucguccaguccggggccgaggugaagaagcccggcagcuccgugaaggucagcugcaaggccuccgggggcacguucaaccgguacaccgugaacugggugcgccaggccccggggcagggccuggaguggaugggcgggaucauccccaucuucggcaccgccaacuacgcgcagcgguuccaggggcgccugaccaucacggccgacgagagcaccuccaccgccuacauggagcucagcucccugcggagcgacgacaccgccgucuacuucugcgcgcgcgagaaccuggacaacuccggcacguacuacuacuucagcgggugguucgaccccuggggccagggcacccucgugaccguguccagcgccuccaccaaggggcccagcgucuucccgcuggcccccuccagcaaguccacgagcggcgggaccgccgcccugggcugccucgugaaggacuacuuccccgagcccgugaccgucagcuggaacuccggcgcgcugaccagcggggugcacacguucccggccgugcugcaguccagcggccucuacucccugagcuccgucgugaccgugcccagcuccagccuggggacccagaccuacaucugcaacgucaaccacaagcccuccaacacgaagguggacaagcggguggagcccaagagcugcgacaagacccacaccugcccgcccugccccgccccggagcuccugggcgggccguccgucuuccuguucccucccaagcccaaggacacccucaugaucagccgcacgccggaggugaccugcgucgugguggacgugucccacgaggaccccgaggugaaguucaacugguacguggacggcgucgaggugcacaacgccaagaccaagccccgggaggagcaguacaacagcaccuaccgcguggucuccgugcugacggugcugcaccaggacuggcucaacggcaaggaguacaagugcaaggucagcaacaaggcccugcccgcgccgaucgagaagaccaucuccaaggccaaggggcagccccgggagccccagguguacacccugcccccgagccgcgaggagaugaccaagaaccaggugagccucacgugccuggucaagggcuucuaccccuccgacaucgccguggagugggagagcaacgggcagcccgagaacaacuacaagaccaccccgcccgugcuggacuccgacggcagcuucuuccucuacuccaagcugaccgucgacaagagccgguggcagcaggggaacguguucuccugcagcgugaugcacgaggcccugcacaaccacuacacgcagaagucccucagccugucccccggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 103 - RNA, 1001 nt, Artificial Sequence
  anti-Rabies-mAb-LC(GC) mRNA Design a-1 (hSL-A100, SapI) R8586
aggagagucccgcagucggcguccagcggcucugcuuguucgugugugugucguugcaggccuuauucaagcuuaccaugagcgugcccaccauggccugggcccugcuccugcugucccuccugacccagggcacggggagcugggcgcaguccgcccugacccagccgcgcagcgucuccggcagccccgggcaguccgugaccaucagcugcaccggcacguccagcgacaucgggggcuacaacuucguguccugguaccagcagcaccccggcaaggcccccaagcucaugaucuacgacgccaccaagcggccgagcgggguccccgaccgcuucuccggcagcaaguccgggaacaccgccagccugaccaucuccggccugcaggcggaggacgaggccgacuacuacugcugcagcuacgccggggacuacacgcccggcgugguguucggcgggggcaccaagcucaccguccuggggcagccgaaggccgcgccguccgugacccuguucccucccagcagcgaggagcuccaggccaacaaggccacgcuggugugccugaucuccgacuucuaccccggcgccgucaccguggccuggaaggcggacagcuccccggugaaggccggcgucgagaccaccacgcccagcaagcaguccaacaacaaguacgccgccagcuccuaccucagccugacccccgagcaguggaagucccaccggagcuacuccugccaggugacccacgaggggagcaccguggagaagacggucgcccccaccgagugcuccugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 104 - RNA, 1708 nt, Artificial Sequence
  anti-Rabies-mAb-HC(GC) mRNA Design a-1 (hSL-A100-N5, EcoRI) R8823
aggagagucccgcagucggcguccagcggcucugcuuguucgugugugugucguugcaggccuuauucaagcuuaccauggacuggaccuggcgcuuccuguucguggucgccgccgcgaccggcgugcagagccaggugcagcucguccaguccggggccgaggugaagaagcccggcagcuccgugaaggucagcugcaaggccuccgggggcacguucaaccgguacaccgugaacugggugcgccaggccccggggcagggccuggaguggaugggcgggaucauccccaucuucggcaccgccaacuacgcgcagcgguuccaggggcgccugaccaucacggccgacgagagcaccuccaccgccuacauggagcucagcucccugcggagcgacgacaccgccgucuacuucugcgcgcgcgagaaccuggacaacuccggcacguacuacuacuucagcgggugguucgaccccuggggccagggcacccucgugaccguguccagcgccuccaccaaggggcccagcgucuucccgcuggcccccuccagcaaguccacgagcggcgggaccgccgcccugggcugccucgugaaggacuacuuccccgagcccgugaccgucagcuggaacuccggcgcgcugaccagcggggugcacacguucccggccgugcugcaguccagcggccucuacucccugagcuccgucgugaccgugcccagcuccagccuggggacccagaccuacaucugcaacgucaaccacaagcccuccaacacgaagguggacaagcggguggagcccaagagcugcgacaagacccacaccugcccgcccugccccgccccggagcuccugggcgggccguccgucuuccuguucccucccaagcccaaggacacccucaugaucagccgcacgccggaggugaccugcgucgugguggacgugucccacgaggaccccgaggugaaguucaacugguacguggacggcgucgaggugcacaacgccaagaccaagccccgggaggagcaguacaacagcaccuaccgcguggucuccgugcugacggugcugcaccaggacuggcucaacggcaaggaguacaagugcaaggucagcaacaaggcccugcccgcgccgaucgagaagaccaucuccaaggccaaggggcagccccgggagccccagguguacacccugcccccgagccgcgaggagaugaccaagaaccaggugagccucacgugccuggucaagggcuucuaccccuccgacaucgccguggagugggagagcaacgggcagcccgagaacaacuacaagaccaccccgcccgugcuggacuccgacggcagcuucuuccucuacuccaagcugaccgucgacaagagccgguggcagcaggggaacguguucuccugcagcgugaugcacgaggcccugcacaaccacuacacgcagaagucccucagccugucccccggcaagugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 105 - RNA, 1006 nt, Artificial Sequence
  anti-Rabies-mAb-LC(GC) mRNA Design a-1 (hSL-A100-N5, EcoRI) R8827
aggagagucccgcagucggcguccagcggcucugcuuguucgugugugugucguugcaggccuuauucaagcuuaccaugagcgugcccaccauggccugggcccugcuccugcugucccuccugacccagggcacggggagcugggcgcaguccgcccugacccagccgcgcagcgucuccggcagccccgggcaguccgugaccaucagcugcaccggcacguccagcgacaucgggggcuacaacuucguguccugguaccagcagcaccccggcaaggcccccaagcucaugaucuacgacgccaccaagcggccgagcgggguccccgaccgcuucuccggcagcaaguccgggaacaccgccagccugaccaucuccggccugcaggcggaggacgaggccgacuacuacugcugcagcuacgccggggacuacacgcccggcgugguguucggcgggggcaccaagcucaccguccuggggcagccgaaggccgcgccguccgugacccuguucccucccagcagcgaggagcuccaggccaacaaggccacgcuggugugccugaucuccgacuucuaccccggcgccgucaccguggccuggaaggcggacagcuccccggugaaggccggcgucgagaccaccacgcccagcaagcaguccaacaacaaguacgccgccagcuccuaccucagccugacccccgagcaguggaagucccaccggagcuacuccugccaggugacccacgaggggagcaccguggagaagacggucgcccccaccgagugcuccugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 106 - RNA, 1679 nt, Artificial Sequence
  anti-Rabies-mAb-HC(GC) mRNA Design a-1 (A100, SapI) R7902
aggagagucccgcagucggcguccagcggcucugcuuguucgugugugugucguugcaggccuuauucaagcuuaccauggacuggaccuggcgcuuccuguucguggucgccgccgcgaccggcgugcagagccaggugcagcucguccaguccggggccgaggugaagaagcccggcagcuccgugaaggucagcugcaaggccuccgggggcacguucaaccgguacaccgugaacugggugcgccaggccccggggcagggccuggaguggaugggcgggaucauccccaucuucggcaccgccaacuacgcgcagcgguuccaggggcgccugaccaucacggccgacgagagcaccuccaccgccuacauggagcucagcucccugcggagcgacgacaccgccgucuacuucugcgcgcgcgagaaccuggacaacuccggcacguacuacuacuucagcgggugguucgaccccuggggccagggcacccucgugaccguguccagcgccuccaccaaggggcccagcgucuucccgcuggcccccuccagcaaguccacgagcggcgggaccgccgcccugggcugccucgugaaggacuacuuccccgagcccgugaccgucagcuggaacuccggcgcgcugaccagcggggugcacacguucccggccgugcugcaguccagcggccucuacucccugagcuccgucgugaccgugcccagcuccagccuggggacccagaccuacaucugcaacgucaaccacaagcccuccaacacgaagguggacaagcggguggagcccaagagcugcgacaagacccacaccugcccgcccugccccgccccggagcuccugggcgggccguccgucuuccuguucccucccaagcccaaggacacccucaugaucagccgcacgccggaggugaccugcgucgugguggacgugucccacgaggaccccgaggugaaguucaacugguacguggacggcgucgaggugcacaacgccaagaccaagccccgggaggagcaguacaacagcaccuaccgcguggucuccgugcugacggugcugcaccaggacuggcucaacggcaaggaguacaagugcaaggucagcaacaaggcccugcccgcgccgaucgagaagaccaucuccaaggccaaggggcagccccgggagccccagguguacacccugcccccgagccgcgaggagaugaccaagaaccaggugagccucacgugccuggucaagggcuucuaccccuccgacaucgccguggagugggagagcaacgggcagcccgagaacaacuacaagaccaccccgcccgugcuggacuccgacggcagcuucuuccucuacuccaagcugaccgucgacaagagccgguggcagcaggggaacguguucuccugcagcgugaugcacgaggcccugcacaaccacuacacgcagaagucccucagccugucccccggcaagugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 107 - RNA, 977 nt, Artificial Sequence
  anti-Rabies-mAb-LC(GC) mRNA Design a-1 (A100, SapI) R7908
aggagagucccgcagucggcguccagcggcucugcuuguucgugugugugucguugcaggccuuauucaagcuuaccaugagcgugcccaccauggccugggcccugcuccugcugucccuccugacccagggcacggggagcugggcgcaguccgcccugacccagccgcgcagcgucuccggcagccccgggcaguccgugaccaucagcugcaccggcacguccagcgacaucgggggcuacaacuucguguccugguaccagcagcaccccggcaaggcccccaagcucaugaucuacgacgccaccaagcggccgagcgggguccccgaccgcuucuccggcagcaaguccgggaacaccgccagccugaccaucuccggccugcaggcggaggacgaggccgacuacuacugcugcagcuacgccggggacuacacgcccggcgugguguucggcgggggcaccaagcucaccguccuggggcagccgaaggccgcgccguccgugacccuguucccucccagcagcgaggagcuccaggccaacaaggccacgcuggugugccugaucuccgacuucuaccccggcgccgucaccguggccuggaaggcggacagcuccccggugaaggccggcgucgagaccaccacgcccagcaagcaguccaacaacaaguacgccgccagcuccuaccucagccugacccccgagcaguggaagucccaccggagcuacuccugccaggugacccacgaggggagcaccguggagaagacggucgcccccaccgagugcuccugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 108 - RNA, 1684 nt, Artificial Sequence
  anti-Rabies-mAb-HC(GC) mRNA Design a-1 (A100-N5, EcoRI) R8824
aggagagucccgcagucggcguccagcggcucugcuuguucgugugugugucguugcaggccuuauucaagcuuaccauggacuggaccuggcgcuuccuguucguggucgccgccgcgaccggcgugcagagccaggugcagcucguccaguccggggccgaggugaagaagcccggcagcuccgugaaggucagcugcaaggccuccgggggcacguucaaccgguacaccgugaacugggugcgccaggccccggggcagggccuggaguggaugggcgggaucauccccaucuucggcaccgccaacuacgcgcagcgguuccaggggcgccugaccaucacggccgacgagagcaccuccaccgccuacauggagcucagcucccugcggagcgacgacaccgccgucuacuucugcgcgcgcgagaaccuggacaacuccggcacguacuacuacuucagcgggugguucgaccccuggggccagggcacccucgugaccguguccagcgccuccaccaaggggcccagcgucuucccgcuggcccccuccagcaaguccacgagcggcgggaccgccgcccugggcugccucgugaaggacuacuuccccgagcccgugaccgucagcuggaacuccggcgcgcugaccagcggggugcacacguucccggccgugcugcaguccagcggccucuacucccugagcuccgucgugaccgugcccagcuccagccuggggacccagaccuacaucugcaacgucaaccacaagcccuccaacacgaagguggacaagcggguggagcccaagagcugcgacaagacccacaccugcccgcccugccccgccccggagcuccugggcgggccguccgucuuccuguucccucccaagcccaaggacacccucaugaucagccgcacgccggaggugaccugcgucgugguggacgugucccacgaggaccccgaggugaaguucaacugguacguggacggcgucgaggugcacaacgccaagaccaagccccgggaggagcaguacaacagcaccuaccgcguggucuccgugcugacggugcugcaccaggacuggcucaacggcaaggaguacaagugcaaggucagcaacaaggcccugcccgcgccgaucgagaagaccaucuccaaggccaaggggcagccccgggagccccagguguacacccugcccccgagccgcgaggagaugaccaagaaccaggugagccucacgugccuggucaagggcuucuaccccuccgacaucgccguggagugggagagcaacgggcagcccgagaacaacuacaagaccaccccgcccgugcuggacuccgacggcagcuucuuccucuacuccaagcugaccgucgacaagagccgguggcagcaggggaacguguucuccugcagcgugaugcacgaggcccugcacaaccacuacacgcagaagucccucagccugucccccggcaagugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 109 - RNA, 982 nt, Artificial Sequence
  anti-Rabies-mAb-LC(GC) mRNA Design a-1 (A100-N5, EcoRI) R8828
aggagagucccgcagucggcguccagcggcucugcuuguucgugugugugucguugcaggccuuauucaagcuuaccaugagcgugcccaccauggccugggcccugcuccugcugucccuccugacccagggcacggggagcugggcgcaguccgcccugacccagccgcgcagcgucuccggcagccccgggcaguccgugaccaucagcugcaccggcacguccagcgacaucgggggcuacaacuucguguccugguaccagcagcaccccggcaaggcccccaagcucaugaucuacgacgccaccaagcggccgagcgggguccccgaccgcuucuccggcagcaaguccgggaacaccgccagccugaccaucuccggccugcaggcggaggacgaggccgacuacuacugcugcagcuacgccggggacuacacgcccggcgugguguucggcgggggcaccaagcucaccguccuggggcagccgaaggccgcgccguccgugacccuguucccucccagcagcgaggagcuccaggccaacaaggccacgcuggugugccugaucuccgacuucuaccccggcgccgucaccguggccuggaaggcggacagcuccccggugaaggccggcgucgagaccaccacgcccagcaagcaguccaacaacaaguacgccgccagcuccuaccucagccugacccccgagcaguggaagucccaccggagcuacuccugccaggugacccacgaggggagcaccguggagaagacggucgcccccaccgagugcuccugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 110 - RNA, 1904 nt, Artificial Sequence
  PpLuc(GC) SapI A100-GGG
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggg

SEQ ID NO: 111 - RNA, 1904 nt, Artificial Sequence
  PpLuc(GC) SapI A100-CCC
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaccc

SEQ ID NO: 112 - RNA, 1904 nt, Artificial Sequence
  PpLuc(GC) SapI A100-AAA
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 113 - RNA, 1904 nt, Artificial Sequence
  PpLuc(GC) SapI A100-UUU
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaauuu

SEQ ID NO: 114 - RNA, 1901 nt, Artificial Sequence
  PpLuc(GC) SapI A100
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 115 - RNA, 1906 nt, Artificial Sequence
  PpLuc(GC) SapI A100-GAAUU
aggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 116 - RNA, 1906 nt, Artificial Sequence; identical to SEQ ID NO: 115
  PpLuc(GC) EcoRI A100-GAAUU

SEQ ID NO: 117 - RNA, 1901 nt, Artificial Sequence; identical to SEQ ID NO: 114
  PpLuc(GC) NsiI A100

SEQ ID NO: 118 - RNA, 1901 nt, Artificial Sequence; identical to SEQ ID NO: 114
  PpLuc(GC) BciVI A100

SEQ ID NO: 119 - RNA, 1872 nt, Artificial Sequence
  PpLuc(GC) SapI HSL-A100
gggagaaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuaguuauaagacugacuagcccgaugggccucccaacgggcccuccuccccuccuugcaccgagauuaauagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 120 - RNA, 1877 nt, Artificial Sequence
  PpLuc(GC) SapI HSL-A100-GAAUU
gggagaaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuaguuauaagacugacuagcccgaugggccucccaacgggcccuccuccccuccuugcaccgagauuaauagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaauu

SEQ ID NO: 121 - RNA, 1877 nt, Artificial Sequence; identical to SEQ ID NO: 120
  PpLuc(GC) EcoRI HSL-A100-GAAUU

SEQ ID NO: 122 - RNA, 1836 nt, Artificial Sequence
  PpLuc(GC) SapI HSL-A64
gggagaaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuaguuauaagacugacuagcccgaugggccucccaacgggcccuccuccccuccuugcaccgagauuaauagaucucaaaggcucuuuucagagccaccaaaaaaaaaaaaaaaaaaaaaaaaaaaaaaaaaaaaaaaaaaaaaaaaaaaaaaaaaaaaaaaaa

SEQ ID NO: 123 - RNA, 1841 nt, Artificial Sequence
  PpLuc(GC) SapI HSL-A64-GAAUU
gggagaaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuaguuauaagacugacuagcccgaugggccucccaacgggcccuccuccccuccuugcaccgagauuaauagaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 124 - RNA, 1841 nt, Artificial Sequence; identical to SEQ ID NO: 123
  PpLuc(GC) EcoRI HSL-A64-GAAUU

SEQ ID NO: 125 - RNA, 1865 nt, Artificial Sequence
  PpLuc(GC) NsiII A64
gggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaa

SEQ ID NO: 126 - RNA, 1865 nt, Artificial Sequence; identical to SEQ ID NO: 125
  PpLuc(GC) SapI A64

SEQ ID NO: 127 - RNA, 1865 nt, Artificial Sequence; identical to SEQ ID NO: 125
  PpLuc(GC) BbsI A64

SEQ ID NO: 128 - RNA, 1870 nt, Artificial Sequence
  PpLuc(GC) EcoRI A64-GAAUU
gggagagucccgcagucggcguccagcggcucugcuuguucgugugugugucguugcaggccuuauucaagcuuaccauggaggacgccaagaacaucaagaagggcccggcgcccuucuacccgcuggaggacgggaccgccggcgagcagcuccacaaggccaugaagcgguacgcccuggugccgggcacgaucgccuucaccgacgcccacaucgaggucgacaucaccuacgcggaguacuucgagaugagcgugcgccuggccgaggccaugaagcgguacggccugaacaccaaccaccggaucguggugugcucggagaacagccugcaguucuucaugccggugcugggcgcccucuucaucggcguggccgucgccccggcgaacgacaucuacaacgagcgggagcugcugaacagcauggggaucagccagccgaccgugguguucgugagcaagaagggccugcagaagauccugaacgugcagaagaagcugcccaucauccagaagaucaucaucauggacagcaagaccgacuaccagggcuuccagucgauguacacguucgugaccagccaccucccgccgggcuucaacgaguacgacuucgucccggagagcuucgaccgggacaagaccaucgcccugaucaugaacagcagcggcagcaccggccugccgaaggggguggcccugccgcaccggaccgccugcgugcgcuucucgcacgcccgggaccccaucuucggcaaccagaucaucccggacaccgccauccugagcguggugccguuccaccacggcuucggcauguucacgacccugggcuaccucaucugcggcuuccgggugguccugauguaccgguucgaggaggagcuguuccugcggagccugcaggacuacaagauccagagcgcgcugcucgugccgacccuguucagcuucuucgccaagagcacccugaucgacaaguacgaccugucgaaccugcacgagaucgccagcgggggcgccccgcugagcaaggaggugggcgaggccguggccaagcgguuccaccucccgggcauccgccagggcuacggccugaccgagaccacgagcgcgauccugaucacccccgagggggacgacaagccgggcgccgugggcaagguggucccguucuucgaggccaaggugguggaccuggacaccggcaagacccugggcgugaaccagcggggcgagcugugcgugcgggggccgaugaucaugagcggcuacgugaacaacccggaggccaccaacgcccucaucgacaaggacggcuggcugcacagcggcgacaucgccuacugggacgaggacgagcacuucuucaucgucgaccggcugaagucgcugaucaaguacaagggcuaccagguggcgccggccgagcuggagagcauccugcuccagcaccccaacaucuucgacgccggcguggccgggcugccggacgacgacgccggcgagcugccggccgcggugguggugcuggagcacggcaagaccaugacggagaaggagaucgucgacuacguggccagccaggugaccaccgccaagaagcugcggggcggcgugguguucguggacgaggucccgaagggccugaccgggaagcucgacgcccggaagauccgcgagauccugaucaaggccaagaagggcggcaagaucgccgugugaggacuagucccuguucccagagcccacuuuuuuuucuuuuuuugaaauaaaauagccugucuuucagaucuaaaaaaaaaaaaaaaaaaaaaaaaaaaaaaaaaaaaaaaaaaaaaaaaaaaaaaaaaaaaaaaagaauu

SEQ ID NO: 129 - RNA, 1792 nt, Artificial Sequence
  RABV-G  EcoRI A64-N5-C30-HSL-N5
gggagaaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 130 - RNA, 1757 nt, Artificial Sequence
  RABV-G  EcoRI HSL-A64-N5
aggagaaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguuauaagacugacuagcccgaugggccucccaacgggcccuccuccccuccuugcaccgagauuaaucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 131 - RNA, 1788 nt, Artificial Sequence
  RABV-G  SapI HSL-A100
aggagaaagcuuaccauggugccccaggcccugcuguucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguuauaagacugacuagcccgaugggccucccaacgggcccuccuccccuccuugcaccgagauuaaucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 132 - RNA, 1757 nt, Artificial Sequence; identical to SEQ ID NO: 130
  RABV-G  EcoRI HSL-A64-N5

SEQ ID NO: 133 - RNA, 1788 nt, Artificial Sequence; identical to SEQ ID NO: 131
  RABV-G  SapI HSL-A100

SEQ ID NO: 134 - RNA, 1757 nt, Artificial Sequence; identical to SEQ ID NO: 130
  RABV-G  EcoRI HSL-A64-N5

SEQ ID NO: 135 - RNA, 1788 nt, Artificial Sequence; identical to SEQ ID NO: 131
  RABV-G  SapI HSL-A100

SEQ ID NO: 136 - RNA, 1841 nt, Artificial Sequence
  RABV-G  SapI HSL-A100
aggagagucccgcagucggcguccagcggcucugcuuguucgugugugugucguugcaggccuuauucaagcuuaccauggugccccaggcccugcuguucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuagucccuguucccagagcccacuuuuuuuucuuuuuuugaaauaaaauagccugucuuuccaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 137 - RNA, 1816 nt, Artificial Sequence
  RABV-G  EcoRI HSL-A64-N5
aggagagucccgcagucggcguccagcggcucugcuuguucgugugugugucguugcaggccuuauucaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 138 - RNA, 1816 nt, Artificial Sequence; identical to SEQ ID NO: 137
  RABV-G  EcoRI HSL-A64-N5

SEQ ID NO: 139 - RNA, 1847 nt, Artificial Sequence
  RABV-G  SapI HSL-A100
aggagagucccgcagucggcguccagcggcucugcuuguucgugugugugucguugcaggccuuauucaagcuuaccauggugccccaggcccugcuguucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 140 - RNA, 1816 nt, Artificial Sequence; identical to SEQ ID NO: 137
  RABV-G  EcoRI HSL-A64-N5

SEQ ID NO: 141 - RNA, 1847 nt, Artificial Sequence; identical to SEQ ID NO: 139
  RABV-G  SapI HSL-A100

SEQ ID NO: 142 - RNA, 1913 nt, Artificial Sequence
  RABV-G  EcoRI HSL-A64-N5
aggagagggcgcuuggcuugcaaggacccugagcugcggcauugaagcacacccaacccaacucgacugaagucagccucacugaaccggaucugagaaucuucucucucugggcuugccagggcucuccgaaccuagcuagcauccucuucaauuccaacuagaaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuagucccuguucccagagcccacuuuuuuuucuuuuuuugaaauaaaauagccugucuuucagaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 143 - RNA, 1938 nt, Artificial Sequence
  RABV-G  SapI HSL-A100
aggagagggcgcuuggcuugcaaggacccugagcugcggcauugaagcacacccaacccaacucgacugaagucagccucacugaaccggaucugagaaucuucucucucugggcuugccagggcucuccgaaccuagcuagcauccucuucaauuccaacuagaaagcuuaccauggugccccaggcccugcuguucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuagucccuguucccagagcccacuuuuuuuucuuuuuuugaaauaaaauagccugucuuuccaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 144 - RNA, 1913 nt, Artificial Sequence; identical to SEQ ID NO: 142
  RABV-G  EcoRI HSL-A64-N5

SEQ ID NO: 145 - RNA, 1938 nt, Artificial Sequence; identical to SEQ ID NO: 143
  RABV-G  SapI HSL-A100

SEQ ID NO: 146 - RNA, 1913 nt, Artificial Sequence; identical to SEQ ID NO: 142
  RABV-G  EcoRI HSL-A64-N5

SEQ ID NO: 147 - RNA, 1938 nt, Artificial Sequence; identical to SEQ ID NO: 143
  RABV-G  SapI HSL-A100

SEQ ID NO: 148 - RNA, 1925 nt, Artificial Sequence
  RABV-G  EcoRI HSL-A64-N5
aggagagucccgcagucggcguccagcggcucugcuuguucgugugugugucguugcaggccuuauucaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguaaagagcgcagguccaccugguggacacgucugauuagcuuagaaccugucuugucucaucuucaaaagguaacuuauuaaaaguccuuugcgucugaagccuuucuccuuuucugucacuugcaaauuccaaauuauagcuaauaaagaugacuagauaauuugcagaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 149 - RNA, 1925 nt, Artificial Sequence; identical to SEQ ID NO: 148
  RABV-G  EcoRI HSL-A64-N5

SEQ ID NO: 150 - RNA, 1956 nt, Artificial Sequence
  RABV-G  SapI HSL-A100
aggagagucccgcagucggcguccagcggcucugcuuguucgugugugugucguugcaggccuuauucaagcuuaccauggugccccaggcccugcuguucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguaaagagcgcagguccaccugguggacacgucugauuagcuuagaaccugucuugucucaucuucaaaagguaacuuauuaaaaguccuuugcgucugaagccuuucuccuuuucugucacuugcaaauuccaaauuauagcuaauaaagaugacuagauaauuugcagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 151 - RNA, 1956 nt, Artificial Sequence; identical to SEQ ID NO: 150
  RABV-G  SapI HSL-A100

SEQ ID NO: 152 - RNA, 1989 nt, Artificial Sequence
  RABV-G  EcoRI HSL-A64-N5
aggagacggagacggccugcaggaccugcucucucagcccucagccgaggccuacgccgagccgagugcgcagccgacgaccgggaggagccgcagccuucaacucugagguacugugauccgcgcugcccgccgggccgccccaguccgcugcugcggcaccuccuucccucgcgcccucuucgcucgccagcgccuucccugugagccugcgucaccgcggccgccaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguguccaccugucccuccugggcugcuggauugucucguuuuccugccaaauaaacaggaucagcgcuuuacagaucucaaaggcucuuuucagagccaccaaaaaaaaaaaaaaaaaaaaaaaaaaaaaaaaaaaaaaaaaaaaaaaaaaaaaaaaaaaaaaaaagaauu

SEQ ID NO: 153 - RNA, 1989 nt, Artificial Sequence; identical to SEQ ID NO: 152
  RABV-G  EcoRI HSL-A64-N5

SEQ ID NO: 154 - RNA, 2020 nt, Artificial Sequence
  RABV-G  SapI HSL-A100
aggagacggagacggccugcaggaccugcucucucagcccucagccgaggccuacgccgagccgagugcgcagccgacgaccgggaggagccgcagccuucaacucugagguacugugauccgcgcugcccgccgggccgccccaguccgcugcugcggcaccuccuucccucgcgcccucuucgcucgccagcgccuucccugugagccugcgucaccgcggccgccaagcuuaccauggugccccaggcccugcuguucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguguccaccugucccuccugggcugcuggauugucucguuuuccugccaaauaaacaggaucagcgcuuuacagaucucaaaggcucuuuucagagccaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 155 - RNA, 2020 nt, Artificial Sequence; identical to SEQ ID NO: 154
  RABV-G  SapI HSL-A100

SEQ ID NO: 156 - RNA, 9 nt, Artificial Sequence
  optimized Kozak sequence
gccaccaug

SEQ ID NO: 157 - RNA, 54 nt, Artificial Sequence
  5 UTR from human alpha-globin RNA with optimized Kozak sequence
gagaauaaacuaguauucuucugguccccacagacucagagagaacccgccacc

SEQ ID NO: 158 - RNA, 32 nt, Artificial Sequence
  5 UTR from human hemoglobin
ucuucugguccccacagacucagagagaaccc

SEQ ID NO: 159 - RNA, 54 nt, Artificial Sequence
  5 UTR from human alpha-globin RNA with optimized Kozak sequence
gagauuaaacuaguauucuucugguccccacagacucagagagaacccgccacc

SEQ ID NO: 160 - RNA, 57 nt, Artificial Sequence
  5 UTR synthetic UTR with GC-rich element and Kozak
gggaaauaagagagaaaagaagaguaagaagaaauauaagaccccggcgccgccacc

SEQ ID NO: 161 - RNA, 113 nt, Artificial Sequence
  3 UTR from HBA1 with 2 nt exchanges compared to NM_000558.4
uaggcuggagccucgguggccuagcuucuugccccuugggccuccccccagccccuccuccccuuccugcacccguacccccguggucuuugaauaaagucugagugggcggc

SEQ ID NO: 162 - RNA, 295 nt, Artificial Sequence
  3 UTR from amino-terminal enhancer of split (AES) mRNA and the mitochondrial encoded 12S ribosomal RNA
cucgagcugguacugcaugcacgcaaugcuagcugccccuuucccguccuggguaccccgagucucccccgaccucgggucccagguaugcucccaccuccaccugccccacucaccaccucugcuaguuccagacaccucccaagcacgcagcaaugcagcucaaaacgcuuagccuagccacacccccacgggaaacagcagugauuaaccuuuagcaauaaacgaaaguuuaacuaagcuauacuaaccccaggguuggucaauuucgugccagccacacccuggagcuagc

SEQ ID NO: 163 - DNA, 52 bp, Artificial Sequence
  RPS10 3′-UTR
aattggagaggattcttttgcattgaataaacttacagccaaaaaaccttaa

SEQ ID NO: 164 - RNA, 52 nt, Artificial Sequence
  RPS10 3′-UTR
aauuggagaggauucuuuugcauugaauaaacuuacagccaaaaaaccuuaa

(19) United States
(12) Patent Application Publication
(10) Pub. No.: US 2024/0102065 A1
(43) Pub. Date: Mar. 28, 2024

(54) METHOD OF REDUCING THE IMMUNOSTIMULATORY PROPERTIES OF IN VITRO TRANSCRIBED RNA

(71) Applicant: CUREVAC AG, Tübingen (DE)

(72) Inventors: Moritz THRAN, Tübingen (DE); Andreas THESS, Tübingen (DE); Fabian EBER, Tübingen (DE); Dipankar BHANDARI, Tübingen (DE)

(73) Assignee: CureVac SE, Tübingen (DE)

(21) Appl. No.: 18/262,912

(22) PCT Filed: Jan. 26, 2022
(86) PCT No.: PCT/EP2022/051807

§ 371 Date: Jul. 25, 2023

(30) Foreign Application Priority Data
Jan. 27, 2021 (WO) PCT/EP2021/051873

(51) Int. Cl. C12P 19/34; A61K 39/015; A61K 39/205; A61K 48/00; A61P 37/04; C07K 16/10

(52) U.S. Cl. CPC C12P 19/34; A61K 39/015; A61K 39/205; A61K 48/005; A61P 37/04; C07K 16/10; A61K 2039/575; C12N 2760/20122

(57) ABSTRACT

The present invention provides a method of reducing the immunostimulatory properties of an in vitro transcribed RNA by producing the in vitro transcribed RNA comprising a 3′ terminal A nucleotide. Hereby, the circular DNA template used to generate the in vitro transcribed RNA has been linearized using a type IIS endonuclease. The invention further provides pharmaceutical compositions comprising the vitro transcribed RNA comprising a 3′ terminal A nucleotide according to the invention for use in therapy.

### INTRODUCTION

RNA-based therapeutics can be used in e.g. passive and active immunotherapy, protein replacement therapy, or genetic engineering. Accordingly, therapeutic RNA has the potential to provide highly specific and individual treatment options for the therapy of a large variety of diseases, disorders, or conditions.

Besides used as vaccines, RNA molecules may also be used as therapeutics for replacement therapies, such as e.g. protein replacement therapies for substituting missing or mutated proteins such as growth factors or enzymes, in patients. However, a successful development of safe and efficacious RNA-based replacement therapies are based on different preconditions compared to vaccines. When applying coding RNA for protein replacement therapies, the therapeutic coding RNA should confer sufficient expression of the protein of interest in terms of expression level and duration and minimal stimulation of the innate immune system to avoid inflammation in the patient to be treated, and to avoid specific immune responses against the administered RNA molecule and the encoded protein. Protocols currently described in the literature (Conry et al., 1995b; Teufel et al., 2005; Strong et al., 1997; Carralot et al., 2004; Boczkowski et al., 2000) are based on a plasmid vector to generate RNA with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription (IVT), the circular plasmid is linearized downstream of the polyadenyl cassette by type II restriction endonucleases (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end.

It has been attempted to stabilize in vitro-transcribed RNA (IVT RNA) by various modifications in order to achieve prolonged expression of transferred IVT RNA. A basic requirement for translation is the presence of a 3′ poly(A) sequence, with the translation efficiency correlating with the length of poly(A) (Preiss and Hentze, 1998). The 5′ cap and 3′ poly(A) sequence synergistically activate translation in vivo (Gallie, 1991). Furthermore, the use of type IIS restriction endonucleases for linearization of the template plasmid results in an increased transcript stability and translation efficiency of in vitro-transcribed RNA (WO2007/036366, WO2016/057850).

Early IVT RNAs had an unstable structure, which resulted in low translational activity and induced an innate immune response. When IVT RNAs are delivered to the cells, they are recognized as exogenous RNAs, similar to viral RNAs. This activates pattern recognition receptors (PRRs) which induce a subsequent innate immune response. Therefore, it is expected that the IVT RNA stimulates the innate immune system by being recognized by PRRs, including Toll-like receptors (TLRs) and cytoplasmic RNA sensors (Mu, 2018). TLRs are transmembrane proteins located on the plasma membrane or the endosomal compartment of immune cells. Several types of TLRs are activated by endocytosed RNA molecules, such as viral RNAs and IVT RNAs. Once activated, TLRs mediate the secretion of type I interferons (IFNs). Different types of TLRs sense specific RNA structures. For instance, a single-stranded RNA (ssRNA) is recognized by TLR7, TLR8, while double-stranded RNA (dsRNA) is recognized by TLR3. Heil et al. (2004) reported that TLR7 and TLR8 are responsible for the specific recognition of single-strand RNA oligonucleotides, wherein uridine residues specifically activate TLR7. In addition, it has been shown that GU- and AU-rich RNA strands activate TLR7 and TLR8 (Kwon et al., 2018). This immune response of IVT RNAs may be beneficial in activating immune cells when the cells are applied as a vaccine system. However, IVT RNAs can additionally induce an innate immune response through the various cytoplasmic RNA sensors, which may lead to a shut-down of the protein expression machinery (Sahin et al., 2014). This hinders the clinical applications of IVT RNAs in protein replacement therapy. This is especially the case for the treatment of chronic diseases in which the RNA therapeutic needs to be administered repeatedly over an extended period of time. In addition, an overshooting innate immune response in vaccination approaches must also be prevented. The potential capacity of therapeutic RNA to elicit innate immune responses may represent limitations to its in vivo application.

In the art, that problem has been partially addressed by using modified RNA nucleotides (Kariko, 2008). By introducing modified nucleotides, the therapeutic RNA can show reduced innate immune stimulation in vivo. However, therapeutic RNA comprising modified nucleotides often shows reduced expression or reduced activity in vivo because modifications can also prevent recruitment of beneficial RNA-binding proteins and thus impede activity of the therapeutic RNA, e.g. protein translation.

Summarizing the above, it is problematic to reduce immunostimulatory properties of a therapeutic RNA and, at the same time, to retain the efficacy, e.g. translatability of such an RNA in a cell and/or inducing an adaptive immune response. However, in most therapeutic settings, both features (reduced or low immunostimulatory properties, high translation rates in vivo) are of paramount importance for an RNA medicament.

The objects outlined above are solved by the claimed subject matter of the invention.

### Definitions

For the sake of clarity and readability the following definitions are provided. Any technical feature mentioned for these definitions may be read on each and every embodiment of the invention.

Percentages in the context of numbers should be understood as relative to the total number of the respective items.

In other cases, and depending on the context, percentages should be understood as percentages by weight (wt.-%).

About: The term “about” is used when parameters or values do not necessarily need to be identical, i.e. 100% the same. Accordingly, “about” means, that a parameter or values may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. The skilled person will know that e.g. certain parameters or values may slightly vary based on the method how the parameter was determined. For example, if a certain parameter or value is defined herein to have e.g. a length of “about 1000 nucleotides”, the length may diverge by 0.1% to 20%, preferably by 0.1% to 10%; in particular, by 0.5%, 1%, 2%, 3%, 4%, 5%, 6%, 7%, 8%, 9%, 10%, 11%, 12%, 13%, 14%, 15%, 16%, 17%, 18%, 19%, 20%. Accordingly, the skilled person will know that in that specific example, the length may diverge by 1 to 200 nucleotides, preferably by 1 to 100 nucleotides; in particular, by 5, 10, 20, 30, 40, 50, 60, 70, 80, 90, 100, 110, 120, 130, 140, 150, 160, 170, 180, 190, 200 nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells). In the context of the invention, an antigen may be provided by the at least one therapeutic RNA of the inventive combination/composition.

Antigen: The term “antigen” as used herein will be recognized and understood by the person of ordinary skill in the art, and is e.g. intended to refer to a substance which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein, which may be presented by the MHC to T-cells. Also fragments, variants and derivatives of peptides or proteins derived from e.g. cancer antigens comprising at least one epitope may be understood as antigens. In the context of the present invention, an antigen may be the product of translation of the generated in vitro transcribed RNA comprising a 3′ terminal A nucleotide (e.g. coding RNA, replicon RNA, mRNA). The term “antigenic peptide or protein” will be recognized and understood by the person of ordinary skill in the art, and is e.g. intended to refer to a peptide or protein derived from a (antigenic) protein which may stimulate the body's adaptive immune system to provide an adaptive immune response. Therefore an “antigenic peptide or protein” comprises at least one epitope or antigen of the protein it is derived from (e.g. a tumor antigen, a viral antigen, a bacterial antigen, a protozoan antigen.

Cationic, cationisable: Unless a different meaning is clear from the specific context, the term “cationic” means that the respective structure bears a positive charge, either permanently or not permanently but in response to certain conditions such as e.g. pH. Thus, the term “cationic” covers both “permanently cationic” and “cationisable”. The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation, which is well known to a person skilled in the art. E.g., if a compound or moiety is cationisable, it is preferred that it is positively charged at a pH value of about 1 to 9, preferably 4 to 9, 5 to 8 or even 6 to 8, more preferably of a pH value of or below 9, of or below 8, of or below 7, most preferably at physiological pH values, e.g. about 7.3 to 7.4, i.e. under physiological conditions, particularly under physiological salt conditions of the cell in vivo. In embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7 particularly under physiological salt conditions of the cell in vivo. In embodiments, it is preferred that the cationisable compound or moiety is predominantly neutral at physiological pH values, e.g. about 7.0-7.4, but becomes positively charged at lower pH values. In some embodiments, the preferred range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be a DNA sequence, preferably an RNA sequence, consisting of a number of nucleotides that may be divided by three, which starts with a start codon and which preferably terminates with a stop codon.

CRISPR-associated protein: The term “CRISPR-associated protein” will be recognized and understood by the person of ordinary skill in the art. The term “CRISPR-associated protein” refers to RNA-guided endonucleases that are part of a CRISPR (Clustered Regularly Interspaced Short Palindromic Repeats) system (and their homologs, variants, fragments or derivatives), which is used by prokaryotes to confer adaptive immunity against foreign DNA elements. CRISPR-associated proteins include, without limitation, Cas9, Cpf1 (Cas12), C2c1, C2c3, C2c2, Cas13, CasX and CasY. As used herein, the term “CRISPR-associated protein”includes wild-type proteins as well as homologs, variants, fragments and derivatives thereof. Therefore, when referring to artificial nucleic acid molecules encoding Cas9, Cpf1 (Cas12), C2c1, C2c3, and C2c2, Cas13, CasX and CasY, said artificial nucleic acid molecules may encode the respective wild-type proteins, or homologs, variants, fragments and derivatives thereof. Besides Cas9 and Cas12 (Cpf1), several other CRISPR-associated protein exist that are suitable for genetic engineering in the context of the invention, including Cas13, CasX and CasY; e.g. Cas13 i.e. WP15770004, WP18451595, WP21744063, WP21746774, ERK53440, WP31473346, CVRQ01000008, CRZ35554, WP22785443, WP36091002, WP12985477, WP13443710, ETD76934, WP38617242, WP2664492, WP4343973, WP44065294, ADAR2DD, WP47447901, ER181700, WP34542281, WP13997271, WP41989581, WP47431796, WP14084666, WP60381855, WP14165541, WP63744070, WP65213424, WP45968377, EH006562, WP6261414, EKB06014, WP58700060, WP13446107, WP44218239, WP12458151, ERJ81987, ERJ65637, WP21665475, WP61156637, WP23846767, ERJ87335, WP5873511, WP39445055, WP52912312, WP53444417, WP12458414, WP39417390, EOA10535, WP61156470, WP13816155, WP5874195, WP39437199, WP39419792, WP39431778, WP46201018, WP39442171, WP39426176, WP39418912, WP39434803, WP39428968, WP25000926, EFU31981, WP4343581, WP36884929, BAU18623, AFJ07523, WP14708441, WP36860899, WP61868553, KJJ86756, EGQ18444, EKY00089, WP36929175, WP7412163, WP44072147, WP42518169, WP44074780, WP15024765, WP49354263, WP4919755, WP64970887, WP61710138); CasX (i.e. OGP07438, OHB99618); CasY(i.e. OJ108769, OGY82221, OJ106454, APG80656, OJI07455, OJ109436, PIP58309).

Guide RNA: As used herein, the term “guide RNA” (gRNA) thus relates to any RNA molecule capable of targeting a CRISPR-associated protein as defined above to a target DNA sequence of interest. In the context of the invention, the term guide RNA has to be understood in its broadest sense, and may comprise two-molecule gRNAs (“tracrRNA/crRNA”) comprising crRNA (“CRISPR RNA” or “targeter-RNA” or “crRNA” or “crRNA repeat”) and a corresponding tracrRNA (“trans-acting CRISPR RNA” or “activator-RNA” or “tracrRNA”) molecule, or single-molecule gRNAs. A “sgRNA” typically comprises a crRNA connected at its 3′ end to the 5′ end of a tracrRNA through a “loop” sequence.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least about 70%, 80, 90%, 91%, 92%, 93%, 94%, 95%, 96%, 97%, 98%, or about 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing U by T throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. Preferably, a nucleic acid “derived from” a nucleic acid also refers to nucleic acid, which is modified in comparison to the nucleic acid from which it is derived, e.g. in order to increase RNA stability even further and/or to prolong and/or increase protein production. In the context of amino acid sequences, the term “derived from” means that the amino acid sequence, which is derived from (another) amino acid sequence, shares e.g. at least about 70%, 80, 90%, 91%, 92%, 93%, 94%, 95%, 96%, 97%, 98%, or about 99% sequence identity with the amino acid sequence from which it is derived.

DNA: The term “DNA” is the usual abbreviation for deoxy-ribonucleic-acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerise by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single-stranded or double-stranded. In the double stranded form, the nucleotides of the first strand typically hybridize with the nucleotides of the second strand, e.g. by A/T-base-pairing and G/C-base-pairing.

DNA template: The term “DNA (descoxyribonucleic acid) template” provides the nucleic acid sequence which is transcribed into the RNA by the process of in vitro transcription and which therefore comprises a nucleic acid sequence which is complementary to the RNA sequence which is transcribed therefrom. In addition to the nucleic acid sequence which is transcribed into the RNA the DNA template comprises a promoter to which the RNA polymerase used in the in vitro transcription process binds with high affinity.

Fragment: The term “fragment” as used throughout the present specification in the context of a nucleic acid sequence or an amino acid (aa) sequence may typically be a shorter portion of a full-length sequence of e.g. a nucleic acid sequence or an amino acid sequence. A fragment typically consists of a sequence that is identical to the corresponding stretch within the full-length sequence. The term “fragment” as used throughout the present specification in the context of proteins or peptides may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the aa level or correspondingly on the nucleic acid level. A sequence identity with respect to such a fragment as defined herein may therefore preferably refer to the entire protein or peptide as defined herein or to the entire (coding) nucleic acid molecule of such a protein or peptide. Fragments of antigenic proteins or peptides may comprise at least one epitope of those proteins or peptides. Furthermore, also domains of a protein, like the extracellular domain, the intracellular domain or the transmembrane domain and shortened or truncated versions of a protein may be understood to comprise a fragment of a protein.

Heterologous: The terms “heterologous” or “heterologous sequence” as used throughout the present specification in the context of a nucleic acid sequence or an amino acid sequence refers to a sequence (e.g. DNA, RNA, amino acid) that will be recognized and understood by the person of ordinary skill in the art, and is intended to refer to a sequence that is derived from another gene, from another allele, from another species. Two sequences are typically understood to be “heterologous” if they are not derivable from the same gene or in the same allele. I.e., although heterologous sequences may be derivable from the same organism, they naturally (in nature) do not occur in the same nucleic acid molecule, such as e.g. in the same RNA or protein.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, preferably the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions, which are only occupied in one sequence. The percentage to which two sequences are identical can be determined using an algorithm, e.g. an algorithm integrated in the BLAST program.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Immune system: The term “immune system” will be recognized and understood by the person of ordinary skill in the art, and is e.g. intended to refer to a system of the organism that may protect the organisms from infection. If a pathogen succeeds in passing a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts typically contains so called humoral and cellular components.

Innate immune response: The term “innate immune response” is the first line of defense of the innate immune system against pathogens. It provides a fast response to pathogens by many mechanisms, including cytokine production and complement activation.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be activated by ligands of pattern recognition receptor e.g. Toll-like receptors, NOD-like receptors, or RIG-I like receptors etc.

In vitro transcription: The terms “in vitro transcription” or “RNA in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which according to the present invention is preferably a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In a preferred embodiment of the present invention the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Isoschizomers: The term “isoschizomers” are pairs of restriction enzymes specific to the same recognition sequence.

The first example discovered is called a prototype and all subsequent enzymes that recognize the same sequence are isoschizomers of the prototype.

Lipidoid compound: A lipidoid compound, also simply referred to as lipidoid, is a lipid-like compound, i.e. an amphiphilic compound with lipid-like physical properties. In the context of the present invention the term lipid is considered to encompass lipidoid compounds.

Messenger RNA (mRNA): The term “messenger RNA” (mRNA) refers to one type of RNA molecule. In vivo, transcription of DNA usually results in the so-called premature RNA, which has to be processed into so-called messenger RNA, usually abbreviated as mRNA. Typically, an mRNA comprises a 5-cap, a 5′-UTR, an open reading frame/coding sequence, a 3′-UTR and a poly(A).

Nucleic acid sequence, RNA sequence: The terms “nucleic acid sequence” or “RNA sequence” will be recognized and understood by the person of ordinary skill in the art, and are e.g. intended to refer to particular and individual order of the succession of its nucleotides or amino acids respectively.

Nucleic acid template: The nucleic acid template provides the nucleic acid sequence which is transcribed into the RNA by the process of in vitro transcription and which therefore comprises a nucleic acid sequence which is complementary to the RNA sequence which is transcribed therefrom. In addition to the nucleic acid sequence which is transcribed into the RNA the nucleic acid template comprises a promoter to which the RNA polymerase used in the in vitro transcription process binds with high affinity.

Reactogenicity: In clinical trials, the term reactogenicity refers to the property of a vaccine of being able to produce common, “expected” adverse reactions, especially excessive immunological responses and associated signs and symptoms, including fever and sore arm at the injection site. Other manifestations of reactogenicity typically identified in such trials include bruising, redness, induration, and swelling. Mainly the induction of cytokines (such as IFNa) seems to be the main reason of reactogenicity.

Reference in vitro transcribed RNA: A reference in vitro transcribed RNA means a corresponding in vitro transcribed RNA with the same nucleic acid sequence as the in vitro transcribed RNA according to the invention except of the 3′-terminal A nucleotide which has been generated by a 5-terminal T overhang. Additionally, this reference in vitro transcribed RNA has not been purified to remove dsRNA.

Restriction endonuclease: the term “restriction endonuclease” or “restriction enzyme” is an enzyme that recognize and bind DNA at or near specific recognition nucleotide sequences so that it can cut at a restriction cleavage sites.

RNA: The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. RNA can be obtained by transcription of a DNA sequence, e.g., inside a cell. In eukaryotic cells, transcription is typically performed inside the nucleus or the mitochondria. In vivo, transcription of DNA usually results in the so-called premature RNA which has to be processed into so-called messenger-RNA, usually abbreviated as mRNA. Processing of the premature RNA, e.g. in eukaryotic organisms, comprises a variety of different posttranscriptional modifications such as splicing, 5-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5-cap, optionally a 5′UTR, a coding sequence, optionally a 3′UTR and a poly(A) sequence. If RNA molecules are of synthetic origin, the RNA molecules are meant not to be produced in vivo, i.e. inside a cell or purified from a cell, but in an in vitro method. An examples for a suitable in vitro method is in vitro transcription.

In addition to messenger RNA, several non-coding types of RNA exist which may be involved in regulation of transcription and/or translation, and immunostimulation and which may also be produced by in vitro transcription.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is preferably a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotide of such nucleic acid sequence.

The term “variant” as used throughout the present specification in the context of proteins or peptides will be recognized and understood by the person of ordinary skill in the art, and is e.g. intended to refer to a proteins or peptide variant having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined herein may comprise conservative amino acid substitution(s) compared to their native, i.e. non-mutated physiological, sequence. A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Preferably, a variant of a protein comprises a functional variant of the protein, which means that the variant exerts the same effect or functionality or at least 40%, 50%, 60%, 70%, 80%, 90%, or 95% of the effect or functionality as the protein it is derived from.

Short Description of the Invention

The present invention is based on the inventor's surprising finding that linearization of a circular DNA template using type IIS endonucleases lead to an in vitro transcribed RNA comprising a 3′ terminal A nucleotide which displays reduced immunostimulatory properties compared to a corresponding reference in vitro transcribed RNA not comprising a 3′ terminal A nucleotide.

RNA molecules used as therapeutics have to be safe and efficient. When applying mRNA for example for protein replacement therapies, the RNA should confer sufficient expression of the encoded protein of interest in terms of expression level and duration and minimal stimulation of the innate immune system to avoid general immune responses by the patient to be treated such as inflammation and specific immune responses against the administered mRNA molecule or the encoded protein. The inherent immunostimulatory property of in vitro transcribed RNA is considered to be threatening especially in case of treatment of chronic diseases in which the RNA therapeutic needs to be administered repeatedly over an extended period of time to patients. But also in vaccination approaches an overshooting innate immune response must be prevented.

In a first aspect, the present invention relates to a method of reducing the immunostimulatory properties of an in vitro transcribed RNA by producing the in vitro transcribed RNA, comprising the steps i) providing a linear DNA template comprising a template DNA strand encoding the RNA, wherein the template DNA strand comprises a 5′ terminal T nucleotide; ii) incubating the linear DNA template under conditions to allow (run-off) RNA in vitro transcription; iii) obtaining the in vitro transcribed RNA comprising a 3′ terminal A nucleotide. In particular, the linear DNA template has been generated from a circular nucleic acid vector which was linearized using a restriction endonuclease, preferably a type IIS restriction endonuclease. In preferred embodiments the method of producing an in vitro transcribed RNA with reduced immunostimulatory properties can also lead to less double strand RNA (dsRNA) as side product compared to the production of a corresponding reference in vitro transcribed RNA not comprising a 3′ terminal A nucleotide.

In a second aspect, the present invention relates to an in vitro transcribed RNA comprising a 3′ terminal A nucleotide having reduced immunostimulatory properties obtainable by the method described by the first aspect. In particular, the obtained in vitro transcribed RNA comprising a 3′ terminal A nucleotide is a coding RNA, preferably an mRNA which comprises at least one coding sequence encoding at least one peptide or protein.

In a third aspect, the present invention relates to a pharmaceutical composition comprising the in vitro transcribed RNA comprising a 3′ terminal A nucleotide as described by the second aspect and optionally comprising one or more pharmaceutically acceptable excipients, carriers, diluents and/or vehicles.

In a fourth aspect, the present invention relates to a kit or kit of parts comprising the in vitro transcribed RNA comprising a 3′ terminal A nucleotide as described by the second aspect and optionally comprising a liquid vehicle for solubilizing, and, optionally, technical instructions providing information on administration and/or dosage of the components.

In further aspects, the present invention relates to the in vitro transcribed RNA comprising a 3′ terminal A nucleotide of the second aspect, the pharmaceutical composition of the third aspect or the kit or kit of parts of the fourth aspect for use as a medicament. Other aspects relate to methods of treating or preventing a disease, disorder, or condition.

In preferred embodiments the in vitro transcribed RNA comprising a 3′ terminal A nucleotide obtainable by the method from the first aspect has reduced immunostimulatory properties and further has a reduced content of double stranded RNA and/or prolongs the expression of a peptide or protein encoded by the in vitro transcribed RNA comprising a 3′ terminal A compared to a corresponding reference in vitro transcribed RNA not comprising a 3′ terminal A nucleotide.

### DETAILED DESCRIPTION OF THE INVENTION

The present application is filed together with a sequence listing in electronic format, which is part of the description of the present application (WIPO standard ST.25). The information contained in the electronic format of the sequence listing filed together with this application is incorporated herein by reference in its entirety. For many sequences, the sequence listing also provides additional detailed information, e.g. regarding certain structural features, sequence modifications, GenBank identifiers, or additional detailed information. In particular, such information is provided under numeric identifier <223>in the WIPO standard ST.25 sequence listing. Accordingly, information provided under said numeric identifier <223>is explicitly included herein in its entirety and has to be understood as integral part of the description of the underlying invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments, which combine the explicitly described embodiments with any number of the disclosed and/or preferred elements. Furthermore, any permutations and combinations of all described elements in this application should be considered as disclosed by the description of the present application, unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The present invention is based on the finding that linearization of a circular DNA template using type IIS restriction enzymes lead to an in vitro transcribed RNA comprising a 3′ terminal A nucleotide which displays reduced immunostimulatory properties due to less dsRNA formation.

First Aspect: Method of Reducing the Immunostimulatory Properties of an In Vitro Transcribed RNA

According to the first aspect the present invention relates to a method according to the following steps:
i) providing a linear DNA template comprising a template DNA strand encoding the RNA, wherein the template DNA strand comprises a 5′ terminal T nucleotide;
ii) incubating the linear DNA template under conditions to allow RNA in vitro transcription;
iii) obtaining the in vitro transcribed RNA comprising a 3′ terminal A nucleotide.

Suitably, the method is configured for reducing the immunostimulatory properties of an in vitro transcribed RNA by producing the in vitro transcribed RNA.

According to preferred embodiment, the present invention relates to a method of reducing the immunostimulatory properties of an in vitro transcribed RNA by producing the in vitro transcribed RNA according to the following steps:
i) providing a linear DNA template comprising a template DNA strand encoding the RNA, wherein the template DNA strand comprises a 5′ terminal T nucleotide;
ii) incubating the linear DNA template under conditions to allow (run-off) RNA in vitro transcription;
iii) obtaining the in vitro transcribed RNA comprising a 3′ terminal A nucleotide.

In preferred embodiments further steps following step iii) may be selected from
iv) purifying the obtained in vitro transcribed RNA after RNA in vitro transcription;
v) formulating the obtained in vitro transcribed RNA with a cationic compound to obtain an RNA formulation; and
vi) purifying the obtained in vitro transcribed RNA after formulating.

In particularly preferred embodiments, the method comprises the following steps
i) providing a linear DNA template comprising a template DNA strand encoding the RNA, wherein the template DNA strand comprises a 5′ terminal T nucleotide;
ii) incubating the linear DNA template under conditions to allow RNA in vitro transcription;
ii) obtaining the in vitro transcribed RNA comprising a 3′ terminal A nucleotide;
iv) purifying the obtained in vitro transcribed RNA comprising a 3′ terminal A nucleotide to remove double-stranded RNA;
wherein step ii) comprises incubating the linear DNA template with an RNA polymerase and a nucleotide mixture under conditions to allow RNA in vitro transcription, and wherein the 5′ terminal T nucleotide is a 5′ terminal T overhang and wherein the 5′ terminal T overhang comprises at least 3 consecutive T nucleotides.

In preferred embodiments, the provided linear DNA template leads to reduced double stranded RNA content in the obtained and/or purified in vitro transcribed RNA

“Template DNA strand” as defined herein, refers to the one strand of DNA that is used as a template for RNA synthesis and can also be referred to as the “noncoding strand.” During the process of transcription, the RNA polymerase traverses the template strand and uses base pairing complementarity with the template strand to create an RNA copy. The RNA polymerase traverses the template strand in a 3′ to 5′ direction producing an RNA molecule from 5′ to 3′ as an exact copy of the coding strand, in the exception that the thymines are replaced with uracils in the RNA strand.

In preferred embodiments the linear DNA template comprises a template DNA strand encoding the RNA which is transcribed into the RNA by the process of in vitro transcription and which therefore comprises a nucleic acid sequence which is complementary to the RNA sequence which is transcribed therefrom.

i) Providing a Linear DNA Template

According to the invention step i) of the method of reducing the immunostimulatory properties of an in vitro transcribed RNA comprises providing a linear DNA template comprising a step of digestion of a circular DNA template with a restriction endonuclease to generate the linear DNA template comprising a 5′ terminal T nucleotide.

Accordingly, the DNA template is in a closed circular conformation and prior to in vitro transcription, the circular DNA template is linearized by a restriction endonuclease, preferably suitable for in vitro transcription of RNA.

In other embodiments, the circular DNA template may be selected from a synthetic double stranded DNA construct, a single-stranded DNA template with a double-stranded DNA region comprising the promoter to which the RNA polymerase binds, a cyclic double-stranded DNA template with promoter and terminator sequences or a linear DNA template amplified by PCR or isothermal amplification.

Suitable circular DNA templates that comprise the DNA template strand are described in WO2017/025447A1, claim 1 to claim 25, the disclosure relating to such DNA vectors herewith incorporated by reference. The circular DNA templates are transformed and propagated in bacteria using common protocols known in the art, preferably using a fermentation procedure as described in WO2017025447A1, claims 26 to claims 37, the disclosure relating to the fermentation procedure herewith incorporated by reference.

In a preferred embodiment, the DNA template may be a linearized plasmid DNA template. The linear DNA template is obtained by contacting the circular DNA template with a restriction endonuclease under suitable conditions so that the restriction endonuclease cuts the circular nucleic acid vector at its recognition site(s) and disrupts the circular plasmid structure. The circular DNA template is preferably cut immediately after the end of the sequence, which is to be transcribed into RNA. Hence, the linear DNA template comprises a free 5′ end and a free 3′ end, which are not linked to each other. If the circular DNA template contains only one recognition site for the restriction enzyme, the linear DNA template has the same number of nucleotides as the circular DNA template. If the circular DNA template vector contains more than one recognition site for the restriction enzyme, the linear DNA template has a smaller number of nucleotides than the circular DNA template. The linear DNA template is then the fragment of the circular DNA template which contains the elements necessary for in vitro transcription, that is a promotor element for RNA transcription and the template DNA element. The RNA encoding sequence of the linear template DNA determines the sequence of the transcribed RNA by the rules of base-pairing.

In some embodiments, the plasmid DNA template constructs comprising the DNA template, preferably the generated circular plasmid DNA template, are transformed and propagated in bacteria using common protocols known in the art, preferably using a fermentation procedure as described in WO2017025447A1, claims 26 to claims 37, the disclosure relating to the fermentation procedure herewith incorporated by reference.

In other embodiments, the plasmid DNA template constructs comprising the circular DNA template are isolated from bacterial cells, purified, and used for subsequent steps. DNA isolation may be performed by a step of continuous bacterial lysis. Preferably, purification of the isolated DNA involves at least one step of ion exchange chromatography using common protocols known in the art and/or at least one step of hydrophobic interaction chromatography using common protocols known in the art and/or at least one step of tangential flow filtration using common protocols known in the art.

Accordingly, the DNA template encoding an RNA comprising a 3′ terminal A nucleotide sequence provided herein is a purified DNA template.

In specific preferred embodiments, the circular DNA template is linearized by a restriction endonuclease. The circular DNA template is linearized in a linearization reaction, e.g. a linearization reaction using a restriction enzyme, to obtain a linear DNA template suitable for performing (run-off) RNA in vitro transcription. The linearization reaction may be terminated. The termination of the linearization may be performed by adding an agent that inhibits the activity of the restriction enzyme for example by adding an effective amount of EDTA. In another example the restriction enzyme is inactivated by heat inactivation e.g. by incubation at a temperature of at least about 65° C.

In preferred embodiments, the circular DNA template comprises a recognition sequence for a restriction endonuclease and a cleavage site for a restriction endonuclease. Suitably, the recognition sequence and the cleavage site for a restriction endonuclease are functionally connected to each other and belong to the same restriction endonuclease.

Restriction endonucleases are a conglomeration of many different proteins that, by definition, have the common ability to cleave duplex DNA at a fixed position within, or close to, their recognition sequence. This cleavage generates reproducible DNA fragments.

A “recognition site” as described herein, refers to a sequence on a nucleic acid site that is recognized and bound by a restriction enzyme, nuclease, or a restriction endonuclease.

A “cleavage site” as described herein, is a site on a nucleic acid that is cleaved by a restriction endonuclease or endonuclease.

In a preferred embodiment, the cleavage site for the restriction endonuclease is located outside of the recognition sequence.

In some preferred embodiments, the restriction site is upstream of the recognition site. In most preferred embodiments, the recognition sequence is located within the polyT sequence of the DNA template strand of the linear DNA template.

In related aspects a “polyT sequence” as described herein means a plurality of thymine nucleotides or a stretch of thymidines. A polyT sequence as referred herein is a sequence of consecutive T nucleotides.

Accordingly, the “cleavage sites” or “recognition sequence,” are locations on the circular DNA template that contain specific sequences of nucleotides that are recognized and cleaved by restriction enzymes and such restriction recognition sites can vary from 4 to 10 bases in length. Restriction sites can be palindromic, and depending on the type of restriction endonuclease, the restriction endonuclease can cut the sequence between two nucleotides within the recognition site, or it can cut upstream or downstream from the recognition site, such that the endonuclease cleavage site is a specific distance away from an endonuclease recognition site.

A “palindromic sequence” as described herein, refers to a nucleic acid sequence on double-stranded DNA or RNA wherein reading 5′ (five-prime) to 3′ (three prime) forward on one strand matches the sequence reading backward 5′ to 3′ on the complementary strand or on itself. Complementary strands can bind to the complement in a palindromic sequence leading to a “hairpin” structure.

“Upstream” and “downstream” as used herein, refers to relative positions in either DNA or RNA. Each strand of DNA or RNA has a 5′ end and a 3′ end, so named for the carbon position on the deoxyribose (or ribose) ring. Upstream and downstream relate to the 5′ to 3′ direction in which RNA transcription takes place. Upstream is toward the 5′ end of the RNA molecule and downstream is toward the 3′ end. When considering double-stranded DNA, upstream is toward the 5′ end of the coding strand for the gene in question and downstream is toward the 3′ end. Due to the anti-parallel nature of DNA, this means the 3′ end of the template strand is upstream of the gene and the 5′ end is downstream.

In some embodiment, the linear DNA template which was generated using a restriction endonuclease comprises a 5′ terminal T nucleotide. In a preferred embodiment the 5′ terminal T nucleotide of the DNA template is a 5′ terminal T overhang.

“5′ terminal T overhang” has to be understood as a stretch of unpaired nucleotides in the end of a DNA molecule. These unpaired nucleotides can be in either strand, creating either 3′ or 5′ overhangs. These overhangs are in most cases palindromic. The simplest case of an overhang is a single nucleotide. This is most often adenosine and is created as a 3′ overhang by some DNA polymerases. Most commonly this is used in cloning PCR products created by such an enzyme. The product is joined with a linear DNA molecule with a 3′ thymine overhang. Since adenine and thymine form a base pair, this facilitates the joining of the two molecules by a ligase, yielding a circular molecule. Longer overhangs are called cohesive ends or sticky ends. They are most often created by restriction endonucleases when they cut DNA. Very often they cut the two DNA strands four base pairs from each other, creating a four-base 5′ overhang in one molecule and a complementary 5′ overhang in the other. These ends are called cohesive since they are easily joined back together by a ligase.

In particularly preferred embodiments, the 5′ terminal T overhang of the linear DNA template consists of 20, 15, 10, 9, 8, 7, 6, 5, 4, 3, 2 or 1 nucleotide/s; most preferably at least 3 consecutive nucleotides.

In particularly preferred embodiments, the 5′ terminal T overhang of the linear DNA template consists of 20, 15, 10, 9, 8, 7, 6, 5, 4, 3, 2 or 1 nucleotide/s; most preferably at least 4 consecutive nucleotides.

In preferred embodiments in that context, the 5′ terminal T overhang comprises at least 1, 2, 3, 4, 5 or 6 consecutive T nucleotides.

In preferred embodiments in that context, the 5′ terminal T overhang comprises at least 3 or 4 consecutive T nucleotides, preferably at least 3 consecutive T nucleotides.

In most preferred embodiments, the 5′ terminal T overhang comprises at least 3 consecutive T nucleotides, preferably (exactly) 3 consecutive T nucleotides.
In another preferred embodiment, the 5′ terminal T nucleotide is part of a polyT sequence. Accordingly, the 5′ terminal T nucleotide is a part of the 5′ terminal T overhang. In most preferred embodiments the 5′ terminal T overhang comprises at least 3 consecutive nucleotides, preferably T nucleotides.

In an embodiment, the linear DNA template comprises a RNA polymerase promotor sequence, for example a phage-derived DNA dependent RNA polymerase promoter sequence.

The term “promoter” or “promoter region” refers to a DNA sequence upstream (5′) of the coding sequence of a gene, which controls expression of said coding sequence by providing a recognition and binding site for RNA polymerase. The promoter region may include further recognition or binding sites for further factors involved in regulating transcription of said gene. A promoter may control transcription of a prokaryotic or eukaryotic gene. A promoter may be “inducible” and initiate transcription in response to an inducer, or may be “constitutive” if transcription is not controlled by an inducer. An inducible promoter is expressed only to a very small extent or not at all, if an inducer is absent. In the presence of the inducer, the gene is “switched on” or the level of transcription is increased. This is usually mediated by binding of a specific transcription factor. Examples of promoters preferred according to the invention are promoters for SP6, T3 or T7 polymerase.

Preferably, the RNA polymerase promotor sequence is selected from a T3, T7, Sny5 or SP6 RNA polymerase promotor sequence.

In preferred embodiments, the RNA polymerase promotor sequence is selected from a T7 RNA polymerase promotor sequence.

In some embodiments, the restriction endonuclease is a type 11 restriction endonuclease.

Type II restriction endonucleases are components of restriction modification systems that protect bacteria and archaea against invading foreign DNA. Most are homodimeric or tetrameric enzymes that cleave DNA at defined sites of 4-8 bp in length and require Mg2+ ions for catalysis. They differ in the details of the recognition process and the mode of cleavage, indicators that these enzymes are more diverse than originally thought (Pingoud et al 2005). These enzymes recognize specific 4 to 8 nucleotide sequences that are typically palindromic and cleave within the recognition site leaving sticky (5′ or 3′ overhangs) or blunt ends.

Restriction endonucleases are traditionally classified into four types on the basis of subunit composition, cleavage position, sequence specificity and cofactor requirements. Restriction enzymes can be isolated from bacterial cells and used in the laboratory to manipulate fragments of DNA, such as those that contain genes; for this reason, they are indispensable tools of recombinant DNA technology (genetic engineering).

However, amino acid sequencing has uncovered extraordinary variety among restriction enzymes and revealed that at the molecular level, there are many more than four different types. Type I enzymes are complex, multisubunit, combination restriction-and-modification enzymes that cut DNA at random far from their recognition sequences. Originally thought to be rare, we now know from the analysis of sequenced genomes that they are common. Type I enzymes are of considerable biochemical interest, but they have little practical value since they do not produce discrete restriction fragments or distinct gel-banding patterns.

Type II enzymes cut DNA at defined positions close to or within their recognition sequences (Pingoud, 2014). They produce discrete restriction fragments and distinct gel banding patterns, and they are the only class used in the laboratory for routine DNA analysis and gene cloning. Rather than forming a single family of related proteins, Type II enzymes are a collection of unrelated proteins of many different sorts. Type II enzymes frequently differ so completely in amino acid sequence from one another, and indeed from every other known protein, that they exemplify the class of rapidly evolving proteins that are often indicative of involvement in host-parasite interactions. The most common Type II enzymes are those like HhaI, HindIII, and NotI that cleave DNA within their recognition sequences. Enzymes of this kind are the principal ones available commercially. Most recognize DNA sequences that are symmetric, because they bind to DNA as homodimers, but a few, (e.g., BbvCI: CCTCAGC) recognize asymmetric DNA sequences, because they bind as heterodimers. Some enzymes recognize continuous sequences (e.g., EcoRI: GAATTC) in which the two half-sites of the recognition sequence are adjacent, while others recognize discontinuous sequences (e.g., BgII: GCCNNNNNGGC) in which the half-sites are separated. Cleavage leaves a 3-hydroxyl on one side of each cut and a 5′-phosphate on the other. They require only magnesium for activity and the corresponding modification enzymes require only S-adenosylmethionine. Many can use Mn2+ in place of Mg2+, and a few can use a variety of cations including Co2+, Zn2+, Ni2+ and Cu2+ instead. Type II endonucleases tend to be small, with subunits in the 200-350 amino acid range.

Type IIP is the most important subtype, accounting for over 90% of the enzymes used in molecular biology. Type IIP enzymes recognize symmetric (or “palindromic”) DNA sequences 4 to 8 base pairs in length and generally cleave within that sequence. They are the simplest and smallest of all restriction enzymes, typically 250-350 amino acids in length. Type IIP enzymes specific for 6-8 bp sequences mainly act as homodimers, composed of two identical protein chains that associate with each other in opposite orientations (Examples: EcoRI, HindIII, BamHI, NotI, PacI.) Each protein subunit binds roughly one-half of the recognition sequence and cleaves one DNA strand. Since the two subunits are identical, the enzyme is symmetric, and so the overall recognition sequence, and the positions of cleavage, are also symmetric. Usually, these enzymes cleave both DNA strands at once, each catalytic site acting independently of the other.

The next most common type II enzymes, usually referred to as “type IIS” are those like FokI and Alwl that cleave outside of their recognition sequence to one side. These enzymes are intermediate in size, 400-650 amino acids in length, and they recognize sequences that are continuous and asymmetric. When type IS enzymes bind to DNA, the catalytic domain is positioned to one side of, and several bases away from, the sequence bound by the recognition domain, and so cleavage is “shifted” to one side of the sequence. They comprise two distinct domains, one for DNA binding, and the other for DNA cleavage. They are thought to bind to DNA as monomers for the most part, but to cleave DNA cooperatively, through dimerization of the cleavage domains of adjacent enzyme molecules. For example, the Type IIS enzyme FokI recognizes the asymmetric sequence GGATG in duplex DNA and cleaves this (“top”) strand 9 bases to the right, and the complementary (“bottom”) strand four bases further down, producing 4-base 5′-overhanging ends. For this reason, some type IIS enzymes are much more active on DNA molecules that contain multiple recognition sites. In contrast, in type IIC enzymes, restriction and modification activities are combined into a single, composite, enzyme. Whereas type IIS enzymes comprise two domains, recognition and cleavage. Type IIC/IIG enzymes comprise three domains: one for cleavage, one for methylation, and another for sequence-recognition that is shared by both enzyme activities. The additional domain makes type IIC enzymes larger than type IIS enzymes, typically 800-1200 amino acids in length. Some bind as monomers, others as homodimers, and yet others assemble into complex oligomers with molecular masses exceeding 500 kDa (New England BioLabs). The cleavage domain of Type IIC enzymes forms the N-terminal 200 amino acids of the protein. A connector joins this to an adenine-specific DNA-methyltransferase domain of around 400 amino acids. The sequence motifs within this domain places it the “gamma”-class of methyltransferases, and so type IIC enzymes are alternatively referred to as “type IIG”. These enzymes cleave outside of their recognition sequences and can be classified as those that recognize continuous sequences (e.g., AcuI: CTGAAG) and cleave on just one side; and those that recognize discontinuous sequences (e.g., BcgI: CGANNNNNNTGC) and cleave on both sides releasing a small fragment containing the recognition sequence. The amino acid sequences of these enzymes are varied, but their organization is consistent. They comprise an N-terminal DNA-cleavage domain joined to a DNA-modification domain and one or two DNA sequence-specificity domains forming the C-terminus or present as a separate subunit. When these enzymes bind to their substrates, they switch into either restriction mode to cleave the DNA, or modification mode to methylate it.

Regardless of whether they act as monomers, homodimers or higher-order oligomers, all of the restriction enzymes discussed so far, belonging to the type IIP, S, C, G and B subclasses, use one catalytic site for DNA cleavage. If this site is disrupted by mutation, the enzyme becomes inactive and cleaves neither strand. Type IIT enzymes, in contrast, use two different catalytic sites for cleavage, each of which is specific for one particular strand. Type IIT enzymes combine features of both type IIP and type IIS enzymes, and so they are intermediate in size, between 350-450 amino acids. Disrupting either catalytic site of a type IIT enzyme does not inactivate it, but rather turns it into a strand-specific “nicking” enzyme. These cleave one DNA strand normally, but cannot cleave the other. Type IIT enzymes recognize asymmetric sequences. Some cleave within the sequence, others cleave on the periphery, and appear to be type IIS enzymes with a very short reach.

Type II enzymes are also large combination restriction-and-modification enzymes. They cleave outside of their recognition sequences and require two such sequences in opposite orientations within the same DNA molecule to accomplish cleavage; they rarely give complete digests.

Type IV enzymes recognize modified, typically methylated DNA and are exemplified by the McrBC and Mrr systems of E. coli.

“Blunt end” as referred herein, refers of a blunt-ended molecule in which both strands of in a nucleic acid terminate in a base pair.

“Sticky end” as referred herein refers to an overhang, which is a stretch of unpaired nucleotides in the end of a DNA molecule. Sticky ends can be created with an endonuclease, such as a restriction endonuclease. For example some endonucleases cleave a palindromic sequence and can leave an overhang, or a sticky end.

In preferred embodiments, the restriction endonuclease is a type IIS restriction endonuclease. Accordingly, the restriction endonuclease, which is used to linearize the circular DNA template to generate a linear DNA template comprising a template DNA strand which comprises a 5′ terminal T nucleotide, is a type IIS restriction endonuclease.

Type IIS restriction endonucleases comprise a specific group of enzymes, which recognize asymmetric DNA sequences and cleave at a defined distance outside of their recognition sequence, usually within 1 to 20 nucleotides. Further characteristics of type IIs endonucleases are described above.

In most preferred embodiments, the recognition sequence is placed in reverse orientation after the PolyA sequence to enable a cleavage prior to the recognition sequence. Hereby, the cleavage site for the restriction endonuclease is preferably GAGAGC.

In all aspects of the methods according to the invention, cleavage is preferably carried out with the aid of a restriction cleavage site, which is preferably a restriction cleavage site for a type IIS (table 1) restriction endonuclease.

TABLE I
Type IIS restriction enzymes recognize
asymmetric DNA sequences and cleave outside
of their recognition sequence

| | Recog- | Recog- | | |
|---|---|---|---|---|
| | nition | nition | | |
| | se- | se- | Over- | |
| | quence | quence | hang | Enzymatic |
| Enzyme | sequence | length | length | subtype |
| SapI | GCTCTTC(1/4) | 7 | 3 | HIT |
| MmeI | TCCRAC(20/18) | 6 | 2 | NIC |
| BsgI | GTGCAG(16/14) | 6 | 2 | NIC |
| BcoDI | GTCTC(1/5) | 5 | 4 | IIT |
| BsmAI | GTCTC(1/5) | 5 | 4 | |
| BciVI | GTATCC(6/5) | 6 | 1 | |
| HphI | GGTGA(8/7) | 5 | 1 | |
| BsaI | GGTCTC(1/5) | 6 | 4 | IIT |
| BsaI- | GGTCTC(1/5) | 6 | 4 | IIT |
| HF®v2 | | | | |
| BsmFI | GGGAC(10/14) | 5 | 4 | NIC |
| EciI | GGCGGA(11/9) | 6 | 2 | NIC |
| FokI | GGATG(9/13) | 5 | 4 | |
| BtsCI | GGATG(2/0) | 5 | 2 | |
| AIwI | GGATC(4/5) | 5 | 1 | |
| BspQI | GCTCTTC(1/4) | 7 | 3 | IIT |
| BtgZI | GCGATG(10/14) | 6 | 4 | NIC |
| NmeAIII | GCCGAG(21/19) | 6 | 2 | NIC |
| SfaNI | GCATC(5/9) | 5 | 4 | |
| BtsI-v2 | GCAGTG(2/0) | 6 | 2 | IIT |
| BbvI | GCAGC(8/12) | 5 | 4 | |
| BsrDI | GCAATG(2/0) | 6 | 2 | IIT |
| MIy! | GAGTC(5/5) | 5 | 0 | |
| PIeI | GAGTC(4/5) | 5 | 1 | |
| BseRI | GAGGAG(10/8) | 6 | 2 | IIC |
| HgaI | GACGC(5/10) | 5 | 5 | |
| BsmI | GAATGC(1/−1) | 6 | 2 | IIT |
| BbsI | GAAGAC(2/6) | 6 | 4 | IIT |
| BbsI-HF | GAAGAC(2/6) | 6 | 4 | IIT |
| MboII | GAAGA(8/7) | 5 | 1 | |
| BpuEI | CTTGAG(16/14) | 6 | 2 | NIC |
| BpmI | CTGGAG(16/14) | 6 | 2 | HIC |
| AcuI | CTGAAG(16/14) | 6 | 2 | NIC |
| EarI | CTCTTC(1/4) | 6 | 3 | HIT |
| BspCNI | CTCAG(9/7) | 5 | 2 | IIC |
| BsmBI | CGTCTC(1/5) | 6 | 4 | IIT |
| BsmBI-v2 | CGTCTC(1/5) | 6 | 4 | IIT |
| Esp31 | CGTCTC(1/5) | 6 | 4 | HIT |
| HpyAV | CCTTC(6/5) | 5 | 1 | |
| MnII | CCTC(7/6) | 4 | 1 | |
| FauI | CCCGC(4/6) | 5 | 2 | |
| BccI | CCATC(4/5) | 5 | 1 | |
| BtsIMutI | CAGTG(2/0) | 5 | 2 | IIT |
| BmrI | ACTGGG(5/4) | 6 | 1 | |
| BsrI | ACTGG(1/−1) | 5 | 2 | HIT |
| BceAI | ACGGC(12/14) | 5 | 2 | |
| BfuAI | ACCTGC(4/8) | 6 | 4 | |
| BspMI | ACCTGC(4/8) | 6 | 4 | |
| BsaXI | (9/12)ACNN | 6 | 3 & 3 | IIC |
| | NNNCTCC | | | |
| | (10/7) | | | |
| CspCI | (11/13)CAA | 7 | 2 & 2 | NIC |
| | NNNNNGTGG | | | |
| | (12/10) | | | |
| BaeI | (10/15)ACN | 7 | 5 & 5 | IIC |
| | NNNGTAYC | | | |
| | (12/7) | | | |
| BcgI | (10/12)CGA | 6 | 2 & 2 | IIC |
| | NNNNN | | | |
| | NTGC(12/10) | | | |

In preferred embodiments the type IS restriction endonuclease is selected from the group consisting of SapI, BSpQI, EciI, BpiI, AarI, AceIII, Acc36I, AloI, BaeI, BbvCI, PpiI and PsrI, BsrDI, BtsI, EarI, BmrI, BsaI, BsmBI, FauI, FaqI, BbsI, BciVI, BfuAI, Bse3DI, BspMI, BciVI, BseRI, BfuII, BfiII, BmrI, EciI, BtgZI, BpuEI, BsgI, MmeI, CspCI, BaeI, BsaMI, BveI, Mva12691, FOKL, PctI, Bse3DI, BseMI, Bst6l, Eam11041, Ksp6321, BfiI, Bso31I, BspTNI, Eco311, Esp31, BfuI, Acc36I, AarI, Eco57I, Eco57MI, GsuI, AloI, Hin4I, PpiI, and PsrI or corresponding isoschizomers.

Restriction endonucleases that recognize the same sequence are called isoschizomers (iso=equal; skhizo=split). The first example discovered is called a prototype and all subsequent enzymes that recognize the same sequence are isoschizomers of the prototype. For example for SapI type IIS restriction enzyme, other isoschizomers would be BspQI, LguI, BbsI or PciSI. Neoschizomers are a subset of isoschizomers that recognize the same sequence, but cleave at different positions from the prototype (Pingoud et al., 2014). Thus, AatII (recognition sequence: GACGT(C) and Zral (recognition sequence: GAC)GTC) are neoschizomers of one another, while HpaII (recognition sequence: C jCGG) and Mspl (recognition sequence: CJ.CGG) are isoschizomers. Analogous designations are not appropriate for MTases, where the differences between enzymes are not so easily defined and usually have not been well characterized.

Further examples of type IIS restriction enzymes can include but are not limited to AciI, MnII, AlwI, BbvI, BccI, BceAI, BsmAI, BsmFI, BspCNI, BsrI, BtsCI, FokI, HgaI, HphI, HpyAV, MboII, MlyI, PleI, SfaNI, AcuI, BciVI, BfuAI, BmgBI, BmrI, BpmI, BpuEI, BsaI, BseRI, BsgI, BsmI, BspMI, BsrBI, BsrDI, BtgZI, BtsI, EarI, EciI, MmeI, NmeAIII, BbvCI, Bpu10I, BspQI, SapI, BaeI, BsaXI, CspCI, AfaI, AluBI, AspLEI, BscFI, Bsh1236I, BshFI, BshI, BsiSI, BsnI, Bsp143I, BspANI, BspFNI, BssMI, BstENII, BstFNI, BstHHI, BstKTI, BstMBI, BsuRI, CfoI, Csp6I, FaeI, FaiI, FnuDII, FspBI, GlaI, HapII, HiniII, R9529, Hsp921I, HspAI, MaeI, MaeII, MvnI, PaII, RsaNI, SetI, SgeI, Sse9I, Tru1I, Tru9I, TscI, TspEI, TthHB8I, XspI, AfI, AgsI, AspS9I, AsuC2I, AsuI, BcefI, BcnI, BisI, BIsI, Bme1390I, Bme18I, BmrFI, BscGI, BseBI, BsiLI, BstZI, Bs1FI, BspMAI, BspNCI, Bst2UI, Bst71I, BstDEI, BstOI, BstSCI, CauII, CdiI, Cfr13I, Eco47I, EcoRII, FaqI, FinI, Fsp4HI, GluI, Hin4II, HpyF3I, ItaI, MaeIII, MspR9I, MvaI, NmuCI, Psp6I, PspPI, SatI, SinI, TscAI, VpaK11BI, AanI, AatI, AauI, Acc113I, Acc16I, AccB1I, AceIII, AcsI, AcvI, AcyI, AhII, Alw21I, Alw44I, Ama87I, Aor51HI, AsiAI, AsnI, Asp718I, AspHI, AsuII, AsuNHI, Main, AviII, BanIII, BauI, BbeI, BbrPI, BbuI, Bbv12I, BbvII, Bce83I, BcoI, BcuI, BfmI, BfrBI, BfrI, BInI, BmcAI, BmeT110I, BmiI, BmuI, BmyI, Bpu14I, BpvUI, Bsa29I, BsaOI, BsbI, BscBI, BscCI, Bse118I, BseAI, BseCI, BseDI, BsePI, BseSI, BseX3I, Bsh1285I, BshNI, BshTI, BshVI, BsiCI, BsiHKCI, BsiMI, BsiQI, BsiXI, Bsp106I, Bsp119I, Bsp120I, Bspl3I, Bsp1407I, Bsp1431I, Bsp19I, Bsp68I, BspA2I, BspCI, BspGI, BspLU11I, BspMAI, BspMII, BspOI, BspT104I, BspT107I, BspTI, BspXI, BssAI, BssHI, BssNAI, BssNI, BssT1I, Bst1107I, Bst98I, BstACI, BstAFI, BstAUI, BstBAI, BstC8I, BstDSI, BstH2I, BstHPI, BstNSI, BstSFI, BstSLI, BstSNI, BstX2I, BstZI, BsuTUI, BtuMI, BveI, CciI, Cfr10I, Cfr42I, Cfr9I, CfrI, Csp45I, CspAI, DinI, DrdII, DsaI, Ecl1361I, EclXI, Eco105I, Eco130I, Eco147I, Eco24I, Eco32I, Eco47II, Eco52I, Eco72I, Eco88I, EcoICRI, EcoT14I, EcoT22I, EcoT38I, EgeI, EheI, ErhI, FauNDI, FbaI, FbII, FriO, FunI, FunII, GdiII, GsaI, HaeI, HgiAI, HinII, Hindli, Hpy17811I, Hpy8I, Hsp92I, Kpn2I, Ksp22I, KspAI, KspI, MfII, MhII, MIsI, MIuNI, Mly113I, Mph1103I, MroI, MroNI, Msp20I, MspCI, MstI, MunI, MvrI, NgoAIV, NsbI, NspiII, NspV, PaeI, PagI, PauI, PceI, Pfl231I, PinAI, Ple19I, PmaCI, PshBI, Psp124BI, Psp1406I, PspAI, PspLI, PspN4I, PsuI, RcaI, SduI, Sfr274I, Sfr303I, SfuI, SgrBI, SlaI, SpaHi, SseBI, SspBI, Sstl, SstII, SunI, TatI, Vha4641, Vnel, XapI, XhoII, XmaCl, XmaIII, XmaJI, XmiI, ZhoI, Zsp21, AocI, Axyl, Bpu11021, Bse211, Bspl7201, BstPI, CeIII, CpoI, CspI, DraII, EcoO651, Eco811, Eco911, EspI, KfII, LguI, MabI, PpuXI, Psp511, PspEI, Rsr2l, SauI, AbsI, CciNI, FspAI, MauBI, MreI, MssI, RgaI, SdaI, SfaAl, Sgfl, SmiI, Sse2321, Adel, AspI, CaiI, Psyl, TeII, Asp7001, BoxI, Bse8l, BseRI, BsiBI, BsrBRI, BstPAI, CjeNIl, Maml, MroXI, OliI, Pdml, Rsel, SmiMI, 5 AccB71, AspEI, BasI, BmeRl, Bp11, Bsc41, BseLI, BsiYI, BstENI, BstMWI, CjeI, CjuI, CjuII, DriI, Eaml1051, EcIHKI, FaII, HpyF10VI, NgoAVIII, NruGI, PfIBI, UbaF141, Xagl, AasI, Bdal, Bsp241, CjePI, DseDI, UbaF91, ArsI, Bari, Pcsl and UbaF13L. The type IIS restriction endonuclease will recognize a recognition sequence and can cut away from the recognition sequence, in the cleavage site.

In preferred embodiments the type IIS restriction endonuclease is SapI, LguI, PciSI, BbsI or BspQI, or corresponding isoschizomerln some embodiments the type IIS restriction endonuclease is BSpQI. BSpQI is a thermostable type IIS endonuclease with the recognition sequence 5′ GCTCTTC N1/N4 3′.

In some embodiments the type IIS restriction endonuclease is BbsI. BbsI is a thermostable type IIS endonuclease with the recognition sequence 5′ GAAGAC N2/N6) 3′.

In most preferred embodiments the type IIS restriction endonuclease is SapI, or corresponding isoschizomer The type IIS restriction endonuclease SapI recognizes the DNA sequence 5′-GCTCTTC-3′ (top strand by convention) and cleaves downstream (N1/N4) indicating top- and bottom-strand spacing, respectively. In general, SapI recognize GCTCTTC and its complement GAAGAGC.

Restriction cleavage by SapI (type IIS) or EcoRI (type IIP) endoculeases at the type 11 restriction cleavage site enables a circular DNA template to be linearized within the poly(T) sequence. The linearized plasmid can then be used as template for RNA in vitro transcription (FIG. 1). The resulting transcript after linearization using SapI endonuclease ends in an unmasked poly(A) sequence (FIG. 1A). The resulting transcript after linearization using EcoRI ends with additional nucleotides after the PolyA (FIG. 1B).

Preferably, the linearization of the circular DNA template by the type IIS restriction endonuclease SapI leads to the 5′ terminal T overhang of the linear DNA template. SapI restriction enzyme recognizes the recognition sequence on the linear DNA template strand and cuts outside of the recognition sequence of both pDNA strands generating a 5′ terminal T overhang on the linear DNA template.

In an additional embodiment, the linearization of the circular DNA template by the type IIS restriction endonuclease SapI can leave a spacer nucleotide on the linear DNA template strand. The spacer nucleotide is selected from the group of A, C, G or T, preferably the spacer nucleotide is a T nucleotide (FIG. 1A).

In an additional embodiment, the linearization of the circular DNA template by the type IIS restriction endonuclease can leave a spacer nucleotide on the linear DNA template strand. The spacer nucleotide is selected from the group of A, C, G or T, preferably the spacer nucleotide is a C nucleotide.

Purification of Linearized DNA Template

In some embodiments, one or more steps of RP-HPLC are performed after the linearization reaction to purify the linearized DNA template.

In some embodiments one or more steps of cellulose purification are performed after the linearization reaction to purify the linearized DNA template.

In some embodiments one or more steps of oligo d(T) purification are performed after the linearization reaction to purify the linearized DNA template.

In some embodiments one or more steps of filtration step and at least one salt are performed after the linearization reaction to purify the linearized DNA template.

Accordingly, an HPLC (abbreviation for “High Performance (High Pressure) Liquid Chromatography”) is an established method of separating mixtures of substances, which is widely used in biochemistry, analytical chemistry and clinical chemistry. An HPLC apparatus consists in the simplest case of a pump with eluent reservoir containing the mobile phase, a sample application system, a separation column containing the stationary phase, and the detector. In addition, a fraction collector may also be provided, with which the individual fractions may be separately collected after separation and are thus available for further applications. RNA analysis using ion pair reversed phase HPLC (RP-HPLC) is known from A. Azarani and K. H. Hecker (Nucleic Acids Research, vol. 29, no. 2 e7). RP-HPLC involves the separation of molecules on the basis of hydrophobicity. The separation depends on the hydrophobic binding of the solute molecule from the mobile phase to the immobilized hydrophobic ligands attached to the stationary phase, i.e., the sorbent. To improve the quality of the of the linearized DNA template, after the linearization reaction one or more steps of RP-HPLC are performed. In preferred embodiments, the RP-HPLC to purify the linearized DNA template is performed as described in WO 2008/077592.

In a preferred embodiment, one or more steps of TFF are performed after the linearization reaction. Accordingly, Tangential Flow Filtration (TFF) or Crossflow Filtration is a type of filtration. Crossflow filtration is different from dead-end filtration in which the feed is passed through a membrane or bed, the solids being trapped in the filter and the filtrate being released at the other end. Cross-flow filtration gets its name because the majority of the feed flow travels tangentially across the surface of the filter, rather than into the filter. The principal advantage of this is that the filter cake (which can blind the filter) is substantially washed away during the filtration process, increasing the length of time that a filter unit can be operational. It can be a continuous process, unlike batch-wise dead-end filtration. This type of filtration is typically selected for feeds containing a high proportion of small particle size solids (where the permeate is of most value) because solid material can quickly block (blind) the filter surface with dead-end filtration. Applied pressure causes one portion of the flow stream to pass through the membrane (filtrate/permeate) while the remainder (retentate) is recirculated back to the feed reservoir. The general working principle of TFF can be found in literature, see e.g. WO2016/193206 or Fernandez et al. (A BIOTECHNOLOGICA, Bd. 12, 1992, Berlin, Pages 49-56) or Rathore, AS et al (Prep Biochem Biotechnol. 2011; 41(ψ):398-421).

The one or more steps of TFF may either be performed as a diafiltration step for i) exchange the solvent of the linearized DNA template to conditions required for the transcription and/or for ii) purifying the linearized DNA template; and/or as a concentration step for concentrating the linearized DNA template. The conditioning may be performed by at least one step of diafiltration using TFF to a diafiltration solution or buffer.

Preferably, the at least one step of TFF may comprise at least one diafiltration step using TFF and/or at least one concentration step using TFF. The diafiltration and concentration step may be performed separately but they may also at least partially overlap.

In a preferred embodiment, the at least one step of TFF comprises at least one diafiltration step, preferably performed with water or an aqueous salt solution as diafiltration solution. Particularly preferred is a diafiltration step with water.

According to a particularly preferred embodiment, the at least one step of TFF comprises at least one concentration step and at least one diafiltration step.

The TFF may be carried out using any suitable filter membrane. For example, TFF may be carried out using a TFF hollow fibre membrane or a TFF membrane cassette.

Particularly preferred in this context is a TFF membrane cassette comprising a cellulose-based membrane or a PES or mPES-based filter membrane with a MWCO of 100 kDa, e.g., a commercially available TFF membrane cassette such as NovaSep mPES with a MWCO of 100 kDa, or a cellulose-based membrane cassette with a MWCO of 100 kDa, e.g. a commercially available TFF membrane cassette such as Hydrosart (Sartorius).

In a preferred embodiment, the at least one step of TFF is performed using from about 1 to about 20 diafiltration volumes (DV) diafiltration solution or buffer, preferably from about 1 to about 15 DV diafiltration solution or buffer and more preferably from about 5 to about 12 DV diafiltration solution or buffer and even more preferably from about 6 to about 10 DV diafiltration solution or buffer. In a particularly preferred embodiment, the at least one step of TFF is performed using about 10 DV diafiltration solution or buffer, particularly water.

The at least one or more steps of TFF performed after linearization of the DNA template may efficiently remove contaminants, such as high molecular weight (HMWC) and low molecular weight (LMWC) contaminants, e.g. EDTA, DNA fragments, organic solvents, buffer components such as salts and detergents, and the restriction endonuclease, bacterial DNA, bacterial RNA, ect.

In preferred embodiments, the TFF of the circular DNA template and/or the linearized DNA template are preferably performed as described in published patent application WO2016/193206, the disclosure relating to TFF of the circular DNA and/or the linearized DNA disclosed in WO2016/193206 is herewith incorporated by reference. Exemplary parameters for TFF of the circular DNA and/or the linearized DNA are provided in Example 14, e.g. Table 16 of WO2016/193206. In other embodiments, the DNA template encoding the RNA is obtained by polymerase chain reaction (PCR). In embodiments where the DNA template has been generated by PCR, one or more steps of RP-HPLC and/or TFF may be performed after PCR to purify the linear DNA template.

The obtained purified linear DNA template encoding the RNA sequence is subsequently used for (run-oft) RNA in vitro transcription (see step ii).

Reduction of dsRNA

In particularly preferred embodiments, the in vitro transcription in step ii) leads to the formation of less double stranded RNA side products as compared to an in vitro transcription performed with a linear DNA template that does not comprise a 5′ terminal T nucleotide on the template DNA strand encoding the RNA.

Double stranded (ds)RNA that results from the pairing in cis or in trans of two complementary RNA strands has been postulated to be the earliest form of life (Gilbert, 1986; Joyce, 1989). Double-stranded RNA (dsRNA) are recognized as PAMPs (pathogen-associated molecular patterns) in the cytoplasm of mammalian cells by different PRRs (pattern recognition receptors). Specifically, dsRNA is detected by TLR-3 which can trigger interferon-beta production (Sandor F. and Buc, M., 2005, Folia Biologica (Praha) 51, 188-197).

dsRNA molecules are normally the result of a viral infection, although some endogenous dsRNA can be found mainly in the cell nucleus. The formation of double stranded RNA as side products during in vitro transcription can lead to an induction of the innate immune response. Hereby, current techniques for immunoblotting of dsRNA (via dot Blot, serological specific electron microscopy (SSEM) or ELISA for example) are used for detecting and sizing dsRNA species from a mixture of nucleic acids.

In general, common purification processes to purify the in vitro transcribed RNA to remove contaminants or double-stranded RNA are incorporated within this invention. For example removal of such contaminants by high performance liquid chromatography (HPLC) resulted in reduced IFN and inflammatory cytokine levels and in turn, higher expression levels in primary cells (Kariko et al. (2011) Nuc.Acids Res. 39:el42).

In a preferred embodiment, the in vitro transcription in step ii) leads to formation of about 50%, 40%, 30%, 20%, 10%, 5% less double stranded RNA side products as compared to an in vitro transcription performed with a linear DNA template that does not comprise a 5′ terminal T nucleotide on the template DNA strand encoding the RNA.

In a most preferred embodiment, the in vitro transcription in step ii) leads to formation of about 10% less double stranded RNA side products as compared to an in vitro transcription performed with a linear DNA template that does not comprise a 5′ terminal T nucleotide on the template DNA strand encoding the RNA.

In another preferred embodiment the in vitro transcription in step ii) leads to formation of about 5% less double stranded RNA side products as compared to an in vitro transcription performed with a linear DNA template that does not comprise a 5′ terminal T nucleotide on the template DNA strand encoding the RNA.

ii) RNA In Vitro Transcription (IVT)

IVT

According to the invention step ii) of the method of reducing the immunostimulatory properties of an in vitro transcribed RNA comprises incubating the linear DNA template with an RNA polymerase and a nucleotide mixture under conditions to allow (run-off) RNA in vitro transcription. The nucleotide mixture is part of an in vitro transcription mix (IVT-mix).

In preferred embodiments, the RNA polymerase is a T7 RNA polymerase.

In this context, the RNA is preferably produced by RNA in vitro transcription, wherein the nucleotide mixture is sequence optimized, preferably as described in WO2015/188933.

In that context, the nucleotide mixture used in RNA in vitro transcription may additionally contain modified nucleotides as defined below. In preferred embodiments, the nucleotide mixture (i.e. the fraction of each nucleotide in the mixture) used for RNA in vitro transcription reactions is essentially optimized for the given RNA sequence (optimized NTP mix), preferably as described WO2015/188933. RNA obtained by a process using an optimized NTP mix is characterized by reduced immune stimulatory properties, which is preferred in the context of the invention.

Sequence-optimized reaction mix: A reaction mix for use in an in vitro transcription reaction of an RNA molecule of a given sequence comprising the four nucleoside triphosphates (NTPs) GTP, ATP, CTP and UTP, wherein the fraction (2) of each of the four nucleoside triphosphates (NTPs) in the sequence-optimized reaction mix corresponds to the fraction (1) of the respective nucleotide in said RNA molecule, a buffer, a DNA template, and an RNA polymerase. In that context, fraction (1) and fraction (2) may differ by not more than 25%, 20%, 15%, 10%, 7%, 5% or by a value between 0.1% and 5%.

In preferred embodiments, the sequence-optimized nucleotide mixture is composed of (chemically) non-modified ribonucleoside triphosphates (NTPs) GTP, ATP, CTP and UTP.

Further conditions to allow (run-off) RNA in vitro transcription may include the presence of at least one cap analog, preferably a cap1 trinucleotide cap analog, m7G(5′)ppp(5′)(2′OMeA)pG or m7G(5′)ppp(5′)(2′OMeG)pG, preferably m7G(5′)ppp(5′)(2′OMeA)pG or m7(3′OMeG)(5′)ppp(5′)(2′OMeA)pG.

In other embodiments, a 5′-cap structure is formed via enzymatic capping using capping enzymes (e.g. vaccinia virus capping enzymes and/or cap-dependent 2′-0 methyltransferases) to generate capO or cap1 or cap2 structures. The 5′-cap structure (cap0 or cap1) may also be added using immobilized capping enzymes and/or cap-dependent 2′-0 methyltransferases using methods and means disclosed in WO2016/193226.

The ratio of cap analog:nucleotide and preferably cap:GTP may be varied from 10:1 to 1:1 to balance the percentage of capped products with the efficiency of the transcription reaction, preferably a ratio of cap:GTP of 4:1-5:1 is used. In some embodiment the preferably ratio of cap:GTP is less than 1:1. Most preferred is a ratio of cap:GTP of 1:1.

MgCl2 may be added to the transcription reaction which supplies Mg2+ ions as a co-factor for the polymerase. Preferred is a concentration of 1 mM to 100 mM. Particularly preferred is a concentration of 5 mM to 30 mM.

In other embodiments, a part or all of at least one (ribo)nucleoside triphosphate is replaced by a modified nucleoside triphosphate, preferably wherein the modified nucleoside triphosphate comprises a modification as defined in the context of the first aspect. For example, the vitro transcription mix may comprise chemically modified nucleotides selected from pseudouridine (ψ), N1-methylpseudouridine (m1ψ), 5-methylcytosine, and 5-methoxyuridine. In embodiments, uracil nucleotides in the nucleotide mixture are replaced (either partially or completely) by pseudouridine (ψ) and/or N1-methylpseudouridine (m1ψ) to obtain a modified RNA.

In preferred embodiments, the chemically modified nucleotide is pseudouridine (ψ). In another preferred embodiment the chemically modified nucleotide is N1-methylpseudouridine (m1ψ).

In preferred embodiments, in the course of the RNA in vitro transcription, the sequence-optimized nucleotide mixture is supplemented as a feeding step. Preferably, the sequence-optimized nucleotide mixture that is used for feeding does not comprise a cap analog.

In a particularly preferred embodiment, the transcription reaction comprises polycationic aliphatic amines, preferably spermidine. The polycationic aliphatic amines may interact with the negatively charged nucleic acids. The presence of the polycationic aliphatic amine, preferably spermidine, is known to assist the RNA in vitro transcription process. However, residual spermidine has to be depleted from the RNA solution in purification steps (see step iii and iv).

After RNA in vitro transcription, the linear DNA template is preferably digested using a DNAse digestion step (in the presence of a buffer comprising CaCl₂, which supplies Ca2+ ions as a co-factor for the polymerase). To digest DNA template, DNAse and a CaCl₂) solution (0.1 M/μg plasmid DNA) may be added to the transcription reaction, and incubated for 1-4 h at about 37° C. However, residual DNA fragments have to be depleted from the RNA solution in purification steps (see step iii and iv).

Accordingly, the in vitro transcribed RNA obtained in step ii) typically comprises the desired RNA product comprising a 3′ terminal A nucleotide and the other components of the RNA in vitro transcription reaction such as e.g. proteins (e.g. RNA polymerase, DNAse, RNAse inhibitor, pyrophosphatase, ect), BSA, HEPES or Tris, nucleotides, cap analog, salts (e.g. MgCl₂, CaCl₂), spermidine, DNA template or fragments of DNA template, and (short) RNA by-products.

Modified Nucleotides

In some preferred embodiments, the nucleotide mixture comprises at least one modified nucleotide and/or at least one nucleotide analogue or nucleotide derivative.

In this context, the modified nucleotide as defined herein are nucleotide analogs/modifications, e.g. backbone modifications, sugar modifications or base modifications. A backbone modification in connection with the present invention is a modification, in which phosphates of the backbone of the nucleotides are chemically modified. A sugar modification in connection with the present invention is a chemical modification of the sugar of the nucleotides. Furthermore, a base modification in connection with the present invention is a chemical modification of the base moiety of the nucleotides. In this context nucleotide analogs or modifications are preferably selected from nucleotide analogs which are applicable for transcription and/or translation.

In preferred embodiments the nucleotide mixture comprises least one modified nucleotide and/or at least one nucleotide analogues is selected from a backbone modified nucleotide, a sugar modified nucleotide and/or a base modified nucleotide, or any combination thereof.

Sugar Modification:

The modified nucleosides and nucleotides, which may be included in the nucleotide mixture and incorporated into the obtained in vitro transcribed RNA comprising a 3′ terminal A nucleotide as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R=H, alkyl, cycloalkyl, aryl, aralkyl, heteroaryl or sugar); polyethyleneglycols (PEG), —O(CH2CH₂O)nCH2CH2OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

Backbone Modifications:

The phosphate backbone may further be modified in the modified nucleosides and nucleotides, which may be included in the nucleotide mixture and incorporated into a modified in vitro transcribed RNA comprising a 3′ terminal A nucleotide as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base Modifications:

The modified nucleosides and nucleotides, which may be included in the nucleotide mixture and incorporated into the obtained in vitro transcribed RNA comprising a 3′ terminal A nucleotide as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments, the nucleotide analogues/modifications which may be incorporated into a in vitro transcribed RNA comprising a 3′ terminal A nucleotide as described herein are preferably selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

Preferably, at least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl- 2′-O-methyluridine, 5-carboxymethylaminomethyl-2-thiouridine, 5-(isopentenylaminomethyl)uridine, 5-(isopentenylaminomethyl)- 2-thiouridine, or 5-(isopentenylaminomethyl)- 2′-O-methyluridine.

In some embodiments, the at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyluridine.

In preferred embodiments, 100% of the uracil in the coding sequence as defined herein have a chemical modification, preferably a chemical modification is in the 5-position of the uracil.

In embodiments, 100% of the uracil in the cds of the obtained in vitro transcribed RNA comprising a 3′ terminal A nucleotide have a chemical modification, preferably a chemical modification that is in the 5-position of the uracil. In other embodiments, at least 10%, 20%, 30%, 40%, 50%, 60%, 70%, 80%, or 90% of the uracil nucleotides in the cds have a chemical modification, preferably a chemical modification that is in the 5-position of said uracil nucleotides. Such modifications are suitable in the context of the invention, as a reduction of natural uracil may reduce the stimulation of the innate immune system (after in vivo administration of the RNA comprising such a modified nucleotide) potentially caused by the first component upon administration to a cell.

The terms “cds” or “coding sequence” or “coding region” as used herein will be recognized and understood by the person of ordinary skill in the art, and are e.g. intended to refer to a sequence of several nucleotide triplets, which may be translated into a peptide or protein

In preferred embodiments, at least one modified nucleotide is selected from pseudouridine (ψ), N1-methylpseudouridine (m1ψ), 5-methylcytosine, and/or 5-methoxyuridine Suitably, the obtained in vitro transcribed RNA comprising a 3′ terminal A nucleotide, in particular, the cds of said RNA comprising a 3′ terminal A nucleotide, may comprise at least one modified nucleotide, wherein said at least one modified nucleotide may be selected from pseudouridine (ψ), N1-methylpseudouridine (m1ψ), 5-methylcytosine, and 5-methoxyuridine, wherein pseudouridine (ψ) is preferred.

In a preferred embodiment the obtained in vitro transcribed RNA comprising a 3′ terminal A nucleotide, in particular, the cds of said RNA comprising a 3′ terminal A nucleotide, and comprises at least one modified nucleotide, wherein said at least one modified nucleotide is pseudouridine (ψ).

In a preferred embodiment the obtained in vitro transcribed RNA comprising a 3′ terminal A nucleotide, in particular, the cds of said RNA comprising a 3′ terminal A nucleotide, and comprises at least one modified nucleotide, wherein said at least one modified nucleotide is N1-methylpseudouridine (m1ψ).

In a preferred embodiment the obtained in vitro transcribed RNA comprising a 3′ terminal A nucleotide, in particular, the cds of said RNA encoding a therapeutic protein for protein replacement therapy, and comprises at least one modified nucleotide,

In a preferred embodiment the obtained in vitro transcribed RNA comprising a 3′ terminal A nucleotide, in particular, the cds of said RNA encoding a therapeutic protein for therapy requiring frequent and repeated administration, and comprises at least one modified nucleotide.

In alternative embodiments, the nucleotide mixture is composed of (chemically) non-modified ribonucleoside triphosphates (NTPs) GTP, ATP, CTP and UTP.

In the context of the invention, the terms “modified nucleotides” or “chemically modified nucleotides” do not encompass 5′ cap structures (e.g. cap0, cap1 as defined herein). Additionally, the term “modified nucleotides” does not relate to modifications of the codon usage of e.g. a respective coding sequence. The terms “modified nucleotides” or “chemically modified nucleotides” do encompass all potential natural and non-natural chemical modifications of the building blocks of an RNA, namely the ribonucleotides A, G, C, U.

Accordingly, the obtained in vitro transcribed RNA comprising a 3′ terminal A nucleotide is not a (chemically) modified RNA, wherein the modification may refer to chemical modifications comprising backbone modifications as well as sugar modifications or base modifications.

Cap

In various embodiments the nucleotide mixture comprises a cap analog.

Accordingly, in preferred embodiments the cap analog is a cap0, cap1, cap2, a modified cap0 or a modified cap1 analog, preferably a cap1 analog.

The term “cap analog” or “5′-cap structure” as used herein is intended to refer to the 5′ structure of the RNA, particularly a guanine nucleotide, positioned at the 5′-end of an RNA, e.g. an mRNA. Preferably, the 5′-cap structure is connected via a 5′-5′-triphosphate linkage to the RNA. Notably, a “5′-cap structure” or a “cap analogue” is not considered to be a “modified nucleotide” or “chemically modified nucleotides” in the context of the invention. 5′-cap structures which may be suitable in the context of the present invention are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modARCA (e.g. phosphothioate modARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A 5′-cap (cap0 or cap1) structure may be formed in chemical RNA synthesis, using capping enzymes, or in RNA in vitro transcription (co-transcriptional capping) using cap analogs.

The term “cap analog” as used herein is intended to refer to a non-polymerizable di-nucleotide or tri-nucleotide that has cap functionality in that it facilitates translation or localization, and/or prevents degradation the RNA when incorporated at the 5′-end of the RNA. Non-polymerizable means that the cap analogue will be incorporated only at the 5′-terminus because it does not have a 5′ triphosphate and therefore cannot be extended in the 3′-direction by a template-dependent polymerase, (e.g. a DNA-dependent RNA polymerase). Examples of cap analogues include m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008/016473, WO2008/157688, WO2009/149253, WO2011/015347, and WO2013/059475). Further suitable cap analogues in that context are described in WO2017/066793, WO2017/066781, WO2017/066791, WO2017/066789, WO2017/053297, WO2017/066782, WO2018/075827 and WO2017/066797 wherein the disclosures relating to cap analogues are incorporated herewith by reference.

In particularly preferred embodiments, a cap1 structure is generated using tri-nucleotide cap analogue as disclosed in WO2017/053297, WO2017/066793, WO2017/066781, WO2017/066791, WO2017/066789, WO2017/066782, WO2018/075827 and WO2017/066797. In particular, any cap analog derivable from the structure disclosed in claim 1-5 of WO2017/053297 may be suitably used to co-transcriptionally generate a cap1 structure. Further, any cap analog derivable from the structure defined in claim 1 or claim 21 of WO2018/075827 may be suitably used to co-transcriptionally generate a cap1 structure.

In preferred embodiments, the cap1 analog is a cap1 trinucleotide cap analog.

In preferred embodiments, the cap1 structure of the obtained in vitro transcribed RNA comprising a 3′ terminal A nucleotide is formed using co-transcriptional capping using tri-nucleotide cap analog m7G(5′)ppp(5′)(2′OMeA)pG or m7G(5′)ppp(5′)(2′OMeG)pG.

A preferred cap1 analog in that context is m7G(5′)ppp(5′)(2′OMeA)pG.

In principle, 5′ cap structures can be introduced into the obtained in vitro transcribed RNA comprising a 3′ terminal A nucleotide by using one of two protocols.

In the first protocol, capping occurs concurrently with the initiation of transcription (co-transcriptional capping). In this approach, a dinucleotide cap analog such as m7G(5′)ppp(5′)G (m7G) is added to the reaction mixture. The DNA template is usually designed in such a way that the first nucleotide transcribed is a guanosine. The cap analog directly competes with GTP for incorporation as initial nucleotide and is incorporated as readily as any other nucleotide (WO2006/004648). A molar excess of the cap analog relative to GTP facilitates the incorporation of the cap dinucleotide at the first position of the transcript. However, this approach always yields a mixture of capped and uncapped RNAs. Uncapped mRNAs can usually not be translated after transfection into eukaryotic cells, thus reducing the efficacy of the RNA therapeutic. The effective concentration of co-transcriptionally capped mRNAs with the standard cap analog (m7GpppG) is further reduced because the analog can be incorporated in the reverse orientation (Gpppm7G), which is less competent for translation (Stepinski et al., 2001. RNA 7(10):1 486-95). The issue of cap analog orientation can be solved by using anti-reverse cap analogs (ARCA) such as (3′-O-methyl)GpppG which cannot be incorporated in the reverse orientation (Grudzien et al., 2004. RNA 10(9): 1479-87). In the second protocol, capping is done in a separate enzymatic reaction after in vitro transcription (post-transcriptional or enzymatic capping). Vaccinia Virus Capping Enzyme (VCE) possesses all three enzymatic activities necessary to synthesize a m7G cap structure (RNA 5′-triphosphatase, ganylyltransferase, and guanine-7-methyltransferase). Using GTP as substrate the VCE reaction yields RNA caps in the correct orientation. In addition, a type 1 cap can be created by adding a second Vaccinia enzyme, 2′-O-methyltransferase, to the capping reaction (Tcherepanova et al., 2008. BMC Mol. Biol. 9:90).

In some embodiments, the method of this invention additionally comprises a step of enzymatic capping after step ii) to generate a cap0 and/or a cap1 structure.

The 5′ cap structure can be formed after step ii) via enzymatic capping using capping enzymes (e.g. vaccinia virus capping enzymes and/or cap-dependent 2′-O-methyltransferases) to generate cap0 or cap1. The 5′ cap structure (cap0 or cap1) may be added using immobilized capping enzymes and/or cap-dependent 2′-O-methyltransferases using methods and means disclosed in WO2016/193226.

Accordingly, the obtained in vitro transcribed RNA comprising a 3′ terminal A nucleotide comprises a 5′-cap structure, preferably a cap1 structure. Hereby, the 5′ cap structure can improve stability and/or expression of the in vitro transcribed RNA comprising a 3′ terminal A nucleotide. A cap1 structure comprising vitro transcribed RNA has several advantageous features in the context of the invention including an increased translation efficiency and a reduced stimulation of the innate immune system.

In some embodiments, about 70%, 75%, 80%, 85%, 90%, 95% of the obtained in vitro transcribed RNA comprising a 3′ terminal A nucleotide comprises a cap1 structure as determined by using a capping detection assay. In most preferred embodiments, less than about 20%, 15%, 10%, 5%, 4%, 3%, 2%, 1% of the obtained in vitro transcribed RNA comprising a 3′ terminal A nucleotide does not comprises a cap structure as determined using a capping assay.

In preferred embodiments, at least 70%, 80%, or 90% of the obtained vitro transcribed RNA comprising a 3′ terminal A nucleotide comprise a cap1 structure.

For determining the presence/absence of a cap0 or a cap1 structure, a capping assays as described in published PCT application WO2015/101416, in particular, as described in claims 27 to 46 of published PCT application WO2015/101416 may be used. Other capping assays that may be used to determine the presence/absence of a cap0 or a cap1 structure of an RNA are described in PCT/EP2018/08667, or published PCT applications WO2014/152673 and WO2014/152659.

In preferred embodiments, the obtained in vitro transcribed RNA comprising a 3′ terminal A nucleotide comprises an m7G(5′)ppp(5′)(2′OMeA) cap structure. In such embodiments, the RNA comprises a 5-terminal m7G cap, and an additional methylation of the ribose of the adjacent nucleotide of m7GpppN, in that case, a 2′0 methylated adenosine.

Preferably, about 70%, 75%, 80%, 85%, 90%, 95% of the obtained in vitro transcribed RNA comprising a 3′ terminal A nucleotide comprises such a cap1 structure as determined using a capping assay. Preferably, about 95% of the obtained in vitro transcribed RNA comprises a cap1 structure in the correct orientation (and less that about 5% in reverse orientation) as determined using a capping assay.

In other preferred embodiments, the obtained in vitro transcribed RNA comprises an m7G(5′)ppp(5′)(2′OMeG) cap structure. In such embodiments, the RNA comprises a 5-terminal m7G cap, and an additional methylation of the ribose of the adjacent nucleotide, in that case, a 2′O methylated guanosine. Preferably, about 70%, 75%, 80%, 85%, 90%, 95% of the in vitro transcribed RNA comprises such a cap1 structure as determined using a capping assay.

Accordingly, the first nucleotide of said in vitro transcribed RNA sequence, that is, the nucleotide downstream of the m7G(5′)ppp structure, may be a 2′0 methylated guanosine or a 2′0 methylated adenosine.

In one embodiment, the method according to this invention additionally comprises a step of enzymatic polyadenylation after step ii).

Accordingly, within the step of enzymatic polyadenylation polyA sequence which is a nucleic acid molecules comprising about 100 (+/−20) to about 500 (+/−50), preferably about 250 (+/−20) adenosine nucleotides is enzymatically added using commercially available polyadenylation kits and corresponding protocols known in the art, or alternatively, by using immobilized poly(A)polymerases e.g. using a methods and means as described in WO2016/174271. The poly(A) sequence of the RNA is preferably obtained from a linear DNA template during RNA in vitro transcription in step ii). Enzymatic Polyadenylation can be performed either before or after further purification of the RNA transcript. The RNA transcript is incubated with a bacterial poly (A) polymerase (polynucleotide adenylyltransferase) e.g., from E. coli together with ATP in the respective buffers. The poly (A) polymerase catalyzes the template independent addition of AMP from ATP to the 3′ end of RNA. In a preferred embodiment the RNA transcript is reacted with E. coli poly(A) polymerase (e.g. from Cellscript) using 1 mM ATP at 37° C. for at least 30 min. Immediately afterwards, the RNA is purified according to the purification methods as described herein (e.g. LiCl purification). RNA is run on an agarose gel to assess RNA extension.

Coding RNA

In preferred embodiments, the obtained in vitro transcribed RNA comprising a 3′ terminal A nucleotide comprises at least one coding sequence encoding at least one peptide or protein.

The terms “coding sequence”, “coding region”, or “cds” as used herein will be recognized and understood by the person of ordinary skill in the art, and are e.g. intended to refer to a sequence of several nucleotides which may be translated into a peptide or protein. In the context of the present invention a cds is preferably an RNA sequence, consisting of a number of nucleotide triplets, starting with a start codon and preferably terminating with one stop codon. In embodiments, the cds of the RNA may terminate with one or two or more stop codons. The first stop codon of the two or more stop codons may be TGA or UGA and the second stop codon of the two or more stop codons may be selected from TAA, TGA, TAG, UAA, UGA or UAG.

According to further embodiments at least one coding sequence of the obtained in vitro transcribed RNA comprising a 3′ terminal A nucleotide of the invention may encode at least two, three, four, five, six, seven, eight and more, preferably distinct, (poly)peptides or proteins of interest linked with or without an amino acid linker sequence, wherein said linker sequence may comprise rigid linkers, flexible linkers, cleavable linkers (e.g., self-cleaving peptides) or a combination thereof.

In embodiments, the length the coding sequence may be at least or greater than about 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, 4000, 5000, or 6000 nucleotides. In embodiments, the length of the coding sequence may be in a range of from about 300 to about 2000 nucleotides.

A coding RNA can be any type of RNA construct (for example a double stranded RNA, a single stranded RNA, a circular double stranded RNA, or a circular single stranded RNA) characterized in that said coding RNA comprises at least one coding sequence (cds) that is translated into at least one amino-acid sequence (upon administration to e.g a cell).

In preferred embodiments, the obtained in vitro transcribed RNA is a coding RNA. Most preferably, said coding RNA may be selected from an mRNA, a (coding) self-replicating RNA (replicon RNA), a (coding) circular RNA, or a (coding) viral RNA.

A viral RNA is defined as the genetic material of an RNA virus. This nucleic acid is usually single-stranded RNA (ssRNA) but may be double-stranded RNA (dsRNA). A retroviral RNA is defined as a ssRNA of retroviruses In some embodiments, the obtained in vitro transcribed RNA comprising a 3′ terminal A nucleotide is a circular RNA. As used herein, the terms “circular RNA” or “circRNAs” have to be understood as a circular polynucleotide constructs that may encode at least one peptide or protein. Preferably, such a circRNA is a single stranded RNA molecule. In preferred embodiments, said circRNA comprises at least one coding sequence encoding at least one peptide or protein as defined herein, or a fragment or variant thereof.

In other embodiments, the obtained in vitro transcribed RNA comprising a 3′ terminal A nucleotide is a replicon RNA. The term “replicon RNA” is e.g. intended to be an optimized self-replicating RNA. Such constructs may include replicase elements derived from e.g. alphaviruses (e.g. SFV, SIN, VEE, or RRV) and the substitution of the structural virus proteins with the nucleic acid of interest (that is, the coding sequence encoding an antigenic peptide or protein as defined herein). Alternatively, the replicase may be provided on an independent coding RNA construct or a coding DNA construct. Downstream of the replicase may be a sub-genomic promoter that controls replication of the replicon RNA.

In particularly preferred embodiments the obtained in vitro transcribed RNA comprising a 3′ terminal A nucleotide is not a self-replicating RNA or replicon RNA.

In an additional embodiment the obtained in vitro transcribed RNA comprising a 3′ terminal A nucleotide comprises at least one coding sequence encoding at least one peptide or protein as defined above, and additionally at least one further heterologous peptide or protein element.

Suitably, the at least one further heterologous peptide or protein element may be selected from secretory signal peptides, transmembrane elements, multimerization domains, VLP (virus-like particles) forming sequence, a nuclear localization signal (NLS), peptide linker elements, self-cleaving peptides, immunologic adjuvant sequences or dendritic cell targeting sequences.

In preferred embodiments the obtained in vitro transcribed RNA comprising a 3′ terminal A nucleotide comprises at least one coding sequence encoding at least one peptide or protein which is or is derived from a therapeutic peptide or protein.

The term “therapeutic” in that context has to be understood as “providing a therapeutic function” or as “being suitable for therapy or administration”. However, “therapeutic” in that context should not at all to be understood as being limited to a certain therapeutic modality. Examples for therapeutic modalities may be the provision of a coding sequence (via said obtained in vitro transcribed RNA comprising a 3′ terminal A nucleotide) that encodes for a peptide or protein (wherein said peptide or protein has a certain therapeutic function, e.g. an antigen for a vaccine, or an enzyme for protein replacement therapies). A further therapeutic modality may be genetic engineering, wherein the RNA provides or orchestrates factors to e.g. manipulate DNA and/or RNA in a cell or a subject.

In the context of the invention, the obtained in vitro transcribed RNA comprising a 3′ terminal A nucleotide may provide at least one coding sequence encoding a peptide or protein that is translated into a (functional) peptide or protein after administration (e.g. after administration to a subject, e.g. a human subject).

In preferred embodiments, the obtained in vitro transcribed RNA comprising a 3′ terminal A nucleotide comprises at least one coding sequence encoding at least one peptide or protein suitable for use in treatment or prevention of a disease, disorder or condition.

In preferred embodiments, the length of the cds may be at least or greater than about 50, 60, 70, 80, 90, 100, 150, 200, 250, 300, 350, 400, 450, 500, 600, 700, 800, 900, 1000, 1200, 1400, 1600, 1800, 2000, 2500, 3000, 3500, 4000, 5000, or 6000 nucleotides. In embodiments, the length of the cds may be in a range of from about 300 to about 2000 nucleotides.

According to further preferred embodiments, the obtained in vitro transcribed RNA comprising a 3′ terminal A nucleotide comprises at least one coding sequence which encodes at least one (therapeutic) peptide or protein as defined below, and additionally at least one further heterologous peptide or protein element.

In various embodiments, the length of the encoded peptide or protein, e.g. the therapeutic peptide or protein, may be at least or greater than about 20, 50, 100, 150, 200, 300, 400, 500, 600, 700, 800, 900, 1000, or 1500 amino acids. According to certain embodiments, the obtained in vitro transcribed RNA comprising a 3′ terminal A nucleotide is mono-, bi-, or multicistronic, as defined herein. The coding sequences is preferably bi- or multicistronic. The obtained in vitro transcribed RNA comprising a 3′ terminal A nucleotide preferably encodes a distinct peptide or protein as defined herein or a fragment or variant thereof.

The term “monocistronic” will be recognized and understood by the person of ordinary skill in the art, and is e.g. intended to refer to an obtained in vitro transcribed RNA that comprises only one coding sequences. The terms “bicistronic”, or “multicistronic” as used herein will be recognized and understood by the person of ordinary skill in the art, and are e.g. intended to refer to an vitro transcribed RNA comprising a 3′ terminal A that may have two (bicistronic) or more (multicistronic) coding sequences.

In other embodiments, the obtained in vitro transcribed RNA comprising a 3′ terminal A nucleotide is monocistronic and the cds of said RNA encodes at least two different peptides or proteins as defined herein. Accordingly, said coding regions may e.g. encode at least two, three, four, five, six, seven, eight and more therapeutic peptides or proteins, linked with or without an peptide linker sequence, wherein said linker sequence can comprise rigid linkers, flexible linkers, cleavable linkers, or a combination thereof. Such constructs are herein referred to as “multi-protein-constructs”.

In further embodiments, the obtained in vitro transcribed RNA comprising a 3′ terminal A nucleotide may be bicistronic or multicistronic and comprises at least two coding sequences, wherein the at least two coding sequences encode two or more peptides or proteins as defined herein. Accordingly, the coding sequences in a bicistronic or multicistronic RNA suitably encode distinct peptides or proteins as defined herein. Preferably, the coding sequences in said bicistronic or multicistronic constructs may be separated by at least one IRES (internal ribosomal entry site) sequence. In that context, suitable IRES sequences may be selected from the list of nucleic acid sequences according to SEQ ID NOs: 1566-1662 of the patent application WO2017/081082, or fragments or variants of these sequences. In this context, the disclosure of WO2017/081082 relating to IRES sequences is herewith incorporated by reference.

In preferred embodiments, the A/U (A/T) content in the environment of the ribosome binding site of the obtained in vitro transcribed RNA comprising a 3′ terminal A nucleotide may be increased compared to the A/U (A/T) content in the environment of the ribosome binding site of its respective wild or reference type nucleic acid. This modification (an increased A/U (A/T) content around the ribosome binding site) increases the efficiency of ribosome binding to the RNA. An effective binding of the ribosomes to the ribosome binding site in turn has the effect of an efficient translation the RNA. Accordingly, in a particularly preferred embodiment, the obtained in vitro transcribed RNA comprising a 3′ terminal A nucleotide comprises a ribosome binding site, also referred to as “Kozak sequence” identical to or at least 80%, 85%, 90%, 95% identical to any one of the sequences SEQ ID NOs: 180 or 181 of PCT/EP2020/052775, or fragments or variants thereof.

In a preferred embodiment the Kozak sequence is optimized, also referred to as optimized Kozak sequence identical to or at least 80%, 85%, 90%, 95% identical to SEQ ID NO: 156, or fragments or variants thereof.

In particularly preferred embodiments the obtained in vitro transcribed RNA comprising a 3′ terminal A nucleotide contains a ribosome binding site, also referred to as “Kozak sequence” identical to or at least 80%, 85%, 90%, 95% identical to any one of the sequences SEQ ID NOs: 59 or 60, or fragments or variants thereof.

In a preferred embodiment the therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen, a bacterial antigen, a pathogen antigen, a protozoan antigen, an allergen, a tumor antigen, or fragments, variants, or combinations of any of these.

In a preferred embodiment the therapeutic peptide or protein is selected or derived from a viral antigen.

In preferred embodiments, the peptide or protein may be selected from an antigen or epitope of a pathogen selected or derived from List 1 provided below.

List 1: Suitable Pathogens of the Invention

Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, 0111 and 0104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neissera meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, SARS-CoV-2 coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-bome encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

In preferred embodiments the obtained in vitro transcribed RNA comprising a 3′ terminal A nucleotide comprises at least one codon modified coding sequence, wherein the amino acid sequence encoded by the at least one codon modified coding sequence is preferably not being modified compared to the amino acid sequence encoded by the corresponding reference coding sequence.

The term “codon modified coding sequence” relates to coding sequences that differ in at least one codon (triplets of nucleotides coding for one amino acid) compared to the corresponding wild type or reference coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (Table 1l) to optimize/modify the coding sequence for in vivo applications as outlined above.

In preferred embodiments, the at least one cds of the obtained in vitro transcribed RNA comprising a 3′ terminal A nucleotide is a codon modified cds, wherein the amino acid sequence encoded by the at least one codon modified cds is preferably not being modified compared to the amino acid sequence encoded by the corresponding wild type or reference cds.

TABLE II
Human codon usage with respective codon frequencies
indicated for each amino acid

| Amino | | fre- | Amino | | fre- |
|---|---|---|---|---|---|
| acid | codon | quency | acid | codon | quency |
| Ala | GCG | 0.10 | Pro | CCG | 0.11 |
| Ala | GCA | 0.22 | Pro | CCA | 0.27 |
| Ala | GCT | 0.28 | Pro | CCT | 0.29 |
| Ala | GCC* | 0.40 | Pro | CCC* | 0.33 |
| Cys | TGT | 0.42 | Gln | CAG* | 0.73 |
| Cys | TGC* | 0.58 | Gln | CAA | 0.27 |
| Asp | GAT | 0.44 | Arg | AGG | 0.22 |
| Asp | GAC* | 0.56 | Arg | AGA* | 0.21 |
| Glu | GAG* | 0.59 | Arg | CGG | 0.19 |
| Glu | GAA | 0.41 | Arg | CGA | 0.10 |
| Phe | TTT | 0.43 | Arg | CGT | 0.09 |
| Phe | TTC* | 0.57 | Arg | CGC | 0.19 |
| Gly | GGG | 0.23 | Ser | AGT | 0.14 |
| Gly | GGA | 0.26 | Ser | AGC* | 0.25 |
| Gly | GGT | 0.18 | Ser | TCG | 0.06 |
| Gly | GGC* | 0.33 | Ser | TCA | 0.15 |
| His | CAT | 0.41 | Ser | TCT | 0.18 |
| His | CAC* | 0.59 | Ser | TCC | 0.23 |
| Ile | ATA | 0.14 | Thr | ACG | 0.12 |
| Ile | ATT | 0.35 | Thr | ACA | 0.27 |
| Ile | ATC | 0.52 | Thr | ACT | 0.23 |
| Lys | AAG* | 0.60 | Thr | ACC* | 0.38 |
| Lys | AAA | 0.40 | Va | GTG* | 0.48 |
| Leu | TTG | 0.12 | Val | GTA | 0.10 |
| Leu | TTA | 0.06 | Va | GTT | 0.17 |
| Leu | CTG* | 0.43 | Val | GTC | 0.25 |
| Leu | CTA | 0.07 | Trp | TGG* | 1 |
| Leu | CTT | 0.12 | Ty | TAT | 0.42 |
| Leu | CTC | 0.20 | Tyr | TAC* | 0.58 |
| Met | ATG* | 1 | Stop | TGA* | 0.61 |
| Asn | AAT | 0.44 | Stop | TAG | 0.17 |
| Asn | AAC* | 0.56 | Stop | TAA | 0.22 |

*most frequent human codon for a certain amino acid

In other preferred embodiments the obtained in vitro transcribed RNA comprising a 3′ terminal A nucleotide comprises at least one codon modified coding sequence wherein the cds is selected from a C increased coding sequence, a CAI increased coding sequence, a human codon usage adapted coding sequence, a 0/C content modified coding sequence, or a 0/C optimized coding sequence, or any combination thereof.

In preferred embodiments in that context, the at least one codon modified coding sequence is selected from 0/C optimized coding sequence.

In preferred embodiments, the obtained in vitro transcribed RNA comprising a 3′ terminal A nucleotide may be codon modified, wherein the C content of the at least one coding sequence may be increased, preferably maximized, compared to the C content of the corresponding wild type or reference coding sequence (herein referred to as “C maximized coding sequence”). The amino acid sequence encoded by the C maximized coding sequence of the nucleic acid is preferably not modified compared to the amino acid sequence encoded by the respective wild type or reference coding sequence. The generation of a C maximized RNA sequences be carried out using a modification method according to WO2015/062738. In this context, the disclosure of WO2015/062738 is included herewith by reference.

In other preferred embodiments, the obtained in vitro transcribed RNA comprising a 3′ terminal A nucleotide may be codon modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the RNA is preferably modified such that the frequency of the codons encoding the same amino acid corresponds to the naturally occurring frequency of that codon according to the human codon usage. Such a procedure may be applied for each amino acid encoded by the coding sequence of the RNA to obtain sequences adapted to human codon usage.

In further preferred embodiments, the obtained in vitro transcribed RNA comprising a 3′ terminal A nucleotide may be codon modified, wherein the codon adaptation index (CAI) may be increased or preferably maximised in the at least one coding sequence (herein referred to as “CAI maximized coding sequence”). It is preferred that all codons of the wild type or reference sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table II, most frequent human codons are marked with asterisks). Suitably, the RNA may comprise at least one coding sequence, wherein the codon adaptation index (CAI) of the at least one coding sequence is at least 0.5, at least 0.8, at least 0.9 or at least 0.95. Most preferably, the codon adaptation index (CAI) of the at least one coding sequence is 1 (CAI=1). Such a procedure (as exemplified for Ala) may be applied for each amino acid encoded by the coding sequence of the nucleic acid to obtain CAI maximized coding sequences.

In preferred embodiments, the obtained in vitro transcribed RNA comprising a 3′ terminal A nucleotide may be codon modified, wherein the G/C content of the at least one coding sequence may be optimized compared to the G/C content of the corresponding wild type or reference coding sequence (herein referred to as “G/C content optimized coding sequence”). “Optimized” in that context refers to a coding sequence wherein the G/C content is preferably increased to the essentially highest possible G/C content. The amino acid sequence encoded by the G/C content optimized coding sequence of the RNA is preferably not modified as compared to the amino acid sequence encoded by the respective wild type or reference coding sequence. The generation of a G/C content optimized RNA sequences may be carried out using a method according to WO2002/098443. In this context, the disclosure of WO2002/098443 is included in its full scope in the present invention.

In preferred embodiments, the obtained in vitro transcribed RNA comprising a 3′ terminal A nucleotide may be codon modified, wherein the G/C content of the at least one coding sequence may be modified compared to the G/C content of the corresponding wild type or reference coding sequence (herein referred to as “G/C content modified coding sequence”). In this context, the terms “G/C optimization” or “G/C content modification” relate to an RNA that comprises a modified, preferably an increased number of guanosine and/or cytosine nucleotides as compared to the corresponding wild type or reference coding sequence. Such an increased number may be generated by substitution of codons containing adenosine or thymidine nucleotides by codons containing guanosine or cytosine nucleotides. Advantageously, RNA sequences having an increased G/C content may be more stable or may show a better expression than sequences having an increased A/U. The amino acid sequence encoded by the G/C content modified coding sequence of the RNA is preferably not modified as compared to the amino acid sequence encoded by the respective wild type or reference sequence.

Suitably, the G/C content of the coding sequence of the obtained in vitro transcribed RNA comprising a 3′ terminal A nucleotide is increased by at least 10%, 20%, 30%, preferably by at least 40% compared to the G/C content of the corresponding wild type or reference coding sequence.

In various embodiments, the obtained in vitro transcribed RNA comprising a 3′ terminal A nucleotide has a GC content of about 50% to about 80%. In preferred embodiments, the obtained in vitro transcribed RNA has a GC content of at least about 50%, preferably at least about 55%, more preferably of at least about 60%. In specific embodiments, the obtained in vitro transcribed RNA has a GC content of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, or about 70%.

In various embodiments, the coding sequence of the obtained in vitro transcribed RNA comprising a 3′ terminal A nucleotide has a GC content of about 60% to about 90%. In preferred embodiments, the coding sequence of the obtained in vitro transcribed RNA has a GC content of at least about 60%, preferably at least about 65%, more preferably of at least about 70%. In specific embodiments, the RNA of the composition has a GC content of about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%.

PolyA/PolyC

In various embodiments the obtained in vitro transcribed RNA comprising a 3′ terminal A nucleotide comprises at least one poly(A) sequence, and/or at least one poly(C) sequence, and/or at least one histone stem-loop sequence/structure.

In preferred embodiments, the obtained in vitro transcribed RNA comprising a 3′ terminal A nucleotide comprises at least one poly(A) sequence.

The terms “poly(A) sequence”, “poly(A) tail” or “3′-poly(A) tail” as used herein will be recognized and understood by the person of ordinary skill in the art, and are e.g. intended to be a sequence of adenosine nucleotides, typically located at the 3′-end of an RNA of up to about 1000 adenosine nucleotides. Preferably, said poly(A) sequence is essentially homopolymeric, e.g. a poly(A) sequence of e.g. 100 adenosine nucleotides has essentially the length of 100 nucleotides. In other embodiments, the poly(A) sequence may be interrupted by at least one nucleotide different from an adenosine nucleotide, e.g. a poly(A) sequence of e.g. 100 adenosine nucleotides may have a length of more than 100 nucleotides (comprising 100 adenosine nucleotides and in addition said at least one nucleotide—or a stretch of nucleotides—different from an adenosine nucleotide). For example, the poly(A) sequence may comprise about 100 A nucleotides being interrupted by at least one nucleotide different from A (e.g. a linker (ψ), typically about 2 to 20 nucleotides in length), e.g. A30-L-A70 or A70-L-A30.

The poly(A) sequence may comprise about 10 to about 500 adenosine nucleotides, about 10 to about 200 adenosine nucleotides, about 40 to about 200 adenosine nucleotides, or about 40 to about 150 adenosine nucleotides. Suitably, the length of the poly(A) sequence may be at least about or even more than about 10, 30, 50, 64, 70, 75, 100, 110, 200, 300, 400, or 500 adenosine nucleotides. In preferred embodiments, the at least one nucleic acid comprises at least one poly(A) sequence comprising about 30 to about 200 adenosine nucleotides. In particularly preferred embodiments, the poly(A) sequence comprises about 64 adenosine nucleotides (A64). In other particularly preferred embodiments, the poly(A) sequence comprises about 100 adenosine nucleotides (A100). In other embodiments, the poly(A) sequence comprises about 150 adenosine nucleotides.

In preferred embodiments in that context, the at least one poly(A) sequence comprises about 30, about 60, about 64, about 70, about 100, about 101, about 110 or about 120 adenosine nucleotides.

In preferred embodiments in that context, the at least one poly(A) sequence comprises at least 60, at least 80, at least 100, at least 110 or at least 120 adenosine nucleotides.

In preferred embodiments in that context, the at least one poly(A) sequence comprises about 60 to about 120 120 adenosine nucleotides.

In preferred embodiments in that context, the at least one poly(A) sequence mayis be interrupted by at least one nucleotide different from an adenosine nucleotide.

The poly(A) sequence as defined herein may be located directly at the 3′ terminus of the at least one RNA, preferably directly located at the 3′ terminus of an RNA. In such embodiments, the 3′-terminal nucleotide (that is the last 3T-terminal nucleotide in the polynucleotide chain) is the 3′-terminal A nucleotide of the at least one poly(A) sequence. The term “directly located at the 3′ terminus” has to be understood as being located exactly at the 3′ terminus—in other words, the 3′ terminus of the nucleic acid consists of a poly(A) sequence terminating with an A nucleotide.

It has to be understood that “poly(A) sequence” as defined herein typically relates to RNA-however in the context of the invention, the term likewise relates to corresponding sequences in a DNA molecule (e.g. a “poly(T) sequence”).

In preferred embodiments, the obtained in vitro transcribed RNA comprising a 3′ terminal A nucleotide may comprise a poly(A) sequence obtained by enzymatic polyadenylation, wherein the majority of nucleic acid molecules comprise about 100 (+/−20) to about 500 (+/−50), preferably about 250 (+/−20) adenosine nucleotides.

In embodiments, the obtained in vitro transcribed RNA comprising a 3′ terminal A nucleotide comprises a poly(A) sequence derived from a template DNA and additionally comprises at least one poly(A) sequence generated by enzymatic polyadenylation, e.g. as described in WO2016/091391.

In embodiments, the obtained in vitro transcribed RNA comprising a 3′ terminal A nucleotide comprises at least one polyadenylation signal.

In further embodiments, the obtained in vitro transcribed RNA comprising a 3′ terminal A nucleotide comprises at least one poly(C) sequence.

The term “poly(C) sequence” as used herein is intended to be a sequence of cytosine nucleotides of up to about 200 cytosine nucleotides. In preferred embodiments, the poly(C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In a particularly preferred embodiment, the poly(C) sequence comprises about 30 cytosine nucleotides.

In further embodiments, the obtained in vitro transcribed RNA comprising a 3′ terminal A nucleotide comprises at least one histone stem-loop (hSL) or histone stem loop structure.

The term “histone stem-loop” (abbreviated as “hSL” in e.g. the sequence listing) is intended to refer to nucleic acid sequences that form a stem-loop secondary structure predominantly found in histone mRNAs.

Histone stem-loop sequences/structures may suitably be selected from histone stem-loop sequences as disclosed in WO2012/019780, the disclosure relating to histone stem-loop sequences/histone stem-loop structures incorporated herewith by reference. A histone stem-loop sequence may preferably be derived from formulae (I) or (II) of WO2012/019780. According to a further preferred embodiment, the obtained in vitro transcribed RNA comprises at least one histone stem-loop sequence derived from at least one of the specific formulae (Ia) or (IIa) of the patent application WO2012/019780.

In preferred embodiments, the obtained in vitro transcribed RNA comprising a 3′ terminal A nucleotide comprises at least one histone stem-loop, wherein said histone stem-loop (hSL) comprises or consists a nucleic acid sequence identical or at least 70%, 80%, 90%, 91%, 92%, 93%, 94%, 95%, 96%, 97%, 98%, or 99% identical to SEQ ID NOs: 178 or 179 of PCT/EP2020/052775, or fragments or variants thereof.

In other preferred embodiments, the obtained in vitro transcribed RNA comprising a 3′ terminal A nucleotide comprises a 3′-terminal sequence element. Said 3′-terminal sequence element comprises a poly(A) sequence and a histone-stem-loop sequence. Accordingly, the obtained in vitro transcribed RNA comprises at least one 3′-terminal sequence element comprising or consisting of a nucleic acid sequence being identical or at least 70%, 80%, 90%, 91%, 92%, 93%, 94%, 95%, 96%, 97%, 98%, or 99% identical to SEQ ID NOs: 182 to 230 of PCT/EP2020/052775, or a fragment or variant thereof. In preferred embodiments, the obtained in vitro transcribed RNA comprising a 3′ terminal A nucleotide comprises at least one histone stem-loop, wherein said histone stem-loop (hSL) comprises or consists a nucleic acid sequence identical or at least 70%, 80%, 90%, 91%, 92%, 93%, 94%, 95%, 96%, 97%, 98%, or 99% identical to SEQ ID NOs: 61 or 62, or fragments or variants thereof.

UTR

In preferred embodiments the obtained in vitro transcribed RNA comprising a 3′ terminal A nucleotide comprises at least one heterologous 5′-UTR and/or at least one heterologous 3′-UTR.

Notably, UTRs may harbor regulatory sequence elements that determine nucleic acid, e.g. RNA turnover, stability, and localization. Moreover, UTRs may harbor sequence elements that enhance translation. In medical application of nucleic acid sequences (including DNA and RNA), translation of the RNA into at least one peptide or protein is of paramount importance to therapeutic efficacy. Certain combinations of 3′-UTRs and/or 5′-UTRs may enhance the expression of operably linked coding sequences encoding peptides or proteins of the invention. Nucleic acid molecules harboring said UTR combinations advantageously enable rapid and transient expression of antigenic peptides or proteins after administration to a subject, preferably after intramuscular administration. Suitably, the in vitro transcribed RNA comprising a 3′ terminal A nucleotide of the invention comprises at least one heterologous 5′-UTR and/or at least one heterologous 3′-UTR. Said heterologous 5′-UTRs or 3′-UTRs may be derived from naturally occurring genes or may be synthetically engineered. In preferred embodiments, the nucleic acid, preferably the RNA comprises at least one coding sequence operably linked to at least one (heterologous) 3T-UTR and/or at least one (heterologous) 5′-UTR.

In preferred embodiments, the at least one heterologous 3′-UTR comprises a nucleic acid sequence derived from a 3′-UTR of a gene selected from PSMB3, ALB7, human alpha-globin, CASP1, COX6B1, GNAS, NDUFA1, RSP10, human mitochondrial 12S rRNA (mtRNR1), human AES/TLE5 gene, FIG. 4.1, and RPS9, or from a homolog, a fragment or a variant of any one of these genes.

The term “3′-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of a nucleic acid, e.g. a DNA or an RNA, located between a coding sequence and an (optional) terminal poly(A) sequence. A 3′-UTR may comprise elements for controlling gene expression, also called regulatory elements. Such regulatory elements may be, e.g., ribosomal binding sites, miRNA binding sites etc.

Preferably, the in vitro transcribed RNA comprising a 3′ terminal A nucleotide comprises a 3′-UTR, which may be derivable from a gene that relates to an RNA with enhanced half-life (i.e. that provides a stable RNA).

In some embodiments, a 3′-UTR comprises one or more polyadenylation signals, a binding site for proteins that affect nucleic acid stability or location in a cell, or one or more miRNA or binding sites for miRNAs.

MicroRNAs (or miRNA) are 19-25 nucleotide long noncoding RNAs that bind to the 3′-UTR of nucleic acid molecules and down-regulate gene expression either by reducing nucleic acid molecule stability or by inhibiting translation. E.g., microRNAs are known to regulate RNA, and thereby protein expression, e.g. in liver (miR-122), heart (miR-Id, miR-149), endothelial cells (miR-17-92, miR-126), adipose tissue (let-7, miR-30c), kidney (miR-192, miR-194, miR-204), myeloid cells (miR-142-3p, miR-142-5p, miR-16, miR-21, miR-223, miR-24, miR-27), muscle (miR-133, miR-206, miR-208), and lung epithelial cells (let-7, miR-133, miR-126). The RNA may comprise one or more microRNA target sequences, microRNA sequences, or microRNA seeds. Such sequences may e.g. correspond to any known microRNA such as those taught in US2005/0261218 and US2005/0059005.

Accordingly, miRNA, or binding sites miRNAs as defined above may be removed from the 3′-UTR or introduced into the 3′-UTR in order to tailor the expression of the nucleic acid, e.g. the RNA to desired cell types or tissues (e.g. muscle cells).

In preferred embodiments, the obtained in vitro transcribed RNA comprising a 3′ terminal A nucleotide comprises at least one heterologous 3′-UTR, wherein the at least one heterologous 3′-UTR comprises a nucleic acid sequence derived from a 3′-UTR of a gene selected from PSMB3, ALB7, human alpha-globin (referred to as “muag”), CASP1, COX6B1, GNAS, NDUFA1, RSP10, human mitochondrial 12S rRNA (mtRNR1), human AES/TLE5 gene, FIG. 4 and RPS9, or from a homolog, a fragment or variant of any one of these genes, preferably according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 33-56 and SEQ ID NO: 161-164 or a fragment or a variant of any of these. Particularly preferred nucleic acid sequences in that context can be derived from published PCT application WO2019/077001, in particular, claim 9 of WO2019/077001. The corresponding 3′-UTR sequences of claim 9 of WO2019/077001 are herewith incorporated by reference (e.g., SEQ ID NOs: 23-34 of WO2019/077001, or fragments or variants thereof).

In some embodiments, the obtained in vitro transcribed RNA comprising a 3′ terminal A nucleotide may comprise a 3′-UTR derived from an alpha-globin gene. Said 3′-UTR derived from a alpha-globin gene (“muag”) may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 33 or 34 or a fragment or a variant thereof. In preferred embodiments, the in vitro transcribed RNA comprising a 3′ terminal A nucleotide may comprise a 3′-UTR derived from a PSMB3 gene. Said 3′-UTR derived from a PSMB3 gene may comprise or consist of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 35 or 36 or 161 or a fragment or a variant thereof.

In other embodiments, the obtained in vitro transcribed RNA comprising a 3′ terminal A nucleotide may comprise a 3′-UTR as described in WO2016/107877, the disclosure of WO2016/107877 relating to 3′-UTR sequences herewith incorporated by reference. Suitable 3′-UTRs are SEQ ID NOs: 1-24 and SEQ ID NOs: 49-318 of WO2016/107877, or fragments or variants of these sequences. In other embodiments, the nucleic acid comprises a 3-UTR as described in WO2017/036580, the disclosure of WO2017/036580 relating to 3′-UTR sequences herewith incorporated by reference. Suitable 3′-UTRs are SEQ ID NOs: 152-204 of WO2017/036580, or fragments or variants of these sequences. In other embodiments, the nucleic acid comprises a 3′-UTR as described in WO2016/022914, the disclosure of WO2016/022914 relating to 3′-UTR sequences herewith incorporated by reference. Particularly preferred 3′-UTRs are nucleic acid sequences according to SEQ ID NOs: 20-36 of WO2016/022914, or fragments or variants of these sequences.

In preferred embodiments, the at least one heterologous 5′-UTR comprises a nucleic acid sequence derived from a 5′-UTR of a gene selected from HSD17B4, human alpha-globin, RPL32, ASAH1, ATP5A1, MP68, NDUFA4, NOSIP, RPL31, SLC7A3, TUBB4B, and UBQLN2, or from a homolog, a fragment or variant of any one of these genes.

The terms “5′-untranslated region” or “5′-UTR” or “5′-UTR element” will be recognized and understood by the person of ordinary skill in the art, and are e.g. intended to refer to a part of the in vitro transcribed RNA comprising a 3′ terminal A nucleotide located 5′ (i.e. “upstream”) of a coding sequence and which is not translated into protein. A 5′-UTR may be part of a nucleic acid located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′ cap structure (e.g. for mRNA as defined above).

Preferably, the obtained in vitro transcribed RNA comprising a 3′ terminal A nucleotide comprises a 5′-UTR, which may be derivable from a gene that relates to an RNA with enhanced half-life (i.e. that provides a stable RNA). In preferred embodiments the 5′UTR comprising a GC rich element and/or an optimized Kozak sequence.

In preferred embodiments, the in vitro transcribed RNA comprising a 3′ terminal A nucleotide comprises at least one heterologous 5′-UTR, wherein the at least one heterologous 5′-UTR comprises a nucleic acid sequence derived from a 5′-UTR of gene selected from HSD17B4, human alpha-globin, RPL32, ASAH1, ATP5A1, MP68, NDUFA4, NOSIP, RPL31, SLC7A3, TUBB4B, and UBQLN2, or from a homolog, a fragment or variant of any one of these genes according to nucleic acid sequences being identical or at least 70%, 80%, 85%, 86%, 87%, 88%, 89%, 90%, 91%, 92%, 93%, 94%, 95%, 96%, 97%, 98%, or 99% identical to SEQ ID NOs: 1-32, SEQ ID NOs: 157-160 or a fragment or a variant of any of these. Particularly preferred nucleic acid sequences in that context can be selected from published PCT application WO2019/077001, in particular, claim 9 of WO2019/077001. The corresponding 5′-UTR sequences of claim 9 of WO2019/077001 are herewith incorporated by reference (e.g. SEQ ID NOs: 1-20 of WO2019/077001, or fragments or variants thereof).

In preferred embodiments, the in vitro transcribed RNA comprising a 3′ terminal A nucleotide may comprise a 5′-UTR derived from a HSD17B4 gene, wherein said 5′-UTR derived from a HSD17B4 gene comprises or consists of a nucleic acid sequence being identical or at least 70%, 80%, 85%, 86%, 87%, 88%, 89%, 90%, 91%, 92%, 93%, 94%, 95%, 96%, 97%, 98%, or 99% identical to SEQ ID NOs: 1 or 2 or a fragment or a variant thereof.

In other embodiments, the obtained in vitro transcribed RNA comprising a 3′ terminal A nucleotide comprises a 5′-UTR as described in WO2013/143700, the disclosure of WO2013/143700 relating to 5′-UTR sequences herewith incorporated by reference. Particularly preferred 5′-UTRs are nucleic acid sequences derived from SEQ ID NOs: 1-1363, SEQ ID NO: 1395, SEQ ID NO: 1421 and SEQ ID NO: 1422 of WO2013/143700, or fragments or variants of these sequences. In other embodiments, the coding RNA comprises a 5′-UTR as described in WO2016/107877, the disclosure of WO2016/107877 relating to 5′-UTR sequences herewith incorporated by reference. Particularly preferred 5′-UTRs are nucleic acid sequences according to SEQ ID NOs: 25-30 and SEQ ID NOs: 319-382 of WO2016/107877, or fragments or variants of these sequences. In other embodiments, the obtained in vitro transcribed RNA comprising a 3′ terminal A nucleotide comprises a 5′-UTR as described in WO2017/036580, the disclosure of WO2017/036580 relating to 5′-UTR sequences herewith incorporated by reference. Particularly preferred 5′-UTRs are nucleic acid sequences according to SEQ ID NOs: 1-151 of WO2017/036580, or fragments or variants of these sequences. In other embodiments, the obtained in vitro transcribed RNA comprising a 3′ terminal A nucleotide comprises a 5′-UTR as described in WO2016/022914, the disclosure of WO2016/022914 relating to 5′-UTR sequences herewith incorporated by reference. Particularly preferred 5′-UTRs are nucleic acid sequences according to SEQ ID NOs: 3-19 of WO2016/022914, or fragments or variants of these sequences.

In various embodiments, the obtained in vitro transcribed RNA comprising a 3′ terminal A nucleotide may comprise a 5′-terminal sequence element according to SEQ ID NOs: 176 or 177 of PCT/EP2020/052775, or a fragment or variant thereof. Such a 5-terminal sequence element comprises e.g. a binding site for T7 RNA polymerase. Further, the first nucleotide of said 5-terminal start sequence may preferably comprise a 2′0 methylation, e.g. 2′0 methylated guanosine or a 2′O methylated adenosine (which is an element of a Cap1 structure).

In particularly preferred embodiments, the obtained in vitro transcribed RNA comprising a 3′ terminal A nucleotide comprises at least one coding sequence as defined wherein said coding sequence is operably linked to a HSD17B4 5′-UTR and a PSMB3 3′-UTR (HSD17B4/PSMB3).

In particularly preferred embodiments, the obtained in vitro transcribed RNA comprising a 3′ terminal A nucleotide comprises at least one coding sequence as defined herein, wherein said coding sequence is operably linked to an alpha-globin (“muag”) 3′-UTR.

In particularly preferred embodiments, the obtained in vitro transcribed RNA comprising a 3′ terminal A nucleotide comprises at least one coding sequence as defined wherein said coding sequence is operably linked to a SLC7A3 5′-UTR and a PSMB3 3′-UTR (SLC7A3//PSMB3).

In particularly preferred embodiments, the obtained in vitro transcribed RNA comprising a 3′ terminal A nucleotide comprises at least one coding sequence as defined wherein said coding sequence is operably linked to a HSD17B4 5′-UTR and a FIG. 4.1 3′-UTR (HSD17B4/FIG. 4.1).

In particularly preferred embodiments, the obtained in vitro transcribed RNA comprising a 3′ terminal A nucleotide comprises at least one coding sequence as defined wherein said coding sequence is operably linked to a UBQLN2 5′-UTR and a RPS9.1 3′-UTR (UBQLN2/RPS9.1).

In particularly preferred embodiments the obtained in vitro transcribed RNA comprising a 3′ terminal A nucleotide is an mRNA.

The terms “RNA” and “mRNA” are e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. The specific succession of the monomers is called the RNA-sequence. The mRNA (messenger RNA) provides the nucleotide coding sequence that may be translated into an amino-acid sequence of a particular peptide or protein.

In vivo, transcription of DNA usually results in the so-called premature RNA which has to be processed into so-called messenger RNA, usually abbreviated as mRNA. Processing of the premature RNA, e.g. in eukaryotic organisms, comprises a variety of different posttranscriptional modifications such as splicing, 5-capping, polyadenylation, export from the nucleus or the mitochondria and the like. The sum of these processes is also called maturation of mRNA. The mature messenger RNA usually provides the nucleotide sequence that may be translated into an amino acid sequence of a particular peptide or protein. Typically, a mature mRNA comprises a 5′-cap, a 5′-UTR, an open reading frame, a 3′-UTR and a poly(A) or optionally a poly(C) sequence. In the context of the present invention, an mRNA may also be an artificial molecule, i.e. a molecule not occurring in nature. This means that the mRNA in the context of the present invention may, e.g., comprise a combination of a 5′-UTR, open reading frame, 3′-UTR and poly(A) sequence, which does not occur in this combination in nature. A typical mRNA (messenger RNA) in the context of the invention provides the coding sequence that is translated into an amino-acid sequence of a peptide or protein after e.g. in vivo administration to a cell.

In various embodiments the obtained in vitro transcribed RNA comprising a 3′ terminal A nucleotide, preferably the mRNA comprises the following elements preferably in 5′- to 3′-direction
A) 5′-cap structure, preferably as specified herein;
B) 5-terminal start element, preferably as specified herein;
C) optionally, a 5′-UTR, preferably as specified herein;
D) a ribosome binding site, preferably as specified herein;
E) at least one coding sequence, preferably as specified herein;
F) 3′-UTR, preferably as specified herein;
G) optionally, poly(A) sequence, preferably as specified herein;
H) optionally, poly(C) sequence, preferably as specified herein;
I) optionally, histone stem-loop preferably as specified herein;
J) optionally, 3′-terminal sequence element, preferably as specified herein.

In particularly preferred embodiments the obtained in vitro transcribed RNA comprising a 3′ terminal A nucleotide, preferably the mRNA, comprises the following elements preferably in 5′- to 3-direction:
A) cap1 structure as defined herein;
B) 5-terminal start element, preferably as specified herein;
C) coding sequence as specified herein;
D) 3′-UTR derived from a 3′-UTR of a muag gene as defined herein, preferably according to SEQ ID NO: 267 or 268 of PCT/EP2020/052775;
E) poly(A) sequence comprising about 64 A to about 200 A nucleotides.
F) poly(C) sequence comprising about 10 to about 100 cytosines;
G) histone stem-loop selected from SEQ ID NOs: 178 or 179 of PCT/EP2020/052775.

In particularly preferred embodiments, the obtained in vitro transcribed RNA comprising a 3′ terminal A nucleotide, preferably the mRNA, comprises the following elements preferably in 5′- to 3′-direction:
A) 5′ cap structure selected from m7G(5′), m7G(5′)ppp(5′)(2′OMeA), or m7G(5′)ppp(5′)(2′OMeG);
B) 5′-terminal start element selected from SEQ ID NOs: 57 or 58 or fragments or variants thereof;
C) optionally, a 5′-UTR derived from a HSD17B4 gene;
D) a ribosome binding site selected from SEQ ID NOs: 59, 60 or 156 or fragments or variants thereof;
E) at least one coding sequence encoding at least one therapeutic peptide or protein as specified herein;
F) 3′-UTR derived from a 3′-UTR of a PSMB3 gene or an alpha-globin gene (“muag”);
G) optionally, poly(A) sequence comprising about 30 to about 500 adenosines;
H) optionally, poly(C) sequence comprising about 10 to about 100 cytosines;
I) optionally, histone stem-loop selected from SEQ ID NOs: 61 or 62;
J) optionally, 3′ terminal sequence element selected from SEQ ID NOs: 63-92.

iii) Obtaining the In Vitro Transcribed RNA Comprising a 3′ Terminal a Nucleotide

The method according to this invention comprises a step iii) obtaining the in vitro transcribed RNA comprising a 3′ terminal A nucleotide.

According to the invention the obtained in vitro transcribed RNA comprising a 3′ terminal A nucleotide has reduced immunostimulatory properties compared to a corresponding reference in vitro transcribed RNA not comprising a 3′-terminal A nucleotide.

In preferred embodiments the method according to the invention leads to the formation of less double stranded RNA side products as compared to an in vitro transcription performed with a linear DNA template that does not comprise a 5′ terminal T nucleotide on the template DNA strand encoding the RNA.

In other preferred embodiments the obtained in vitro transcribed RNA comprising a 3′ terminal A nucleotide leads to improved expression of a therapeutic protein as compared to a corresponding reference in vitro transcribed RNA not comprising a 3′-terminal A nucleotide.

In preferred embodiments, the (non-purified) in vitro transcribed RNA obtained in step iii) is subjected to at least one purification step.

In other preferred embodiments the obtained in vitro transcribed RNA comprising a 3′ terminal A nucleotide is purified as described in step iv).

iv) Purifying the Obtained In Vitro Transcribed RNA Comprising a 3′ Terminal a Nucleotide

In particularly preferred embodiments, the method of this invention comprises a step iv) of purifying the obtained in vitro transcribed RNA comprising a 3′ terminal A nucleotide.

Accordingly, the method of reducing the immunostimulatory properties of an in vitro transcribed RNA by producing the in vitro transcribed RNA comprises the following steps:
i) providing a linear DNA template comprising a template DNA strand encoding the RNA, wherein the template DNA strand comprises a 5′ terminal T nucleotide;
ii) incubating the linear DNA template under conditions to allow (run-off) RNA in vitro transcription;
iii) obtaining the in vitro transcribed RNA comprising a 3′ terminal A nucleotide.
iv) purifying the obtained in vitro transcribed RNA after RNA in vitro transcription.

Thus, the obtained in vitro transcribed RNA comprising a 3-terminal A nucleotide is a purified RNA (e.g. a purified, in vitro transcribed mRNA).

The term “purified RNA” or “purified mRNA” as used herein has to be understood as RNA which has a higher purity after certain purification steps (e.g. HPLC, TFF, oligo d(T) purification, cellulose purification, precipitation, filtration, AEX) than the starting material (e.g. in vitro transcribed RNA). Typical impurities that are essentially not present in purified RNA comprise peptides or proteins (e.g. enzymes derived from RNA in vitro transcription, e.g. RNA polymerases, RNases, pyrophosphatase, restriction endonuclease, DNase), spermidine, BSA, short abortive RNA sequences, RNA fragments (short double stranded RNA fragments, short single stranded RNA fragments, abortive RNA sequences etc.), free nucleotides (modified nucleotides, conventional NTPs, cap analogue), template DNA fragments, buffer components (HEPES, TRIS, MgCl₂, CaCl₂) etc. Other potential impurities may be derived from e.g. fermentation procedures and comprise bacterial impurities (bioburden, bacterial DNA, bacterial RNA) or impurities derived from purification procedures (organic solvents etc.). Accordingly, it is desirable in this regard for the “degree of RNA purity” to be as close as possible to 100%.

Accordingly, “purified RNA” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing all the by-products. Alternatively, the degree of purity may for example be determined by an analytical agarose gel electrophoresis or capillary gel electrophoresis.

The obtained RNA may typically be produced by RNA in vitro transcription (IVT) of a (linear) DNA template. Common RNA in vitro transcription buffers comprise large amounts of MgCl₂ (e.g. 5 mM, 15 mM or more) which is a co-factor of the RNA polymerase. Accordingly, the obtained in vitro transcribed RNA may comprise Mg²⁺ ions as a contamination. After RNA in vitro transcription, the DNA template is typically removed by means of DNAses. Common buffers for DNAse digest comprise large amounts of CaCl₂ (e.g. 1 mM, 5 mM or more) which is a co-factor of the DNAse. Accordingly, the obtained in vitro transcribed RNA may comprise Ca²⁺ as a contamination.

In some preferred embodiments the method of this invention comprises a step iv) of purifying the obtained in vitro transcribed RNA comprising a 3′ terminal A nucleotide of iii), preferably to remove double-stranded RNA, non-capped RNA and/or RNA fragments.

In preferred embodiments the method according to this invention comprises a step iv) of purifying the obtained in vitro transcribed RNA comprising a 3′ terminal A nucleotide to remove double-stranded RNA.

Since dsRNA induces inflammatory cytokines and activates effector enzymes (cf. Kariko et al., Curr. Opin. Drug Discov. Devel. 10 (2007), 523-532) leading to protein synthesis inhibition, it is important to remove dsRNA from the IVT mRNA that will be used as therapeutic.

Accordingly, standard methods to remove double-stranded RNA to purify the obtained in vitro transcribed RNA comprising a 3′ terminal A nucleotide are incorporated within for example purification of IVT mRNA by ion-pair reversed phase HPLC using a non-porous (cf. Weissman et al., Methods Mol. Biol. 969 (2013), 43-54) or porous (cf. U.S. Pat. No. 8,383,340 B2)C-18 polystyrene-divinylbenzene (PS-DVB) matrix or an enzymatic based method has been established using E. coli RNaseIII that specifically hydrolyzes dsRNA but not ssRNA, thereby eliminating dsRNA contaminants from IVT mRNA preparations (cf. WO 2013/102203).

Another preferred embodiment the step iv) of purifying the obtained in vitro transcribed RNA to remove double-stranded RNA may comprise at least one step of cellulose purification as further described in detail in WO2017/182524.

Another preferred embodiment the step iv) of purifying the obtained in vitro transcribed RNA to remove double-stranded RNA may comprise at least one step of filtration step including a salt treatment as further described in detail in WO2021/255297 according to claim 1-14.

Moreover, various RNA purification steps (e.g. RP-HPLC, tangential flow filtration (TFF)) may be employed and combined to remove various contaminations including divalent metal ions. Typically, Ion Chromatography (IC) coupled with Inductively Coupled Plasma Mass Spectrometry (IC-ICP-MS) may be used for determination of divalent cations.

Accordingly, in various embodiments, the step iv) comprises at least one step selected from the list comprising RP-HPLC, AEX, TFF, oligo d(T) purification, cellulose purification, filtration step including a salt treatment, RNaseIII treatment, precipitation step, core-bead flow through chromatography step to reduce the immunostimulatory properties of an in vitro transcribed RNA.

In various embodiments, the step iv) comprises at least one step of RP-HPLC and/or at least one step of AEX, and/or at least one step of TFF and/or at least one step of oligo d(T) purification and/or at least one step of cellulose purification and/or at least one filtration step including a salt treatment and/or at least one step of RNaseIII treatment and/or at least one precipitation step and/or at least one core-bead flow through chromatography step.

In various preferred embodiments, step iv) comprises a combination of different purification steps as defined herein

The combination of different purification steps in the context of the invention is particularly preferred and advantageous as the immunostimulatory properties of an in vitro transcribed RNA can be further reduced.

Preferably, any of the purification steps mentioned herein are performed as defined herein or as typically performed by the skilled artisan. If certain purification steps are to be combined to further reduce the immunostimulatory properties of the RNA, the skilled person is aware of certain steps in between (e.g. buffer exchange steps) or to adapt the methods to make them compatible with each other.

Suitably, the step iv) comprises a combination of at least two different purification steps at outlined herein.

In preferred embodiments in that context, the step iv) comprises at least one step of RP-HPLC and at least one step selected from the list comprising at least one step of AEX, at least one step of TFF, at least one step of oligo d(T) purification, at least one step of cellulose purification, at least one filtration step including a salt treatment, at least one step of RNaseIII treatment, at least one precipitation step, or at least one core-bead flow through chromatography step.

In preferred embodiments in that context, the step iv) comprises at least one step of oligo d(T) purification and at least one step selected from the list comprising at least one step of AEX, at least one step of TFF, at least one step of RP-HPLC, at least one step of cellulose purification, at least one filtration step including a salt treatment, at least one step of RNaseIII treatment, at least one precipitation step, or at least one core-bead flow through chromatography step.

In preferred embodiments in that context, the step iv) comprises at least one step of cellulose purification and at least one step selected from the list comprising at least one step of AEX, at least one step of TFF, at least one step of RP-HPLC, at least one step of oligo d(T) purification, at least one filtration step including a salt treatment, at least one step of RNaseIII treatment, at least one precipitation step, at least one core-bead flow through chromatography step.

In preferred embodiments in that context, the step iv) comprises at least one AEX step, and at least one step selected from the list comprising at least one step of cellulose purification at least one step of TFF, at least one step of RP-HPLC, at least one step of oligo d(T) purification, at least one filtration step including a salt treatment, at least one step of RNaseIII treatment, at least one precipitation step, at least one core-bead flow through chromatography step.

In preferred embodiments in that context, the step iv) comprises at least one filtration step including a salt treatment, and at least one step selected from the list comprising at least one step of cellulose purification at least one step of TFF, at least one step of RP-HPLC, at least one step of oligo d(T) purification, at least AEX step, at least one step of RNaseIII treatment, at least one precipitation step, at least one core-bead flow through chromatography step.

In preferred embodiments in that context, the step iv) comprises at least one step of RNaseIII treatment, and at least one step selected from the list comprising at least one step of cellulose purification at least one step of TFF, at least one step of RP-HPLC, at least one step of oligo d(T) purification, at least AEX step, at least one filtration step including a salt treatment, at least one precipitation step, at least one core-bead flow through chromatography step.

In preferred embodiments in that context, the step iv) comprises at least one precipitation step, and at least one step selected from the list comprising at least one step of cellulose purification at least one step of TFF, at least one step of RP-HPLC, at least one step of oligo d(T) purification, at least AEX step, at least one filtration step including a salt treatment, at least one step of RNaseIII treatment, at least one core-bead flow through chromatography step.

In preferred embodiments in that context, the step iv) comprises at least one core-bead flow through chromatography step, and at least one step selected from the list comprising at least one step of cellulose purification at least one step of TFF, at least one step of RP-HPLC, at least one step of oligo d(T) purification, at least AEX step, at least one filtration step including a salt treatment, at least one step of RNaseIII treatment, at least one precipitation step.

In one embodiment, the step iv) comprises at least one step of RP-HPLC and at least one step of AEX.

In preferred embodiments, the step iv) comprises at least one step of RP-HPLC and at least one step of TFF.

In preferred embodiments, the step iv) comprises at least one step of RP-HPLC and at least one step of oligo d(T) purification.

In preferred embodiments, the step iv) comprises at least one step of RP-HPLC and at least one step of cellulose purification.

In one embodiment, the step iv) comprises at least one step of RP-HPLC and at least one step of filtration step including a salt treatment.

In one embodiment, the step iv) comprises at least one step of RP-HPLC and at least one step of RNaseIII treatment.

In one embodiment, the step iv) comprises at least one step of RP-HPLC and at least one precipitation step.

In one embodiment, the step iv) comprises at least one step of RP-HPLC and at least one core-bead flow through chromatography step.

In one embodiment, the step iv) comprises at least one step of oligo d(T) purification and at least one step of AEX.

In one embodiment, the step iv) comprises at least one step of oligo d(T) purification and at least one step of TFF.

In particularly preferred embodiments, the step iv) comprises at least one step of oligo d(T) purification and at least one step of cellulose purification.

In one embodiment, the step iv) comprises at least one step of oligo d(T) purification and at least one filtration step including a salt treatment.

In one embodiment, the step iv) comprises at least one step of oligo d(T) purification and at least one step of RNaseIII treatment.

In one embodiment, the step iv) comprises at least one step of oligo d(T) purification and at least one precipitation step.

In preferred embodiments, the step iv) comprises at least one step of oligo d(T) purification and at least one core-bead flow through chromatography step.

In one embodiment the step iv) comprises and at least one step of cellulose purification and at least one step of AEX.

In preferred embodiments, the step iv) comprises and at least one step of cellulose purification and at least one step of TFF.

In one embodiment, the step iv) comprises at least one step of cellulose purification and at least one filtration step including a salt treatment.

In one embodiment the step iv) comprises at least one step of cellulose purification and at least one step of RNAseIII purification.

In one embodiment, the step iv) comprises at least one step of cellulose purification and at least one precipitation step.

In preferred embodiments, the step iv) comprises at least one step of cellulose purification and at least one core-bead flow through chromatography step.

Suitably, the step iv) comprises at least three different purification steps at outlined herein.

In preferred embodiments in that context, the step iv) comprises at least one step of RP-HPLC and at least one step of oligo d(T) purification and at least one step selected from the list comprising at least one step of AEX, at least one step of TFF, at least one step of cellulose purification, at least one filtration step including a salt treatment, at least one step of RNaseIII treatment, at least one precipitation step, or at least one core-bead flow through chromatography step.

In preferred embodiments in that context, the step iv) comprises at least one step of RP-HPLC and at least one step of cellulose purification and at least one step selected from the list comprising at least one step of AEX, at least one step of TFF, at least one step of oligo d(T) purification, at least one filtration step including a salt treatment, at least one step of RNaseIII treatment, at least one precipitation step, or at least one core-bead flow through chromatography step.

In preferred embodiments in that context, the step iv) comprises at least one step of oligo d(T) purification and at least one step of cellulose purification and at least one step selected from the list comprising at least one step of AEX, at least one step of TFF, at least one step of RP-HPLC, at least one filtration step including a salt treatment, at least one step of RNaseIII treatment, at least one precipitation step, or at least one core-bead flow through chromatography step.

In preferred embodiments, the step iv) comprises at least one step of RP-HPLC and at least one step of oligo d(T) purification and at least one step of AEX.

In preferred embodiments, the step iv) comprises at least one step of RP-HPLC and at least one step of oligo d(T) purification and at least one step of TFF.

In preferred embodiments, the step iv) comprises at least one step of RP-HPLC and at least one step of oligo d(T) purification and at least one step of cellulose purification.

In preferred embodiments, the step iv) comprises at least one step of RP-HPLC and at least one step of oligo d(T) purification and at least one step of filtration step including a salt treatment.

In preferred embodiments, the step iv) comprises at least one step of RP-HPLC and at least one step of oligo d(T) purification and at least one step of RNaseIII treatment.

In preferred embodiments, the step iv) comprises at least one step of RP-HPLC and at least one step of oligo d(T) purification and at least one precipitation step.

In preferred embodiments, the step iv) comprises at least one step of RP-HPLC and at least one step of oligo d(T) purification and at least one core-bead flow through chromatography step.

In preferred embodiments, the step iv) comprises at least one step of RP-HPLC and at least one step of cellulose purification and at least one step of AEX

In preferred embodiments, the step iv) comprises at least one step of RP-HPLC and at least one step of cellulose purification and at least one step of TFF

In preferred embodiments, the step iv) comprises at least one step of RP-HPLC and at least one step of cellulose purification and at least one step of filtration step including a salt treatment

In preferred embodiments, the step iv) comprises at least one step of RP-HPLC and at least one step of cellulose purification and at least one step of RNaseIII treatment.

In preferred embodiments, the step iv) comprises at least one step of RP-HPLC and at least one step of cellulose purification and at least one precipitation step.

In preferred embodiments, the step iv) comprises at least one step of RP-HPLC and at least one step of cellulose purification and at least one core-bead flow through chromatography step.

In preferred embodiments, the step iv) comprises at least one step of oligo d(T) purification and at least one step of cellulose purification and at least one step of AEX

In preferred embodiments, the step iv) comprises at least one step of oligo d(T) purification and at least one step of cellulose purification and at least one step of TFF

In preferred embodiments, the step iv) comprises at least one step of oligo d(T) purification and at least one step of cellulose purification and at least one step of filtration step including a salt treatment

In preferred embodiments, the step iv) comprises at least one step of oligo d(T) purification and at least one step of cellulose purification and at least one step of RNaseIII treatment.

In preferred embodiments, the step iv) comprises at least one step of oligo d(T) purification and at least one step of cellulose purification and at least one precipitation step.

In preferred embodiments, the step iv) comprises at least one step of oligo d(T) purification and at least one step of cellulose purification and at least one core-bead flow through chromatography step.

Suitably, the step iv) comprises at least four different purification steps at outlined herein.

In various preferred embodiments in that context, any of the above described combination of purification steps additionally comprises at least one step of TFF.

In various preferred embodiments in that context, any of the above described combination of purification steps additionally comprises at least one step of DNA digestion, preferably DNAse treatment.

In various preferred embodiments in that context, any of the above described combination of purification steps additionally comprises at least one step of protein digestion, preferably proteinase K treatment.

In various preferred embodiments in that context, any of the above described combination of purification steps additionally comprises at least one step of 5′ dephosphorylation of RNA or RNA impurities. Linear RNA may carry 5 triphosphate ends (e.g. RNA species that do not carry a cap structure) that should be removed to avoid e.g. immunostimulation. Accordingly, in preferred embodiments, the step of 5′ dephosphorylation of RNA may further reduce the immunostimulatory properties of the obtained RNA.

The dephsphorylation may be carried out using an enzyme that converts a 5′ triphosphate of the linear RNA into a 5′ monophosphate. Accordingly, in some embodiments, the circular RNA preparation is contacted with RNA 5′ pyrophosphohydrolase (RppH) or an ATP diphosphohydrolase (apyrase) to convert a 5′ triphosphate of the linear RNA into a 5′ monophosphate.

Preferably, the dephsphorylation may be carried out using an enzyme that removes all three 5′ phosphate groups. Accordingly, in preferred embodiments the circular RNA preparation is contacted with a phosphatase (e.g., Antarctic Phosphatase, Shrimp Alkaline Phosphatase, or Calf Intestinal Phosphatase to remove all three phosphates.

In the following, specific embodiments relating to some preferred purification steps are provided.

In preferred embodiments, step iv) of the method comprises at least one step of RP-HPLC. Hereby, the obtained in vitro transcribed RNA comprising a 3′-terminal A nucleotide is purified using a method as described in published patent application WO2008/077592 and WO2017/137095, the specific disclosure relating to the published PCT claims 1 to 28 is herewith incorporated by reference.

In preferred embodiments of step iv), the at least one further purification method is a reversed phase chromatography method, preferably a reversed phase HPLC (RP-HPLC) method. Preferably, the reversed phase chromatography comprises using a porous reserved phase as stationary phase.

In preferred embodiments of the RP-HPLC, the porous reversed phase material is provided with a particle size of 8.0 μm to 50 μm, in particular 8.0 to 30 μm, still more preferably about 30 μm. The reversed phase material may be present in the form of small spheres. The method according to the invention may be performed particularly favorably with a porous reversed phase with this particle size, optionally in bead form, wherein particularly good separation results are obtained.

In preferred embodiments of the RP-HPLC, the reversed phase has a pore size of 1000 Å to 5000 Å, in particular a pore size of 1000 Å to 4000 Å, more preferably 1500 Å to 4000 Å, 2000 Å to 4000 Å or 2500 Å to 4000 Å. Most preferred is a pore size of 4000 Å.

In preferred embodiments of the RP-HPLC, the material for the reversed phase is a porous polystyrene polymer, a (non-alkylated) (porous) polystyrenedivinylbenzene polymer, porous silica gel, porous silica gel modified with non-polar residues, particularly porous silica gel modified with alkyl containing residues, more preferably with butyl-, octyl and/or octadecyl containing residues, porous silica gel modified with phenylic residues, porous polymethacrylates, wherein in particular a porous polystyrene polymer or a non-alkylated (porous) polystyrenedivinylbenzene may be used.

In a preferred embodiment of the RP-HPLC, a non-alkylated porous polystyrenedivinylbenzene is used that may have a particle size of 8.0±1.5 μm, in particular 8.0±0.5 μm, and a pore size of 3500 to 4500A and most preferably of 4000 Å. In a preferred embodiment of the RP-HPLC, a mixture of an aqueous solvent and an organic solvent is used as the mobile phase for eluting the RNA. It is favorable for a buffer to be used as the aqueous solvent which has in particular a pH of 6.0-8.0, for example of about 7, for example. 7.0; preferably the buffer is triethylammonium acetate (TEAA), particularly preferably a 0.02 M to 0.5 M, in particular 0.08 M to 0.12 M, very particularly an about 0.1 M TEAA buffer, which, as described above, also acts as a counter ion to the RNA in the ion pair method.

In a preferred embodiment of the RP-HPLC, the organic solvent which is used in the mobile phase comprises acetonitrile, methanol, ethanol, 1-propanol, 2-propanol and acetone or a mixture thereof, very particularly preferably acetonitrile. With these organic solvents, in particular acetonitrile, purification of the RNA proceeds in a particularly favorable manner with the method according to the invention.

In particularly preferred embodiments of the RP-HPLC, the mobile phase is a mixture of 0.1 M triethylammonium acetate, pH 7, and acetonitrile.

In other preferred embodiments of the RP-HPLC, the mobile phase to comprises 7.5 vol. % to 17.5 vol. % organic solvent, relative to the mobile phase, and for this to be made up to 100 vol. % with the aqueous buffered solvent.

In preferred embodiments of the RP-HPLC, gradient separation is performed. In this respect, the composition of the eluent is varied by means of a gradient program. The equipment necessary for gradient separation is known to a person skilled in the art.

In preferred embodiments of the RP-HPLC, the proportion of the organic solvent is increased relative to the aqueous solvent during gradient separation. The above-described agents may here be used as the aqueous solvent and the likewise above-described agents may be used as the organic solvent.

For example, the proportion of organic solvent in the mobile phase may be increased in the course of HPLC separation from 5.0 vol. % to 20.0 vol. %, in each case relative to the mobile phase. In particular, the proportion of organic solvent in the mobile phase may be increased in the course of HPLC separation from 7.5 vol. % to 17.5 vol. %, in particular 9.5 to 14.5 vol. %, in each case relative to the mobile phase.

In a particularly preferred embodiment, the RP-HPLC purification is performed under denaturing conditions. Preferably, the RP-HPLC purification step is performed at a temperature of about 60° C. or more, particularly preferably at a temperature of about 70° C. or more, in particular up to about 80° C. or more. Suitably, the temperature is maintained and kept constant during the RP-HPLC purification procedure.

In preferred embodiments, the RP-HPLC step is performed as described in WO2008/077592, in particular according to PCT claims 1 to 26. Accordingly, the disclosure of WO2008/077592, in particular the disclosure relating to PCT claims 1 to 26 are herewith incorporated by reference.

As described above, the use of reversed phase chromatography methods typically requires the use of organic solvents such as acetonitrile (ACN), methanol, ethanol, 1-propanol, 2-propanol, trifluoroacetic acid (TFA), trifluoroethanol (TFE) or combinations thereof. However, these organic solvents may need to be removed from the RNA-containing pool afterwards.

Furthermore, other contaminations derived from prior production or purification steps may still be present in the RNA-containing pool after RP-HPLC and need to be removed (for example divalent cations such as Mg2+ and/or Ca2+ have a negative impact on temperature stability of RNA).

In other preferred embodiments, step iv) of the method comprises at least one purification step of TFF preferably against a salt buffer, preferably against an NaCl buffer. In preferred embodiments, a tangential flow filtration method as described in published patent application WO2016/193206 may be used, the specific disclosure relating to the published PCT claims 1 to 48 is herewith incorporated by reference.

In other embodiments, step iv) of the method comprises at least one purification step of TFF in the presence of Ammonium sulfate. In other embodiments, step iv) of the method comprises at least one purification step of TFF in the presence of chaotropic agents, preferably in the presence of guanidinium thiocyanate.

Thus, in preferred embodiments, step iv) of the method comprises conditioning and/or purifying the solution comprising transcribed RNA obtained in step iii) by one or more steps of TFF. The one or more steps of TFF may comprise at least one diafiltration step and/or at least one concentration step. The diafiltration and concentration steps may be performed separately, but they may also at least partially overlap. The one or more steps of TFF may efficiently remove contaminants, such as HMWC and LMWC, e.g. RNA fragments; DNA fragments, proteins, organic solvents, nucleoside triphosphates, spermidine and buffer components such as salts and detergents.

In a preferred embodiment, the one or more steps of TFF comprises at least one diafiltration step, preferably a diafiltration step which is preferably performed with water and/or with an aqueous salt solution.

In a preferred embodiment, the aqueous salt solution comprises NaCl. In a more preferred embodiment, the aqueous salt solution comprises from about 0.1 M NaCl to about 1 M NaCl, more preferably from about 0.2 to about 0.5 M NaCl.

In another preferred embodiment, the diafiltration solution is water, preferably distilled and sterile water, more preferably water for injection.

The one or more steps of TFF may be carried out using any suitable filter membrane. For example, the one or more steps of TFF may be carried out using a TFF hollow fibre membrane or a TFF membrane cassette. Particularly preferred in this context is a TFF membrane cassette comprising a cellulose-based membrane or a PES or mPES-based filter membrane with a MWCO of 100 kDa.

In some embodiments, the feed flow rate in one or more steps of TFF is 100 to 1.500 I/h/m2, preferably 150 to 1.300 I/h/m2, more preferably 200 to 1.100 I/h/m2 and most preferably 300 to 1.050 I/h/m2.

In preferred embodiments, a the one or more steps of TFF for conditioning and/or purifying the RNA is preferably performed as described in published patent application WO2016/193206, the disclosure relating to TFF for conditioning and/or purifying the RNA disclosed in WO2016/193206 herewith incorporated by reference. Exemplary parameters for TFF of the RNA are provided in Example 14, e.g. Table 17 of WO2016/193206.

In preferred embodiments of step iv), the method comprises at least one further purification method before or after the one or more steps of TFF.

Accordingly, in preferred embodiments, the step iv) comprises purification methods using PureMessenger® (CureVac, Tubingen, Germany; RP-HPLC according to WO2008/077592) and/or tangential flow filtration (as described in WO2016/193206) and/or oligo d(T) purification (see WO2016/180430).

In preferred embodiments, step iv) of the method comprises one or more steps of TFF and at least one step of RP-HPLC.

In preferred embodiments, at least one step of TFF in step C may be performed after performing the at least one further purification method, e.g. after the RP-HPLC. Suitably, the at least one step of TFF performed after the RP-HPLC is configured to remove organic solvents from the RP-HPLC pool, and to further remove RNA by-products or to further remove divalent cations.

This at least one step of TFF performed after the RP-HPLC may comprise at least a first step of diafiltration. Preferably, the first diafiltration step is performed with an aqueous salt solution as diafiltration solution. In a preferred embodiment, the aqueous salt solution comprises NaCl. In a more preferred embodiment, the aqueous salt solution comprises about 0.1 M NaCl to about 1 M NaCl, more preferably from about 0.2 to about 0.5 M NaCl. In a particularly preferred embodiment, the aqueous salt solution comprises 0.2 M NaCl. The presence of NaCl may be advantageous for removing contaminating spermidine from the RNA-pool and for removing Mg2+ and/or Ca2+ ions from the RNA-pool. In a preferred embodiment, the first diafiltration step is performed using from about 1 to about 20 DV diafiltration solution, preferably from about 1 to about 15 DV diafiltration solution and more preferably from about 5 to about 12 DV diafiltration solution and even more preferably from about 7 to about 10 DV diafiltration solution. In a particularly preferred embodiment, the first diafiltration step is performed using about 10 DV diafiltration solution. Particularly preferred in this context is a TFF membrane cassette comprising a cellulose-based membrane or a PES or mPES-based filter membrane with a MWCO of 100 kDa.

In preferred embodiments, the TFF performed after the RP-HPLC is preferably performed as described in published patent application WO2016/193206, the disclosure relating to TFF for conditioning and/or purifying RP-HPLC purified RNA disclosed in WO2016/193206 herewith incorporated by reference. Exemplary parameters for TFF of the RP-HPLC purified RNA are provided in Example 14, e.g. Table 18 of WO2016/193206.

In a preferred embodiment of step iv), the method comprises the following steps, preferably in the given order:
conditioning and/or purifying of the solution comprising the in vitro transcribed RNA by one or more steps of TFF, preferably wherein least one TFF step is diafiltration of at least 10 diafiltration volumes (DV) against a diafiltration buffer, suitably water for injection; and
purifying the RNA by reversed phase chromatography, preferably RP-HPLC using a non-alkylated porous polystyrenedivinylbenzene matrix (suitably with a pore size of about 4000 Å) preferably performed at a temperature of about 70° C. or more; and
concentrating and/or purifying of the solution comprising the RP-HPLC purified RNA by one or more steps of TFF using a TFF membrane cassette (suitably a 100 kDa TFF membrane cassette), wherein at least one TFF step is diafiltration of at least 10 diafiltration volumes (DV) wherein the diafiltration solution is an aqueous salt solution, preferably wherein the aqueous salt solution comprises NaCl (suitably from about 0.2 to about 0.5 M NaCl); and, optionally
conditioning of the TFF purified RNA comprised in an aqueous salt solution by one or more steps of TFF preferably wherein at least one step of TFF is diafiltration of at least 10 diafiltration volumes (DV) against a diafiltration buffer, suitably water for injection; and, optionally
filtration of the purified RNA filtration using a 0.22 μm pore size filter.

The purification procedure as outlined herein may efficiently remove by-products and impurities from the in vitro transcribed RNA comprising a 3-terminal A nucleotide obtained in step ii). Advantageously, the purification procedure as outlined herein may also remove divalent cations including Ca2+ and Mg+. Without wishing to be bound to theory, the purification procedure as outlined herein improves the thermal stability of the RNA (when stored encapsulated in the lipid-based carriers of the invention at temperatures above around 5° C.).

In some embodiments the step iv) comprises at least one step of AEX. Accordingly, Anion exchange (AEX) chromatography is a method of purification and analysis that leverages ionic interaction between positively charged sorbents and negatively charged molecules. AEX sorbents consist of a charged functional group (e.g. quatemary amine, polyethylenimine, diethylaminoethyl, dimethylaminopropyl etc.), cross-linked to solid phase media. There are two categories of anion exchange media, “strong” and “weak” exchangers. Strong exchangers maintain a positive charge over a broad pH range, while weak exchangers only exhibit charge over a specific pH range. Anion exchange resins facilitate RNA capture due to the interaction with the negatively charged phosphate backbone of the RNA providing an ideal mode of separation. The mechanism of purification or analysis can involve binding the RNA under relatively low ionic strength solution to an AEX sorbent. Further details are described in the patent application WO2017/137095 and herewith incorporated by reference.

In preferred embodiments, the step iv) comprises at least one step of oligo d(T) purification.

In embodiments the step iv) comprises at least one step of oligo d(T) purification. Hereby the obtained in vitro transcribed RNA may be purified using a unit for affinity purification via oligo dT functionalized matrices or beads or columns (e.g. as described in WO2014152031A1, WO2017205477, WO2016/180430 and WO2021030533).

Suitably in that context, the oligo d(T) purification is performed with an oligo dT ranging from about T15 to about T100, preferably ranging from about T15 to about T80, more preferably ranging from about T50 to about T80, e.g. T60.

In preferred embodiments in that context, the oligo dT is immobilized on a solid support, preferably wherein the solid support is a bead or a column.

The shape, form, materials, and modifications of the solid support can be selected from a range of options depending on the desired application or scale. Exemplary materials that can be used as a solid support include, but are not limited to acrylics, carbon (e.g., graphite, carbon-fiber), cellulose (e.g., cellulose acetate), ceramics, controlled-pore glass, cross-linked polysaccharides (e.g., agarose or SEPHAROSE™), gels, glass (e.g., modified or functionalized glass), gold (e.g., atomically smooth Au(111)), graphite, inorganic glasses, inorganic polymers, latex, metal oxides (e.g., SiO2, TiO2, stainless steel), metalloids, metals (e.g., atomically smooth Au(1 111), mica, molybdenum sulfides, nanomaterials (e.g., highly oriented pyrolitic graphite (HOPG) nanosheets), nitrocellulose, NYLON™, optical fiber bundles, organic polymers, paper, plastics, polacryloylmorpholide, poly(4-methylbutene), polyethylene terephthalate), poly(vinyl butyrate), polybutylene, polydimethylsiloxane (PDMS), polyethylene, polyformaldehyde, polymethacrylate, polypropylene, polysaccharides, polystyrene, polyurethanes, polyvinylidene difluoride (PVDF), quartz, rayon, resins, rubbers, semiconductor material, silica, silicon (e.g., surface-oxidized silicon), sulfide, and TEFLON™. A single material or mixture of several different materials can form a solid support useful in the context of the invention.

In preferred embodiments, the solid support comprises sepharose. For example, the solid support may be a sepharose bead or a sepharose column.

In preferred embodiments, the solid support comprises silica. For example, the solid support may be a silica bead or a silica column.

In preferred embodiments, the solid support is a monolithic material, e.g. a methacrylate monolith.

The terms “monolith,” “monolithic matrix” and “monolithic column” are used interchangeably herein to refer to a solid support (e.g. a chromatography column) composed of a continuous stationary phase made of a polymer matrix. In contrast to particle-based chromatography columns, monolithic columns are made of a porous polymer material with highly interconnected channels and large pore size. While particle-based columns rely on diffusion through pores, separation by monolithic columns occurs primarily by convective flow through relatively large channels (about 1 micron or more).

A suitable monolithic matrix may be derived from a variety of materials, such as but not limited to, polymethacrylate, polyacrylamide, polystyrene, silica and cryogels.

In one embodiment, the solid support is modified to contain chemically modified sites that can be used to attach, either covalently or non-covalently, the oligo dT to discrete sites or locations on the surface. “Chemically modified sites” in this context includes, but is not limited to, the addition of a pattern of chemical functional groups including amino groups, carboxy groups, oxo groups and thiol groups, that can be used to covalently attach the oligo dT oligonucleotide, which generally also contain corresponding reactive functional groups. Examples of surface functionalizations are: Amino derivatives, Thiol derivatives, Aldehyde derivatives, Formyl derivatives, Azide Derivatives (click chemistry), Biotin derivatives, Alkyne derivatives, Hydroxyl derivatives, Activated hydroxyls or derivatives, Carboxylate derivatives, activated carboxylate derivates, Activated carbonates, Activated esters, NHS Ester (succinimidyl), NHS Carbonate (succinimidyl), Imidoester or derivated, Cyanogen Bromide derivatives, Maleimide derivatives, Haloacteyl derivatives, Iodoacetamide/iodoacetyl derivatives, Epoxide derivatives, Streptavidin derivatives, Tresyl derivatives, Diene/conjugated diene derivatives (diels alder type reaction), Alkene derivatives, Substituted phosphate derivatives, Bromohydrin/halohydrin, Substituted disulfides, Pyridyl-disulfide Derivatives, Aryl azides, Acyl azides, Azlactone, Hydrazide derivatives, Halobenzene derivatives, Nucleoside derivatives, Branching/multi functional linkers, Dendrimeric functionalities, and/or Nucleoside derivatives; or any combination thereof.

In some embodiments, the oligo dT is linked directly to the solid support.

In some embodiments, the oligo dT is linked to the solid support via a linker.

In some embodiments, a solid support and/or the oligo dT can be attached to a linker.

The term “linker” can refer to a connection between two molecules or entities, for example, the connection between the oligo dT oligonucleotide and a spacer or the connection between the oligo dT oligonucleotide and a solid support.

The linker can be formed by the formation of a covalent bond or a non-covalent bond. Suitable covalent linkers can include, but are not limited to the formation of an amide bond, an oxime bond, a hydrazone bond, a triazole bond, a sulfide bond, an ether bond, an enol ether bond, an ester bond, or a disulfide bond.

Suitable linkers include alkyl and aryl groups, including heteroalkyl and heteroaryl, and substituted derivatives of these. In some instances, linkers can be amino acid based and/or contain amide linkages. Examples of linkers are: Amino derivatives, Thiol derivatives, Aldehyde derivatives, Formyl derivatives, Azide Derivatives (click chemistry), Biotin derivatives, Alkyne derivatives, Hydroxyl derivatives, Activated hydroxyls or derivatives, Carboxylate derivatives, activated carboxylate derivates, Activated carbonates, Activated esters, NHS Ester (succinimidyl), NHS Carbonate (succinimidyl), Imidoester or derivated, Cyanogen Bromide derivatives, Maleimide derivatives, Haloacteyl derivatives, lodoacetamide/iodoacetyl derivatives, Epoxide derivatives, Streptavidin derivatives, Tresyl derivatives, Diene/conjugated diene derivatives (diels alder type reaction), Alkene derivatives, Substituted phosphate derivatives, Bromohydrin/halohydrin, Substituted disulfides, Pyridyl-disulfide Derivatives, Aryl azides, Acyl azides, Azlactone, Hydrazide derivatives, Halobenzene derivatives, Nucleoside derivatives, Branching/multi functional linkers, Dendrimeric funcationalities, and/or Nucleoside derivatives; or any combination thereof.

In particularly preferred embodiments, the oligo dT is linked to a sepharose bead that comprises streptavidin.

In preferred embodiments, the method comprises a step of subjecting the composition comprising RNA and to the oligo dT oligonucleotide (as defined herein) under conditions that allow nucleic acid hybridization.

In some embodiments, the conditions that allow nucleic acid hybridization is a temperature of about 20° C. to about 60° C., preferably about 30° C.

In some embodiments, the conditions that allow nucleic acid hybridization is at a pH of about 7.0.

In some embodiments, the conditions that allow nucleic acid hybridization is a buffer condition, wherein the buffer is a hybridization buffer, e.g. an saline sodium citrate buffer (SSC).

Suitably, the hybridization buffer comprises 100 mM to 1M sodium chloride and 10 mM to 100 mM trisodium citrate. For example, the hybridization buffer comprises 300 mM sodium chloride, 30 mM trisodium citrate.

In particularly preferred embodiments, the linear RNA precursor and the oligo dT oligonucleotide bind one another via non-covalent bonding, e.g. nucleic acid hybridization.

In some embodiments of oligo d(T) purification to purify the RNA using oligodT column, a specific amount of RNA may be incubated with e.g. 1.5× molar excess of oligodT₆₀ in a binding buffer (e.g. 2×SSC buffer). In one embodiment, streptavidin sepharose beads may be equilibrated in the binding buffer (e.g. 2×SSC buffer). In one embodiment, equilibrated beads may be added to RNA-oligodT₆₀ mix and incubated to allow hybridization (e.g. for 15 min at room temperature with intermittent mixing by tapping the tube). In preferred embodiment, the bound RNA may be eluted in nuclease free water and optionally precipitated with sodium acetate and isopropanol. Precipitated RNA may be recovered by centrifugation and dissolved in nuclease free water.

In a preferred embodiment the step iv) comprises at least one step of cellulose purification (e.g. as described in WO2017/182524).

Hereby, the purification step are conducted under conditions which allow binding of dsRNA to the cellulose material and do not allow binding of ssRNA to the cellulose material. The condition allows the selective binding of dsRNA to the cellulose material, whereas ssRNA remains unbound.

In one embodiment of the cellulose purification procedure the purification step comprises mixing the in vitro transcribed RNA with the cellulose material under shaking and/or stirring, preferably for at least 5 min, more preferably for at least 10 min.

In one embodiment of the cellulose purification procedure, the in vitro transcribed RNA is provided as a liquid comprising ssRNA and a first buffer and/or the cellulose material is provided as a suspension in a first buffer, wherein the first buffer comprises water, ethanol and a salt, preferably sodium chloride, in a concentration which allows binding of dsRNA to the cellulose material and which does not allow binding of ssRNA to the cellulose material. In one embodiment, the concentration of ethanol in the first buffer is 14 to 20% (v/v), preferably 14 to 16% (v/v). In one embodiment, the concentration of the salt in the first buffer is 15 to 70 mM, preferably 20 to 60 mM. In one embodiment, the first buffer further comprises a buffering substance, preferably tris(hydroxymethyl)aminomethane (TRIS), and/or a chelating agent, preferably EDTA.

In a preferred embodiment the cellulose purification step comprises
(1) mixing the cellulose material to which dsRNA and ssRNA are bound with a first buffer under shaking and/or stirring, wherein the first buffer comprises water, ethanol and a salt in a concentration which allows binding of dsRNA to the cellulose material and does not allow binding of ssRNA to the cellulose material; and
(2) separating the liquid phase comprising ssRNA from the cellulose material;
and the concentration of ethanol in the first buffer is 14 to 20% (v/v) and the concentration of the salt in the first buffer is 15 to 70 mM.

In one embodiment of the cellulose purification procedure the mixture of the in vitro transcribed RNA, the cellulose material, and the first buffer is provided in a tube and comprises applying gravity or centrifugal force to the tube such that the liquid and solid phases are separated; and either collecting the supernatant comprising ssRNA or removing the cellulose material.

In an alternative embodiment the mixture of the in vitro transcribed RNA, the cellulose material, and the first buffer is provided in a spin column or filter device and comprises applying gravity, centrifugal force, pressure, or vacuum to the spin column or filter device such that the liquid and solid phases are separated; and collecting the flow through comprising ssRNA.

In a preferred embodiment, cellulose purification of RNAs is performed in a single cellulose spin column. In another preferred embodiment cellulose purification is performed in several cellulose spin columns. In particularly preferred embodiments, cellulose purification is performed in a cellulose column suitable for large-scale purification, e.g. for purification of at least 1 g to at least 100 g RNA.

In a preferred embodiment, the cellulose column is prepared with cellulose (e.g. C6288, sigma) and mixed with a cellulose purification buffer (e.g. 10 mM HEPES (pH 7.2), 0.1 mM EDTA, 125 mM NaCl, and 16% (v/v) ethanol)) and incubated (e.g. at room temperature). In one embodiment, after the cellulose slurry is loaded on an empty column (e.g. spin column or large scale column) the slurry is optionally centrifuged. In one embodiment, the cellulose column is washed before use with a cellulose purification buffer. In one embodiment, a defined amount of RNA, e.g. 450 μg RNA, is added to the column in cellulose purification buffer and incubated (for example at room temperature for about 30 min). In one embodiment, after incubation and/or centrifugation the purified RNA is recovered e.g. as flow-through. In one embodiment, the flow-through is loaded again on a column containing equilibrated cellulose slurry and incubated. In one embodiment purified RNA is recovered as a flow-through and optionally precipitated with sodium acetate and isopropanol. In one embodiment precipitated RNA is recovered by centrifugation and dissolved in nuclease free water.

In a preferred embodiment the step iv) comprises at least one step of core bead chromatography or cor-bead flow through chromatography (e.g. as described in WO2017/182524).

An exemplary core bead flow-through chromatography medium is Capto™ Core (e.g. Capto™ Core 700 beads) from GE Healthcare. Preferably, RNA is selectively recovered from the column in the flow-through. Proteins and short nucleic acids (including dsRNA) are retained in the beads. Flow-through fractions containing RNA may be identified by measuring UV absorption at 260 nm. The composition comprising the RNA is collected in the flow-through is highly purified relative to the preparation before the core bead chromatography step. Multiple eluted fractions containing the RNA may be combined before further treatment. Suitable chromatography setups are known in the art, for example liquid chromatography systems such as the AKTA liquid chromatography systems from GE Healthcare.

In various embodiments, the degree of purity or the amount of full-length RNA may for example be determined by an analytical HPLC, wherein the percentages provided above correspond to the ratio between the area of the peak for the desired RNA and the total area of all peaks in the chromatogram. Alternatively, the degree of purity may be determined by other means for example by an analytical agarose gel electrophoresis or capillary gel electrophoresis.

In preferred embodiments the obtained in vitro transcribed RNA comprising a 3′ terminal A nucleotide has an RNA integrity of at least 60%.

Preferably, the RNA obtained in step iii) has an RNA integrity of at least about 50%, preferably of at least about 60%, more preferably of at least about 70%, most preferably of at least about 80%. Preferably, RNA obtained in step iii) comprises less than about 100 nM divalent cations per g RNA, preferably less than about 50 nM divalent cations Mg2+ and/or Ca2+ per g RNA, more preferably less than about 10 nM divalent cations Mg2+ and/or Ca2+ per g RNA. Preferably, the RNA obtained in step iii) has a purity of more than 75%, 80%, 85%, very particularly 90%, 91%, 92%, 93%, 94%, 95%, 96%, 97%, 98% and most favorably 99% or more. RNA integrity is suitably determined using analytical HPLC, preferably analytical RP-HPLC.

RNA integrity as part of quality controls and may be implemented during or following production of the in vitro transcribed RNA. For RNA mixture based therapeutics it is required that the different components (different RNA molecule species, complexed or free) of the drug product can be characterized, in terms of presence, integrity, ratio and quantity (quality control parameter). Such quality controls may be implemented during or following the RNA sample production, and/or during or following complexation of the RNA sample and/or as a batch release quality control.

The term “RNA integrity” generally describes whether the complete RNA sequence is present in the liquid composition. Low RNA integrity could be due to, amongst others, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, the functionality of the RNA.

The skilled person can choose from a variety of different chromatographic or electrophoretic methods for determining an RNA integrity. Chromatographic and electrophoretic methods are well-known in the art. In case chromatography is used (e.g. RP-HPLC), the analysis of the integrity of the obtained in vitro transcribed RNA comprising a 3′ terminal A nucleotide RNA may be based on determining the peak area (or “area under the peak”) of the full length RNA in a corresponding chromatogram. The peak area may be determined by any suitable software which evaluates the signals of the detector system. The process of determining the peak area is also referred to as integration. The peak area representing the full length RNA is typically set in relation to the peak area of the total RNA in a respective sample. The RNA integrity may be expressed in % RNA integrity.

In the context of the invention, RNA integrity may be determined using analytical (RP)HPLC. Typically, a test sample of the liquid composition comprising lipid based carrier encapsulating RNA may be treated with a detergent (e.g. about 2% Triton X100) to dissociate the lipid based carrier and to release the encapsulated RNA. The released RNA may be captured using suitable binding compounds, e.g. Agencourt AMPure XP beads (Beckman Coulter, Brea, CA, USA) essentially according to the manufacturer's instructions. Following preparation of the RNA sample, analytical (RP)HPLC may be performed to determine the integrity of RNA. Typically, for determining RNA integrity, the RNA samples may be diluted to a concentration of 0.1 g/I using e.g. water for injection (WFI). About 10 μl of the diluted RNA sample may be injected into an HPLC column (e.g. a monolithic poly(styrene-divinylbenzene) matrix). Analytical (RP)HPLC may be performed using standard conditions, for example: Gradient 1: Buffer A (0.1 M TEAA (pH 7.0)); Buffer B (0.1 M TEAA (pH 7.0) containing 25% acetonitrile). Starting at 30% buffer B the gradient extended to 32% buffer B in 2 min, followed by an extension to 55% buffer B over 15 minutes at a flow rate of 1 ml/min. HPLC chromatograms are typically recorded at a wavelength of 260 nm. The obtained chromatograms may be evaluated using a software and the relative peak area may be determined in percent (%) as commonly known in the art. The relative peak area indicates the amount of RNA that has 100% RNA integrity. Since the amount of the RNA injected into the HPLC is typically known, the analysis of the relative peak area provides information on the integrity of the RNA. Thus, if e.g. 100 ng RNA have been injected in total, and 100 ng are determined as the relative peak area, the RNA integrity would be 100%. If, for example, the relative peak area would correspond to 80 ng, the RNA integrity would be 80%. Accordingly, RNA integrity in the context of the invention is determined using analytical HPLC, preferably analytical RP-HPLC.

In some embodiments, the obtained in vitro transcribed RNA comprising a 3′ terminal A nucleotide has an RNA integrity ranging from about 40% to about 100%. In other embodiments, the obtained in vitro transcribed RNA comprising a 3′ terminal A has an RNA integrity ranging from about 50% to about 100%. In embodiments, the RNA has an RNA integrity ranging from about 60% to about 100%. In embodiments, the obtained in vitro transcribed RNA comprising a 3′ terminal A has an RNA integrity ranging from about 70% to about 100%. In other embodiments, the RNA integrity is for example about 50%, about 60%, about 70%, about 80%, or about 90%. RNA integrity is suitably determined using analytical HPLC, preferably analytical RP-HPLC.

In preferred embodiments, the obtained in vitro transcribed RNA comprising a 3′ terminal A nucleotide has an RNA integrity of at least about 50%, preferably of at least about 60%, more preferably of at least about 70%, most preferably of at least about 80%. RNA integrity is suitably determined using analytical HPLC, preferably analytical RP-HPLC.

After step iv) of the purification of the in vitro transcribed RNA comprising a 3′ terminal A nucleotide, the obtained RNA may be adjusted to a desired concentration that is in a range from about 100 μg/ml to about 1 mg/ml.

In embodiments, the adjustment to a concentration is performed using a citrate buffer or an acetate buffer. A suitable citrate buffer or acetate buffer may comprise about 10 mM to about 100 mM citrate or acetate, and may have a PH ranging from about pH 3.0 to about 5.0. A preferred buffer may comprise 50 mM citrate, pH 4.0.

Preferably, an purified RNA solution obtained after step iv) is adjusted to a desired concentration with a citrate buffer to obtain a buffered RNA solution comprising about 100 μg/ml to about 1 mg/ml RNA in a 50 mM citrate buffer pH 4.0.

The step of adjusted to a desired concentration with a citrate buffer or an acetate buffer may comprise at least one step of TFF.

Immunostimulatory Properties

According to the invention the obtained in vitro transcribed RNA comprising a 3′ terminal A nucleotide has reduced immunostimulatory properties compared to a corresponding reference in vitro transcribed RNA not comprising a 3′-terminal A nucleotide.

In this context, it is particularly preferred that the obtained or purified in vitro transcribed RNA comprising a 3′ terminal A nucleotide has at least 10%, 20% or at least 30% lower immunostimulatory properties compared to a corresponding reference in vitro transcribed RNA not comprising a 3′-terminal A nucleotide.

In some preferred embodiments, the obtained or purified in vitro transcribed RNA comprising a 3′ terminal A nucleotide has at least 40%, 50% or at least 60% lower immunostimulatory properties compared to a corresponding reference in vitro transcribed RNA not comprising a 3′-terminal A nucleotide.

A corresponding reference in vitro transcribed RNA not comprising a 3′-terminal A nucleotide is defined as a comparable reference RNA encoding the same amino acid sequence.

In preferred embodiments, the obtained or purified in vitro transcribed RNA is characterized by a lower affinity to a pattern recognition receptor preferably selected from the group consisting of TLR3, TLR7, TLR8, PKR, MDA5, RIG-I, LGP2 or 2′-5-oligoadenylate synthetase compared to a corresponding in vitro transcribed RNA not comprising a 3′ terminal A nucleotide.

The term “Pattern recognition receptor” (PRR) as used throughout the present specification will be recognized and understood by the person of ordinary skill in the art, and is e.g. intended to refer to receptors that are part of the innate immune system. Germline-encoded PRRs are responsible for sensing the presence of microbe-specific molecules (such as bacterial or viral DNA or RNA) via recognition of conserved structures, which are called pathogen-associated molecular patterns (PAMPs). Recent evidence indicates that PRRs are also responsible for recognizing endogenous molecules released from damaged cells, termed damage-associated molecular patterns (DAMPs). Currently, four different classes of PRR families have been identified. These families include transmembrane proteins such as the Toll-like receptors (TLRs) and C-type lectin receptors (CLRs), as well as cytoplasmic proteins such as the Retinoic acid-inducible gene (RIG)-I-like receptors (RLRs) and NOD-like receptors (NLRs). Based on their localization, PRRs may be divided into membrane-bound PRRs and cytoplasmic PRRs and are expressed not only in macrophages and DCs but also in various nonprofessional immune cells. (Takeuchi and Akira 2010. Pattern Recognition Receptors and Inflammation, Cell, Volume 140, ISSUE 6, P805-820).

PRRs can be activated by a broad variety of pathogen associated molecular patterns (PAMPs) for example PAMPs derived from viruses, bacteria, fungi, protozoa, ranging from lipoproteins, carbohydrates, lipopolysaccharides, and various types of nucleic acids (DNA, RNA, dsRNA, non-capped RNA or 5′ ppp RNA). PPRs may be present in different compartments of a cell (e.g. located in the membrane of an endosome or located in the cytoplasm). Upon sensing PAMPs, the PRRs trigger signaling cascades leading inter alia to expression of e.g. cytokines, chemokines. For example, toll like receptor 3 (TLR-3) typically detects long double-stranded RNA (>40 bp) and is also expressed on the surface of certain cell types. The expression of TLR7 in the human immune system is typically restricted to B cells and PDC, TLR8 is preferentially expressed in myeloid immune cells. Consequently, TLR7 ligands drive B cell activation and the production of large amounts of IFNalpha in plasmacytoid dendritic cells (PDC), while TLR8 induces the secretion of high amounts of IL-12p70 in myeloid immune cells. It has been demonstrated in the art that TLR8 selectively detects ssRNA, while TLR7 primarily detects short stretches of dsRNA but can also accommodate certain ssRNA oligonucleotides. TLR9 receptors are predominantly expressed in human B cells and plasmacytoid dendritic cells and detect single-stranded DNA containing unmethylated CpG dinucleotides. Additionally to the induction of cytokines, some RNA sensing pattern recognition receptors of the innate immune system can inhibit protein translation upon binding of its agonist (e.g. dsRNA, 5′ ppp RNA), such as e.g. PKR and OAS1. For example, binding of a long double-stranded RNA is taught to activate PKR to phosphorylate elF2a leading to inhibition of translation of an mRNA molecule. IFIT1 and IFIT5 is taught to bind to 5′ ppp RNA leads to a blockade of elF2a, thereby inhibiting translation of an mRNA molecule (reviewed in Hartmann, G. “Nucleic acid immunity.” Advances in immunology. Vol. 133. Academic Press, 2017. 121-169).

Typical Pattern recognition receptor” (PRR) in the context of the invention are Toll-like receptors, NOD-like receptors, RIG-I like receptors, PKR, OAS1, IFIT1 and IFIT5.

In preferred embodiments the immunostimulatory properties are defined as the induction of an innate immune response which is determined by measuring the induction of cytokines.

The term “innate immune system”, also known as non-specific (or unspecific) immune system, as used throughout the present specification will be recognized and understood by the person of ordinary skill in the art, and is e.g. intended to refer to a system that typically comprises the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be, e.g., activated by ligands (e.g. PAMPs) of “Pattern recognition receptors” (PRR) or other auxiliary substances such as lipopolysaccharides, TNFalpha, CD40 ligand, or cytokines, monokines, lymphokines, interleukins or chemokines, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFNalpha, IFNbeta, IFNgamma, GM-CSF, G-CSF, M-CSF, LTbeta, TNFalpha, growth factors, and hGH, a ligand of human Toll-like receptor TLR1, TLR2, TLR3, TLR4, TLR5, TLR6, TLR7, TLR8, TLR9, TLR10, a ligand of murine Toll-like receptor TLR1, TLR2, TLR3, TLR4, TLR5, TLR6, TLR7, TLR8, TLR9, TLR10, TLR11, TLR12 or TLR13, a ligand of a NOD-like receptor, a ligand of a RIG-I like receptor, an immunostimulatory nucleic acid, an immunostimulatory RNA (isRNA), a CpG-DNA, an antibacterial agent, an anti-viral agent, a ligand of PKR and OAS1 (e.g. long double stranded RNA) or a ligand of IFIT1 and IFIT5 (5′ ppp RNA).

Typically, a response of the innate immune system (after e.g. sensing an RNA) includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system; and/or acting as a physical and chemical barrier to infectious agents. Typically, protein synthesis is also reduced during the innate immune response. The inflammatory response is orchestrated by pro-inflammatory cytokines such as tumor necrosis factor (TNF), interleukin (IL)-1, and IL-6. These cytokines are pleiotropic proteins that regulate the cell death of inflammatory tissues, modify vascular endothelial permeability, recruit blood cells to inflamed tissues and induce the production of acute-phase proteins.

Accordingly the induction of an innate immune response may be determined by measuring the induction of cytokines.

In particular embodiments the cytokines are selected from the group consisting of IFNalpha (IFNa), TNFalpha (TNFα), IP-10, IFNgamma (IFNγ), IL-6, IL-12, IL-8, MIG, Rantes, MIP-1alpha (MIP1α), MIP-1beta (MIP1β), McP1, or IFNbeta (IFNβ).

The induction or activation or stimulation of an innate immune response as described above is usually determined by measuring the induction of cytokines.

Preferably, a reduction of the immunostimulatory properties is characterized by a reduced level of at least one cytokine preferably selected from Rantes, MIP-1 alpha, IP-10, MIP-1 beta, McP1, TNFalpha, IFNgamma, IFNalpha, IFNbeta, IL-12, IL-6, or IL-8.

The term “reduced level of at least one cytokine” has to be understood as that the administration of the in vitro transcribed RNA according to the invention reduces the induction of cytokines compared to a control (e.g. corresponding reference in vitro transcribed RNA not comprising a 3′ terminal A nucleotide) to a certain percentage.

Accordingly, reduction of the immunostimulatory properties in the context of the invention is characterized by a reduced level of at least one cytokine preferably selected from Rantes, MIP-1 alpha, MIP-1 beta, McP1, TNFalpha, IFNgamma, IFNalpha, IFNbeta, IL-12, IL-6, or IL-8, wherein the reduced level of at least one cytokine is a reduction of at least 10%, 15%, 20%, 25%, 30%, 35%, 40%, 45%, 50%, 55%, 60%, 65%, 70%, 75%, 80%, 85%, 90%, or 95%. Preferably, the reduced level of at least one cytokine is a reduction of at least 30%.

Methods to evaluate the (innate) immune stimulation (that is, the induction of e.g. Rantes, MIP-1 alpha, MIP-1 beta, IP-10, McP1, TNFalpha, IFNgamma, IFNalpha, IFNbeta, IL-12, IL-6, or IL-8) by the in vitro transcribed RNA comprising a 3′ terminal A nucleotide in specific cells/organs/tissues are well known in the art for the skilled artisan.

Methods to evaluate the reduction of immunostimulatory properties (that is, the reduction of the (innate) immune response of e.g. Rantes, MIP-1 alpha, MIP-1 beta, McP1, IP-10, TNFalpha, IFNgamma, IFNalpha, IFNbeta, IL-12, IL-6, or IL-8) by the in vitro transcribed RNA comprising a 3′ terminal A nucleotide in specific cells/organs/tissues are well known in the art for the skilled artisan. Typically, the (innate) immune stimulation of the in vitro transcribed RNA comprising a 3′ terminal A nucleotide is compared with a corresponding reference in vitro transcribed RNA not comprising a 3′-terminal A nucleotide. The same conditions (e.g. the same cell lines, same organism, same application route, the same detection method, the same amount of in vitro transcribed RNA, the same RNA sequence etc.) have to be used (if feasible) to allow a valid comparison. The person of skill in the art understands how to perform a comparison of the inventive in vitro transcribed RNA comprising a 3′ terminal A nucleotide and a respective corresponding reference in vitro transcribed RNA not comprising a 3′-terminal A nucleotide.

In some embodiments the induction of cytokines is measured by administration of the obtained in vitro transcribed RNA to cells, a tissue or an organism, preferably hPBMCs, Hela cells or HEK cells.

In the context of the invention, the induction of cytokines is measured after administration of the obtained in vitro transcribed RNA to cells, a tissue or an organism, preferably hPBMCs, Hela cells or HEK cells. Preferred in that context are hPBMCs. Upon administration of the obtained in vitro transcribed RNA (or the corresponding control) to hPBMCs, Hela cells or HEK cells, an assay for measuring cytokine levels is performed. Cytokines secreted into culture media or supernatants can be quantified by techniques such as bead based cytokine assays (e.g. cytometric bead array (CBA), ELISA, FACS, quantitative mass spectrometry and Western blot).

In detail, such a step comprises the e.g. in vitro sub-steps of transfecting competent cells, e.g. PBMCs, with the obtained in vitro transcribed RNA according to one or more embodiments of the inventive method, cultivating the cells, e.g. for 8 h-24 h, preferably for 12 h-48 h, preferably for 18 h-24 h, preferably for 24-48 h, preferably for at least 12 hours, preferably for at least 18 h, more preferably for at least 20 h and determining the amount of pro-inflammatory cytokines in the cell supernatant. The amount of pro-inflammatory cytokines present in the supernatant of the cells transfected with the obtained in vitro transcribed RNA according to the invention is compared to the amount of pro-inflammatory cytokines present in the supernatant of cells transfected with the corresponding reference in vitro transcribed RNA not comprising a 3-terminal A nucleotide. Appropriate techniques for determining the immunogenicity and/or immunostimulatory capacity of a nucleic acid, such as that of e.g. the obtained in vitro transcribed RNA, are known in the art and are readily available to the skilled person (Robbins et al., 2009. Oligonucleotides 19(2):89-102). The nature and the extent of the cytokine response to RNA depends on several factors including timing, cell type, delivery vehicle and route of administration. The absence of immunostimulation at a single time point for a single cytokine does not necessarily demonstrate the absence of immunostimulation in general, such that assessment of several cytokine responses at multiple time points may be required. Antibodies and ELISA kits for the determination of interferons (e.g. IFNalpha and IFN) and a variety of pro-inflammatory cytokines, such as e.g. TNFalpha, TGFbeta, IL-1 and IL-6, are commercially available.

If it were desired to carry out in vivo studies for testing for IFNalpha and/or suitable pro-inflammatory cytokines, such as e.g. TNFalpha and IL-6, their presence in the plasma of treated animals can be used to monitor the systemic activation of the immune response. Measurement of the immune response at an appropriate time point after RNA administration is critical for a valid assessment. Systemic administration of RNA formulations to mice leads to detectable elevations of serum cytokines within 1 to 2 hours, depending on the type of delivery vehicle and the cytokine of interest. Typically, the increase of cytokine levels in the serum is transient and may decrease again after 12 to 24 hours of treatment. For example, mice can be injected with complexed obtained in vitro transcribed RNA and serum levels of, e.g., IFNalpha, TNFalpha and IL-6 may be measured 6 hours post injection by using suitable ELISA assays (Kariko et al., 2012. Mol. Ther. 20(5):948-53).

According to the invention the administration of the obtained or purified in vitro transcribed RNA to a cell, tissue, or organism results in a reduction of the immunostimulatory properties as compared to administration of the corresponding reference in vitro transcribed RNA not comprising a 3′ terminal A nucleotide.

Preferably, a bead based cytokine assays, most preferably a cytometric bead array (CBA) is performed to measure the induction of cytokines in cells after administration of the obtained in vitro transcribed RNA comprising a 3′ terminal A nucleotide (and their corresponding controls, in this case the corresponding reference in vitro transcribed RNA not comprising a 3′ terminal A nucleotide).

CBA can quantify multiple cytokines from the same sample. The CBA system uses a broad range of fluorescence detection offered by flow cytometry and antibody-coated beads to capture cytokines. Each bead in the array has a unique fluorescence intensity so that beads can be mixed and acquired simultaneously. A suitable CBA assay in that context is described in a BD Bioscience application note of 2012, “Quantification of Cytokines Using BD™ Cytometric Bead Array on the BD™ FACSVerse System and Analysis in FCAP Array™ Software”, from Reynolds et al. An exemplary CBA assay for determining cytokine levels is described in the examples section of the present invention.

In a preferred embodiment the obtained or purified in vitro transcribed RNA comprising a 3′ terminal A nucleotide is more stable and/or the optionally encoded peptide or protein is more efficiently expressed compared to a corresponding reference in vitro transcribed RNA not comprising a 3′-terminal A nucleotide.

A more stable and/or more efficiently expression as described herein has to be understood as the additional duration of protein expression wherein expression of the obtained in vitro transcribed RNA comprising a 3′ terminal A nucleotide is still detectable in comparison to a corresponding control (in vitro transcribed RNA not comprising a 3′ terminal A nucleotide) which can be determined by various well-established expression assays (e.g. antibody-based detection methods) as described above.

Accordingly, administration of the in vitro transcribed RNA comprising a 3′ terminal A nucleotide to a cell, tissue, or organism results in a prolonged protein expression compared to administration of the corresponding in vitro transcribed RNA not comprising a 3′ terminal A nucleotide, wherein the additional duration of protein expression in said cell, tissue, or organism is at least 5 h, 10 h, 20 h, 25 h, 30 h, 35 h, 40 h, 45 h, 50 h, 55 h, 60 h, 65 h, 70 h, 75 h, 80 h, 85 h, 90 h, 95 h, or 10 h or even longer.

Methods to evaluate the expression (that is, protein expression) of the in vitro transcribed RNA comprising a 3′ terminal A nucleotide in specific cells/organs/tissues, and methods to determine the duration of expression are well known in the art for the skilled artisan. For example, protein expression can be determined using antibody-based detection methods (western blots, FACS, ELISA, cytometric bead array (CBA)) or quantitative mass spectrometry. Exemplary methods are provided in the examples section. The same conditions (e.g. the same cell lines, same organism, same application route, the same detection method, the same amount of therapeutic RNA, the same RNA sequence) have to be used (if feasible) to allow a valid comparison. The person of skill in the art understands how to perform a comparison of the inventive combination and a respective reference or control RNA (e.g. in vitro transcribed RNA not comprising a 3′ terminal A nucleotide).

The “more efficiently expressed” in vitro transcribed RNA comprising a 3′ terminal A nucleotide of the invention has to be understood as percentage increase of expression compared to a corresponding control (in vitro transcribed RNA not comprising a 3′ terminal A nucleotide) which can be determined by various well-established expression assays (e.g. antibody-based detection methods) as described above.

Accordingly, administration of the obtained or purified in vitro transcribed RNA comprising a 3′ terminal A nucleotide to a cell, tissue, or organism results in an increased expression as compared to administration of the corresponding reference in vitro transcribed RNA not comprising a 3′ terminal A nucleotide, wherein the percentage increase in expression in said cell, tissue, or organism is at least 10%, 20%, 30%, 40%, 50%, 60%, 70%, 80%, 90%, 100%, 200%, 300%, 400%, 500% or even more.

By the method according to the invention, an optimum of increased RNA expression on the one hand and reduced immunostimulatory properties on the other hand is achieved. The level of cytokine expression (secretion), e.g. TNFalpha and IFNalpha (e.g. by PBMCs) is reduced by at least 10%, at least 20%, preferably by at least 40%, as compared to the immunostimulatory properties of a corresponding reference in vitro transcribed RNA not comprising a 3′ terminal A nucleotide immune response triggered by the wild type or reference equivalent. Such a reduction is measurable under in vivo and in vitro conditions.

v) Formulation of the Obtained In Vitro Transcribed RNA

In preferred embodiments the method according to this invention comprises a further step v) formulating the obtained in vitro transcribed RNA with a cationic compound to obtain an RNA formulation.

Accordingly, the method of reducing the immunostimulatory properties of an in vitro transcribed RNA by producing the in vitro transcribed RNA comprises the following steps:
i) providing a linear DNA template comprising a template DNA strand encoding the RNA, wherein the template DNA strand comprises a 5′ terminal T nucleotide;
ii) incubating the linear DNA template under conditions to allow (run-off) RNA in vitro transcription; iii) obtaining the in vitro transcribed RNA comprising a 3′ terminal A nucleotide.
iv) purifying the obtained in vitro transcribed RNA after RNA in vitro transcription
v) formulating the obtained in vitro transcribed RNA with a cationic compound to obtain an RNA formulation.

Hereby, possible formulations are described below.

Accordingly, unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”.

In preferred embodiments the cationic compound comprises one or more lipids suitable to form liposomes, lipid nanoparticles (LNP), lipoplexes, and/or nanoliposomes.

Therefore the obtained in vitro transcribed RNA comprising a 3′ terminal A nucleotide may be provided in the form of a lipid-based formulation, in particular in the form of liposomes, lipoplexes, and/or lipid nanoparticles comprising said vitro transcribed RNA.

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of RNA. For example, a liposome, a lipid complex, a lipoplex and the like are within the scope of a lipid nanoparticle (LNP).

LNPs may include any cationic lipid suitable for forming a lipid nanoparticle. Preferably, the cationic lipid carries a net positive charge at about physiological pH.

Vi) Purification Step after Formulating the Obtained In Vitro Transcribed RNA

In preferred embodiments, the method according to this invention comprises a further step vi) comprising a purification step after formulating the obtained in vitro transcribed RNA comprising a 3′ terminal A nucleotide.

Accordingly, the method of reducing the immunostimulatory properties of an in vitro transcribed RNA by producing the in vitro transcribed RNA comprises the following steps:
i) providing a linear DNA template comprising a template DNA strand encoding the RNA, wherein the template DNA strand comprises a 5′ terminal T nucleotide;
ii) incubating the linear DNA template under conditions to allow (run-off) RNA in vitro transcription;
iii) obtaining the in vitro transcribed RNA comprising a 3′ terminal A nucleotide.
iv) purifying the obtained in vitro transcribed RNA after RNA in vitro transcription
v) formulating the obtained in vitro transcribed RNA with a cationic compound to obtain an RNA formulation.
vi) purifying the obtained in vitro transcribed RNA after formulating

In step vi) of the method according to this invention the obtained in vitro transcribed RNA may be purified after the formulation as described in step v). Thus the formulated in vitro transcribed RNA is purified and/or clarifying and/or concentrated.

In a preferred embodiment, step vi) comprises a step of concentrating the composition comprises lipid-based carries encapsulating an RNA by tangential flow filtration (TFF; ultrafiltration). Preferably, the concentrating step is performed until a desired concentration is achieved.

In a preferred embodiment, step vi) comprises a step of buffer exchange. In embodiments, the non-purified composition comprises lipid-based carries encapsulating an RNA is in a buffer comprising citrate/ethanol. The step of buffer exchange is performed by tangential flow filtration to exchange the buffer to a suitable storage buffer.

Suitably, the storage buffer comprises a sugar, preferably a disaccharide. In embodiments, the concentration of the sugar is in a range from about 50 mM to about 300 mM, preferably about 150 mM. In embodiments, the sugar comprised in the composition is sucrose, preferably in a concentration of about 150 mM.

Suitably, the storage buffer comprises a salt, preferably NaCl. In embodiments, the concentration of the salt comprised in the composition is in a range from about 10 mM to about 200 mM, preferably about 75 mM. In embodiments, the salt comprised in composition is NaCl, preferably in a concentration of about 75 mM.

Suitably, the storage buffer comprises a buffering agent, preferably selected from Tris, HEPES, NaPO4 or combinations thereof. In embodiments, the buffering agent is in a concentration ranging from about 1 mM to about 100 mM. In embodiments, the buffering agent is NaPO4, preferably in a concentration of about 10 mM.

Suitably, the storage and/or administration buffer has a pH in a range of about pH 7.0 to about pH 8.0. In preferred embodiments, the composition has a pH of about pH 7.4.

In preferred embodiments, step vi) comprises a step of buffer exchange/conditioning to a storage buffer comprising 150 mM sucrose/75 mM sodium chloride/10 mM sodium phosphate; pH 7.4) via diafiltration and/or TFF.

In a preferred embodiments, step vi) comprises a step of clarifying filtration, preferably prior to the TFF purification steps. The step of clarifying filtration is suitably performed using a dual membrane filter cartridge (0.45 μm and 0.22 μm pore size).

In embodiments where polyadenylated RNA is to be produced, the poly(A) sequence of the RNA is preferably obtained from a linear DNA template during RNA in vitro transcription in step ii). In other embodiments, poly(A) sequences are generated by enzymatic polyadenylation of the RNA (after RNA in vitro transcription in step ii) using commercially available polyadenylation kits and corresponding protocols known in the art, or alternatively, by using immobilized poly(A)polymerases e.g. using a methods and means as described in WO2016/174271.

In embodiments, the capping degree of the RNA may be determined using capping assays as described in published PCT application WO2015/101416, in particular, as described in Claims 27 to 46 of published PCT application WO2015/101416 can be used. Alternatively, a capping assay described in published PCT application WO2020127959 may be used, in particular, as described in Claims 1 to 54 of published PCT application WO2020127959. The disclosure relating to respective capping assays provided in WO2015/101416 or WO2020127959 is herewith incorporated by reference.

It may be required to provide GMP-grade RNA using a manufacturing process approved by regulatory authorities. Accordingly, in a particularly preferred embodiment, the method of manufacturing is performed under current good manufacturing practice (GMP), implementing various quality control steps on DNA and RNA level, preferably according to WO2016/180430. In preferred embodiments, the lipid-based carrier encapsulating the RNA obtained by the method of manufacturing is a GMP-grade lipid-based carrier encapsulating the RNA.

Second Aspect: In Vitro Transcribed RNA Comprising a 3′ Terminal a Nucleotide

In the following, advantageous embodiments and features of the in vitro transcribed RNA comprising a 3′ terminal A nucleotide obtained by the method of the first aspect are described. Notably, all described embodiments and features of said in vitro transcribed RNA comprising a 3′ terminal A nucleotide that are described in the context of the inventive method of producing an in vitro transcribed RNA with reduced immunostimulatory properties (first aspect) are likewise be applicable to the to the in vitro transcribed RNA comprising a 3′ terminal A nucleotide (second aspect).

Additionally they are likewise applicable to the in vitro transcribed RNA comprising a 3 terminal A nucleotide of the pharmaceutical composition (third aspect), or the kit or kit of parts (fourth aspect), and to further aspects of the invention.

According to the second aspect of this invention an in vitro transcribed RNA comprising a 3′ terminal A nucleotide having reduced immunostimulatory properties is obtainable by the method according to this invention.

The term “RNA” will be recognized and understood by the person of ordinary skill in the art, and is e.g. intended to be a ribonucleic acid molecule, i.e. a polymer consisting of nucleotides. These nucleotides are usually adenosine-monophosphate, uridine-monophosphate, guanosine-monophosphate and cytidine-monophosphate monomers which are connected to each other along a so-called backbone. The backbone is typically formed by phosphodiester bonds between the sugar, i.e. ribose, of a first monomer and a phosphate moiety of a second, adjacent monomer. The specific succession of monomers is called the RNA-sequence.

In various embodiments, the in vitro transcribed RNA comprising a 3′ terminal A is selected from a coding RNA, a non-coding RNA, a circular RNA (circRNA), an RNA oligonucleotide, a small interfering RNA (siRNA), a small hairpin RNA (shRNA), an antisense RNA (asRNA), a CRISPR/Cas9 guide RNAs, an mRNA, a riboswitch, an immunostimulating RNA (isRNA), a ribozyme, an RNA aptamer, a ribosomal RNA (rRNA), a transfer RNA (tRNA), a viral RNA (vRNA), a retroviral RNA, a small nuclear RNA (snRNA), a self-replicating RNA, a replicon RNA, a small nucleolar RNA (snoRNA), a microRNA (miRNA), and a Piwi-interacting RNA (piRNA).

In embodiments, the in vitro transcribed RNA comprising a 3′ terminal A is a non-coding RNA preferably selected from RNA oligonucleotide, a small interfering RNA (siRNA), a small hairpin RNA (shRNA), an antisense RNA (asRNA), a CRISPR/Cas9 guide RNAs, a riboswitch, a ribozyme, an RNA aptamer, a ribosomal RNA (rRNA), a transfer RNA (tRNA), a small nuclear RNA (snRNA), a small nucleolar RNA (snoRNA), a microRNA (miRNA), and a Piwi-interacting RNA (piRNA).

As used herein, the term “guide RNA” (gRNA) relates to any RNA molecule capable of targeting a CRISPR-associated protein/CRISPR-associated endonuclease to a target DNA sequence of interest. In the context of the invention, the term guide RNA has to be understood in its broadest sense, and may comprise two-molecule gRNAs (“tracrRNA/crRNA”) comprising crRNA (“CRISPR RNA” or “targeter-RNA” or “crRNA” or “crRNA repeat”) and a corresponding tracrRNA (“trans-acting CRISPR RNA” or “activator-RNA” or “tracrRNA”) molecule, or single-molecule gRNAs. A “sgRNA” typically comprises a crRNA connected at its 3′ end to the 5′ end of a tracrRNA through a “loop” sequence. In the context of the invention, a guide RNA may be provided by the at least one therapeutic RNA of the inventive combination/composition.

An in vitro transcribed RNA is an RNA, which has been prepared by the process of in vitro transcription. Briefly, an in vitro transcribed RNA is an RNA molecule that has been synthesized from a template DNA, commonly a linearized and purified plasmid template DNA, a PCR product, or a polynucleotide/oligonucleotide. In vitro transcription requires a purified linear DNA template containing an RNA polymerase promoter, ribonucleoside triphosphates or nucleotides, a buffer system and magnesium ions, and an appropriate RNA polymerase. The exact conditions used in the transcription reaction depend on the amount of RNA needed for a specific application. Basic laboratory protocols for in vitro transcription, as well as, commercial kits can be used in order to synthesize nucleic acid, for example RNA. Commercial kits for synthesizing RNA can include, for example, MEGA script© Kits and TranscriptAid T7 High Yield Transcription Kit (Thermo Scientific), HiScribe™ T7 and MiniV™ In Vitro Transcription Kit (Epicentre or NEB). Other transcription kits can be used for making RNA and are known to those skilled in the art. RNA synthesis occurs in a cell free (“in vitro”) system catalyzed by DNA dependent RNA polymerases. According to this invention the in vitro transcribed RNA comprising a 3′ terminal A nucleotide has reduced immunostimulatory properties compared to a corresponding RNA not comprising a 3′ terminal A nucleotide.

Preferably, the vitro transcribed RNA comprising a 3′ terminal A nucleotide comprises a length of about 50 to about 20000, or 100 to about 20000 nucleotides, preferably of about 250 to about 20000 nucleotides, more preferably of about 500 to about 10000, even more preferably of about 500 to about 5000.

In particularly preferred embodiments the in vitro transcribed RNA comprising a 3′ terminal A nucleotide is an mRNA, most preferred a coding mRNA.

A typical mRNA (messenger RNA) in the context of the invention provides the coding sequence that is translated into an amino-acid sequence of a peptide or protein after e.g. in vivo administration to a cell.

Accordingly, in preferred embodiments, the in vitro transcribed RNA comprising a 3′ terminal A nucleotide, is an mRNA, wherein the in vitro transcribed RNA is obtainable by RNA in vitro transcription using a sequence optimized nucleotide mixture.

In preferred embodiments, the in vitro transcribed RNA comprising a 3′ terminal A nucleotide, e.g. the coding RNA or the mRNA, comprises at least one coding sequence (cds) encoding at least one peptide or protein.

Advantageously, the expression of the encoded at least one peptide or protein of the vitro transcribed RNA comprising a 3′ terminal A is increased or prolonged upon administration into cells, a tissue or an organism compared to the expression of the encoded at least one peptide or protein of the vitro transcribed RNA not comprising a 3′ terminal A nucleotide.

Methods to evaluate the expression (that is, protein expression) of the therapeutic RNA in specific cells/organs/tissues, and methods to determine the duration of expression are well known in the art for the skilled artisan. For example, protein expression can be determined using antibody-based detection methods (western blots, FACS) or quantitative mass spectrometry. Exemplary methods are provided in the examples section. The same conditions (e.g. the same cell lines, same organism, same application route, the same detection method, the same amount of the corresponding in vitro transcribed RNA not comprising a 3′ terminal A nucleotide.

According to the invention, the in vitro transcribed RNA comprising a 3′ terminal A nucleotide leads to a reduction of the immunostimulatory properties upon administration to a subject and/or cell. Accordingly, the immune response of a subject and/or cell is reduced upon administration of the in vitro transcribed RNA comprising a 3′ terminal A nucleotide to a subject and/or cell.

In this context, it is particularly preferred that the innate immune response upon administration of the in vitro transcribed RNA comprising a 3′ terminal A nucleotide to a subject and/or cell is at least 10%, 20% or at least 30% reduced compared to the innate immune response upon administration of the corresponding reference in vitro transcribed RNA not comprising a 3′-terminal A nucleotide.

In other preferred embodiments the innate immune response upon administration of the in vitro transcribed RNA comprising a 3′ terminal A nucleotide to a subject and/or cell is at least 40%, 50% or at least 60% reduced compared to the innate immune response upon administration of the corresponding reference in vitro transcribed RNA not comprising a 3′-terminal A nucleotide.

Third Aspect: Pharmaceutical Composition

In the following, advantageous embodiments and features regarding the formulation/complexation of the obtained in vitro transcribed RNA comprising a 3′ terminal A nucleotide are described. All described embodiments and features regarding formulation in the context of the inventive method of producing an in vitro transcribed RNA with reduced immunostimulatory properties (first aspect) are likewise be applicable to the “in vitro transcribed RNA comprising a 3′ terminal A nucleotide” (second aspect). Additionally, they are likewise applicable to the in vitro transcribed RNA comprising a 3′ terminal A nucleotide of the pharmaceutical composition (third aspect), or the kit or kit of parts (fourth aspect), and to further aspects of the invention.

In a third aspect the present invention provides a pharmaceutical composition comprising the in vitro transcribed RNA comprising a 3′ terminal A nucleotide as defined herein or a composition obtained by the method according to this invention optionally comprising one or more pharmaceutically acceptable excipients, carriers, diluents and/or vehicles.

In the context of the invention, a “composition” refers to any type of composition in which the specified ingredients (e.g. in vitro transcribed RNA comprising a 3′ terminal A nucleotide, e.g. in association with a polymeric carrier or LNP), may be incorporated, optionally along with any further constituents, usually with at least one pharmaceutically acceptable carrier or excipient. The composition may be a dry composition such as a powder or granules, or a solid unit such as a lyophilized form. Alternatively, the composition may be in liquid form, and each constituent may be independently incorporated in dissolved or dispersed (e.g. suspended or emulsified) form.

The term “pharmaceutically acceptable carrier” or “pharmaceutically acceptable excipient” as used herein preferably includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or preferably a buffer, more preferably an aqueous buffer, may be used, containing a sodium salt, preferably at least 50 mM of a sodium salt, a calcium salt, preferably at least 0.01 mM of a calcium salt, and optionally a potassium salt, preferably at least 3 mM of a potassium salt. According to preferred embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, Nal, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂, Cal₂, CaBr₂, CaCO₃, CaSO₄, Ca(OH)₂.

Furthermore, organic anions of the aforementioned cations may be in the buffer. Accordingly, in embodiments, the nucleic acid composition may comprise pharmaceutically acceptable carriers or excipients using one or more pharmaceutically acceptable carriers or excipients to e.g. increase stability, increase cell transfection, permit the sustained or delayed, increase the translation of encoded protein in vivo, and/or alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one nucleic acid and, optionally, a plurality of nucleic acids of the composition, in such a manner that no interaction occurs, which would substantially reduce the biological activity or the pharmaceutical effectiveness of the composition under typical use conditions (e.g., intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The pharmaceutical composition suitably comprises a safe and effective amount of the in vitro transcribed RNA comprising a 3′ terminal A nucleotide as specified herein. As used herein, “safe and effective amount” means an amount of the therapeutic RNA, preferably the mRNA, sufficient to result in expression and/or activity of the encoded protein after administration. At the same time, a “safe and effective amount” is small enough to avoid serious side-effects caused by administration of said in vitro transcribed RNA comprising a 3′ terminal A nucleotide.

Further advantageous embodiments and features of the pharmaceutical composition of the invention are described below. Notably, embodiments and features described in the context of the pharmaceutical composition may likewise be applicable to the kit or kit of parts of the fourth aspect.

Pharmaceutical compositions of the present invention may suitably be sterile and/or pyrogen-free.

The choice of a pharmaceutically acceptable carrier as described above is determined in particular by the mode in which the pharmaceutical composition according to the invention is administered.

In a preferred embodiment the pharmaceutical composition does not comprises an adjuvant.

The term “adjuvant” as used herein will be recognized and understood by the person of ordinary skill in the art, and is for example intended to refer to a pharmacological and/or immunological agent that may modify, e.g. enhance, the effect of other agents (herein: the effect of the in vitro transcribed RNA comprising a 3′ terminal A nucleotide). The term “adjuvant” refers to a broad spectrum of substances. Typically, these substances are able to increase the immunogenicity of antigens. For example, adjuvants may be recognized by the innate immune systems and, e.g., may elicit an innate immune response (that is, a non-specific immune response). “Adjuvants” typically do not elicit an adaptive immune response.

Cationic or Polycationic Peptides and Polymeric Carrier

In preferred embodiments, the in vitro transcribed RNA comprising a 3′ terminal A nucleotide is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof.

Accordingly, the in vitro transcribed RNA comprising a 3′ terminal A nucleotide as defined herein is attached to one or more cationic or polycationic compounds, preferably cationic or polycationic polymers, cationic or polycationic polysaccharide, cationic or polycationic lipid, cationic or polycationic protein, or cationic or polycationic peptide, or any combinations thereof.

The term “cationic or polycationic compound” as used herein will be recognized and understood by the person of ordinary skill in the art, and are for example intended to refer to a charged molecule, which is positively charged at a pH value ranging from about 1 to 9, at a pH value ranging from about 3 to 8, at a pH value ranging from about 4 to 8, at a pH value ranging from about 5 to 8, more preferably at a pH value ranging from about 6 to 8, even more preferably at a pH value ranging from about 7 to 8, most preferably at a physiological pH, e.g. ranging from about 7.2 to about 7.5. Accordingly, a cationic component, e.g. a cationic peptide, cationic protein, cationic polymer, cationic polysaccharide, cationic lipid may be any positively charged compound or polymer which is positively charged under physiological conditions. A “cationic or polycationic peptide or protein” may contain at least one positively charged amino acid, or more than one positively charged amino acid, e.g. selected from Arg, His, Lys or Orn. Accordingly, “polycationic” components are also within the scope exhibiting more than one positive charge under the given conditions.

Cationic or polycationic compounds, being particularly preferred in this context may be selected from the following list of cationic or polycationic peptides or proteins of fragments thereof: protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides, pAntp, plsl, FGF, Lactoferrin, Transportan, Buforin-2, Bac715-24, SynB, SynB(1), pVEC, hCT-derived peptides, SAP, or histones. More preferably, the coding RNA, preferably the mRNA, is complexed with one or more polycations, preferably with protamine or oligofectamine, most preferably with protamine.

Further preferred cationic or polycationic compounds, which can be used as transfection or complexation agent may include cationic polysaccharides, for example chitosan, polybrene etc.; cationic lipids, e.g. DOTMA, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS, DIMRI, DOTAP, DC-6-14, CLIP1, CLIP6, CLIP9, oligofectamine; or cationic or polycationic polymers, e.g. modified polyaminoacids, such as beta-aminoacid-polymers or reversed polyamides, etc., modified polyethylenes, such as PVP etc., modified acrylates, such as pDMAEMA etc., modified amidoamines such as pAMAM etc., modified polybetaaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI, poly(propyleneimine), etc., polyallylamine, sugar backbone based polymers, such as cyclodextrin based polymers, dextran based polymers, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole); etc.

In this context it is particularly preferred that the in vitro transcribed RNA comprising a 3′ terminal A nucleotide is complexed or at least partially complexed with a cationic or polycationic compound and/or a polymeric carrier, preferably cationic proteins or peptides. In this context, the disclosure of WO2010/037539 and WO2012/113513 is incorporated herewith by reference. Partially means that only a part of the nucleic acid is complexed with a cationic compound and that the rest of the nucleic acid is in uncomplexed form (“free”).

Further preferred cationic or polycationic proteins or peptides that may be used for complexation can be derived from formula (Arg)l;(Lys)m;(His)n;(Orn)o;(Xaa)x of the patent application WO2009/030481 or WO2011/026641, the disclosure of WO2009/030481 or WO2011/026641 relating thereto incorporated herewith by reference.

In one embodiment the N/P ratio of the in vitro transcribed RNA comprising a 3′ terminal A nucleotide to the one or more cationic or polycationic compound is in the range of about 0.1 to 10, including a range of about 0.3 to 4, of about 0.5 to 2, of about 0.7 to 2 and of about 0.7 to 1.5.

In some embodiments, the at least one in vitro transcribed RNA comprising a 3′ terminal A nucleotide is complexed, or at least partially complexed, with at least one cationic or polycationic proteins or peptides preferably selected from SEQ ID NOs: 93-97, or any combinations thereof.

In various embodiments, the one or more cationic or polycationic peptides are selected from SEQ ID NOs: 93-97, or any combinations thereof.

Accordingly, in preferred embodiments, the in vitro transcribed RNA comprising a 3′ terminal A nucleotide, is complexed or associated with or at least partially complexed or partially associated with one or more cationic or polycationic peptides selected from SEQ ID NOs: 93-97, or any combinations thereof.

Accordingly, in preferred embodiments, the in vitro transcribed RNA comprising a 3′ terminal A nucleotide is complexed or associated with or at least partially complexed or partially associated with one or more cationic or polycationic peptides selected from SEQ ID NOs: 93-97, or any combinations thereof.

In embodiments, the in vitro transcribed RNA comprising a 3′ terminal A nucleotide as defined herein is complexed or associated with or at least partially complexed or partially associated with one or more cationic or polycationic polymer.

In embodiments, the in vitro transcribed RNA comprising a 3′ terminal A nucleotide is complexed or associated with or at least partially complexed or partially associated with one or more cationic or polycationic polymer.

Accordingly, in embodiments, the in vitro transcribed RNA comprising a 3′ terminal A nucleotide comprises at least one polymeric carrier.

The term “polymeric carrier” as used herein will be recognized and understood by the person of ordinary skill in the art, and are e.g. intended to refer to a compound that facilitates transport and/or complexation of another compound (e.g. cargo nucleic acid). A polymeric carrier is typically a carrier that is formed of a polymer. A polymeric carrier may be associated to its cargo (e.g. DNA, or RNA) by covalent or non-covalent interaction. A polymer may be based on different subunits, such as a copolymer.

Suitable polymeric carriers in that context may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PEGylated PLL and polyethylenimine (PEI), dithiobis(succinimidylpropionate) (DSP), Dimethyl-3,3′-dithiobispropionimidate (DTBP), poly(ethylene imine) biscarbamate (PEIC), poly(L-lysine) (PLL), histidine modified PLL, poly(N-vinylpyrrolidone) (PVP), poly(propylenimine (PPI), poly(amidoamine) (PAMAM), poly(amido ethylenimine) (SS-PAEI), triehtylenetetramine (TETA), poly(β-aminoester), poly(4-hydroxy-L-proine ester) (PHP), poly(allylamine), poly(α-[4-aminobutyl]-L-glycolic acid (PAGA), Poly(D,L-lactic-co-glycolid acid (PLGA), Poly(N-ethyl-4-vinylpyridinium bromide), poly(phosphazene)s (PPZ), poly(phosphoester)s (PPE), poly(phosphoramidate)s (PPA), poly(N-2-hydroxypropylmethacrylamide) (pHPMA), poly(2-(dimethylamino)ethyl methacrylate) (pDMAEMA), poly(2-aminoethyl propylene phosphate) PPE_EA), galactosylated chitosan, N-dodecylated chitosan, histone, collagen and dextran-spermine. In one embodiment, the polymer may be an inert polymer such as, but not limited to, PEG. In one embodiment, the polymer may be a cationic polymer such as, but not limited to, PEI, PLL, TETA, poly(allylamine), Poly(N-ethyl-4-vinylpyridinium bromide), pHPMA and pDMAEMA. In one embodiment, the polymer may be a biodegradable PEI such as, but not limited to, DSP, DTBP and PEIC. In one embodiment, the polymer may be biodegradable such as, but not limited to, histine modified PLL, SS-PAEI, poly(P-aminoester), PHP, PAGA, PLGA, PPZ, PPE, PPA and PPE-EA.

A suitable polymeric carrier may be a polymeric carrier formed by disulfide-crosslinked cationic compounds. The disulfide-crosslinked cationic compounds may be the same or different from each other. The polymeric carrier can also contain further components. The polymeric carrier used according to the present invention may comprise mixtures of cationic peptides, proteins or polymers and optionally further components as defined herein, which are crosslinked by disulfide bonds (via—SH groups).

In this context, polymeric carriers according to formula {(Arg)I;(Lys)m;(His)n;(Orn)o;(Xaa′)x(Cys)y} and formula Cys,{(Arg)I;(Lys)m;(His)n;(Orn)o;(Xaa)x}Cys₂ of the patent application WO2012/013326 are preferred, the disclosure of WO2012/013326 relating thereto incorporated herewith by reference.

In embodiments, the polymeric carrier used to complex the at least one nucleic acid, preferably the at least one in vitro transcribed RNA comprising a 3′ terminal A nucleotide may be derived from a polymeric carrier molecule according formula (L-P¹-S-[S-P²-S]r-S-P³-L) of the patent application WO2011/026641, the disclosure of WO2011/026641 relating thereto incorporated herewith by reference.

In some embodiments, the polymeric carrier compound is formed by, or comprises or consists of the peptide elements CysArg12Cys (SEQ ID NO: 93) or CysArg12 (SEQ ID NO: 94) or TrpArg12Cys (SEQ ID NO: 95). In particularly preferred embodiments, the polymeric carrier compound consists of a (R₁₂C)-(R₁₂C) dimer, a (WR₁₂C)-(WR₁₂C) dimer, or a (CR₁₂)—(CR₁₂C)—(CR₁₂) trimer, wherein the individual peptide elements in the dimer (e.g. (WR₁₂C)), or the trimer (e.g. (CR12)), are connected via—SH groups.

In a preferred embodiment of the third aspect, at least one in vitro transcribed RNA comprising a 3′ terminal A nucleotide of the second aspect is complexed or associated with a polyethylene glycol/peptide polymer comprising HO-PEG5000-S—(S-CHHHHHHRRRRHHHHHHC-S-)7-S-PEG5000-OH (SEQ ID NO: 96 as peptide monomer), HO-PEG5000-S-(S-CHHHHHHRRRRHHHHHHC-S-)4-S-PEG5000-OH (SEQ ID NO: 96 as peptide monomer), HO-PEG5000-S-(S-CGHHHHHRRRRHHHHHGC-S-)7-S-PEG5000-OH (SEQ ID NO: 97 as peptide monomer) and/or a polyethylene glycol/peptide polymer comprising HO-PEG5000-S-(S-CGHHHHHRRRRHHHHHGC-S-)4-S-PEG5000-OH (SEQ ID NO: 97 of the peptide monomer).

In other embodiments, the composition comprises at least one in vitro transcribed RNA comprising a 3′ terminal A nucleotide which is complexed or associated with polymeric carriers and, optionally, with at least one lipid component as described in WO2017/212008, WO2017/212006, WO2017/212007, and WO2017/212009. In this context, the disclosures of WO2017/212008, WO2017/212006, WO2017/212007, and WO2017/212009 are herewith incorporated by reference.

In preferred embodiments, at least one in vitro transcribed RNA comprising a 3′ terminal A nucleotide is complexed or associated with one or more lipids, thereby forming liposomes, lipid nanoparticles (LNP), lipoplexes, and/or nanoliposomes.

In most preferred embodiments, the pharmaceutical composition comprising at least one in vitro transcribed RNA comprising a 3′ terminal A nucleotide which is complexed with one or more lipids thereby forming lipid nanoparticles (LNP).

The liposomes, lipid nanoparticles (LNPs), lipoplexes, and/or nanoliposomes-incorporated nucleic acid (e.g. RNA) may be completely or partially located in the interior space of the liposomes, lipid nanoparticles (LNPs), lipoplexes, and/or nanoliposomes, within the lipid layer/membrane, or associated with the exterior surface of the lipid layer/membrane. The incorporation of a nucleic acid into liposomes/LNPs is also referred to herein as “encapsulation” wherein the nucleic acid, e.g. the in vitro transcribed RNA comprising a 3′ terminal A nucleotide is entirely contained within the interior space of the liposomes, lipid nanoparticles (LNPs), lipoplexes, and/or nanoliposomes. The purpose of incorporating nucleic acid into liposomes, lipid nanoparticles (LNPs), lipoplexes, and/or nanoliposomes is to protect the nucleic acid, preferably RNA from an environment which may contain enzymes or chemicals or conditions that degrade nucleic acid and/or systems or receptors that cause the rapid excretion of the nucleic acid. Moreover, incorporating nucleic acid, preferably RNA into liposomes, lipid nanoparticles (LNPs), lipoplexes, and/or nanoliposomes may promote the uptake of the nucleic acid, and hence, may enhance the therapeutic effect of the nucleic acid, e.g. the RNA encoding antigenic nCoV-2019 proteins. Accordingly, incorporating a nucleic acid, e.g. RNA or DNA, into liposomes, lipid nanoparticles (LNPs), lipoplexes, and/or nanoliposomes may be particularly suitable for a coronavirus vaccine (e.g. a nCoV-2019 vaccine), e.g. for intramuscular and/or intradermal administration.

In this context, the terms “complexed” or “associated” refer to the essentially stable combination of nucleic acid with one or more lipids into larger complexes or assemblies without covalent binding.

LNP

In a specifically preferred embodiment, at least one in vitro transcribed RNA comprising a 3′ terminal A nucleotide is complexed with one or more lipids thereby forming lipid nanoparticles (LNP).

The term “lipid nanoparticle”, also referred to as “LNP”, is not restricted to any particular morphology, and include any morphology generated when a cationic lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP).

Liposomes, lipid nanoparticles (LNPs), lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter.

LNPs of the invention are suitably characterized as microscopic vesicles having an interior aqua space sequestered from an outer medium by a membrane of one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the at least one nucleic acid, preferably the at least one in vitro transcribed RNA comprising a 3′ terminal A nucleotide to a target tissue.

Accordingly, in preferred embodiments, the in vitro transcribed RNA comprising a 3′ terminal A nucleotide is complexed with one or more lipids thereby forming lipid nanoparticles (LNP).

LNPs typically comprise a cationic lipid and one or more excipients selected from neutral lipids, charged lipids, steroids and polymer conjugated lipids (e.g. PEGylated lipid). The coding in vitro transcribed RNA comprising a 3′ terminal A nucleotide may be encapsulated in the lipid portion of the LNP or an aqueous space enveloped by some or the entire lipid portion of the LNP. The coding RNA or a portion thereof may also be associated and complexed with the LNP. An LNP may comprise any lipid capable of forming a particle to which the nucleic acids are attached, or in which the one or more nucleic acids are encapsulated. Preferably, the LNP comprising nucleic acids comprises one or more cationic lipids, and one or more stabilizing lipids. Stabilizing lipids include neutral lipids and PEGylated lipids.

The cationic lipid of an LNP may be cationisable, i.e. it becomes protonated as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Such lipids include, but are not limited to, DSDMA, N,N-dioleyl-N,N-dimethylammonium chloride (DODAC), N,N-distearyl-N,N-dimethylammonium bromide (DDAB), 1,2-dioleoyltrimethyl ammonium propane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (y-DLenDMA), 98N12-5, 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP·Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)-N,N-dimethyl-2,2-di((9Z,12Z)-octadeca-9,12-dienyl)tetrahydro-3aH-cyclopenta[d] [1,3]dioxol-5-amine)), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), NC98-5 (4,7, 13-tris(3-oxo-3-(undecylamino)propyl)-NI,N 16-diundecyl-4,7, 10,13-tetraazahexadecane-1,16-diamide), (6Z,9Z,28Z,31Z)-heptatriaconta-6,9,28,31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010/053572 (and particularly, Cl 2-200 described at paragraph [00225]) and WO2012/170930, both of which are incorporated herein by reference, HGT4003, HGT5000, HGTS001, HGT5001, HGT5002 (see US2015/0140070).

In embodiments, the cationic lipid may be an amino lipid.

Representative amino lipids include, but are not limited to, 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP·Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US2010/0324120).

In embodiments, the cationic lipid may an aminoalcohol lipidoid.

Aminoalcohol lipidoids which may be used in the present invention may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017/075531, hereby incorporated by reference.

In another embodiment, suitable lipids can also be the compounds as disclosed in WO2015/074085 (i.e. ATX-001 to ATX-032 or the compounds as specified in claims 1-26), U.S. Appl. Nos. 61/905,724 and 15/614,499 or U.S. Pat. Nos. 9,593,077 and 9,567,296 hereby incorporated by reference in their entirety.

In other embodiments, suitable cationic lipids can also be the compounds as disclosed in WO2017/117530 (i.e. lipids 13, 14, 15, 16, 17, 18, 19, 20, or the compounds as specified in the claims), hereby incorporated by reference in its entirety.

In other embodiments, suitable cationic lipids may be selected from published PCT patent application WO2017/117530 (i.e. lipids 13, 14, 15, 16, 17, 18, 19, 20, or the compounds as specified in the claims), the specific disclosure hereby incorporated by reference.

In preferred embodiments, ionizable or cationic lipids may also be selected from the lipids disclosed in WO2018/078053 (i.e. lipids derived from formula I, II, and III of WO2018/078053, or lipids as specified in claims 1 to 12 of WO2018/078053), the disclosure of WO2018/078053 hereby incorporated by reference in its entirety. In that context, lipids disclosed in Table 7 of WO2018/078053 (e.g. lipids derived from formula I-1 to 1-41) and lipids disclosed in Table 8 of WO2018/078053 (e.g. lipids derived from formula II-1 to 11-36) may be suitably used in the context of the invention. Accordingly, formula 1-1 to formula 1-41 and formula II-1 to formula II-36 of WO2018/078053, and the specific disclosure relating thereto, are herewith incorporated by reference.

In preferred embodiments, cationic lipids may be derived from formula III of published PCT patent application WO2018/078053. Accordingly, formula III of WO2018/078053, and the specific disclosure relating thereto, are herewith incorporated by reference.

In particularly preferred embodiments, the in vitro transcribed RNA comprising a 3′ terminal A nucleotide as defined herein is complexed with one or more lipids thereby forming LNPs, wherein the cationic lipid of the LNP is selected from structures 111-1 to 111-36 of Table 9 of published PCT patent application WO2018/078053. Accordingly, formula III-1 to 111-36 of WO2018/078053, and the specific disclosure relating thereto, are herewith incorporated by reference.

In particularly preferred embodiment, the in vitro transcribed RNA comprising a 3 terminal A nucleotide as defined herein is complexed with one or more lipids thereby forming LNPs, wherein the LNP comprises the following cationic lipid:

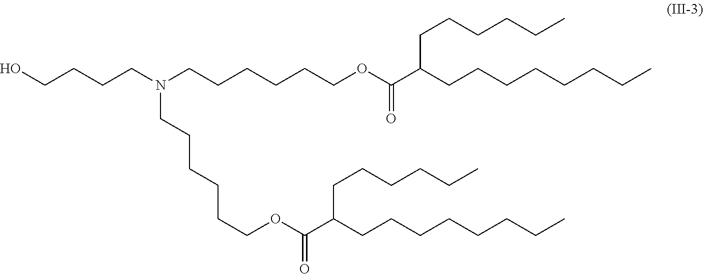 [SMILES: CCCCCCCCC(CCCCCC)C(=O)OCCCCCCN(CCCCO)CCCCCCOC(=O)C(CCCCCC)CCCCCCCC]

In certain embodiments, the cationic lipid as defined herein, more preferably cationic lipid compound 111-3, is present in the LNP in an amount from about 30 to about 95 mol %, relative to the total lipid content of the LNP. If more than one cationic lipid is incorporated within the LNP, such percentages apply to the combined cationic lipids.

In embodiments, the cationic lipid is present in the LNP in an amount from about 30 to about 70 mol %. In one embodiment, the cationic lipid is present in the LNP in an amount from about 40 to about 60 mole percent, such as about 40, 41, 42, 43, 44, 45, 46, 47, 48, 49, 50, 51, 52, 53, 54, 55, 56, 57, 58, 59 or 60 mol %, respectively. In embodiments, the cationic lipid is present in the LNP in an amount from about 47 to about 48 mol %, such as about 47.0, 47.1, 47.2, 47.3, 47.4, 47.5, 47.6, 47.7, 47.8, 47.9, 50.0 mol %, respectively, wherein 47.7 mol % are particularly preferred.

In some embodiments, the cationic lipid is present in a ratio of from about 20 mol % to about 70 or 75 mol % or from about 45 to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to coding in vitro transcribed RNA comprising a 3′ terminal A nucleotide is from about 3 to about 15, such as from about 5 to about 13 or from about 7 to about 11. Other suitable (cationic or ionizable) lipids are disclosed in published patent applications WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, and U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US20130178541, US2013/0225836, US2014/0039032 and WO2017/112865. In that context, the disclosures of WO2009/086558, WO2009/127060, WO2010/048536, WO2010/054406, WO2010/088537, WO2010/129709, WO2011/153493, WO 2013/063468, US2011/0256175, US2012/0128760, US2012/0027803, U.S. Pat. No. 8,158,601, WO2016/118724, WO2016/118725, WO2017/070613, WO2017/070620, WO2017/099823, WO2012/040184, WO2011/153120, WO2011/149733, WO2011/090965, WO2011/043913, WO2011/022460, WO2012/061259, WO2012/054365, WO2012/044638, WO2010/080724, WO2010/21865, WO2008/103276, WO2013/086373, WO2013/086354, U.S. Pat. Nos. 7,893,302, 7,404,969, 8,283,333, 8,466,122 and 8,569,256 and US Patent Publication No. US2010/0036115, US2012/0202871, US2013/0064894, US2013/0129785, US2013/0150625, US20130178541, US2013/0225836 and US2014/0039032 and WO2017/112865 specifically relating to (cationic) lipids suitable for LNPs are incorporated herewith by reference.

In some embodiments, amino or cationic lipids as defined herein have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7. LNPs can comprise two or more (different) cationic lipids as defined herein. Cationic lipids may be selected to contribute to different advantageous properties. For example, cationic lipids that differ in properties such as amine pKa, chemical stability, half-life in circulation, half-life in tissue, net accumulation in tissue, or toxicity can be used in the LNP. In particular, the cationic lipids can be chosen so that the properties of the mixed-LNP are more desirable than the properties of a single-LNP of individual lipids.

The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20. In this context, the N/P ratio is defined as the mole ratio of the nitrogen atoms (“N”) of the basic nitrogen-containing groups of the lipid or lipidoid to the phosphate groups (“P”) of the nucleic acid which is used as cargo. The N/P ratio may be calculated on the basis that, for example, 1 μg RNA typically contains about 3 mmol phosphate residues, provided that the RNA exhibits a statistical distribution of bases. The “N”-value of the lipid or lipidoid may be calculated on the basis of its molecular weight and the relative content of permanently cationic and—if present—cationisable groups.

PEG

In a preferred embodiment, the lipid nanoparticles (LNP) comprise a PEGylated lipid.

In vivo characteristics and behavior of LNPs can be modified by addition of a hydrophilic polymer coating, e.g. polyethylene glycol (PEG), to the LNP surface to confer steric stabilization. Furthermore, LNPs can be used for specific targeting by attaching ligands (e.g. antibodies, peptides, and carbohydrates) to its surface or to the terminal end of the attached PEG chains (e.g. via PEGylated lipids or PEGylated cholesterol).

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like.

In certain embodiments, the LNP comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol)2000)carbamyl]-1,2-dimyristyloxlpropyl-3-amine (PEG-c-DMA). In a preferred embodiment, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-(w-methoxy(polyethoxy)ethyl)butanedioate (PEG-S-DMG), a PEGylated ceramide (PEG-cer), or a PEG dialkoxypropylcarbamate such as w-methoxy(polyethoxy)ethyl-N-(2,3di(tetradecanoxy)propyl)carbamate or 2,3-di(tetradecanoxy)propyl-N-(w-methoxy(polyethoxy)ethyl)carbamate.

In preferred embodiments, the PEGylated lipid is preferably derived from formula (IV) of published PCT patent application WO2018/078053. Accordingly, PEGylated lipids derived from formula (IV) of published PCT patent application WO2018/078053, and the respective disclosure relating thereto, are herewith incorporated by reference.

In a particularly preferred embodiments, the at least one coding RNA of the composition is complexed with one or more lipids thereby forming LNPs, wherein the LNP comprises a PEGylated lipid, wherein the PEG lipid is preferably derived from formula (IVa) of published PCT patent application WO2018/078053. Accordingly, PEGylated lipid derived from formula (IVa) of published PCT patent application WO2018/078053, and the respective disclosure relating thereto, is herewith incorporated by reference.

In a particularly preferred embodiment, the at least one nucleic acid, preferably the at least one RNA is complexed with one or more lipids thereby forming lipid nanoparticles (LNP), wherein the LNP comprises a PEGylated lipid/PEG lipid. Preferably, said PEG lipid is of formula (IVa):

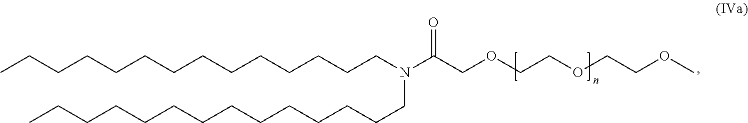

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In a most preferred embodiment n is about 49.

Further examples of PEG-lipids suitable in that context are provided in US2015/0376115 and WO2015/199952, each of which is incorporated by reference in its entirety.

In some embodiments, LNPs include less than about 3, 2, or 1 mol % of PEG or PEG-modified lipid, based on the total moles of lipid in the LNP. In further embodiments, LNPs comprise from about 0.1% to about 20% of the PEG-modified lipid on a molar basis, e.g. about 0.5 to about 10%, about 0.5 to about 5%, about 10%, about 5%, about 3.5%, about 3%, about 2,5%, about 2%, about 1.5%, about 1%, about 0.5%, or about 0.3% on a molar basis (based on 100% total moles of lipids in the LNP). In preferred embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most preferably 1.7% (based on 100% total moles of lipids in the LNP). In various embodiments, the molar ratio of the cationic lipid to the PEGylated lipid ranges from about 100:1 to about 25:1.

In another embodiment, the LNP comprises
(i) at least one cationic lipid;
(ii) at least one neutral lipid;
(iii) at least one steroid or steroid analogue; and
(iv) at least one a PEG-lipid.

In preferred embodiments, the LNP comprises one or more additional lipids which stabilize the formation of particles during their formation or during the manufacturing process (e.g. neutral lipid and/or one or more steroid or steroid analogue).

Suitable stabilizing lipids include neutral lipids and anionic lipids. The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

In embodiments, the LNP comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In other embodiments of the third aspect, the LNP comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In preferred embodiments, the neutral lipid is 1,2-distearoyl-sn-glycero-3-phosphocholine (DSPC). The molar ratio of the cationic lipid to DSPC may be in the range from about 2:1 to about 8:1.

In preferred embodiments, the steroid is cholesterol. The molar ratio of the cationic lipid to cholesterol may be in the range from about 2:1 to about 1:1. In some embodiments, the cholesterol may be PEGylated.

The sterol can be about 10 mol % to about 60 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle). Preferably, lipid nanoparticles (LNPs) comprise: (a) the at least one nucleic acid, preferably the at least one RNA of the first aspect, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the cationic lipids (as defined above), non-cationic lipids (as defined above), cholesterol (as defined above), and/or PEG-modified lipids (as defined above) may be combined at various relative molar ratios. For example, the ratio of cationic lipid to non-cationic lipid to cholesterol-based lipid to PEGylated lipid may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20: 10,40:30:25:5 or 40:32:20:8, 40:32:25:3 or 40:33:25:2, respectively.

In some embodiments, the LNPs comprise a lipid of formula (III), the at least one nucleic acid, preferably the at least one RNA as defined herein, a neutral lipid, a steroid and a PEGylated lipid. In preferred embodiments, the lipid of formula (III) is lipid compound III-3, the neutral lipid is DSPC, the steroid is cholesterol, and the PEGylated lipid is the compound of formula (IVa).

In a preferred embodiment of the third aspect, the LNP consists essentially of (i) at least one cationic lipid; (ii) a neutral lipid; (iii) a sterol, e.g., cholesterol; and (iv) a PEG-lipid, e.g. PEG-DMG or PEG-cDMA, in a molar ratio of about 20-60% cationic lipid: 5-25% neutral lipid: 25-55% sterol; 0.5-15% PEG-lipid.

In some embodiments, the LNP of the pharmaceutical composition comprises (i) at least one cationic lipid; (ii) at least one neutral lipid; (iii) at least one steroid or steroid analogue; and (iv) at least one a PEG-lipid wherein (i) to (iv) are in a molar ratio of about 20-60% cationic lipid, 5-25% neutral lipid, 25-55% sterol, and 0.5-15% PEG-lipid.

Most preferably, the LNP comprises
(i) at least one cationic lipid, preferably a lipid of formula (III), more preferably lipid Ill-3;
(ii) at least one neutral lipid, preferably 1,2-distearoyl-sn-glycero-3-phosphocholine (DSPC);
(iii) at least one steroid or steroid analog, preferably 1,2-distearoyl-sn-glycero-3-phosphocholine (DSPC); and
(iv) at least one aggregation reducing lipid, preferably a PEG-conjugated lipid derived from formula (IVa); and
wherein (i) to (iv) are in a molar ratio of about 47.4% cationic lipid, 10% neutral lipid, 40.9% steroid or steroid analog, and 1.7% aggregation reducing lipid.

In particularly preferred embodiments, the LNP comprises (i) to (iv) in a molar ratio of about 20-60% cationic lipid: 5-25% neutral lipid: 25-55% sterol; 0.5-15% PEG-lipid.

In one preferred embodiment, the lipid nanoparticle comprises: a cationic lipid with formula (III) and/or PEG lipid with formula (IV), optionally a neutral lipid, preferably 1,2-distearoyl-sn-glycero-3-phosphocholine (DSPC) and optionally a steroid, preferably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In a particular preferred embodiment, the LNPs have a molar ratio of approximately 50:10:38.5:1.5, preferably 47.5:10:40.8:1.7 or more preferably 47.4:10:40.9:1.7 (i.e. proportion (mol %) of cationic lipid (preferably lipid 111-3), DSPC, cholesterol and PEG-lipid (preferably PEG-lipid of formula (IVa) with n=49); solubilized in ethanol).

The total amount of nucleic acid in the lipid nanoparticles may vary and is defined depending on the e.g. nucleic acid to total lipid w/w ratio. In one embodiment of the invention the nucleic acid, in particular the RNA to total lipid ratio is less than 0.06 w/w, preferably between 0.03 w/w and 0.04 w/w.

In some embodiments, the lipid nanoparticles (LNPs), which are composed of only three lipid components, namely imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K).

In one embodiment, the lipid nanoparticle of the composition comprises a cationic lipid, a steroid; a neutral lipid; and a polymer conjugated lipid, preferably a pegylated lipid. Preferably, the polymer conjugated lipid is a pegylated lipid or PEG-lipid. In a specific embodiment, lipid nanoparticles comprise a cationic lipid resembled by the cationic lipid COATSOME® SS-EC (former name: SS-33/4PE-15; NOF Corporation, Tokyo, Japan), in accordance with the following structure:

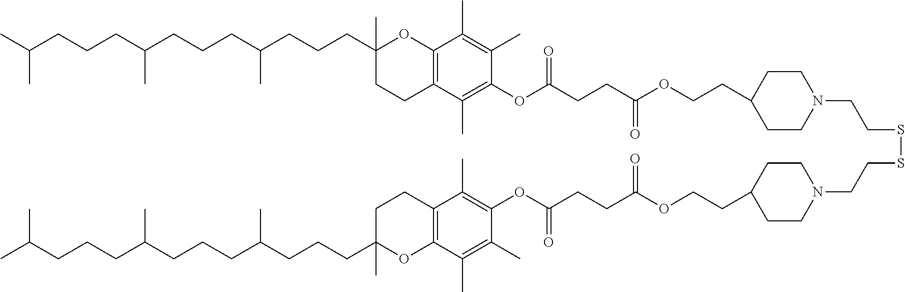 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

As described further below, those lipid nanoparticles are termed “GN01”.

Furthermore, in a specific embodiment, the GN01 lipid nanoparticles comprise a neutral lipid being resembled by the structure 1,2-diphytanoyl-sn-glycero-3-phosphoethanolamine (DPhyPE):

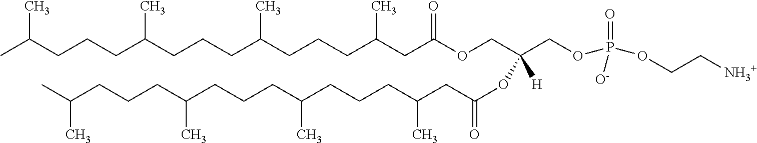 [SMILES: CC(C)CCCC(C)CCCC(C)CCCC(C)CC(=O)OC[C@H](COP(=O)([O-])OCC[NH3+])OC(=O)CC(C)CCCC(C)CCCC(C)CCCC(C)C]

Furthermore, in a specific embodiment, the GN01 lipid nanoparticles comprise a polymer conjugated lipid, preferably a pegylated lipid, being 1,2-dimyristoyl-rac-glycero-3-methoxypolyethylene glycol 2000 (DMG-PEG 2000) having the following structure:

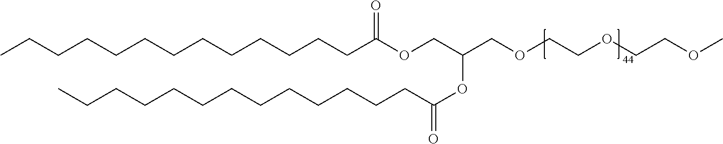

As used in the art, “DMG-PEG 2000” is considered a mixture of 1,2-DMG PEG2000 and 1,3-DMG PEG2000 in ˜97:3 ratio.

Accordingly, GN01 lipid nanoparticles (GN01-LNPs) according to one of the preferred embodiments comprise a SS-EC cationic lipid, neutral lipid DPhyPE, cholesterol, and the polymer conjugated lipid (pegylated lipid) 1,2-dimyristoyl-rac-glycero-3-methoxypolyethylene glycol (PEG-DMG).

In a preferred embodiment, the GN01 LNPs comprise:
(a) cationic lipid SS-EC (former name: SS-33/4PE-15; NOF Corporation, Tokyo, Japan) at an amount of 45-65 mol %;
(b) cholesterol at an amount of 25-45 mol %;
(c) DPhyPE at an amount of 8-12 mol %; and
(d) PEG-DMG 2000 at an amount of 1-3 mol %;
each amount being relative to the total molar amount of all lipidic excipients of the GN01 lipid nanoparticles.

In a further preferred embodiment, the GN01 lipid nanoparticles as described herein comprises 59 mol % cationic lipid, 10 mol % neutral lipid, 29.3 mol % steroid and 1.7 mol % polymer conjugated lipid, preferably pegylated lipid. In a most preferred embodiment, the GN01 lipid nanoparticles as described herein comprise 59 mol % cationic lipid SS-EC, 10 mol % DPhyPE, 29.3 mol % cholesterol and 1.7 mol % DMG-PEG 2000.

The amount of the cationic lipid relative to that of the nucleic acid in the GN01 lipid nanoparticle may also be expressed as a weight ratio (abbreviated f.e. “m/m”). For example, the GN01 lipid nanoparticles comprise the at least one nucleic acid, preferably the at least one RNA at an amount such as to achieve a lipid to RNA weight ratio in the range of about 20 to about 60, or about 10 to about 50. In other embodiments, the ratio of cationic lipid to nucleic acid or RNA is from about 3 to about 15, such as from about 5 to about 13, from about 4 to about 8 or from about 7 to about 11. In a very preferred embodiment of the present invention, the total lipid/RNA mass ratio is about 40 or 40, i.e. about 40 or 40 times mass excess to ensure RNA encapsulation. Another preferred RNA/lipid ratio is between about 1 and about 10, about 2 and about 5, about 2 and about 4, or preferably about 3.

Further, the amount of the cationic lipid may be selected taking the amount of the nucleic acid cargo such as the obtained in vitro transcribed RNA comprising a 3′ terminal A nucleotide into account. In one embodiment, the N/P ratio can be in the range of about 1 to about 50. In another embodiment, the range is about 1 to about 20, about 1 to about 10, about 1 to about 5. In one preferred embodiment, these amounts are selected such as to result in an N/P ratio of the GN01 lipid nanoparticles or of the composition in the range from about 10 to about 20. In a further very preferred embodiment, the N/P is 14 (i.e. 14 times mol excess of positive charge to ensure nucleic acid encapsulation).

In a preferred embodiment, GN01 lipid nanoparticles comprise 59 mol % cationic lipid COATSOME® SS-EC (former name: SS-33/4PE-15 as apparent from the examples section; NOF Corporation, Tokyo, Japan), 29.3 mol % cholesterol as steroid, 10 mol % DPhyPE as neutral lipid/phospholipid and 1.7 mol % DMG-PEG 2000 as polymer conjugated lipid. A further inventive advantage connected with the use of DPhyPE is the high capacity for fusogenicity due to its bulky tails, whereby it is able to fuse at a high level with endosomal lipids. For “GN01”, N/P (lipid to nucleic acid, e.g RNA mol ratio) preferably is 14 and total lipid/RNA mass ratio preferably is 40 (m/m).

In other embodiments, the at least one in vitro transcribed RNA comprising a 3′ terminal A nucleotide, is complexed with one or more lipids thereby forming lipid nanoparticles (LNP), wherein the LNP comprises
I at least one cationic lipid;
Ii at least one neutral lipid;
lii at least one steroid or steroid analogue; and
Iiii at least one PEG-lipid as defined herein,
wherein the cationic lipid is DLin-KC2-DMA (50 mol %) or DLin-MC3-DMA (50 mol %), the neutral lipid is DSPC (10 mol %), the PEG lipid is PEG-DOMG (1.5 mol %) and the structural lipid is cholesterol (38.5 mol %).

In other embodiments, the at least one nucleic acid (e.g. DNA or RNA), preferably the at least one in vitro transcribed RNA comprising a 3′ terminal A nucleotide is complexed with one or more lipids thereby forming lipid nanoparticles (LNP), wherein the LNP comprises SS15/Chol/DOPE (or DOPC)/DSG-5000 at mol % 50/38.5/10/1.5.

In other embodiments, the nucleic acid of the invention may be formulated in liposomes, e.g. in liposomes as described in WO2019/222424, WO2019/226925, WO2019/232095, WO2019/232097, or WO2019/232208, the disclosure of WO2019/222424, WO2019/226925, WO2019/232095, WO2019/232097, or WO2019/232208 relating to liposomes or lipid-based carrier molecules herewith incorporated by reference.

In most preferred embodiment the lipid nanoparticles (LNP) additionally comprise a PEGylated lipid.

In one embodiment the LNP comprises of
(i) at least one cationic lipid;
(ii) at least one neutral lipid;
(iii) at least one steroid or steroid analogue; and
(iv) at least one a PEG-lipid, wherein (i) to (iv) are in a molar ratio of about 20-60% cationic lipid, 5-25% neutral lipid, 25-55% sterol, and 0.5-15% PEG-lipid.

In various embodiments, LNPs that suitably encapsulates the at least one nucleic acid of the invention have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average as determined by dynamic light scattering as commonly known in the art.

The polydispersity index (PDI) of the nanoparticles is typically in the range of 0.1 to 0.5. In a particular embodiment, a PDI is below 0.2. Typically, the PDI is determined by dynamic light scattering.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In another preferred embodiment of the invention the lipid nanoparticles have a hydrodynamic diameter in the range from about 50 nm to about 300 nm, or from about 60 nm to about 250 nm, from about 60 nm to about 150 nm, or from about 60 nm to about 120 nm, respectively.

In embodiments where more than one or a plurality, e.g. 2, 3, 4, 5, 6, 7, 8, 9, 10, 11, 12, 13, 14, 15 of nucleic acid species of the invention are comprised in the composition, said more than one or said plurality e.g. 2, 3, 4, 5, 6, 7, 8, 9, 10, 11, 12, 13, 14, 15 of nucleic acid species of the invention may be complexed within one or more lipids thereby forming LNPs comprising more than one or a plurality, e.g. 2, 3, 4, 5, 6, 7, 8, 9, 10, 11, 12, 13, 14, 15 of different nucleic acid species.

In embodiments, the LNPs described herein may be lyophilized in order to improve storage stability of the formulation and/or the obtained in vitro transcribed RNA comprising a 3′ terminal A nucleotide. In embodiments, the LNPs described herein may be spray dried in order to improve storage stability of the formulation and/or the nucleic acid.

Lyoprotectants for lyophilization and or spray drying may be selected from trehalose, sucrose, mannose, dextran and inulin. A preferred lyoprotectant is sucrose, optionally comprising a further lyoprotectant. A further preferred lyoprotectant is trehalose, optionally comprising a further lyoprotectant.

Accordingly, the composition, e.g. the composition comprising LNPs is lyophilized (e.g. according to WO2016/165831 or WO2011/069586) to yield a temperature stable dried nucleic acid (powder) composition as defined herein (e.g. RNA or DNA). The composition, e.g. the composition comprising LNPs may also be dried using spray-drying or spray-freeze drying (e.g. according to WO2016/184575 or WO2016/184576) to yield a temperature stable composition (powder) as defined herein.

Accordingly, in preferred embodiments, the composition is a dried composition.

The term “dried composition” as used herein has to be understood as composition that has been lyophilized, or spray-dried, or spray-freeze dried as defined above to obtain a temperature stable dried composition (powder) e.g. comprising LNP complexed RNA (as defined above).

In some aspects, the obtained in vitro transcribed RNA comprising a 3′ terminal A nucleotide species of the pharmaceutical composition may encode a different therapeutic peptide or protein as defined.

The term “in vitro transcribed RNA comprising a 3′ terminal A nucleotide species” as used herein is not intended to refer to only one single molecule. The term “in vitro transcribed RNA comprising a 3′ terminal A nucleotide species” has to be understood as an ensemble of essentially identical RNA molecules, wherein each of the RNA molecules of the RNA ensemble, in other words each of the molecules of the RNA species, encodes the same therapeutic protein (in embodiments where the in vitro transcribed RNA comprising a 3′ terminal A nucleotide is a coding RNA), having essentially the same nucleic acid sequence. However, the RNA molecules of the in vitro transcribed RNA comprising a 3′ terminal A nucleotide ensemble may differ in length or quality which may be caused by the enzymatic or chemical manufacturing process.

In some embodiments, the pharmaceutical composition comprises more than one or a plurality of different in vitro transcribed RNA comprising a 3′ terminal A nucleotide species wherein the more than one or a plurality of different in vitro transcribed RNA comprising a 3′ terminal A nucleotide species is selected from coding RNA species each encoding a different protein.

In other embodiments, the pharmaceutical composition comprises more than one or a plurality of different in vitro transcribed RNA comprising a 3′ terminal A nucleotide species of the first component, wherein at least one of the more than one or a plurality of different in vitro transcribed RNA comprising a 3′ terminal A nucleotide species is selected from a coding RNA species (e.g., an mRNA encoding a CRISPR associated endonuclease), and at least one is selected from a non-coding RNA species (e.g., a guide RNA).

In preferred embodiments, the pharmaceutical composition comprises the in vitro transcribed RNA comprising a 3′ terminal A nucleotide, preferably an mRNA, wherein said in vitro transcribed RNA comprising a 3′ terminal A nucleotide is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof. Complexation/association (“formulation”) to carriers as defined herein facilitates the uptake of the in vitro transcribed RNA comprising a 3′ terminal A nucleotide into cells.

Lipidoid

In some embodiments the pharmaceutical composition may comprise least one lipid or lipidoid as described in published PCT applications WO2017/212008, WO2017/212006, WO2017/212007, and WO2017/212009, the disclosures of WO2017/212008, WO2017/212006, WO2017/212007, and WO2017/212009 herewith incorporated by reference.

In particularly preferred embodiments, the polymeric carrier (of the in vitro transcribed RNA comprising a 3′ terminal A nucleotide) is a peptide polymer, preferably a polyethylene glycol/peptide polymer as defined above, and a lipid, preferably a lipidoid.

A lipidoid (or lipidoit) is a lipid-like compound, i.e. an amphiphilic compound with lipid-like physical properties. The lipidoid is preferably a compound which comprises two or more cationic nitrogen atoms and at least two lipophilic tails. In contrast to many conventional cationic lipids, the lipidoid may be free of a hydrolysable linking group, in particular linking groups comprising hydrolysable ester, amide or carbamate groups. The cationic nitrogen atoms of the lipidoid may be cationisable or permanently cationic, or both types of cationic nitrogens may be present in the compound. In the context of the present invention the term lipid is considered to also encompass lipidoids.

In some embodiments of the inventions, the lipidoid may comprise a PEG moiety.

Suitably, the lipidoid is cationic, which means that it is cationisable or permanently cationic. In one embodiment, the lipidoid is cationisable, i.e. it comprises one or more cationisable nitrogen atoms, but no permanently cationic nitrogen atoms. In another embodiment, at least one of the cationic nitrogen atoms of the lipidoid is permanently cationic. Optionally, the lipidoid comprises two permanently cationic nitrogen atoms, three permanently cationic nitrogen atoms, or even four or more permanently cationic nitrogen atoms.

In a preferred embodiment, the lipidoid component may be any one selected from the lipidoids of the lipidoids provided in the table of page 50-54 of published PCT patent application WO2017/212009, the specific lipidoids provided in said table, and the specific disclosure relating thereto herewith incorporated by reference.

In preferred embodiments, the lipidoid component may be any one selected from 3-C12-OH, 3-C12-OH-cat, 3-C12-amide, 3-C12-amide monomethyl, 3-C12-amide dimethyl, RevPEG(10)-3-C12-OH, RevPEG(10)-DLin-pAbenzoic, 3C12amide-TMA cat., 3C12amide-DMA, 3C12amide-NH2, 3C12amide-OH, 3C12Ester-OH, 3C12 Ester-amin, 3C12Ester-DMA, 2C12Amid-DMA, 3C12-lin-amid-DMA, 2C12-sperm-amid-DMA, or 3C12-sperm-amid-DMA (see table of published PCT patent application WO2017/212009 (pages 50-54)). Particularly preferred are 3-012-OH or 3-C12-OH-cat.

In preferred embodiments, the polyethylene glycol/peptide polymer comprising a lipidoid as specified above (e.g. 3-C12-OH or 3-C12-OH-cat), is used to complex the at least one nucleic acid to form complexes having an N/P ratio from about 0.1 to about 20, or from about 0.2 to about 15, or from about 2 to about 15, or from about 2 to about 12, wherein the N/P ratio is defined as the mole ratio of the nitrogen atoms of the basic groups of the cationic peptide or polymer to the phosphate groups of the nucleic acid. In that context, the disclosure of published PCT patent application WO2017/212009, in particular claims 1 to 10 of WO2017/212009, and the specific disclosure relating thereto is herewith incorporated by reference.

Further suitable lipidoids may be derived from published PCT patent application WO2010/053572. In particular, lipidoids derivable from claims 1 to 297 of published PCT patent application WO2010/053572 may be used in the context of the invention, e.g. incorporated into the peptide polymer as described herein, or e.g. incorporated into the lipid nanoparticle (as described below). Accordingly, claims 1 to 297 of published PCT patent application WO2010/053572, and the specific disclosure relating thereto, is herewith incorporated by reference.

In particularly preferred embodiments, the at least one nucleic acid, preferably the at least one in vitro transcribed RNA comprising a 3′ terminal A nucleotide is complexed with one or more lipids thereby forming lipid nanoparticles (LNP), wherein the LNP comprises
(i) at least one cationic lipid as defined herein, preferably a lipid of formula (III), more preferably lipid 111-3;
(ii) at least one neutral lipid as defined herein, preferably 1,2-distearoyl-sn-glycero-3-phosphocholine (DSPC);
(iii) at least one steroid or steroid analogue as defined herein, preferably cholesterol; and
at least one PEG-lipid as defined herein, e.g. PEG-DMG or PEG-cDMA, preferably a PEGylated lipid that is or is derived from formula (IVa).

In various embodiments the pharmaceutical composition comprises Ringer or Ringer-Lactate solution.

Accordingly, the pharmaceutical composition may comprise and/or is administered in Ringer or Ringer-Lactate solution as described in WO2006/122828.

In embodiments, pharmaceutical composition may be provided in lyophilized or dried form (using e.g. lyophilisation or drying methods as described in WO2016/165831, WO2011/069586, WO2016/184575 or WO2016/184576).

Preferably, the lyophilized or dried pharmaceutical composition is reconstituted in a suitable buffer, advantageously based on an aqueous carrier, prior to administration, e.g. Ringer- or Ringer-Lactate solution or a phosphate buffer solution.

In preferred embodiments, the pharmaceutical composition comprises at least one antagonist of at least one RNA sensing pattern recognition receptor.

In preferred embodiments in that context, the pharmaceutical composition comprises at least one antagonist of at least one RNA sensing pattern recognition receptor selected from a Toll-like receptor, preferably a TLR7 antagonist and/or a TLR8 antagonist.

Suitable antagonist of at least one RNA sensing pattern recognition receptor are disclosed in published PCT patent application WO2021028439, the full disclosure herewith incorporated by reference. In particular, the disclosure relating to suitable antagonist of at least one RNA sensing pattern recognition receptors as defined in any one of the claims 1 to 94 of WO2021028439 are incorporated by reference.

In preferred embodiments, the at least one antagonist of at least one RNA sensing pattern recognition receptor is a single stranded oligonucleotide that comprises or consists of a nucleic acid sequence being identical or at least 90%, 91%, 92%, 93%, 94%, 95%, 96%, 97%, 98%, or 99% identical to a nucleic acid sequence selected from the group consisting of SEQ ID NOs: 85-212 of WO2021028439, or fragments of any of these sequences. A particularly preferred antagonist in that context is 5′-GAG CGmG CCA-3′ (SEQ ID NO: 85 of WO2021028439), or a fragment or variant thereof.

In preferred embodiments, the molar ratio of the at least one antagonist of at least one RNA sensing pattern recognition receptor to the at least one RNA suitably ranges from about 20:1 to about 80:1.

In preferred embodiments, the weight to weight ratio of the at least one antagonist of at least one RNA sensing pattern recognition receptor to the at least one RNA suitably ranges from about 1:2 to about 1:10.

In embodiments, the at least one antagonist of at least one RNA sensing pattern recognition receptor and the at least one RNA are separately formulated (e.g. in LNPs) as defined herein or co-formulated (e.g. in LNPs) as defined herein.

Administration

In preferred embodiments, the administration of the pharmaceutical composition to a cell or subject results in a reduced innate immune response compared to an administration of a corresponding composition that comprises an RNA that does not comprise a 3′-terminal A nucleotide.

The term “subject” or “cell” as used herein generally includes humans and non-human animals or cells and preferably mammals, including chimeric and transgenic animals and disease models. Subjects to which administration of the compositions, preferably the pharmaceutical composition, is contemplated include, but are not limited to, humans and/or other primates; mammals, including commercially relevant mammals such as cattle, pigs, horses, sheep, cats, dogs; and/or birds, including commercially relevant birds such as poultry, chickens, ducks, geese, and/or turkeys. Preferably, the term “subject” refers to a non-human primate or a human, most preferably a human.

Comparably, the administration of the pharmaceutical composition to a cell or subject results in an improved expression of a therapeutic protein compared to an administration of a corresponding composition that comprises an RNA that does not comprise a 3′-terminal A nucleotide.

In the context of this invention the administration of the pharmaceutical composition to a cell or subject results in translation of the in vitro transcribed RNA comprising a 3′ terminal A nucleotide into a (functional) peptide or protein.

Suitably, reducing the stimulation of innate immune responses may be advantageous for various medical applications of the pharmaceutical composition. In particular, the in vitro transcribed RNA comprising a 3′ terminal A nucleotide obtainable by the method of this invention may be used for chronic administration or may e.g. enhance or improve the therapeutic effect of a in the in vitro transcribed RNA encoding an antigen (e.g. viral antigen, tumour antigen).

Accordingly, reducing the innate immune responses of the obtained in vitro transcribed RNA of the invention leads to an increased efficiency of a therapeutic RNA (e.g. upon administration to a cell or a subject).

In a preferred embodiment the in vitro transcribed RNA comprising a 3′ terminal A nucleotide obtainable by the method of this invention may be used for vaccination to treat or prevent an infectious disease.

In a preferred embodiment the in vitro transcribed RNA comprising a 3′ terminal A nucleotide obtainable by the method of this invention may be used for protein replacement therapy.

In a preferred embodiment the in vitro transcribed RNA comprising a 3′ terminal A nucleotide obtainable by the method of this invention comprising modified nucleotides may be used for protein replacement therapy.

In some embodiments, detectable levels of the therapeutic protein are produced in the serum of the subject at about 1 to about 72 hours post administration.

Moreover, in that context, the method of this invention allows the reduction of reactogenicity of a coding therapeutic RNA (comprising a cds encoding e.g. an antigen). The term reactogenicity refers to the property of e.g. a vaccine of being able to produce adverse reactions, especially excessive immunological responses and associated signs and symptoms-fever, sore arm at injection site, etc. Other manifestations of reactogenicity typically comprise bruising, redness, induration, and swelling.

Accordingly, the method of reducing or suppressing (innate) immune stimulation of an in vitro transcribed RNA has also be understood as method of reducing or suppressing the reactogenicity of a coding in vitro transcribed RNA, wherein said coding in vitro transcribed RNA comprises a cds encoding an antigen.

In preferred embodiments the administration of the pharmaceutical composition to a human subject results in a reduced innate immune response compared to an administration of a corresponding composition that comprises an RNA that does not comprise a 3-terminal A nucleotide.

In particularly preferred embodiments, the subject is a mammalian subject, preferably a human subject, e.g. new-born human subject, pregnant human subject, immunocompromised human subject, and/or elderly human subject.

In various embodiments the administration of the pharmaceutical composition is systemically or locally.

In preferred embodiments the administration of the pharmaceutical composition is transdermally, intradermally, intravenously, intramuscularly, intranorally, intraaterially, intranasally, intrapulmonally, intracranially, intralesionally, intratumorally, intravitreally, subcutaneously or via sublingual, preferably intramuscularly, intranodally, intradermally, intratumorally or intravenously.

In a preferred embodiment the administration of the pharmaceutical composition is intramuscularly.

In another preferred embodiment the administration of the pharmaceutical composition is intravenously.

In another preferred embodiment the administration of the pharmaceutical composition is intratumorally.

In some embodiments the administration of the pharmaceutical composition is orally, parenterally, by inhalation spray, topically, rectally, nasally, buccally, vaginally or via an implanted reservoir.

The term parenteral, as used herein, includes subcutaneous, intravenous, intramuscular, intra-articular, intra-synovial, intrasternal, intrathecal, intrahepatic, intralesional, intracranial, transdermal, intradermal, intrapulmonal, intraperitoneal, intracardial, intraarterial, intraocular, intravitreal, subretinal, intratumoral.

In other preferred embodiments the administration is more than once, for example once or once more than once a day, once or more than once a week, once or more than once a month.

Advantageously, the pharmaceutical composition is suitable for repetitive administration, e.g. for chronic administration.

In particularly preferred embodiments, administration of the pharmaceutical composition is performed intravenously.

In other particular embodiments, the pharmaceutical composition is administered intravenously as a chronic treatment (e.g. more than once, for example once or more than once a day, once or more than once a week, once or more than once a month).

Fourth Aspect: Kit of Parts

In a fourth aspect, the present invention provides a kit or kit of parts, preferably comprising the individual components of the method of producing an in vitro transcribed RNA with reduced immunostimulatory properties (e.g. as defined in the context of the first aspect) and/or comprising the in vitro transcribed RNA comprising a 3′ terminal A nucleotide (e.g. as defined in the context of the second aspect) and/or comprising the pharmaceutical composition of (e.g. as defined in the context of the third aspect).

Notably, embodiments relating to the first and the second aspect of the invention are likewise applicable to embodiments of the third aspect of the invention, and embodiments relating to the third aspect of the invention are likewise applicable to embodiments of the first and second aspect of the invention.

In addition, the kit or kit of parts may comprise a liquid vehicle for solubilising, and/or technical instructions providing information on administration and dosage of the components.

In a preferred embodiment the kit or kit of parts comprising the in vitro transcribed RNA comprising a 3′ terminal A nucleotide or pharmaceutical composition, optionally comprising a liquid vehicle for solubilizing, and, optionally, technical instructions providing information on administration and/or dosage of the components.

In preferred embodiments, the kit or the kit of parts comprises:
(a) at least one composition as defined in the context of the third aspect;
(b) optionally, a liquid vehicle for solubilizing, and optionally technical instructions providing information on administration and dosage of the components.

In most preferred embodiments, the kit or the kit of parts comprises:
(a) the in vitro transcribed RNA comprising a 3′ terminal A nucleotide as defined herein, preferably an mRNA encoding a therapeutic peptide or protein, e.g. an antibody, an enzyme, an antigen, preferably wherein said mRNA does not comprise modified nucleotides, preferably wherein said mRNA does comprise a cap1 structure, preferably wherein said first component is formulated in a lipid nanoparticle or in a polyethylene glycol/peptide polymer.
(b) optionally, a liquid vehicle for solubilising (a) and/or (b), and optionally technical instructions providing information on administration and dosage of the components.

The technical instructions of said kit or kit of parts may comprise information about administration and dosage and patient groups. Such kits, preferably kits of parts, may be applied e.g. for any of the applications or medical uses mentioned herein.

Preferably, the individual components of the kit or kit of parts may be provided in lyophilised form. The kit may further contain as a part a vehicle (e.g. pharmaceutically acceptable buffer solution) for solubilising the in vitro transcribed RNA comprising a 3′ terminal A nucleotide, and/or the pharmaceutical composition of the third aspect.

In preferred embodiments, the kit or kit of parts comprises Ringer- or Ringer lactate solution.

In preferred embodiments, the kit or kit of parts comprise an injection needle, a microneedle, an injection device, a catheter, an implant delivery device, or a micro cannula.

Any of the above kits may be used in applications or medical uses as defined in the context of the invention.

Medical Use

In further aspects the present invention relates to the medical use of the in vitro transcribed RNA comprising a 3′-terminal A nucleotide having reduced immunostimulatory properties of the second aspect obtainable by the method of the first aspect, the pharmaceutical composition of the third aspect or the kit or kit of parts of the fourth aspect.

Notably, embodiments relating to the in vitro transcribed RNA comprising a 3′-terminal A nucleotide having reduced immunostimulatory properties of the second aspect obtainable by the method of the first aspect, the pharmaceutical composition of the third aspect or the kit or kit of parts of the fourth aspect may likewise be read on and be understood as suitable embodiments of medical uses of the invention.

Accordingly, the invention provides the in vitro transcribed RNA comprising a 3′-terminal A nucleotide of the second aspect obtainable by the method of the first aspect for use as a medicament, the pharmaceutical composition of the third aspect for use as a medicament or the kit or kit of parts as defined in the fifth aspect for use as a medicament.

In embodiments, the in vitro transcribed RNA comprising a 3′-terminal A nucleotide of the second aspect obtainable by the method of the first aspect, the pharmaceutical composition of the third aspect or the kit or kit of parts of the fourth aspect may be used for human medical purposes and also for veterinary medical purposes, preferably for human medical purposes.

In embodiments, the in vitro transcribed RNA comprising a 3′-terminal A nucleotide of the second aspect obtainable by the method of the first aspect, the pharmaceutical composition of the third aspect or the kit or kit of parts of the fourth aspect may be in particular used and suitable for human medical purposes, in particular for young infants, newborns, immunocompromised recipients, pregnant and breast-feeding women, and elderly people.

In yet another aspect, the invention relates to the medical use of the in vitro transcribed RNA comprising a 3-terminal A nucleotide of the second aspect obtainable by the method of the first aspect, the pharmaceutical composition of the third aspect or the kit or kit of parts of the fourth aspect for use in the treatment or prophylaxis of a tumour disease, or of a disorder related to such tumour disease.

Accordingly, in said embodiments, the obtained in vitro transcribed RNA comprising a 3′-terminal A nucleotide may encode at least one tumour or cancer antigen and/or at least one therapeutic antibody (e.g. checkpoint inhibitor).

In yet another aspect, the invention relates to the medical use of the in vitro transcribed RNA comprising a 3′-terminal A nucleotide of the second aspect obtainable by the method of the first aspect, the pharmaceutical composition of the third aspect or the kit or kit of parts of the fourth aspect for use in the treatment or prophylaxis of a genetic disorder or condition.

Such a genetic disorder or condition may be a monogenetic disease, i.e. (hereditary) disease, or a genetic disease in general, diseases which have a genetic inherited background and which are typically caused by a defined gene defect and are inherited according to Mendel's laws.

Accordingly, in said embodiments, the RNA comprising a 3-terminal A nucleotide may encode a CRISPR-associated endonuclease or another protein or enzyme suitable for genetic engineering.

In yet another aspect, the invention relates to the medical use of the in vitro transcribed RNA comprising a 3′-terminal A nucleotide of the second aspect obtainable by the method of the first aspect, the pharmaceutical composition of the third aspect or the kit or kit of parts of the fourth aspect for use in the treatment or prophylaxis of a protein or enzyme deficiency or protein replacement.

Accordingly, in said embodiments, the RNA comprising a 3′-terminal A nucleotide may encode at least one protein or enzyme. “Protein or enzyme deficiency” in that context has to be understood as a disease or deficiency where at least one protein is deficient, e.g. A1AT deficiency.

In yet another aspect, the invention relates to the medical use of the in vitro transcribed RNA comprising a 3-terminal A nucleotide of the second aspect obtainable by the method of the first aspect, the pharmaceutical composition of the third aspect or the kit or kit of parts of the fourth aspect for use in the treatment or prophylaxis of autoimmune diseases, allergies or allergic diseases, cardiovascular diseases, neuronal diseases, diseases of the respiratory system, diseases of the digestive system, diseases of the skin, musculoskeletal disorders, disorders of the connective tissue, neoplasms, immune deficiencies, endocrine, nutritional and metabolic diseases, eye diseases, and ear diseases.

In yet another aspect, the invention relates to the medical use of the in vitro transcribed RNA comprising a 3-terminal A nucleotide of the second aspect obtainable by the method of the first aspect, the pharmaceutical composition of the third aspect or the kit or kit of parts of the fourth aspect for use in the treatment or prophylaxis of an infection, or of a disorder related to such an infection.

In that context, an infection may be caused a pathogen selected from a bacterium, a protozoan, or a virus, for example from a pathogen provided in List 1.

In this context it is particularly preferred that the medical use comprises the prevention of SARS-CoV-2 infections, RSV infections, and/or Influenza virus infections.

In the context of a use in the treatment or prophylaxis of an infection, the in vitro transcribed RNA comprising a 3′-terminal A nucleotide of the second aspect obtainable by the method of the first aspect, the pharmaceutical composition of the third aspect or the kit or kit of parts of the fourth aspect may preferably be administered locally or systemically. In that context, administration may be by an intradermal, subcutaneous, intranasal, or intramuscular route. In embodiments, administration may be by conventional needle injection or needle-free jet injection.

In embodiments, the in vitro transcribed RNA comprising a 3′-terminal A nucleotide of the second aspect obtainable by the method of the first aspect, the pharmaceutical composition of the third aspect or the kit or kit of parts of the fourth aspect is provided in an amount of about 100 ng to about 500 ug, in an amount of about 1 ug to about 200 ug, in an amount of about 1 ug to about 100 ug, in an amount of about 5 ug to about 100 ug, preferably in an amount of about 10 ug to about 50 ug, specifically, in an amount of about 1 ug, 2 ug, 3 ug, 4 ug, 5 ug, 10 ug, 15 ug, 20 ug, 25 ug, 30 ug, 35 ug, 40 ug, 45 ug, 50 ug, 55 ug, 60 ug, 65 ug, 70 ug, 75 ug, 80 ug, 85 ug, 90 ug, 95 ug or 100 ug. Notably, the amount relates to the total amount of in vitro transcribed RNA comprised in the composition or vaccine.

In the context of a use in the treatment or prophylaxis of an infection, the immunization protocol for the treatment or prophylaxis of a subject against at least one pathogen, comprises one single dose. In some embodiments, the effective amount is a dose of 1 ug administered to the subject in one vaccination. In some embodiments, the effective amount is a dose of 2 ug administered to the subject in one vaccination. In some embodiments, the effective amount is a dose of 3 ug administered to the subject in one vaccination. In some embodiments, the effective amount is a dose of 4 ug administered to the subject in one vaccination. In some embodiments, the effective amount is a dose of 5 ug administered to the subject in one vaccination. In some embodiments, the effective amount is a dose of 10 ug administered to the subject in one vaccination. In some embodiments, the effective amount is a dose of 12 ug administered to the subject in one vaccination.

In some embodiments, the effective amount is a dose of 20 ug administered to the subject in one vaccination. In some embodiments, the effective amount is a dose of 30 ug administered to the subject in one vaccination. In some embodiments, the effective amount is a dose of 40 ug administered to the subject in one vaccination. In some embodiments, the effective amount is a dose of 50 ug administered to the subject in one vaccination. In some embodiments, the effective amount is a dose of 100 ug administered to the subject in one vaccination. In some embodiments, the effective amount is a dose of 200 ug administered to the subject in one vaccination. Notably, the effective amount relates to the total amount of nucleic acid comprised in the composition or vaccine.

In the context of a use in the treatment or prophylaxis of an infection, the effective amount is a dose of 1 ug administered to the subject a total of two times. In some embodiments, the effective amount is a dose of 2 ug administered to the subject a total of two times. In some embodiments, the effective amount is a dose of 3 ug administered to the subject a total of two times. In some embodiments, the effective amount is a dose of 4 ug administered to the subject a total of two times. In some embodiments, the effective amount is a dose of 5 ug administered to the subject a total of two times. In some embodiments, the effective amount is a dose of 10 ug administered to the subject a total of two times. In some embodiments, the effective amount is a dose of 12 ug administered to the subject a total of two times. In some embodiments, the effective amount is a dose of 20 ug administered to the subject a total of two times. In some embodiments, the effective amount is a dose of 30 ug administered to the subject a total of two times. In some embodiments, the effective amount is a dose of 40 ug administered to the subject a total of two times. In some embodiments, the effective amount is a dose of 50 ug administered to the subject a total of two times. In some embodiments, the effective amount is a dose of 100 ug administered to the subject a total of two times. In some embodiments, the effective amount is a dose of 200 ug administered to the subject a total of two times. Notably, the effective amount relates to the total amount of RNA comprised in the composition or vaccine.

In preferred embodiments, the vaccination/immunization immunizes the subject against an infection (upon administration as defined herein) for at least 1 year, preferably at least 2 years. In preferred embodiments, the vaccine/composition immunizes the subject against an infection for more than 2 years, more preferably for more than 3 years, even more preferably for more than 4 years, even more preferably for more than 5-10 years.

Method of Treatment

In another aspect the present invention relates to a method of treating or preventing a disorder.

Notably, embodiments relating to the in vitro transcribed RNA comprising a 3′-terminal A nucleotide of the second aspect obtainable by the method of the first aspect, the pharmaceutical composition of the third aspect or the kit or kit of parts of the fourth aspect may likewise be read on and be understood as suitable embodiments of methods of treatment and use as provided herein. Furthermore, specific features and embodiments relating to method of treatments as provided herein may also apply for medical uses of the invention.

Accordingly, the in vitro transcribed RNA comprising a 3′-terminal A nucleotide of the second aspect obtainable by the method of the first aspect, the pharmaceutical composition of the third aspect or the kit or kit of parts of the fourth aspect may be used in the prevention or treatment of cancer, autoimmune diseases, infectious diseases, allergies or protein deficiency disorders.

Preventing (Inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In preferred embodiments, the present invention relates to a method of treating or preventing a disorder, wherein the method comprises applying or administering to a subject in need thereof the in vitro transcribed RNA comprising a 3′-terminal A nucleotide of the second aspect obtainable by the method of the first aspect, the pharmaceutical composition of the third aspect or the kit or kit of parts of the fourth aspect.

In preferred embodiments, the disorder is an infection with a pathogen selected from a bacterium, a protozoan, or a virus, for example from a pathogen provided in List 1.

In other embodiments, the disorder is a tumour disease or a disorder related to such tumour disease, a protein or enzyme deficiency, or a genetic disorder or condition.

In preferred embodiments, the present invention relates to a method of treating or preventing a disorder as defined above, wherein the method comprises applying or administering to a subject in need thereof the thereof the in vitro transcribed RNA comprising a 3′-terminal A nucleotide of the second aspect obtainable by the method of the first aspect, the pharmaceutical composition of the third aspect or the kit or kit of parts of the fourth aspect.

In particularly preferred embodiments, the subject in need is a mammalian subject, preferably a human subject, e.g. new-born human subject, pregnant human subject, immunocompromised human subject, and/or elderly human subject.

In particular, the method of treating or preventing a disorder may comprise the steps of:
a) providing the in vitro transcribed RNA comprising a 3′-terminal A nucleotide of the second aspect obtainable by the method of the first aspect, the pharmaceutical composition of the third aspect or the kit or kit of parts of the fourth aspect;
b) applying or administering said pharmaceutical composition, vaccine, or kit or kit of parts to a subject as a first dose;
c) optionally, applying or administering said pharmaceutical composition, vaccine, or kit or kit of parts to a subject as a second dose or a further dose, preferably at least 1, 2, 3, 4, 5, 6, 7, 8, 9, 10, 11, 12, months after the first dose.

In other embodiments the present invention relates to a chronic medical treatment of a disorder, wherein the method comprises applying or administering to a subject in need thereof the in vitro transcribed RNA comprising a 3′-terminal A nucleotide of the second aspect obtainable by the method of the first aspect, the pharmaceutical composition of the third aspect or the kit or kit of parts of the fourth aspect. The term “chronic medical treatment” relates to treatments that require the administration of the in vitro transcribed RNA comprising a 3′ terminal A nucleotide, the pharmaceutical composition, or the kit or kit of parts more than once, for example once or more than once a day, once or more than once a week, once or more than once a month.

The method of treating or preventing a disorder comprises applying or administering to a subject in need thereof the in vitro transcribed RNA comprising a 3′-terminal A nucleotide of the second aspect which is obtainable by the first aspect, the pharmaceutical composition of the third aspect or the kit or kit of parts of the fourth aspect preferably wherein applying or administering is performed more than once, for example once or more than once a day, once or more than once a week, once or more than once a month.

Accordingly, the administration is subcutaneous, intravenous, intramuscular, intra-articular, intra-synovial, intranasal, oral, intrasternal, intrathecal, intrahepatic, intralesional, intracranial, transdermal, intradermal, intrapulmonal, intraperitoneal, intracardial, intraarterial, intraocular, intravitreal, subretinal, intranodal, or intratumoral.

In preferred embodiments the subject in need treated to prevent a disorder is a mammalian subject, preferably a human subject.

In another aspect, the present invention relates to a method of reducing the induction of an innate immune response induced by an in vitro transcribed RNA upon administration of said RNA to a cell or a subject comprising (i) obtaining the in vitro transcribed RNA comprising a 3′-terminal A nucleotide; and (ii) administering an effective amount of the in vitro transcribed RNA from step (i) having reduced immunostimulatory properties to a cell or a subject.

In this context it is particularly preferred that the in vitro transcribed RNA according to the invention induces less reactogenicity in a subject upon administration compared to a reference in vitro transcribed RNA not comprising the 5′-terminal A nucleotide and not being purified as defined above.

The induction of less reactogenicity against the in vitro transcribed RNA according to the invention leads to the possibility to administer a higher dose of the in vitro transcribed RNA compared to a reference in vitro transcribed RNA.

In another aspect, the present invention relates to a method of inducing a (protective) immune response in a subject, wherein the method comprises applying or administering to a subject in need thereof the in vitro transcribed RNA comprising a 3′-terminal A nucleotide, or the pharmaceutical composition, or the kit or kit of parts as defined above, preferably wherein applying or administering is performed more than once, for example once or more than once a day, once or more than once a week, once or more than once a month.

In a particularly preferred embodiment, a protective immune response against SARS-CoV-2, Influenza virus and/or RSV infections is induced.

Also in this context, the induction of an innate immune response by the in vitro transcribed RNA according to the invention has been reduced by a method as defined above.

### BRIEF DESCRIPTION OF TABLES

List 1: Suitable pathogens of the invention
Table I: Type IIS restriction enzymes recognize asymmetric DNA sequences and cleave outside of their recognition sequence
Table II: Human codon usage with respective codon frequencies indicated for each amino acid
Table IIII: Injection scheme of different mRNA-formats encoding an anti-rabies monoclonal antibody
Table IV: dsRNA analysis results
Table V: mRNA constructs encoding malaria CSP used in the present example
Table VI: Vaccination scheme A of example 2
Table VII: Vaccination scheme B of example 2
Table VIII: IVT mRNA encoding RABV-G used in the example 3
Table IX: Vaccination schedule of example 3
Table X: IVT mRNA encoding PpLuc used in the example 4
Table XI: dsRNA content of IVT RNA digested during IVT step with different restriction endonucleases
Table XII: dsRNA content of purified and non-purified IVT RNA digested during IVT step with different restriction endonucleases
Table XIII: dsRNA content of purified and non-purified IVT RNA comprising different 3′ terminal nucleotides
Table XIV: dsRNA content of purified and non-purified IVT RNA comprising different cap structures and UTR combinations
Table XV: Cytokine IP-10 values after transfection of IVT RNA digested during IVT step with different restriction endonucleases
Table XVI: Cytokine IP-10 values after transfection of purified and non-purified IVT RNA comprising different 3′ terminal nucleotides digested with SapI and EcoRI
Table XVII: Cytokine IP-10 values after transfection of purified and non-purified IVT RNA comprising different cap structures and UTR combinations
Table XVIII: mRNA used in example 5
Table XIX: dsRNA content of in vitro transcribed RNA, cellulose column purified RNA fractions and fraction bound to the cellulose column

### BRIEF DESCRIPTION OF DRAWINGS

The figures shown in the following are merely illustrative and shall describe the present invention in a further way.

These figures shall not be construed to limit the present invention thereto.

FIG. 1 displays a schematic example of linearization of a template DNA strand using type II restriction endonucleases. FIG. 1A: SapI (type IIS restriction endonuclease) leading to an RNA comprising a 3′ terminal A nucleotide or FIG. 1B: EcoRI (type IIP restriction endonuclease). Adapted from Holtkamp et al., 2006, Gene Therapy.

FIG. 2 shows the expression and innate immunity of mRNAs encoding an anti-rabies mAb (human IgG, SO57) that utilize different 3′ ends. FIG. 2A: LNP-formulated mRNA which utilize different 3′ end formats encoding anti-rabies mAb (human IgG, SO57) lead to expression of human IgG in BALB/c mice 4 h and 24 h post intravenous injection, respectively. FIG. 2B-1: Innate immune response (B: IFNa, C: IL-6, D: MIP-1p, E: MCP1,F: Rantes, G:TNF, H: INFγ, I: MIG:) after intravenous injection of LNP-formulated mRNA containing different 3′ end formats encoding anti-rabies mAb. The 3′ end formats for which the IVT template DNAs were linearized using SapI endonuclease lead to a reduced immune response displayed by a reduction of INFα, IL-6, MIP-1β, MCP1, Rantes, TNF, INFγ and MIG. Group A: LNP-formulated mRNA encoding anti-rabies mAb containing the 3′ end hSL-A100 generated by DNA templates linearized using SapI. Group B: LNP-formulated mRNA encoding anti-rabies mAb containing the 3′ end hSL-A100-N5 generated by DNA templates linearized using EcoRI. Group C: LNP-formulated mRNA encoding anti-rabies mAb containing the 3′ end A100 generated by DNA templates linearized using SapI. Group D: LNP-formulated mRNA encoding anti-rabies mAb containing the 3′ end A100-N5 generated by DNA templates linearized using EcoRI. Group E: PBS control. Further details are provided in example 1.

FIG. 3: shows the reduction of total dsRNA measured by dsRNA ELISA. Group A: LNP-formulated mRNA encoding anti-rabies mAb containing the 3′ end hSL-A100 generated by DNA templates linearized using SapI. Group B: LNP-formulated mRNA encoding anti-rabies mAb containing the 3′ end hSL-A100-N5 generated by DNA templates linearized using EcoRI. Group C: LNP-formulated mRNA encoding anti-rabies mAb containing the 3′ end A100 generated by DNA templates linearized using SapI. Group D: LNP-formulated mRNA encoding anti-rabies mAb containing the 3′ end A100-N5 generated by DNA templates linearized using EcoRI. Group E: PBS control. Further details are provided in example 1.7.

FIG. 4: shows that formulated mRNA encoding malaria CSP vaccine which template DNA strand has been linearized using EcoRI (group 1) or SapI (group 2) induces humoral immune responses (IgG1 and IgG2a endpoint titers) in mice, using an ELISA assay. FIG. 4A: IgG1 endpoint titers of GSP at day 21 and day 35 post vaccination. FIG. 48: IgG2a endpoint titers of the GSP at day 21 and day 35 post vaccination. FIG. 4C: Innate immune response (IFNa) of formulated mRNA encoding malaria CSP vaccine. The 3′ end format (group 2) for which the IVT template DNA was linearized using SapI endonuclease lead to a reduced immune response displayed by a reduction of INFα. Group 1: LNP formulated malaria CSP mRNA vaccine having the 3′ end hSL-A64-N5 linearized using EcoRI endonuclease. Group 2: LNP formulated malaria CSP mRNA vaccine having the 3′ end hSL-A100 linearized using SapI endonuclease. Group 3: NaCl buffer. d=day. Further details are provided in example 2.3 and 2.4.

FIG. 5: shows that formulated mRNA encoding malaria CSP vaccine which template DNA strand has been linearized using EcoRI (group A) or SapI (group B) induces humoral immune responses (IgG1 and IgG2a endpoint titers) in mice, using an ELISA assay. FIG. 5A: IgG1 endpoint titers of GSP at day 21 and day 35 post vaccination. FIG. 5B: IgG2a endpoint titers of the GSP at day 21 and day 35 post vaccination. FIG. 5C: Innate immune response (IFNa) of formulated mRNA encoding malaria CSP vaccine. The 3′ end format (group B) for which the IVT template DNA was linearized using SapI endonuclease lead to a reduced innate immune response displayed by a reduction of IFNa. Group A: LNP formulated malaria CSP mRNA vaccine having the 3′ end hSL-A64-N5 linearized using EcoRI endonuclease. Group B: LNP formulated malaria CSP mRNA vaccine having the 3′ end hSL-A100 linearized using SapI endonuclease. Group C: NaCl buffer. d=day. Further details are provided in example 2.3 and 2.4.

FIG. 6: shows the reactogenicity and immunogenicity after intramuscular application of different IVT mRNAs encoding Rabies virus G protein (RABV-G) which template DNA strands have been linearized using SapI (R8438, R8379, R8381, R7488) or EcoRI (R1803, R8437, R8378, R8380). All comprising the 3′UTR muag, except of the mRNA R7488 comprising UTR combination 5′UTR HSD17B4 and 3′UTR PSMB3. Additionally, non-modified mRNAs (R1803, R8437, R8438 and R7488) were compared with mRNAs comprising modified nucleotides, pseudouridine (ψ) (R8378 and R8379) and N1-methylpseudouridine (m1ψ) (R8380 and R8381). Further details are provided in Example 3.

FIG. 6A shows that formulated mRNA linearized using SapI (R8438 and R7488) led to a reduced reactogenicity and innate immune response after i.m. injection, displayed by reduced IFNa levels in the serum. IVT mRNA linearized with EcoRI comprising modified nucleotides showed reduced IFNa levels as well (pseudouridine R8378 and N1-methyl-pseudouridine R8380). LLOS is the abbreviation of “lowest limit of standard”. FIG. 6B shows that formulated mRNA linearized with SapI (R8438 and R7488) led to early VNT production. FIG. 6C shows comparable VNT levels for mRNA linearized with SapI comprising modified (R8378, R8379, R8380 and R8381) and non-modified nucleotides (R8438 and R7488). FIGS. 6D and E show CD4 and CD8 positive T cell responses. The populations of IFNγ and TNFα positive CD4 positive T cells (FIG. 6D) are comparable wherein the non-modified mRNA linearized with SapI comprising the UTR combination HSD17B4/PSMB3 (R7488) showed the highest CD8 positive immune response (FIG. 6E).

FIG. 7: shows the reactogenicity and immunogenicity after intramuscular application of different IVT mRNAs encoding Rabies virus G protein (RABV-G) which template DNA strands have been linearized using SapI (R7488, R8441 and R8442) or EcoRI (R1803, R8323, R8447, R8448). All mRNAs comprising the UTR combination 5′UTR HSD17B4 and the 3′UTR PSMB3, except of R1803 (only 3′UTR of alpha globulin, muag). Additionally non-modified mRNA (R1803, R8323, R7488) were compared with mRNA comprising modified nucleotides, pseudouridine (W) (R8447 and R8441) and N1-methylpseudouridine (m1ψ) (R8448 and R8442). Further details are provided in Example 3.

FIG. 7A shows that formulated non-modified mRNA linearized with EcoRI (R1803 and R8323) led to high reactogenicity and innate immune responses, displayed by high IFNa levels in the serum. LLOS is the abbreviation of “lowest limit of standard”. FIG. 7B shows that non-modified mRNA (R7488) and mRNA comprising pseudouridine (R8441) linearized with SapI had early VNT titers. FIG. 6C shows that all mRNA comprising the 5′UTR HSD17B4 and the 3′UTR PSMB3 led to a late VNT production. FIGS. 7D and E show CD4 and CD8 positive T cell responses measured in an ICS. IVT mRNA linearized with SapI (R7488, R8441 and R8442) showed a slightly better CD4 positive immune response compared to the IVT mRNA linearized with EcoRI (R1803, R8323, R8447, R8448) (Figure FD). The IVT mRNA linearized with SpaI show high CD8 positive immune responses (FIG. 7E).

FIG. 8: shows the reactogenicity and immunogenicity after intramuscular application of different IVT mRNAs encoding Rabies virus G protein (RABV-G) which template DNA strands have been linearized using SapI (R8318, R8321 and R8384) or EcoRI (R1803, R8317, R8320, R8383). All comprising the UTR combination 5′UTR SLC7A3 and the 3′UTR PSMB3, except of R1803 (only 3′UTR of alpha globulin, muag). Additionally non-modified mRNA (R1803, R8317 and R8318) were compared with mRNA comprising modified nucleotides, pseudouridine (ψ) (R8320 and R8321) and N1-methylpseudouridine (m1ψ) (R8383 and R8384). Further details are provided in Example 3.

FIG. 8A shows that formulated non-modified IVT mRNA linearized with EcoRI comprising the UTR combination SLC7A3/PSMB3 (R8317) led to high reactogenicity and innate immune responses, displayed by IFNa levels in the serum. LLOS is the abbreviation of “lowest limit of standard”. FIG. 8B shows early high VNT levels for all constructs, except for the non-modified mRNA (R1803) and the mRNA comprising pseudouridine (R8320). Both IVT mRNA has been linearized using EcoRI. For late VNT levels in FIG. 8C the two mRNA comprising pseudouridine, linearized with EcoRI (R8320) and SapI (R8321) led to lower levels compared to the non-modified mRNA (R1803, R8317 and R8318) or mRNA comprising N1-methylpseudouridine (R8282 and R8384). Figure D shows CD4 positive T cell responses. The non-modified mRNA linearized with SapI (R8318) led to the highest positive CD4 positive T cell population. FIG. 8E shows CD8 positive T cell responses. The non-modified mRNA linearized with SapI (R8318) led to the highest positive CD8 positive T cell population.

FIG. 9: shows the reactogenicity and immunogenicity after intramuscular application of different IVT mRNAs encoding Rabies virus G protein (RABV-G) which template DNA strands have been linearized using SapI (R8462, R8463, R8466, R8467 and R7488) or EcoRI (R1803, R8324, R8444, R8326 and R8446). The mRNA comprising different UTR combinations; 5′UTR HSD17B4 and 3′UTR FIG. 4.1 (R8324, R8444, R8462 and R8463) or the UTR combination 5′UTR UBQLN2 and 3′UTR RPS9.1 (R8326, R8446, R8466 and R8467) or the UTR combination 5′UTR HSD17B4 and 3′UTR PSMB3 (R7488). Additionally non-modified mRNA (R1803, R8324, R8462, R8326, R8466 and R7488) were compared with mRNA comprising modified nucleotides, pseudouridine (tp) (R8444, R8463, R8446 and R8467). Further details are provided in Example 3.

FIG. 9A shows that IVT mRNA linearized with SapI led to reduced reactogenicity and innate immune response, displayed by IFNa levels in the serum. The formulated non-modified IVT mRNA linearized with EcoRI leads to higher innate immune responses, independent of the UTR combination. LLOS is the abbreviation of “lowest limit of standard”. FIG. 8B and FIG. 9C show VNT levels in early (FIG. 9B) and later time points (FIG. 9C). The UTR combination 5′UTR UBQLN2 and 3′UTR RPS9.1 and the UTR combination 5′UTR HSD17B4 and 3′UTR PSMB3 linearized with SapI led to the highest VNT levels (R8446, R8467 and R7488). All IVT mRNA led to CD4 positive T cell responses (FIG. 9D), whereby the IVT mRNA linearized with SapI (R8462, R8463, R8466, R8467 and R7488) led to high CD8 positive T cell responses (FIG. 9E). Non-modified IVT mRNA led to higher responses than IVT mRNA comprising modified nucleotides.

FIG. 10: shows the dsRNA content of two different IVT RNAs (RNA 3 and RNA 4) which template DNA strand has been linearized using SapI and purified with two steps of cellulose purification or one step of oligo d(T) purification. The RNA fractions purified with oligo d(T) purification showed less dsRNA content compared to the fractions purified with cellulose purification.

The asterisk “*” in the figure indicates that the value of measured dsRNA in the RNA 3 fraction purified with oligo d(T) purification was lower than the limit of quantification of the dsRNA ELISA (<0,03 ng dsRNA/μg RNA). Further details are provided in Example 5.

### EXAMPLES

In the following, examples illustrating various embodiments and aspects of the temperature stable composition and/or vaccine of the invention are presented. However, the present invention shall not to be limited in scope by the specific embodiments presented herein, and should rather be understood as being applicable to other temperature stable composition and/or vaccine as for example defined in the specification. Accordingly, the following preparations and examples are given to enable those skilled in the art to more clearly understand and to practice the present invention. The present invention is not limited in scope by the exemplified embodiments, which are merely intended as illustrations of single aspects of the invention, and methods, which are functionally equivalent, are within the scope of the invention.

Indeed, various modifications of the invention in addition to those described herein will become readily apparent to those skilled in the art from the foregoing description, accompanying figures and the examples below.

### Example 1: Immunogenicity after Intravenously Application of Different mRNA-Formats Encoding an Anti-Rabies B Antibody

1.1 Preparation of DNA Templates

For the present examples, DNA sequences encoding different proteins were prepared and used for subsequent in vitro transcription reactions. The DNA sequences encoding the proteins were prepared by introducing an optimized sequence for stabilization. Sequences were introduced into a derived pUC19 vector. For further stabilization and/or increased translation, UTR elements were introduced 5′ and/or 3′ of the coding region. Obtained plasmid DNA was transformed and propagated in E. coli bacteria using common protocols. Plasmid DNA was isolated and purified before subsequent linearization.

1.2 RNA In Vitro Transcription from Plasmid DNA Templates

1.2.1 mRNA Design

Antibody sequences were designed as follows:

Anti-rabies mAb: 5′-UTR from HSD17B4 (hydroxysteroid (17-β) dehydrogenase-GC-enriched coding sequence encoding heavy or light chain of anti-rabies mAb (SO57, GenBank accession numbers AAO17821.1 and AAO17824.1)-3′-UTR derived from PSMB3 (proteasome subunit beta 3) -optionally a histone stem-loop sequence and a stretch of 100 adenosines.

1.2.2 Preparation of mRNA Encoding Anti-Rabies mAb:

DNA plasmids prepared according to section 1.1 were enzymatically linearized using SapI or EcoRI restriction endonucleases, purified and used for DNA-dependent in vitro transcription using T7 RNA polymerase in the presence of a sequence-optimized nucleotide mixture without chemical modification (ATP/GTP/CTP/UTP) and a cap analogue (m7G(5′)ppp(5′)(2′OMeA)pG) under suitable buffer conditions. The obtained in vitro transcribed RNA was purified using RP-HPLC (PureMessenger®; WO2008/077592) and used for in vitro and in vivo experiments. For in vivo studies mRNAs encoding heavy and light chain of an anti-rabies mAb (SO57, Thran et al., 2017) were mixed at a 2:1 molar ratio (heavy chain mRNA: light chain mRNA) before formulation into LNPs.

1.3 Example LNP Formulation

Lipid nanoparticles comprising ionizable or cationic lipids, phospholipids, cholesterol and polymer-conjugated lipids (PEG-lipids) were prepared and tested according to the general procedures described in PCT Pub. Nos. WO2015/199952, WO2017/004143, WO2013/116126, WO2018/078053 and WO2017/075531, the full disclosures of which are incorporated herein by reference. Lipid nanoparticle (LNP)-formulated mRNA was prepared using an ionizable amino lipid (carrying a net positive charge at a selective pH, such as physiological pH), phospholipid, cholesterol and a PEGylated lipid. LNPs were prepared as follows: Cationic lipid, DSPC, cholesterol and PEG-lipid were solubilized in ethanol at a molar ratio of approximately 50:10:38.5:1.5 or 47.4:10:40.9:1.7. LNPs for the Examples included, for example, cationic lipid compound III-3 as disclosed in WO 2018/078053 and the foregoing components. Lipid nanoparticles (LNP) comprising compound II1-3 as disclosed in WO 2018/078053 were prepared at a ratio of mRNA to total lipid of 0.03-0.04 w/w. Briefly, the mRNA was diluted to 0.05 to 0.2 mg/ml in 10 to 50 mM citrate buffer, pH 4. Syringe pumps were used to mix the ethanolic lipid solution with the mRNA aqueous solution at a ratio of about 1:5 to 1:3 (vol/vol) with total flow rates above 15 ml/min. The ethanol was then removed and the external buffer replaced with PBS by dialysis. Finally, the lipid nanoparticles were filtered through a 0.2 μm pore sterile filter. Lipid nanoparticle particle diameter size was 60-90 nm as determined by quasi-elastic light scattering using a Malvern Zetasizer Nano (Malvern, UK). For other cationic lipid compounds mentioned in the present specification, the formulation process is similar.

1.4 Injection of Mice Using Different mRNA-Formats Encoding an Anti-Rabies Monoclonal Antibody

For in vivo studies mRNAs encoding heavy and light chain of an anti-rabies mAb (SO57) were mixed at a 2:1 molar ratio (heavy chain mRNA: light chain mRNA) before formulation into LNPs. For injections, mRNA-LNP were diluted in phosphate-buffered saline pH 7.4.

BALB/c mice were intravenously injected into the tail vein with 10 μg mRNA-LNP in a volume of 100 μl (0.5 mg/kg) according to the injection scheme shown in Table Ill. A total of 5 groups each at 8 mice were treated with 4 mice being injected with phosphate buffered saline (PBS) only. Serum mAb levels were determined at different time points (4 h and 24 h after injection, respectively).

TABLE III
Injection scheme of different mRNA-formats encoding an anti-rabies monoclonal antibody

| | SEQ ID | | | Restriction | | | |
|---|---|---|---|---|---|---|---|
| mRNA | NO: | | | enzyme used | | Dose per | |
| ID | mRNA | Group | 3' end | for linearization | Animals | animal | Injection |
| R8585, | 102, 103 | A | hSL-A100 | Sapl | 8 | 10 μg | mRNA-LNP |
| R8586 | | | | | | (0.5 mg/kg) | |
| R8823, | 104, 105 | B | hSL-A100-N5 | EcoRI | 8 | 10 μg | mRNA-LNP |
| R8827 | | | | | | (0.5 mg/kg) | |
| R7902, | 106, 107 | C | A100 | Sapl | 8 | 10 μg | mRNA-LNP |
| R7908 | | | | | | (0.5 mg/kg) | |
| R8824, | 108, 109 | D | A100-N5 | EcoRI | 8 | 10 μg | mRNA-LNP |
| R8828 | | | | | | (0.5 mg/kg) | |
| | | E | PBS control | / | 4 | / | PBS |

1.5 Antibody Analysis (ELISA)

Antibody analysis to measure IgG titers was performed by ELISA.

Goat anti-human IgG (1 mg/ml; SouthernBiotech; Cat. 2044-01) was diluted 1:1000 in coating buffer (15 mM Na2CO3, 15 mM NaHCO3and 0.02% NaN3, pH 9.6) and used to coat Nunc MaxiSorp® flat bottom 96-well plates (Thermo Fischer) with 100 μl for 4 h at 37° C. After coating, wells were washed three times (PBS pH 7.4 and 0.05% Tween-20) and blocked overnight in 200 μl blocking buffer (PBS, 0.05% Tween-20 and 1% BSA) at 4° C. Human IgG1 control antibody (Erbitux at 5 mg/ml; Merck, PZN 0493528) was diluted in blocking buffer to 100 μg/ml. Starting with this solution, a serial dilution was prepared for generating a standard curve. Samples were diluted appropriately in blocking buffer (PBS, 0.05% Tween-20, and 1% BSA) to allow for quantification. All further incubations were carried out at room temperature. Diluted supernatants or sera were added to the coated wells and incubated for 2 h. Solution was discarded and wells were washed three times. Detection antibody (goat anti-human IgG Biotin, Dianova; Cat. 109065088) was diluted 1:20000 in blocking buffer, 100 μl was added to wells and incubated for 60-90 min. Solution was discarded and wells were washed three times. HRP-streptavidin (BD Pharmingen™, Cat. 554066) was diluted 1:1000 in blocking buffer, 100 μl was added to wells and incubated for 30 min. HRP solution was discarded and wells were washed four times. 100 μl of Tetramethylbenzidine (TMB, Thermo Scientific, Cat. 34028) substrate was added and reaction was stopped by using 100 μl of 20% sulfuric acid.

1.6 Cytokine Analysis

Blood samples of mice were taken 4 h and 24 h from mice after injection of mRNA-LNP encoding anti-rabies mAb (SO57) to determine the inflammation biomarker IFNalpha using VeriKine-HS Mouse IFNalpha. All Subtype ELISA Kit (pbl) according to manufacturer's instructions. Further cytokines (IL-6, MIP-1β, MCP1, Rantes, TNF, INFγ, MIG) were measured by Cytometric Bead Array (CBA) according to the manufacturer's instructions (BD Biosciences).

1.7 dsRNA Analysis (ELISA)

9D5 antibody (absolute antibody) was diluted to 2 μg/ml in PBS and used to coat Nunc MaxiSorp® flat bottom 96-well plates (Thermo Fischer) with 100 μl for 2 h at room temperature. After coating, wells were washed three times using PBS-T (PBS and 0.05% Tween-20). Samples and standards were diluted in 1×TE buffer (AppliChem) and 100 μl were added to each well and incubated over night at 4° C. (approx. 20 h). After incubation, wells were washed three times using PBS-T. K2 antibody (Scicon) was diluted 1:200 in PBST and 100 μl were added to each well and incubated for 2 h at room temperature. Wells were washed three times using PBS-T. Anti-mouse IgM-HRP (Invitrogen) was diluted 1:50 in PBST and 100 μl were added to each well and incubated for 1 h at room temperature. Wells were washed three times using PBS-T. Color reagents A and B (R&D systems) were mixed in equal amounts and 100 μl were added to each well and incubated for 9 minutes. Plates were measured in a plate reader at OD450 and OD540. OD540 values were subtracted from OD450 values and used for the determination of relative dsRNA amounts compared to a standard, an mRNA preparation with apparent dsRNA signals.

TABLE IV
dsRNA analysis results

| | | SEQ ID | Measured | |
|---|---|---|---|---|
| | mRNA | NO | dsRNA | Total |
| Group | ID | mRNA | (%) | (%) |
| A | R8585 | 102 | <0.4 | 0.554 |
| | R8586 | 103 | 1.1 | |
| B | R8823 | 104 | 30.3 | 26.428 |
| | R8827 | 105 | 12.7 | |
| C | R7902 | 106 | 4.4 | 3.52 |
| | R7908 | 107 | <0.4 | |
| D | R8824 | 108 | 42.8 | 55.384 |
| | R8828 | 109 | >100 | |

1.8 Results

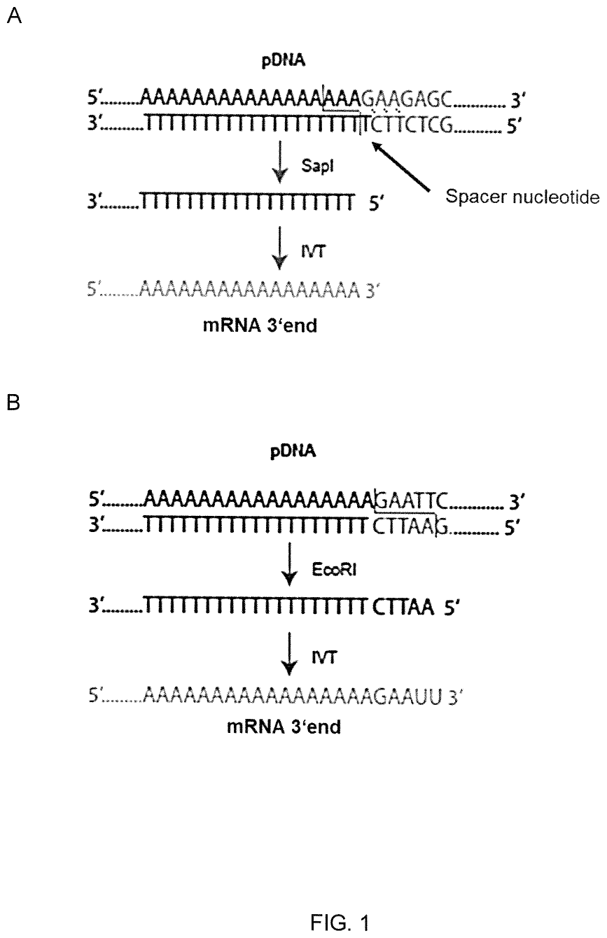

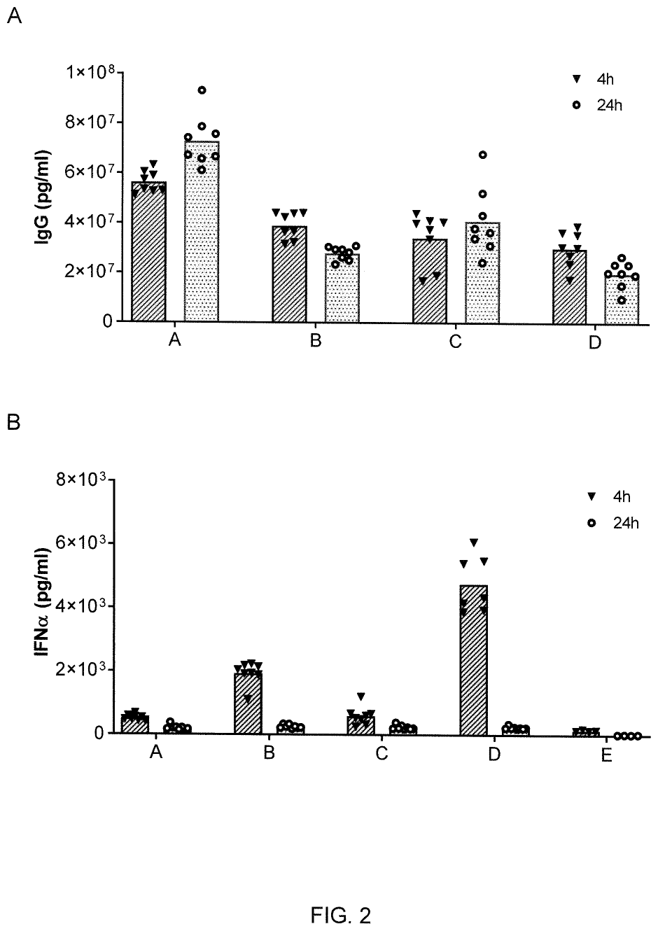

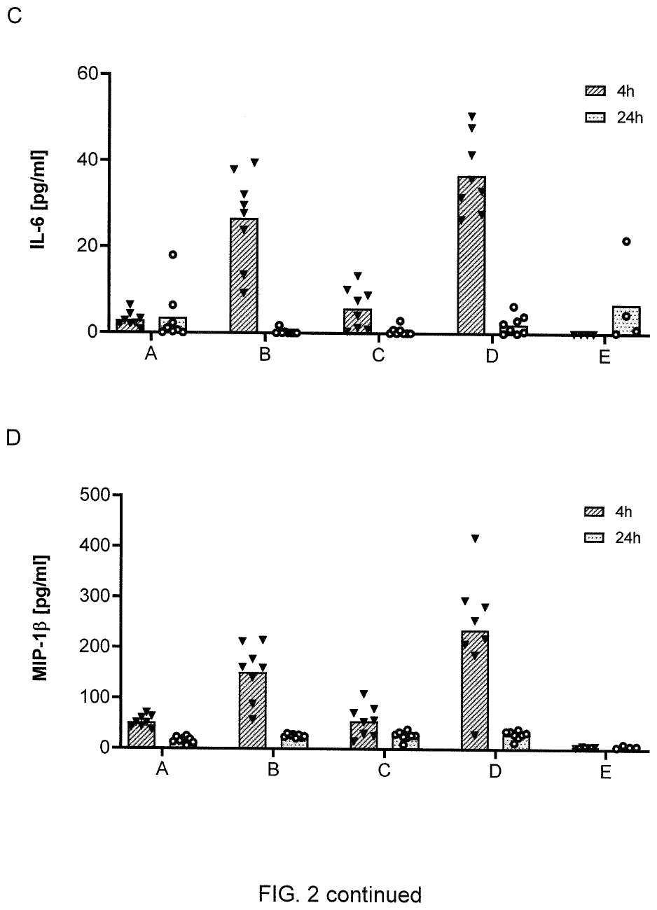

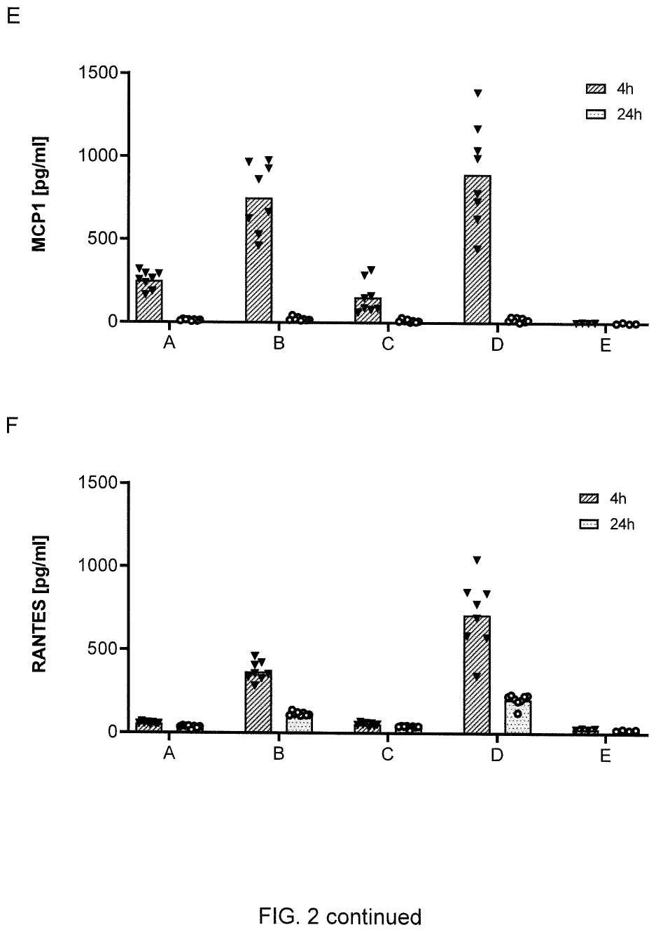

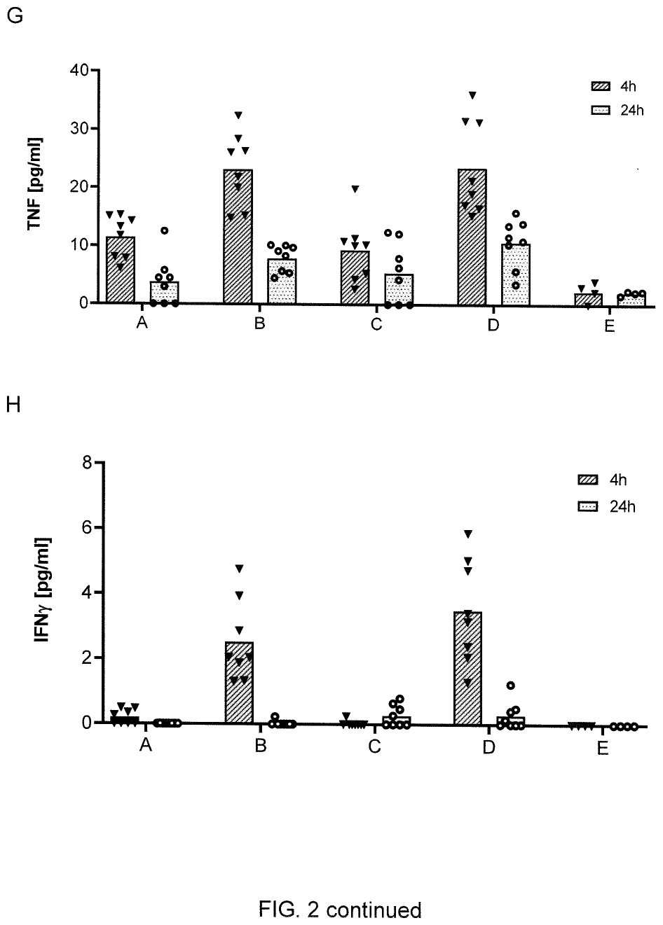

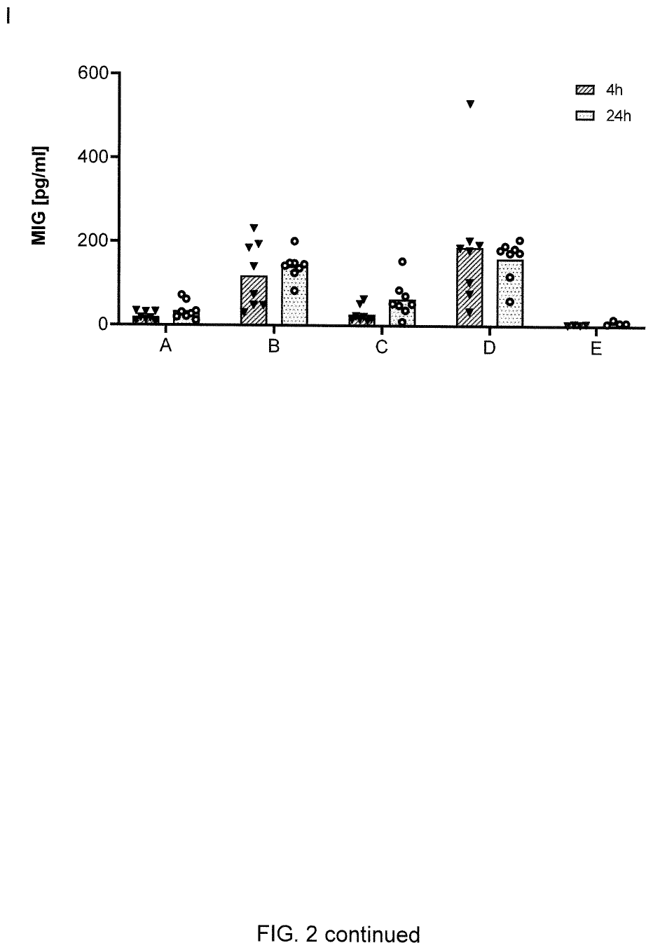

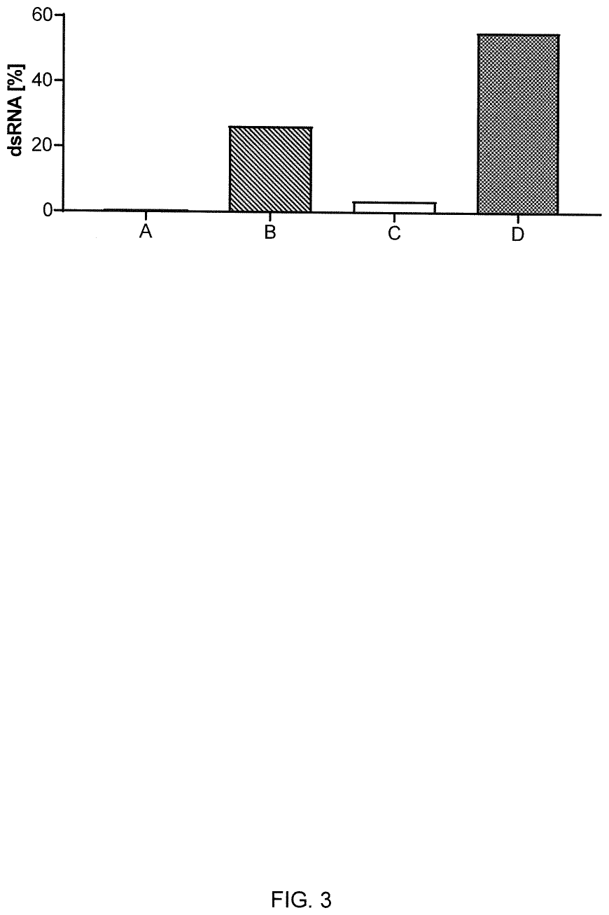

Antibody titer analysis (IgG) is shown in FIG. 2A. The intravenous injection of LNP-formulated RNA encoding anti-rabies mAb (SO57) of mice led to expression of detectable human IgG antibodies for all constructs. A reduction of IFNalpha level can be seen at 4 h and 24 h for the constructs (Group A and C), which were generated by DNA templates linearized using SapI endonuclease. (FIG. 2B). Further cytokines (IL-6, MIP-13, MCP1, Rantes, TNF, INFγ, MIG) showed also a reduction at 4 h and 24 h for the constructs (Group A and C), which were generated by DNA templates linearized using SapI endonuclease (FIG. 2C-1). Measurement of dsRNA content also showed to be reduced by less than 5% for the constructs (Group A and C), which were generated by DNA templates linearized using SapI endonuclease (Table IV and FIG. 3).

### Example 2: Immunogenicity after Intravenously Application of Different mRNA-Formats Encoding Circumsporozoite protein (CSP) of a malaria parasite

2.1 Preparation of RNA and DNA constructs

DNA sequences encoding a short length form (HsALB(1-18)_Pf-CSP(19-397)) of the circumsporozoite protein (CSP) of a malaria parasite (e.g. Plasmodium falciparum) were prepared and used for subsequent RNA in vitro transcription reactions.

Said DNA sequences were prepared by modifying the wild type or reference encoding DNA sequences by introducing a G/C optimized coding for stabilization and expression optimization. Sequences were introduced into a pUC derived DNA vector to comprise stabilizing 3′-UTR sequences and 5′-UTR sequences, additionally comprising a stretch of adenosines (e.g. A64 or A100), and optionally a histone stem-loop (hSL) structure (see table V).

The obtained plasmid DNA constructs were transformed and propagated in bacteria using common protocols known in the art. Eventually, the plasmid DNA constructs were extracted, purified, and used for subsequent RNA in vitro transcription.

2.2 RNA In Vitro Transcription from Plasmid DNA Templates:

DNA plasmids prepared according to paragraph 1.1 were linearized using a restriction enzyme and used for DNA dependent RNA in vitro transcription using T7 RNA polymerase in the presence of a nucleotide mixture (ATP/GTP/CTP/UTP) and cap analog (e.g. m7GpppG) under suitable buffer conditions. m7G(5′)ppp(5′)(2′OMeA)pG cap analog was used for preparation of some RNA constructs to generate a cap1 structure (e.g. R8523, R8520).

Obtained RNA constructs were purified using RP-HPLC (PureMessenger®, CureVac AG, Tubingen, Germany; WO2008/077592) and used for in vitro and in vivo experiments. The generated RNA sequences/constructs are provided in Table V, with the encoded CSP constructs and the respective UTR elements indicated therein (mRNA design a-1 (HSD17B4/PSMB3)). CSP proteins and fragments were derived from Plasmodium falciparum 3D7 (XP_001351122.1, XM_001351086.1; abbreviated herein as “Pf(3D7)”).

Some RNA constructs may be in vitro transcribed in the absence of a cap analog. The cap structure (cap1) may be added enzymatically using capping enzymes as commonly known in the art. In short, in vitro transcribed mRNA may be capped using an m7G capping kit with 2′-O-methyltransferase to obtain cap1-capped RNA.

The obtained mRNAs are purified e.g. using RP-HPLC (PureMessenger®, CureVac AG, Tubingen, Germany; WO2008/077592) and used for in vitro and in vivo experiments.

TABLE V
mRNA constructs encoding malaria CSP used in the present example

| | | Restriction | | | | SEQ ID |
|---|---|---|---|---|---|---|
| mRNA | | enzyme used for | UTR | 3' end | 5' cap | NO: |
| ID | Protein | linearization | Design | | structure | mRNA |
| R8523 | HsALB(1-18)_Pf-CSP(19-397) | EcoRI | a-1 | hSL-A64-N5 | cap1 | 98 |
| R8520 | HsALB(1-18)_Pf-CSP(19-397) | Sapl | a-1 | hSL-A100 | cap1 | 99 |
| R8987 | HsALB(1-18)_Pf-CSP(19-397) | EcoRI | a-1 | hSL-A100-N5 | cap1 | 100 |

2.3 Vaccination of Mice with LNP-Formulated mRNA Encoding CSP

Malaria mRNA vaccine candidates encoding full length and short length form of CSP were prepared according to Example 2, and the mRNA constructs were formulated in lipid nanoparticles (see 1.3 Example LNP Formulation). The LNP formulations were applied on days 0 and 21 (Table VI) and 22 (Table VII) intramuscularly (i.m.; musculus tibialis, Balb/c mice) with doses of RNA formulations, and control groups as shown in Table VI and VII. The negative control group received NaCl buffer. Serum samples were taken at day 21 and day 35 for ELISA.

TABLE VI
Vaccination scheme A of example 2

| mRNA | | | Species/ | | | Route/ | |
|---|---|---|---|---|---|---|---|
| ID | Group | 3' end | Gender/N | Dose | Formulation | Volume | Dosing |
| R8523 | 1 (EcoRI) | hSL-A64-N5 | Balb/c mice, | 1 μg | LNP | i.m./25 μl | day 0 |
| R8520 | 2 (Sapl) | hSL-A100 | female, N = 5 | | | | and |
| | 3 (0.9% NaCl) | | | | | | day 21 |

TABLE VII
Vaccination scheme B of example 2

| mRNA | | | Species/ | | | Route/ | |
|---|---|---|---|---|---|---|---|
| ID | Group | 3\grave{ }end | Gender/N | Dose | Formulation | Volume | Dosing |
| R8987 | A (EcoRI) | hSL-A100-N5 | Balb/c mice, | 1 μg | LNP | i.m./25 μl | day 0 |
| R8520 | B (Sapl) | hSL-A100 | female, N = 8 | | | | and |
| | C (NaCl 0.9%) | | | | | | day 22 |

2.4 Determination of Specific Humoral Immune Responses by ELISA

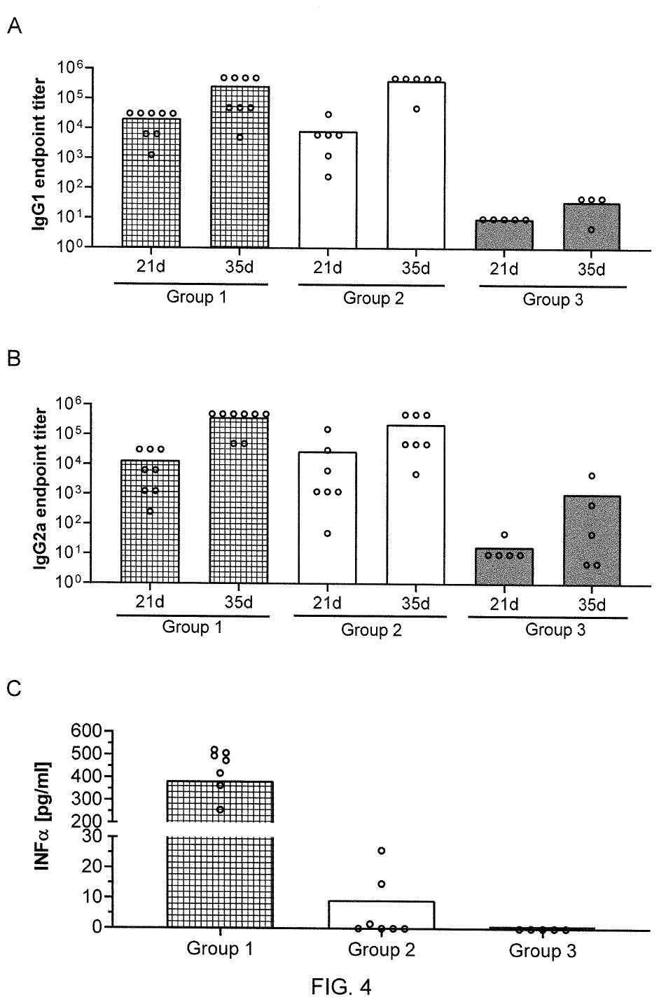

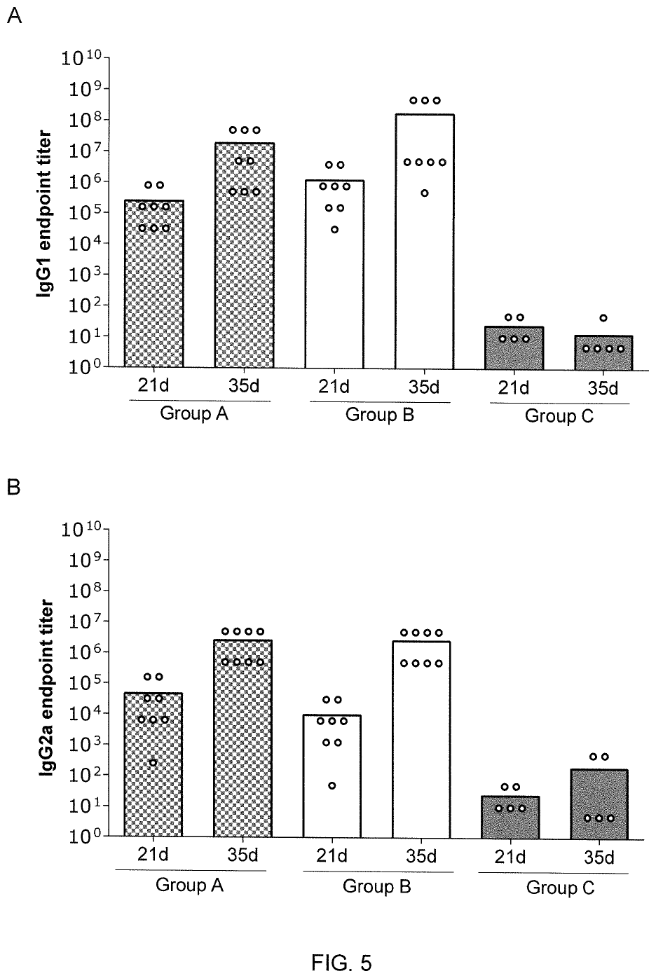

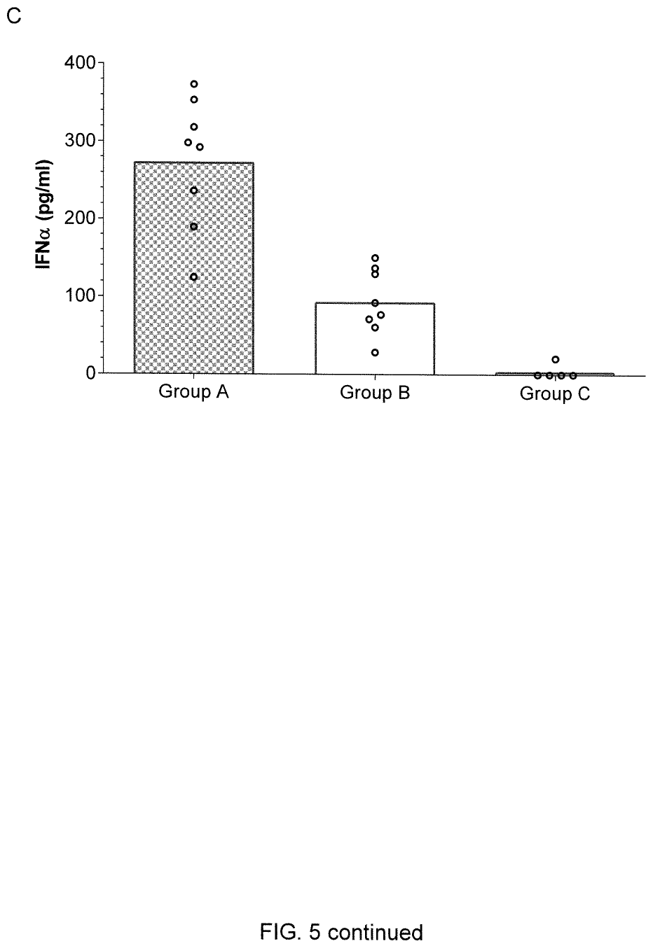

ELISA was performed using malaria [NANP]₇ peptide (according to SEQ ID NO: 101) for coating. Coated plates were incubated using respective serum dilutions, and binding of specific antibodies to the respective malaria [NANP]₇ peptide were detected using biotinylated isotype specific anti-mouse antibodies followed by streptavidin-HRP (horse radish peroxidase) with Amplex as substrate. Endpoint titers of antibodies (IgG1, IgG2a) directed against the malaria [NANP]7 peptide were measured by ELISA on day 21 and day 35 post vaccinations. Results are shown in FIG. 4A (IgG1) and 4B (IgG2a) for group 1, 2 and 3 and FIG. 5A (IgG1) and 5B (IgG2a) for group A, B and C.

2.5 In Vivo Analysis of Cytokines

Appropriate dilutions of sera collected 14 hours after prime vaccination (see Example 2.4) were analyzed by a mouse IFNalpha ELISA kit according to the manufacturer's protocol (PBL, cat.: 42115-1). Tables VI and VII contains mRNA constructs that were used in the experiment. Results are shown in FIG. 4C group 1, 2 and 3 and FIGS. 5C group A, B and C, respectively.

2.6 Results

The results from the binding antibody titers IgG1 and IgG2a are shown in FIG. 4A (IgG1) and 4B (IgG2a) for group 1, 2 and 3 and FIGS. 5A (IgG1) and 5B (IgG2a) for group A, B and C. The intramuscularly vaccination of mice with LNP-formulated malaria mRNA vaccine candidates encoding CSP led to strong induction of binding antibodies already after one vaccination at day 21 and after two vaccinations at day 35. A reduction of IFNalpha levels can be seen for the constructs, which were linearized using SapI endocuclease already after 14 h post vaccination in FIG. 4C group 1, 2 and 3 and FIG. 5C group A, B and C, respectively.

### Example 3: Reactogenicity and Immunogenicity after Intramuscular Application of Different IVT mRNAs Encoding Rabies Virus G Protein (RABV-G)

3.1 Preparation of RNA and DNA Constructs

DNA sequences encoding a transmembrane glycoprotein G of the rabies virus were prepared and used for subsequent RNA in vitro transcription reactions. Transmembrane glycoprotein G derived from the rabies virus, abbreviated herein as “RABV-G”. Said DNA sequences were prepared by modifying the wild type or reference encoding DNA sequences by introducing a G/C optimized coding for stabilization and expression optimization. Sequences were introduced into a pUC derived DNA vector to comprise stabilizing 3′-UTR sequences and optionally 5′-UTR sequences, additionally comprising a stretch of adenosines (e.g. A64 or A100), and optionally a histone stem-loop (hSL) structure (see Table VIII). The obtained plasmid DNA constructs were transformed and propagated in bacteria using common protocols known in the art. Eventually, the plasmid DNA constructs were extracted, purified, and used for subsequent RNA in vitro transcription.

3.2 RNA IN VITRO TRANSCRIPTION FROM PLASMID DNA TEMPLATES:

DNA plasmids prepared according to paragraph 3.1 were enzymatically linearized using SapI or EcoRI restriction endonucleases and used for DNA dependent RNA in vitro transcription using T7 RNA polymerase in the presence of a nucleotide mixture (ATP/GTP/CTP/UTP) and cap analog (e.g. m7GpppG or m7G(5′)ppp(5′)(2′OMeA)pG) under suitable buffer conditions.

To obtain modified mRNA, RNA in vitro transcription was performed in the presence of a modified nucleotide mixture (ATP, GTP, CTP, pseudouridine (ψ) or N1-methylpseudouridine (m1ψ)) and cap analog (m7G(5′)ppp(5′)(2′OMeA)pG) under suitable buffer conditions. Obtained RNA constructs were purified using RP-HPLC (PureMessenger®, CureVac AG, Tübingen, Germany; WO2008/077592, the full disclosures of which are incorporated herein by reference) and used for in vivo experiments. The generated RNA sequences are provided in Table VIII, with the encoded RABV-G constructs, respective UTR elements, cap structures, modifications, restriction enzymes used for linearization and 3′end of the mRNA construct indicated therein.

TABLE VIII
IVT mRNA encoding RABV-G used in the example 3

| | | Restriction | | | | | SEQ ID |
|---|---|---|---|---|---|---|---|
| mRNA | | enzyme used | UTR Design | | 5' cap | Modified | NO: |
| ID | Protein | for linearization | (5‘UTR/3‘UTR) | 3' end | structure | nucleotides | mRNA |
| R1803 | RABV-G | EcoRI | —/muag | A64-N5- | cap0 | — | 129 |
| | | | | C30-HSL- | | | |
| | | | | N5 | | | |
| R8437 | RABV-G | EcoRI | —/muag | HSL-A64- | cap1 | — | 130 |
| | | | | N5 | | | |
| R8438 | RABV-G | Sapl | —/muag | HSL-A100 | cap1 | — | 131 |
| R8378 | RABV-G | EcoRI | —/muag | HSL-A64- | cap1 | ΨU | 132 |
| | | | | N5 | | | |
| R8379 | RABV-G | Sapl | —/muag | HSL-A100 | cap1 | ΨU | 133 |
| R8380 | RABV-G | EcoRI | —/muag | HSL-A64- | cap1 | M1ΨU | 134 |
| | | | | N5 | | | |
| R8381 | RABV-G | Sapl | —/muag | HSL-A100 | cap1 | M1ΨU | 135 |
| R7488 | RABV-G | Sapl | HSD17B4/ | HSL-A100 | cap1 | — | 136 |
| | | | PSMB3 | | | | |
| R8323 | RABV-G | EcoRI | HSD17B4/ | HSL-A64- | cap1 | — | 137 |
| | | | PSMB3 | N5 | | | |
| R8447 | RABV-G | EcoRI | HSD17B4/ | HSL-A64- | cap1 | ΨU | 138 |
| | | | PSMB3 | N5 | | | |
| R8441 | RABV-G | Sapl | HSD17B4/ | HSL-A100 | cap1 | ΨU | 139 |
| | | | PSMB3 | | | | |
| R8448 | RABV-G | EcoRI | HSD17B4/ | HSL-A64- | cap1 | M1ΨU | 140 |
| | | | PSMB3 | N5 | | | |
| R8442 | RABV-G | Sapl | HSD17B4/ | HSL-A100 | cap1 | M1ΨU | 141 |
| | | | PSMB3 | | | | |
| R8317 | RABV-G | EcoRI | SLC7A3/ | HSL-A64- | cap1 | — | 142 |
| | | | PSMB3 | N5 | | | |
| R8318 | RABV-G | Sapl | SLC7A3/ | HSL-A100 | cap1 | — | 143 |
| | | | PSMB3 | | | | |
| R8320 | RABV-G | EcoRI | SLC7A3/ | HSL-A64- | cap1 | ΨU | 144 |
| | | | PSMB3 | N5 | | | |
| R8321 | RABV-G | Sapl | SLC7A3/ | HSL-A100 | cap1 | ΨU | 145 |
| | | | PSMB3 | | | | |
| R8383 | RABV-G | EcoRI | SLC7A3/ | HSL-A64- | cap1 | M1ΨU | 146 |
| | | | PSMB3 | N5 | | | |
| R8384 | RABV-G | Sapl | SLC7A3/ | HSL-A100 | cap1 | M1ΨU | 147 |
| | | | PSMB3 | | | | |
| R8443 | RABV-G | EcoR | HSD17B4/ | HSL-A64- | cap1 | — | 148 |
| | | | FIG4.1 | N5 | | | |
| R8444 | RABV-G | EcoRI | HSD17B4/ | HSL-A64- | cap1 | ΨU | 149 |
| | | | FIG4.1 | N5 | | | |
| R8462 | RABV-G | Sapl | HSD17B4/ | HSL-A100 | cap1 | — | 150 |
| | | | FIG4.1 | | | | |
| R8463 | RABV-G | Sapl | HSD17B4/ | HSL-A100 | cap1 | ΨU | 151 |
| | | | FIG4.1 | | | | |
| R8326 | RABV-G | EcoRI | UBQLN2/ | HSL-A64- | cap1 | — | 152 |
| | | | RPS9.1 | N5 | | | |
| R8446 | RABV-G | EcoRI | UBQLN2/ | HSL-A64- | cap1 | ΨU | 153 |
| | | | RPS9.1 | N5 | | | |
| R8466 | RABV-G | Sapl | UBQLN2/ | HSL-A100 | cap1 | — | 154 |
| | | | RPS9.1 | | | | |
| R8467 | RABV-G | Sapl | UBQLN2/ | HSL-A100 | cap1 | ΨU | 155 |
| | | | RPS9.1 | | | | |

3.3 LNP Formulation

Lipid nanoparticles comprising ionizable or cationic lipids, phospholipids, cholesterol and polymer-conjugated lipids (PEG-lipids) were prepared and tested according to the general procedures described in PCT Pub. Nos. WO2015/199952, WO2017/004143, WO2013/116126, WO2018/078053 and WO2017/075531, the full disclosures of which are incorporated herein by reference. Lipid nanoparticle (LNP)-formulated mRNA was prepared using an ionizable amino lipid (carrying a net positive charge at a selective pH, such as physiological pH), phospholipid, cholesterol and a PEGylated lipid. LNPs were prepared as follows: Cationic lipid, DSPC, cholesterol and PEG-lipid were solubilized in ethanol at a molar ratio of approximately 47.4:10:40.9:1.7. LNPs for the Example included, for example, cationic lipid compound Ill-3 as disclosed in WO 2018/078053 and the foregoing components. Lipid nanoparticles (LNP) comprising compound 111-3 as disclosed in WO 2018/078053 were prepared at a ratio of mRNA to total lipid of 0.03-0.04 w/w. Briefly, the mRNA was diluted to 0.05 to 0.2 mg/ml in 10 to 50 mM citrate buffer, pH 4. Syringe pumps were used to mix the ethanolic lipid solution with the mRNA aqueous solution at a ratio of about 1:5 to 1:3 (vol/vol) with total flow rates above 15 ml/min. The ethanol was then removed and the external buffer replaced with PBS by dialysis. Finally, the lipid nanoparticles were filtered through a 0.2 μm pore sterile filter. Lipid nanoparticle particle diameter size was 60-90 nm as determined by quasi-elastic light scattering using a Malvern Zetasizer Nano (Malvern, UK).

3.4 Vaccination of Mice Using Different Formulated IVT mRNA Encoding RABV-G to Show Immunogenicity after Intramuscular Application

To show immunogenicity of RABV-G-encoding mRNA formulated with LNPs (see Table VII) and are able to induce adaptive immune responses, mice were vaccinated according to vaccination schedule provided in Table IX.

TABLE IX
Vaccination schedule of example 3

| mRNA | | |
|---|---|---|
| Dose | | Dosing |
| [μg] | Route/volume | [day] |
| 1 | i.m./1 × 25 μl | 0.21 |

Serum samples were taken on day 21 and day 35, wherein the serum samples were analyzed for Virus Neutralizing Antibodies (VNA) analysis via FAVN assay. For said immunogenicity assays, the virus neutralizing titers (VNT) was measured as described in standard protocols, i.e. anti-rabies virus neutralizing titers (VNTs) in serum were analyzed by the Eurovir® Hygiene-Labor GmbH, Germany, using the FAVN assay and the Standard Challenge Virus CVS-11 according to WHO protocol.

35 days after the first mRNA administration, mice were sacrificed and blood and organ samples (spleen) were collected for further analysis. In this regard, rabies virus glycoprotein (RABV-G)-specific cellular responses in splenocyte samples obtained in this step were measured as RABV-G-specific T cell activation. Spleen samples were re-stimulated with a RABV-G peptide library and assayed for T cell responses (CD4 and CD8), i.e. CD4 T cell immune response (IFNγ/TNFα producing CD4 T cells) and CD8 T cell immune response (IFNγ/TNFα producing CD8 T cells). Induction of antigen-specific T cells was determined using intracellular cytokine staining (ICS) according to standard protocols as follows: splenocytes were stimulated with a RABV-G peptide cocktail in the presence of anti-CD107a (Biolegend, San Diego, USA) and anti-CD28 (BD Biosciences, San Jose, USA). After the stimulation procedure, splenocytes were stained with fluorophore-conjugated antibodies and analysed by flow cytometry surface and intracellularly.

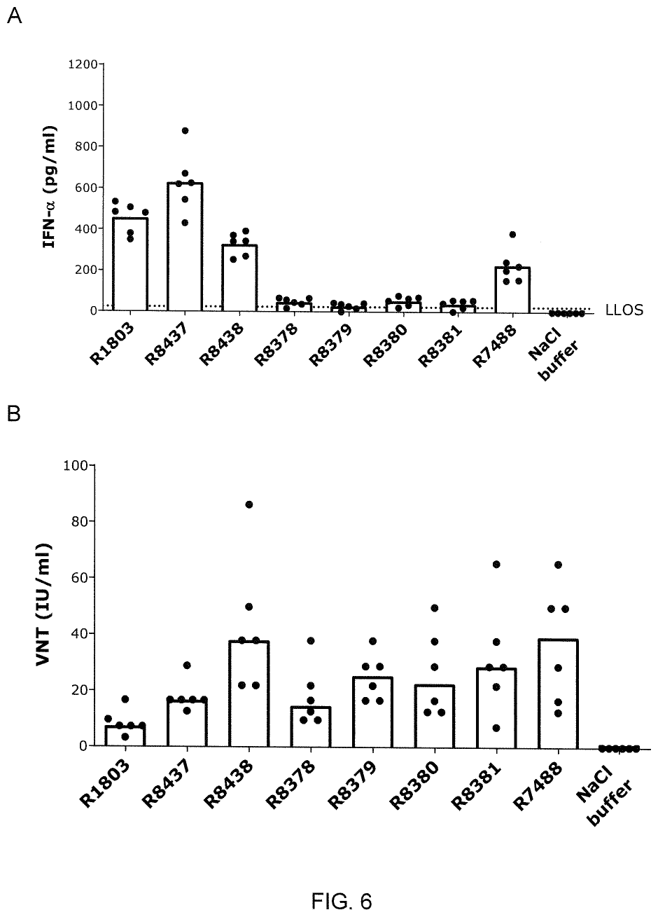

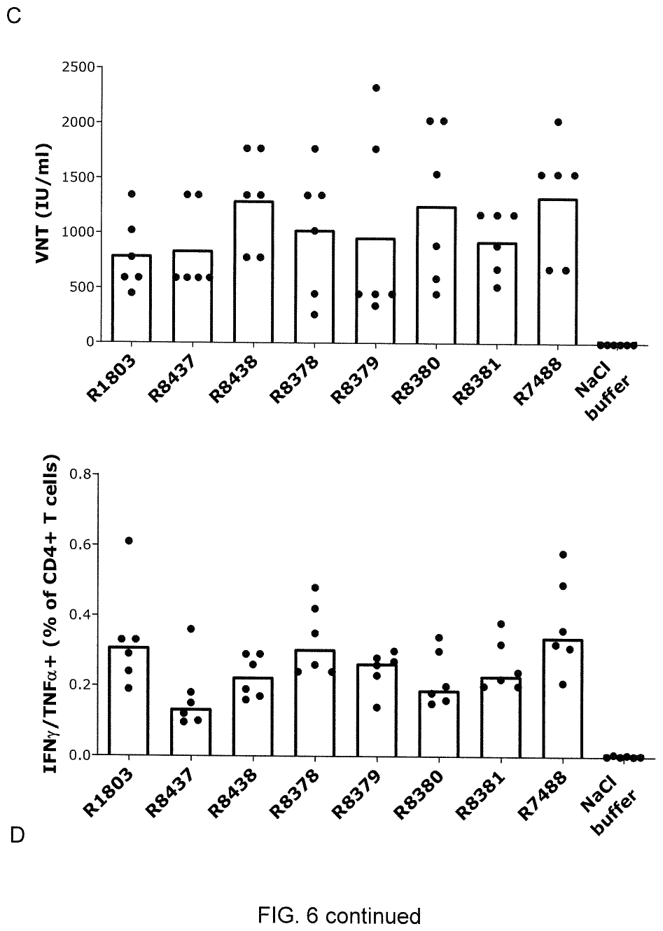

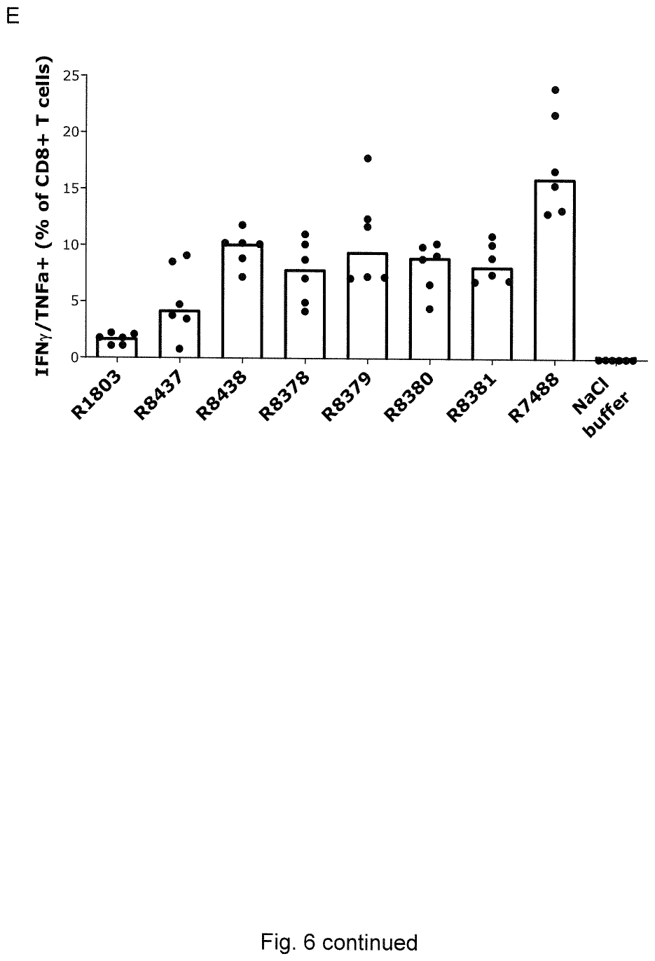

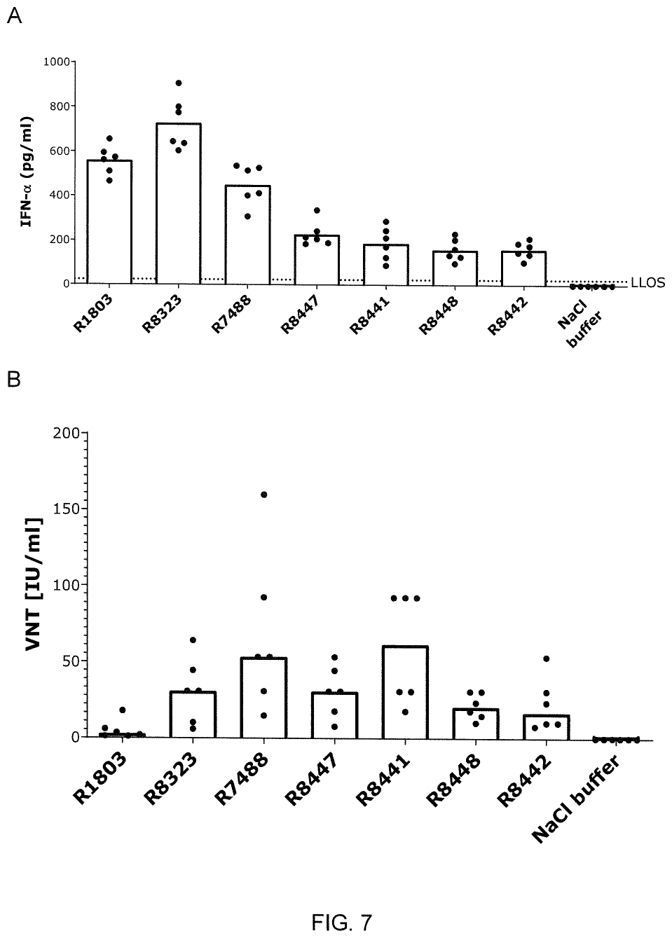

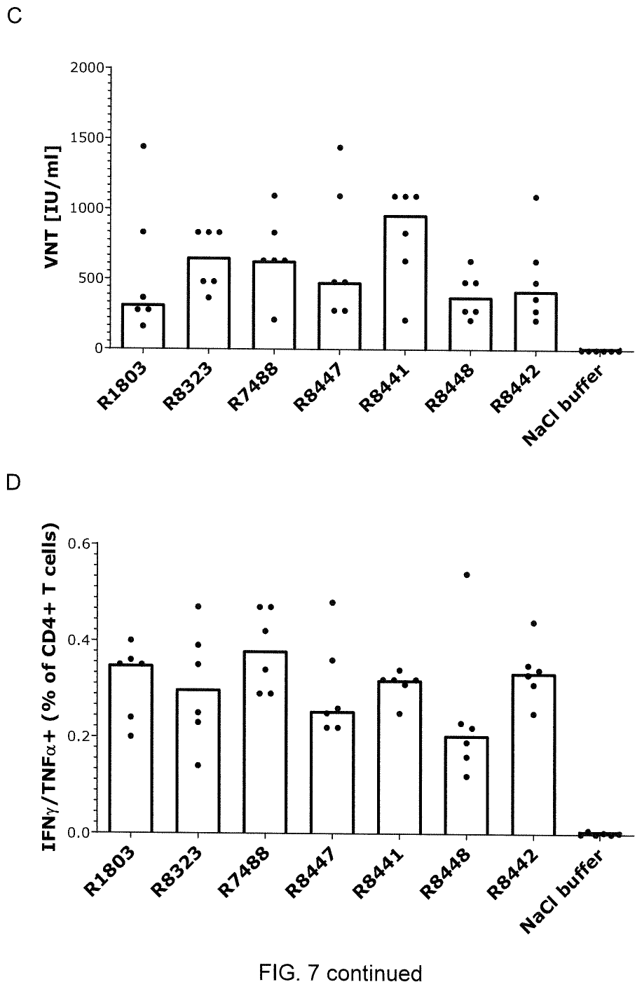

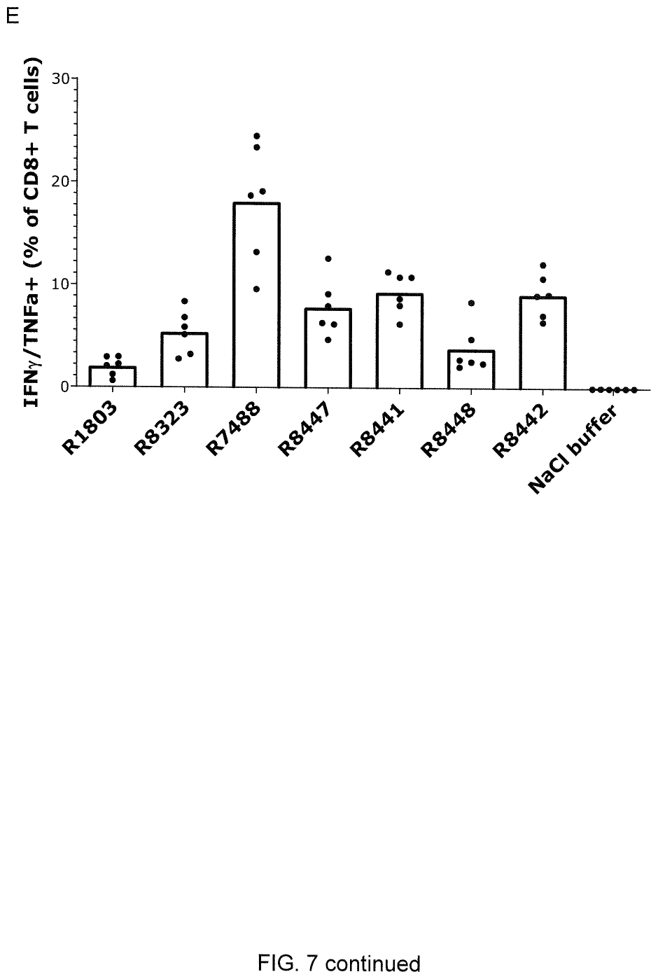

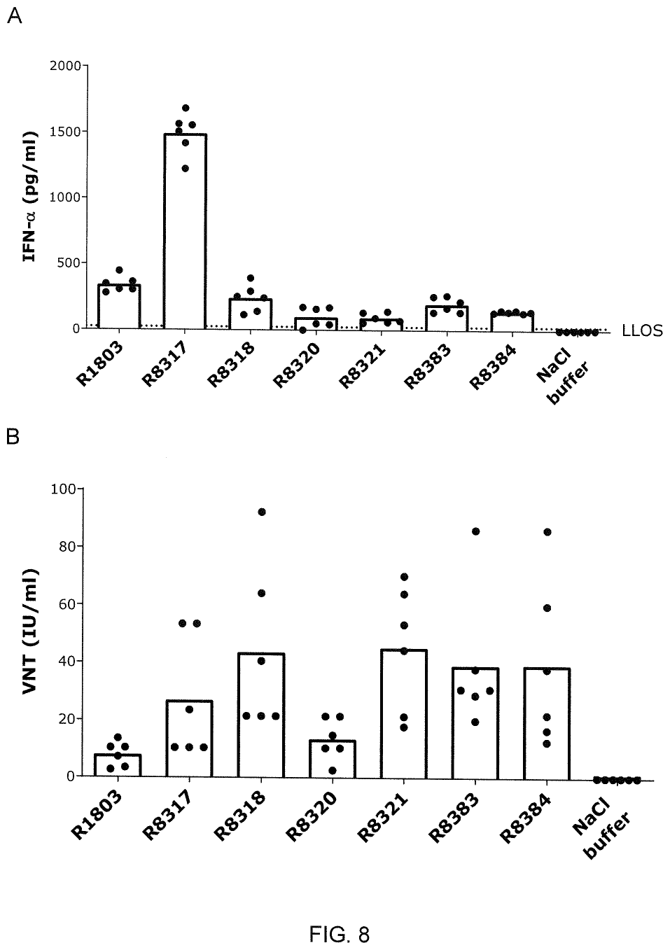

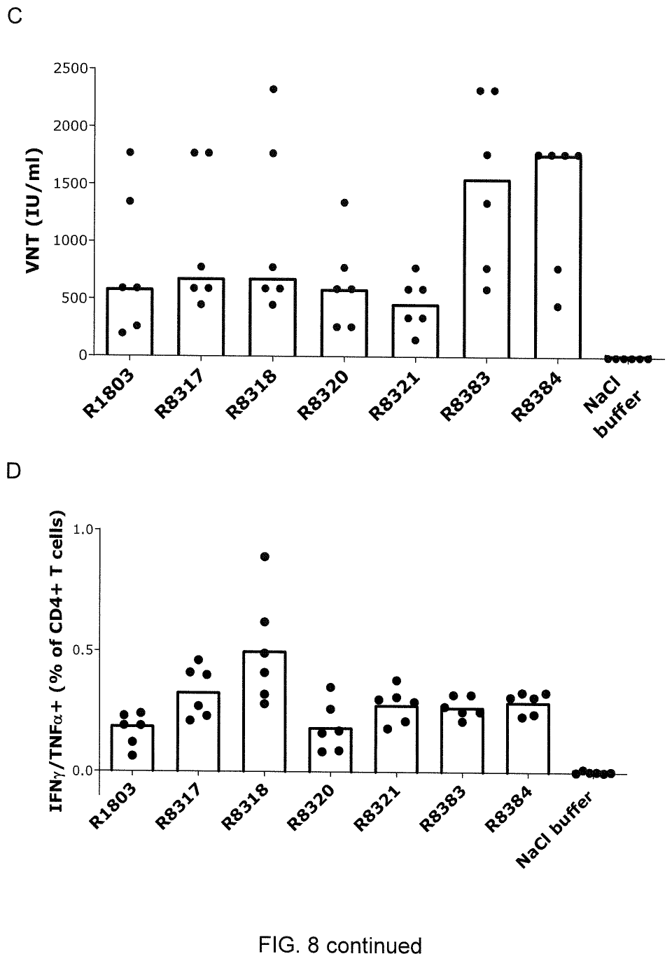

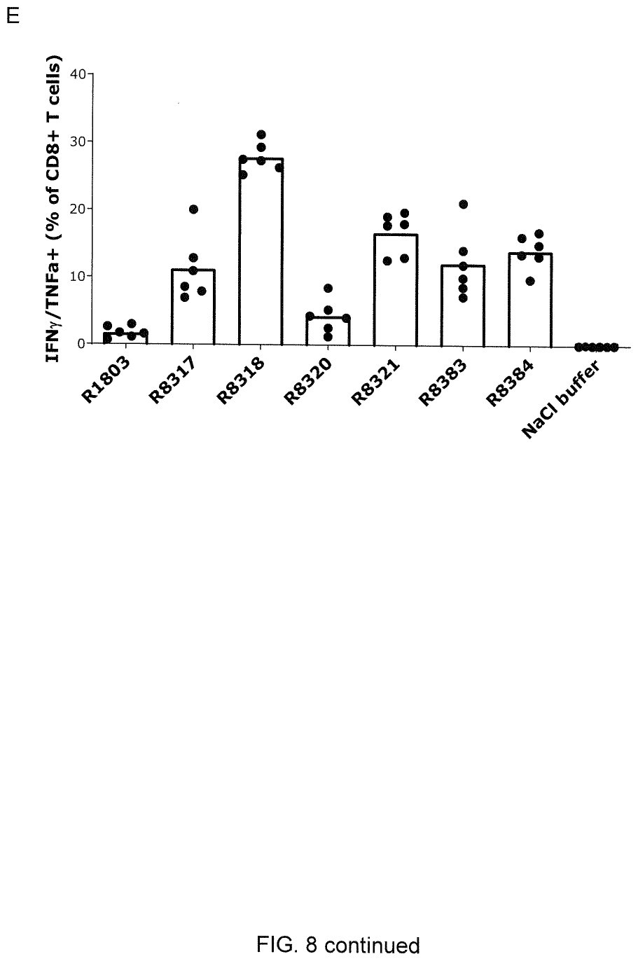

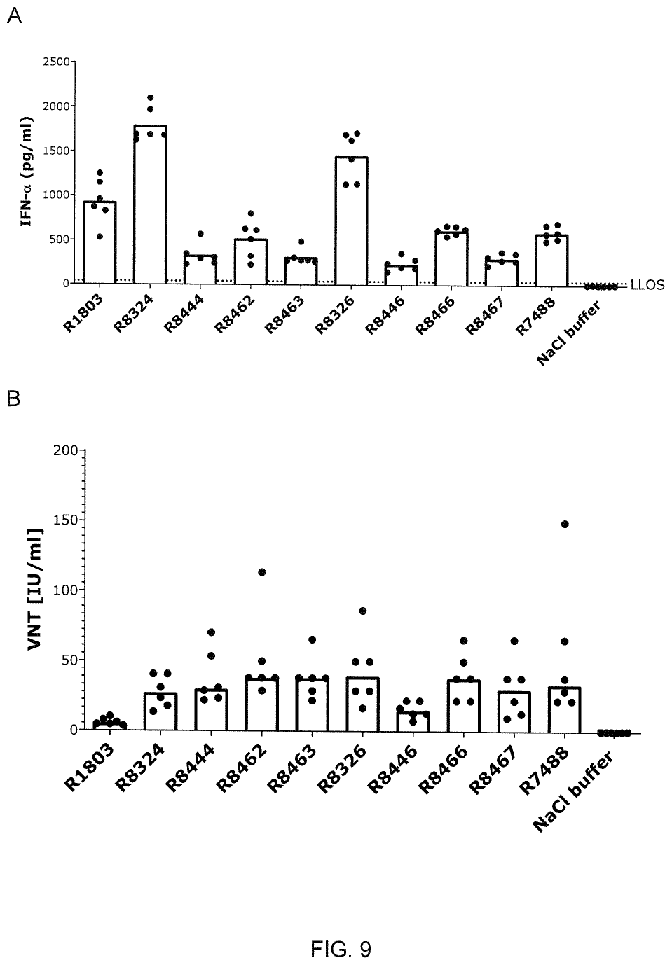

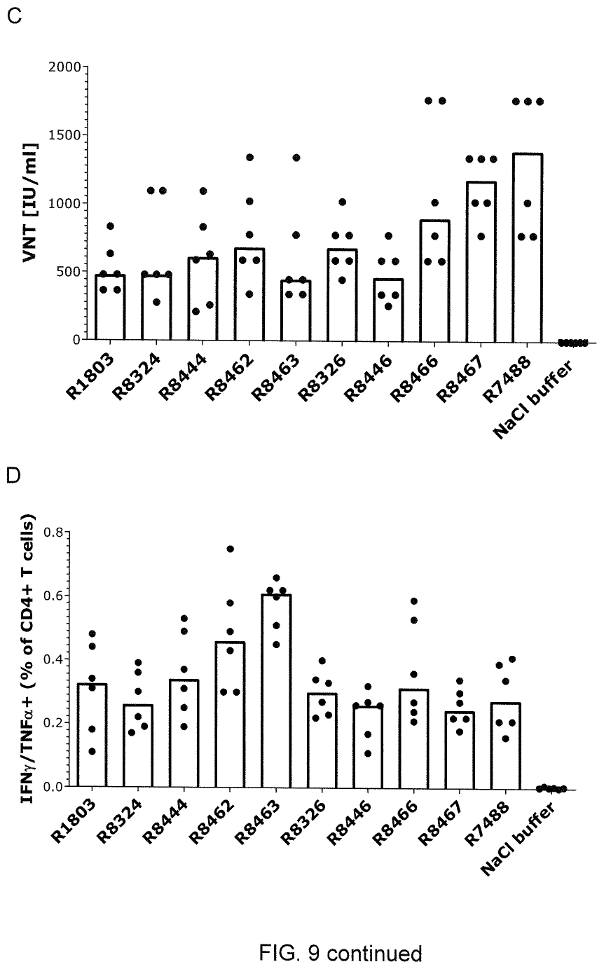

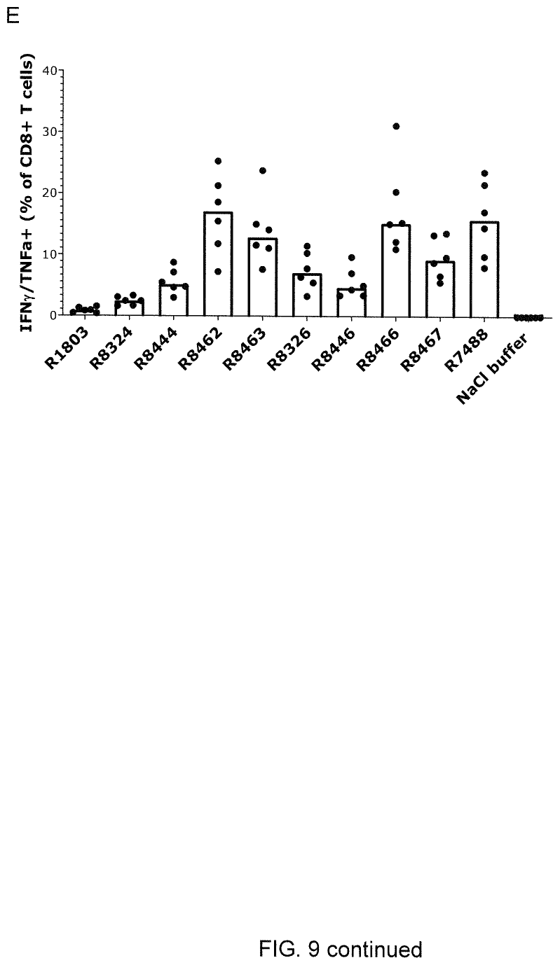

Results are provided in FIG. 6B-FIG. 6E, FIG. 7B-FIG. 7E, FIG. 8B-FIG. 8E and FIG. 9B-FIG. 9E and according Figure descriptions.

3.5 Vaccination of Mice Using Different Formulated IVT mRNA Encoding RABV-G to Show Reactogenicity after Intramuscular Application

In an additional experiment, serum samples were taken 14 hours after i.m. injection of 5 μg RABV-G-encoding mRNA formulated with LNPs (see Table VII) for an analysis of IFNa levels determined by ELISA according to standard protocols.

Results are provided in FIG. 6A, FIG. 7A, FIG. 8A and FIG. 9A and according Figure description.

3.5 Summary of Results

The results of example 3 (FIG. 6-FIG. 9) show that IVT mRNA encoding RABV-G protein which template DNA strand has been linearized using SapI led to reduced reactogenicity and innate immune responses (displayed by IFNa titers in the serum, see FIG. 6A, FIG. 7A, FIG. 8A and FIG. 9A) without reducing immunogenicity or the potential to induce adaptive immune responses (displayed by VNTs and T cell response, see FIG. 6B-FIG. 6E, FIG. 7B-FIG. 7E, FIG. 8B-FIG. 8E and FIG. 9B-FIG. 9E). For the CD8 positive T cell responses non-modified IVT mRNA linearized with SapI showed the highest responses. CD8 T cell responses are very important for the immune system in prevention and treatment of infectious diseases, especially in virus infections.

All mRNA constructs comprising modified nucleotides, pseudouridine or N1-methylpseudouridine, showed reduced reactogenicity and innate immune responses (displayed by IFNa titers in the serum, see FIG. 6A, FIG. 7A, FIG. 8A and FIG. 9A). Particularly, the combination of linearization with a type IIS restriction enzyme (SapI) and the use of pseudouridine or N1-methylpseudouridine led to reduced reactogenicity and innate immune responses (displayed by IFNa titers in the serum, see FIG. 6A, FIG. 7A, FIG. 8A and FIG. 9A).

Reduced reactogenicity (which is particularly induced by IFNa) is of particular importance regarding prophylactic vaccination against infectious diseases. If reactogenicity is only induced to a minor degree the dose of the vaccine can be increased. This is particularly important to induce a strong antigen-specific adaptive immune response.

In e.g. protein replacement therapy reduced or no induction of the innate immune system is necessary and favorable. All IVT mRNAs which template DNA strand has been linearized using SapI and comprising modified nucleotides, pseudouridine or N1-methylpseudouridine, showed nearly no detectable reactogenicity or activation of the innate immune response (displayed by INFa titers in the serum, see FIG. 6A, FIG. 7A, FIG. 8A and FIG. 9A). VT mRNA linearized with SapI led to reduced reactogenicity and innate immune response (displayed by INa levels in the serum, see FIG. 9A) independent of the UTR combination. Some UTR combination (UBQLN2/RPS9.1 and HSD17B4/PSMB3) showed higher VNT levels (see FIG. 9C) and might be therefore beneficial for the use in therapy where high expression is necessary.

### Example 4: Reducing dsRNA Content and the Immunostimulatory Properties of an In Vitro Transcribed RNA Comprising a Step of Digestion of a Circular DNA Template with a Type IIS Restriction Endonuclease

4.1 Preparation of HRNA and DNA Constructs

DNA sequences encoding firefly (Photinus pyralis) luciferase, PpLuc, were prepared and used for subsequent RNA in vitro transcription reactions. Said DNA sequences were prepared by modifying the wild type or reference encoding DNA sequences by introducing a G/C optimized coding for stabilization and expression optimization. Sequences were introduced into a pUC derived DNA vector to comprise stabilizing 3′-UTR sequences and optionally 5′-UTR sequences, additionally comprising a stretch of adenosines (e.g. A64 or A100), and optionally a histone stem-loop (hSL) structure (see Table X). The obtained plasmid DNA constructs were transformed and propagated in bacteria using common protocols known in the art. Eventually, the plasmid DNA constructs were extracted, purified, and used for subsequent RNA in vitro transcription.

TABLE X
IVT mRNA encoding PpLuc used in the example 4

| | Restriction | | | | |
|---|---|---|---|---|---|
| | enzyme | | | | SEQ |
| mRNA | used for | UTR Design | | 5′ cap | ID NO: |
| ID | linearization | (5′ UTR/3′ UTR) | 3′ end | structure | mRNA |
| R10238 | Sapl | HSD17B4/PSMB3 | A100-GGG | Cap1 | 110 |
| R10237 | Sapl | HSD17B4/PSMB3 | A100-CCC | Cap1 | 111 |
| R10236 | Sapl | HSD17B4/PSMB3 | A100-AAA | Cap1 | 112 |
| R10235 | Sapl | HSD17B4/PSMB3 | A100-UUU | Cap1 | 113 |
| R10234 | Sapl | HSD17B4/PSMB3 | A100 | Cap1 | 114 |
| R10243 | EcoRI | HSD17B4/PSMB3 | A100-GAAUU | Cap1 | 115 |
| R10240 | Sapl | HSD17B4/PSMB3 | A100-GAAUU | Cap1 | 116 |
| R10269 | Nsil | HSD17B4/PSMB3 | A100 | Cap1 | 117 |
| R10271 | BciVI | HSD17B4/PSMB3 | A100 | Cap1 | 118 |
| R10250 | Sapl | —/muag | HSL-A100 | Cap0 | 119 |
| R10251 | Sapl | —/muag | HSL-A100-GAAUU | Cap0 | 120 |
| R10252 | EcoRI | —/muag | HSL-A100-GAAUU | Cap0 | 121 |
| R10253 | Sapl | —/muag | HSL-A64 | Cap0 | 122 |
| R10254 | Sapl | —/muag | HSL-A64-GAAUU | Cap0 | 123 |
| R10255 | EcoRI | —/muag | HSL-A64-GAAUU | Cap0 | 124 |
| R6557 | Nsil | HSD17B4/PSMB3 | A64 | Cap0 | 125 |
| R6823 | Sapl | HSD17B4/PSMB3 | A64 | Cap0 | 126 |
| R6825 | Bbsl | HSD17B4/PSMB3 | A64 | Cap0 | 127 |
| R6554 | EcoRI | HSD17B4/PSMB3 | A64-GAAUU | Cap0 | 128 |

4.2 RNA In Vitro Transcription from Plasmid DNA Templates

DNA plasmids prepared according to paragraph 4.1 were enzymatically linearized using different restriction enzymes (e.g. EcoRI, SapI, NsiI, BbsI or BciVI) and used for DNA dependent RNA in vitro transcription using T7 RNA polymerase in the presence of a nucleotide mixture (ATP/GTP/CTP/UTP) and cap analog (e.g., m7GpppG or m7G(5′)ppp(5′)(2′OMeA)pG) under suitable buffer conditions. Obtained RNA constructs were used directly for in vitro experiments or were purified using RP-HPLC (PureMessenger©, CureVac AG, Tübingen, Germany; WO2008/077592). Generated RNA is provided in Table X.

For the RP-HPLC purification, the proportion of organic solvent in the mobile phase were increased in the course of HPLC separation from 5.0 vol. % to 20.0 vol. %, in each case relative to the mobile phase. In particular, the proportion of organic solvent in the mobile phase were increased in the course of HPLC separation from 7.5 vol. % to 17.5 vol. %, in particular 9.5 to 14.5 vol. %, in each case relative to the mobile phase. The RP-HPLC purification were performed under denaturing conditions.

The RP-HPLC purification step was performed at a temperature of about 40° C. (R6557, R6823, R6825 and R6554) or 70° C. (R10238, R10237, R10236, R10235, R10234, R10243, R10240, R10269, R10271, R10250, R10251, R10252, R10253, R10254 and R10255). Suitably, the temperature was maintained and kept constant during the RP-HPLC purification procedure.

4.3 dsRNA Content of In Vitro Transcribed (IVT) RNA Comprising a Step of Digestion of a Circular DNA Template with Different Restriction Endonucleases

The dsRNA content of the generated mRNA (see paragraph 4.1 and 4.2) was measured using dsELISA (detailed description see paragraph 1.7).

4.3.1 Comparison of dsRNA content of IVT RNA digested during IVT step with different restriction endonucleases The dsRNA content was reduced for the IVT RNA generated by DNA templates linearized using type IIS endonucleases SapI and BbsI (R6823, R6825, see Table XI). Both endonucleases led to a template DNA strand comprising a 5′ terminal T nucleotide wherein the 5′ terminal T nucleotide is a 5′ terminal T overhang and wherein the 5′ terminal T overhang comprises 3 consecutive T nucleotides (SapI) or 4 consecutive T nucleotides (BbsI). The temperature of 700 during HPLC purification showed less dsRNA contents for all IVT RNAs. The IVT RNAs generated by DNA templates linearized using type IIS endonucleases SapI and BbsI (R6823, R6825) and were purified with the temperature of 70° C., showed lower values of dsRNA than the Lower Limit of Quantification (LLQ: for input of 10 ng/μl: 0,3 ng dsRNA/μg RNA and for input 100 μg/μl 0,03 ng dsRNA/μg RNA).

TABLE XI
dsRNA content of IVT RNA digested during IVT step with different restriction endonucleases

| | | | | 40° HPLC | | 70° HPLC | |
|---|---|---|---|---|---|---|---|
| | | | | purification | | purification | |
| | | | | Output: ng dsRNA/μg RNA | | | |
| | | | | Input | Input | Input | Input |
| | | | | conc. | conc. | conc. | conc. |
| RNA | Restriction enzyme | 5' cap structure-UTR | | 10 | 100 | 10 | 100 |
| ID | used for linearization | Design (5‘ UTR/3'UTR) | 3' end | ng/μl | ng/μl | ng/μl | ng/μl |
| R6823 | Sapl | Cap0-HSD17B4-PpLuc-PSMB3 | A64 | <0.3 | 0.06 | <0.3 | >0.03 |
| R6825 | Bbsl | Cap0-HSD17B4-PpLuc PSMB3 | A64 | <0.3 | 0.07 | <0.3 | >0.03 |
| R6554 | EcoRI | Cap0-HSD17B4-PpLuc-PSMB3 | A64-GAAUU | 1.51 | 0.34 | 0.39 | 0.10 |
| R6557 | Nsil | Cap0-HSD17B4-PpLuc-PSMB3 | A64 | 2.23 | 0.38 | 1.02 | 0.19 |

4.3.2 Comparison of Purified and Non-Purified/IVT RNA Digested During/IVT Step with Different Restriction PGR-38,T12 Endonucleases

Only the IVT RNA generated by DNA templates linearized using type IIS endonucleases SapI and comprising a 5′ terminal T overhang of 3 T nucleotides (R10234, see Table XII) showed measurable values of dsRNA without HPLC purification. All other VT RNAs contained dsRNA in values above the Upper Limit of Quantification (ULQ: for input of 10 μg/μl: 5 ng dsRNA/μg RNA and for input 100 μg/μl 0,ung dsRNAupg RNA).

After HPLC purification IVT RNA linearized with SapI but comprising an EcoRI 3′end (GAAUU) showed comparable values of dsRNA content to the IVT RNA linearized with EcoRI (R10243). The restriction endonucleases Nsil and BciVUI led to a 3′ terminal overhang in the DNA template and shown high dsRNA contents.

The data shows that the generation of a linear DNA template comprising a 5′ terminal overhang during the method of producing an in vitro transcribed RNA has an influence on the measured dsRNA content.

TABLE XII
dsRNA content of purified and non-purified IVT RNA digested during IVT step with different restriction
endonucleases

| | | | | No HPLC | | 70° C. HPLC | |
|---|---|---|---|---|---|---|---|
| | | | | purification | | purification | |
| | | | | Output: ng dsRNA/μg RNA | | | |
| | | | | Input | Input | Input | Input |
| | Restriction | | | conc. | conc. | conc. | conc. |
| | enzyme used for | 5' cap structure-UTR Design | | 10 | 100 | 10 | 100 |
| RNA ID | linearization | (5‘ UTR/3'UTR) | 3' end | ng/μl | ng/μl | ng/μl | ng/μl |
| R10234 | Sapl | Cap1-HSD17B4-PpLuc-PSMB3 | A100 | 0.87 | 0.36 | <0.3 | <0.03 |
| R10240 | Sapl | Cap1-HSD17B4-PpLuc-PSMB3 | A100-GAAUU | >5 | >0.5 | 0.35 | 0.05 |
| R10243 | EcoRI | Cap1-HSD17B4-PpLuc-PSMB3 | A100-GAAUU | >5 | >0.5 | 0.30 | 0.05 |
| R10269 | Nsil | Cap1-HSD17B4-PpLuc-PSMB3 | A100 | >5 | >0.5 | 1.85 | 0.29 |
| R10271 | BciVI | Cap1-HSD17B4-PpLuc-PSMB3 | A100 | >5 | >0.5 | 1.57 | 0.25 |

4.3.3 Comparison of purified and non-purified IVT RNA comprising different 3′ terminal nucleotides digested with type IIS endonuclease

The lowest dsRNA content for non purified fractions was measured in the IVT RNA sample linearized with Sap (R10234, see Table XIII). Non-purified VT RNA linearized with SapI but comprising a 3′ terminal G (R10238) showed reduced dsRNA values as well.

In vitro transcribed RNA linearized with SapI comprising a 3′ terminal A (R10234 and R10236) nucleotide or G nucleotide (R10238) showed reduced dsRNA contents. Other 3′ terminal nucleotides (eg. 3′ terminal C nucleotide R10237) did not shown reduced values.

TABLE XIII
dsRNA content of purified and non-purified IVT RNA comprising different 3' terminal nucleotides

| | | | | No HPLC | | 70° HPLC | |
|---|---|---|---|---|---|---|---|
| | | | | purification | | purification | |
| | | | | Output: ng dsRNA/μg RNA | | | |
| | | | | Input | Input | Input | Input |
| | Restriction | | | conc. | conc. | conc. | conc. |
| | enzyme used for | 5' cap structure-UTR Design | | 10 | 100 | 10 | 100 |
| RNA ID | linearization | (5‘ UTR/3'UTR) | 3' end | ng/μl | ng/μl | ng/μl | ng/μl |
| R10234 | Sapl | Cap1-HSD17B4-PpLuc-PSMB3 | A100 | 0.87 | 0.36 | <0.3 | <0.03 |
| R10240 | Sapl | Cap1-HSD17B4-PpLuc-PSMB3 | A100-GAAUU | >5 | >0.5 | 0.35 | 0.05 |
| R10243 | EcoRI | Cap1-HSD17B4-PpLuc-PSMB3 | A100-GAAUU | >5 | >0.5 | 0.30 | 0.05 |
| R10238 | Sapl | Cap1-HSD17B4-PpLuc-PSMB3 | A100-GGG | 1.40 | 0.40 | <0.3 | <0.03 |
| R10237 | Sapl | Cap1-HSD17B4-PpLuc-PSMB3 | A100-CCC | 3.80 | >0.5 | 0.48 | 0.06 |
| R10236 | Sapl | Cap1-HSD17B4-PpLuc-PSMB3 | A100-AAA | 1.73 | >0.5 | <0.3 | 0.03 |
| R10235 | Sapl | Cap1-HSD17B4-PpLuc-PSMB3 | A100-UUU | >5 | >0.5 | <0.3 | 0.05 |

4.3.4 dsRNA content of purified and non-purified/VTRNA comprising different cap structures and UTR combinations digested during IVT step with different restriction endonucleases

The VT RNAs linearized with SapI showed independent of different cap structures and UTR combinations (R10234 and R10250, see Table XIV) reduced dsRNA values.

The HPLC purification led to reduced dsRNA levels for all IVT RNAs.

TABLE XIV
dsRNA content of purified and non-purified IVT RNA comprising different cap structures and UTR
combinations

| | | | | No HPLC | | 70° HPLC | |
|---|---|---|---|---|---|---|---|
| | | | | purification | | purification | |
| | | | | Output: ng dsRNA/μg RNA | | | |
| | Restriction | | | Input | Input | Input | Input |
| | enzyme | | | conc. | conc. | conc. | conc. |
| | used for | 5' cap structure-UTR Design | | 10 | 100 | 10 | 100 |
| RNA ID | linearization | (5‘ UTR/3'UTR) | 3' end | ng/μl | ng/μl | ng/μl | ng/μl |
| R10234 | Sapl | Cap1-HSD17B4-PpLuc-PSMB3 | A100 | 0.87 | 0.36 | <0.3 | <0.03 |
| R10250 | Sapl | Cap0-PpLuc-muag | HSL-A100 | 1.49 | >0.5 | <0.3 | <0.03 |
| R10243 | EcoRI | Cap1-HSD17B4-PpLuc-PSMB3 | A100-GAAUU | >5 | >0.5 | 0.30 | 0.05 |
| R10252 | EcoRI | Cap0-PpLuc-muag | HSL-A100-GAAUU | >5 | >0.5 | <0.3 | 0.05 |

4.4 Reduction of Immunostimulatory Properties by Using SapI Linearized mRNAs Displayed by Cytokine Induction in Cells

Human Dermal Fibroblasts (HDF) cells were seeded on 96 well plates (Sarstedt). HDF cells were seeded 24 hours before transfection in a compatible complete cell medium (10,000 cells in 200 μl/well). Cells were maintained at 37° C., 5% C02. The day of transfection, the complete medium on HDF was replaced with serum-free Opti-MEM medium (Gibco). Each RNA was complexed with Lipofectamine2000 at a ratio of 1/1.5 (w/v) for 20 minutes in Opti-MEM. Lipocomplexed mRNAs were then added to cells for transfection with 500 ng of RNA per well in a total volume of 200 μl. 90 minutes post start of transfection, complete supernatant (200 μl/well) of transfection solution was exchanged for 200 μl/well of complete medium. Cells were further maintained at 37° C., 5% C02 before harvesting. 24 hours post start of transfection, supernatants were collected and frozen for later analysis of cytokines.

For analysis the cytokine IP-10 was selected because it is secreted by several cell types in response to IFN-γ and an indicator for innate immune responses.

Supernatants were thawn and used to quantify cytokines using a cytometric bead assay (LegendPlex, Biolegend). To this end, 50 μl of 1:1 (v:v) diluted samples (diluted in assay buffer) were added to plates together with diluted standards (human anti-virus response panel diluted in Matrix B). All following washing steps were carried out by centrifugation at ˜250 g and adding 200 μl of wash buffer followed by another centrifugation at ˜250 g. All following incubations were done at room temperature in the dark at mild agitation at 800 RPM. 25 μl of a mixture of beads containing capture antibodies, each specific for a target cytokine were added to the samples and incubated for two hours. After washing, 25 μl of a biotinylated detection antibody was added to the beads to bind captured cytokines and incubated for one hour. Omitting a wash step, 25 μl of Streptavidin-Phycoerythrin was added to the beads containing captured cytokines and bound detection antibodies and incubated for 30 minutes. After a final wash, beads were resuspended in 150 μl wash buffer. Fluorescent signals proportional to the amount of bound cytokines were detected in a Fortessa LSR flow cytometer (BD). Data was extracted as amount of cytokines in picograms per millilitre using LEGENDplex Data Analysis Software according to manufacturer's instructions and used to plot differences between different IVT RNAs. Data were collected and measured in triplicates.

4.4.1 Comparison of Cytokine Induction of IVT RNA Digested During IVT Step with Different Restriction Endonucleases

Cells transfected with IVT RNA which template DNA strand comprises a 5′ terminal T nucleotide wherein the 5′ terminal T nucleotide is a 5′ terminal T overhang and wherein the 5′ terminal T overhang comprises 3 consecutive T nucleotides (R6823) showed reduced immunostimulatory properties, displayed by measured cytokine levels of IP-10. The temperature of 70° during HPLC purification showed reduced IP-10 values for all IVT RNAs.

TABLE XV
Cytokine IP-10 values after transfection of IVT RNA digested during IVT step with different restriction
endonucleases

| | | | | 40° HPLC | | | 70° HPLC | | |
|---|---|---|---|---|---|---|---|---|---|
| | Restriction | | | purification | | | purification | | |
| | enzyme used | | | HDF cells, input 500 ng RNA | | | | | |
| | for | 5' cap structure-UTR | | (output picograms per milliliter) | | | | | |
| RNA ID | linearization | Design (5‘ UTR/3'UTR) | 3' end | #1 | #2 | #3 | #1 | #2 | #3 |
| Control- | — | — | — | LLQ¹ | LLQ | LLQ | LLQ | LLQ | LLQ |
| only cells | | | | | | | | | |
| R6823 | Sapl | Cap0-HSD17B4-PpLuc- | A64 | 614 | 1624 | 3186 | 188 | 334 | 217 |
| | | PSMB3 | | | | | | | |
| R6557 | Nsil | Cap0-HSD17B4-PpLuc- | A64 | 5837 | 4321 | 3156 | 4714 | 7085 | 4379 |
| | | PSMB3 | | | | | | | |
| R6554 | EcoRI | Cap0-HSD17B4-PpLuc- | A64- | 3766 | 3569 | 3248 | 871 | 1782 | 1287 |
| | | PSMB3 | GAAUU | | | | | | |

¹LLQ: Abbreviation of Lower Limit of Quantification, <101.90 picograms per milliliter

4.4.2 Comparison of Purified and Non-Purified IVT RNA Comprising Different 3′ Terminal Nucleotides Digested with Type IIS Endonuclease

Non-purified in vitro transcribed RNA digested during VT step with Sap and comprising a 3′ terminal A nucleotide showed low IP-10 values (R10236, for R10234 2 of 3 replicates, see Table XVI).

IVT RNA linearized with SapI but comprising an EcoRI 3′end (GAAUU) showed higher values of IP-10 (R10240). The highest IP-value was measured after transfecting VT RNA linearized with EcoR (R10243).

HPLC purification reduces the induction of IP-10 after transfection of IVT RNA:

TABLE XVI
Cytokine IP-10 values after transfection of purified and non-purified IVT RNA comprising different 3'
terminal nucleotides digested with Sapl and EcoRI

| | | | | No HPLC | | | 70° HPLC | | |
|---|---|---|---|---|---|---|---|---|---|
| | | 5' cap structure- | | purification | | | purification | | |
| | Restriction enzyme | UTR Design (5‘ UTR/ | | HDF cells, 500 ng | | | | | |
| RNA ID | used for linearization | 3'UTR) | 3' end | #1 | #2 | #3 | #1 | #2 | #3 |
| Control- | — | — | — | LLQ | LLQ | LLQ | LLQ | LLQ | LLQ |
| Only cells | | | | | | | | | |
| R10234 | Sapl | Cap1-HSD17B4- | A100 | 2554 | 1716 | 727 | LLQ | LLQ | LLQ |
| | | PpLuc-PSMB3 | | | | | | | |
| R10240 | Sapl | Cap1-HSD17B4- | A100- | 2802 | 2893 | 2539 | 769 | 1052 | 656 |
| | | PpLuc-PSMB3 | GAAUU | | | | | | |
| R10238 | Sap | Cap1-HSD17B4- | A100- | 2371 | 2205 | 1452 | 124 | 250 | 214 |
| | | PpLuc-PSMB3 | GGG | | | | | | |
| R10237 | Sapl | Cap1-HSD17B4- | A100- | 2392 | 1808 | 1355 | 621 | 1154 | 373 |
| | | PpLuc-PSMB3 | CCC | | | | | | |
| R10236 | Sapl | Cap1-HSD17B4- | A100- | 1007 | 639 | * | 189 | LLQ | LLQ |
| | | Ppluc-PSMB3 | AAA | | | | | | |
| R10243 | EcoRI | Cap1-HSD17B4- | A100- | 3722 | 3332 | 3753 | 776 | 815 | 503 |
| | | Ppluc-PSMB3 | GAAUU | | | | | | |

1: LLQ-Abbreviation of Lower Limit of Quantification, <101.90 picograms per milliliter
*: missing data point due to technical reasons

4.4.3 Comparison of Purified and Non-Purified IVT RNA Comprising Different Cap Structures and UTR Combinations Digested During IVT Step with Different Restriction Endonucleases

The non-purified IVT RNA linearized with SapI comprising a cap1 structure and the UTR combination 5′ UTR HSD17B4 and 3′UTR PSMB3 (R10234, see Table XVII) showed lower IP-10 values compared to the non-purified IVT RNA linearized with SapI comprising a cap0 structure, the 3′UTR muag and an histone stem loop before the poly(A) sequence (R10250). Both IVT RNAs linearized with SapI showed reduced induction of IP-10 compared to the IVT RNAs linearized with EcoRI (R10243 and R10252).

The HPLC purification led to reduced IP-10 values for all IVT RNAs.

TABLE XVII
Cytokine IP-10 values after transfection of purified and non-purified IVT RNA comprising different cap
structures and UTR combinations

| | | | | No HPLC | | | 70° HPLC | | |
|---|---|---|---|---|---|---|---|---|---|
| | | 5' cap structure- | | purification | | | purification | | |
| | Restriction enzyme | UTR Design (5‘ UTR/ | | HDF cells, 500 ng | | | | | |
| RNA ID | used for linearization | 3'UTR) | 3' end | #1 | #2 | #3 | #1 | #2 | #3 |
| Control- | — | — | — | LLQ¹ | LLQ | LLQ | LLQ | LLQ | LLQ |
| only cells | | | | | | | | | |
| R10234 | Sapl | Cap1-HSD17B4- | A100 | 2554 | 1716 | 727 | LLQ | LLQ | LLQ |
| | | PpLuc-PSMB3 | | | | | | | |
| R10250 | Sapl | Cap0-PpLuc-muag | HSL-A100 | 2684 | 4039 | 3185 | LLQ | LLQ | LLQ |
| R10243 | EcoRI | Cap1-HSD17B4- | A100- | 3722 | 3332 | 3753 | 776 | 815 | 503 |
| | | PpLuc-PSMB3 | GAAUU | | | | | | |
| R10252 | EcoRI | Cap0-PpLuc-muag | HSL-A100- | * | 5634 | 8261 | 491 | 836 | 618 |
| | | | GAAUU | | | | | | |

¹LLQ-Abbreviation of Lower Limit of Quantification;
*: missing data point due to technical reasons

4.5 Summary of results

Restriction endonucleases which led to a template DNA strand comprises a 5′ terminal T nucleotide wherein the 5′ terminal T nucleotide is a 5′ terminal T overhang and wherein the 5′ terminal T overhang comprises 3 consecutive T nucleotides or 4 consecutive T nucleotides reduces the dsRNA content of IVT RNA (see paragraph 4.3.1). Cells transfected with IVT RNA which template DNA strand comprises a 5′ terminal T nucleotide wherein the 5′ terminal T nucleotide is a 5′ terminal T overhang and wherein the 5′ terminal T overhang comprises 3 consecutive T nucleotides showed reduced immunostimulatory properties (see paragraph 4.4.1)

The data show as well that the generation of a linear DNA template comprising a 5′ terminal overhang during the method of producing an in vitro transcribed RNA, reduced the dsRNA content of IVT RNA (see paragraph 4.3.2). In vitro transcribed RNAs comprising a 3′ terminal A nucleotide or G nucleotide showed reduced dsRNA contents (see paragraph 4.3.3). In vitro transcribed RNAs comprising a 3′ terminal A nucleotide showed reduced immunostimulatory properties (see paragraph 4.4.2). The IVT RNA which template DNA strand comprises a 5′ terminal T nucleotide showed independent of different cap structures and UTR combinations a reduced dsRNA content (see paragraph 4.3.4). IVT RNAs, which template DNA strand comprises a 5′ terminal T nucleotide showed independent of different cap structures and UTR combinations reduced immunostimulatory properties compared to IVT RNAs, which template DNA strand not comprises a 5′ terminal T nucleotide.

HPLC purification led to reduced dsRNA values and reduced immunostimulatory properties of IVT RNAs (see paragraph 4.3.1 to paragraph 4.4.3). HPLC purification with higher temperature reduced the dsRNA content and immunostimulatory properties more than lower temperatures (see paragraph 4.3.1 and paragraph 4.4.1).

### Example 5: Cellulose and oligo d(T) purification reduce dsRNA content of in vitro transcribed RNA

5.1 Preparation of RNA and DNA constructs

DNA sequences encoding target proteins were prepared and used for subsequent RNA in vitro transcription reactions. Said DNA sequences were prepared by modifying the wild type or reference encoding DNA sequences by introducing a G/C optimized coding for stabilization and expression optimization. Sequences were introduced into a pUC derived DNA vector to comprise stabilizing 3′-UTR sequences and 5′-UTR sequences, additionally comprising a stretch of adenosines, and a histone stem-loop (hSL) structure (see Table XVIII). The obtained plasmid DNA constructs were transformed and propagated in bacteria using common protocols known in the art. Eventually, the plasmid DNA constructs were extracted, purified, and used for subsequent RNA in vitro transcription.

5.2 RNA In Vitro Transcription from Plasmid DNA Templates:

DNA plasmids prepared according to paragraph 4.1 were enzymatically linearized using the restriction enzyme SapI and used for DNA dependent RNA in vitro transcription using T7 RNA polymerase in the presence of a nucleotide mixture (ATP/GTP/CTP/UTP) and cap analog (m7G(5′)ppp(5′)(2′OMeA)pG) under suitable buffer conditions. To obtain modified mRNA RNA in vitro transcription was performed in the presence of a modified nucleotide mixture (ATP, GTP, CTP, pseudouridine (4P) and cap analog (m7G(5′)ppp(5′)(2′OMeA)pG) under suitable buffer conditions. Optionally the obtained RNA constructs were purified using RP-HPLC (PureMessenger®, CureVac AG, Tübingen, Germany; WO2008/077592) and/or purified using cellulose columns for purification (WO2017/182524) and/or oligo d(T) purification (WO2016/180430) (further details see paragraph 5.3 and 5.4.).

TABLE XVIII
mRNA used in example 5

| | | | GC | | |
|---|---|---|---|---|---|
| RNA | 3′ end | Length | content | Modified | HPLC |
| ID | of RNA | (bps) | (%) | nucleotides | purified |
| 1 | HSL-A100 | 1939 | 60.8 | — | No |
| 2 | HSL-A100 | 1939 | 60.8 | pseudouridine (Ψ) | No |
| 3 | HSL-A100 | 1973 | 58.6 | — | Yes |
| 4 | HSL-A100 | 1682 | 58.2 | — | Yes |

5.3 Cellulose Purification Reduces dsRNA Content of In Vitro Transcribed and SapI Linearized RNA

Two IVT RNAs, RNA 1 and RNA 2, were produced in different batches as described before (see paragraph 5.1 and 5.2). Obtained RNA constructs were purified within 2 cycles of using cellulose columns for purification.

Cellulose purification of RNAs in a spin column was performed as described previously in Baiersdörfer et al 2019 publication, the full disclosure is incorporated herein by reference. 450 μg RNA was used for dsRNA removal in a single cellulose spin column. To prepare the cellulose column, 0.14 g cellulose (C6288, sigma) was mixed with 700 μl cellulose purification buffer (10 mM HEPES (pH 7.2), 0.1 mM EDTA, 125 mM NaCl, and 16% (v/v) ethanol) and incubated at room temperature with vigorous shaking. After 10 min cellulose slurry was loaded on an empty spin column and centrifuge for 1 min at 14000 g. Cellulose column was washed once more with 500 pi cellulose purification buffer. Next, 450 μg RNA was added to the column in 500 μl cellulose purification buffer and incubated at room temperature for 30 min. After 30 min spin column was centrifuged for 1 min and purified RNA was recovered as flow-through. Flow-through was loaded again on a new spin column containing equilibrated cellulose slurry and incubated for 30 min at room temperature with shaking. Purified RNA was recovered as a flow-through and precipitated with sodium acetate and isopropanol. Precipitated RNA was recovered by centrifugation and dissolved in nuclease free water.

As known in the art, dsRNA should remain in the cellulose column while ssRNA should pass through as flow-through. Content of dsRNA were measured using a dsRNA ELISA (further details see paragraph 1.7).

In Table XIX the dsRNA content of the obtained in vitro transcribed RNA (Input) (see paragraph 5.1 and 5.2), the purified flow through fraction (Purified) and the fraction bound to the cellulose column (Bound) is shown.

TABLE XIX
dsRNA content of in vitro transcribed RNA,
cellulose column purified RNA fractions and
fraction bound to the cellulose column

| RNA | Batch | | (Input conc. 10 ng/μl) |
|---|---|---|---|
| ID | No. | Fraction | ng dsRNA/μg RNA |
| 1 | 2 | Input | 5.41 |
| | | Purified | 0.42 |
| | | Bound | ULQ* |
| | 3 | Input | 9.11 |
| | | Purified | 0.24 |
| | | Bound | ULQ* |
| 2 | 5 | Input | 0.19 |
| | | Purified | LLQ* |
| | | Bound | 0.37 |
| | 7 | Input | 0.16 |
| | | Purified | 0.03 |
| | | Bound | 0.34 |

*ULQ/LLQ = Limit of quantification of dsRNA ELISA, ULQ: Upper limit of quantification, 5 ng dsRNA/μg RNA, LLQ: Lower limit of quantification, 0.3 ng dsRNA/μg RNA, higher or lower values as the ULQ or LLQ could observed due to curve fitting.

The dsRNA content was reduced in all purified flow-through fractions. The cellulose purification steps could reduce high values (RNA ID 1) of dsRNA input. The cellulose purification method reduced dsRNA values in IVT RNAs comprising modified nucleotides (RNA 2) and non-modified (RNA 1) nucleotides.

5.4 Cellulose and Oligo d(T) Purification Reduce dsRNA Content of HPLC Purified In Vitro Transcribed RNA

Two RNA constructs, RNA 3 and RNA 4, were produced in different batches as described before (see paragraph 5.1 and 5.2) and purified using RP-HPLC (PureMessenger®, CureVac AG, Tübingen, Germany; WO2008/077592). For the RP-HPLC purification, the proportion of organic solvent in the mobile phase were increased in the course of HPLC separation from 5.0 vol. % to 20.0 vol. %, in each case relative to the mobile phase. In particular, the proportion of organic solvent in the mobile phase were increased in the course of HPLC separation from 7.5 vol. % to 17.5 vol. %, in particular 9.5 to 14.5 vol. %, in each case relative to the mobile phase. The RP-HPLC purification were performed under denaturing conditions. The RP-HPLC purification step was performed at a temperature of about 70° C. Suitably, the temperature was maintained and kept constant during the RP-HPLC purification procedure.

The HPLC Purified RNA Constructs were Purified within 2 Cycles of Using Cellulose Columns (Further Details See Paragraph 5.3) or 1 Cycle of Oligo d(T) Purification.

To purify the RNA using oligodT column, 500 μg RNA was incubated with 1.5× molar excess of oligodT₆₀ in 200 μl 2×SSC buffer for 15 min at room temperature. In the meantime 200 μl streptavidin sepharose beads were equilibrated in 2×SSC buffer. Equilibrated beads were added to RNA- oligodT₆₀ mix and incubated for another 15 min at room temperature with intermittent mixing by tapping the tube. After 15 min RNA-bead mixture was loaded on a 0.2 micron filter containing empty spin column. Beads were washed subsequently with 2×SSC and 0.1×SSC. Each SSC wash was repeated thrice. In the end bound RNA material was eluted in nuclease free water and precipitated with sodium acetate and isopropanol. Precipitated RNA was recovered by centrifugation and dissolved in nuclease free water and measured. Content of dsRNA were measured using a dsRNA ELISA (further details see paragraph 1.7).

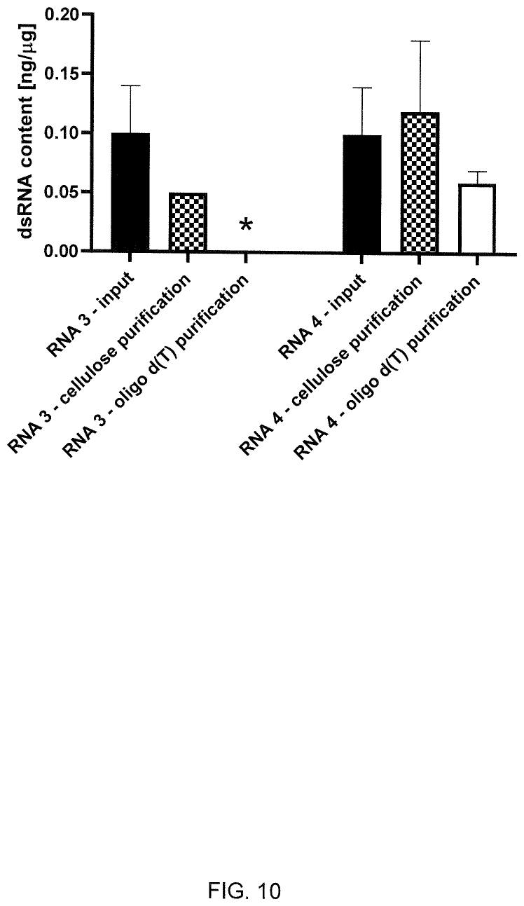

FIG. 10 shows the dsRNA content of two different IVT RNAs (RNA 3 and RNA 4) which template DNA strand has been linearized using SapI and purified with HPLC and additional two steps of cellulose purification or one step of oligo d(T) purification. The RNA fractions purified with an oligo d(T) column led to less dsRNA content compared to the fractions purified with two cycles of cellulose purification.

ITEMS

The present invention may be characterized by the following items:

1. Method of reducing the immunostimulatory properties of an in vitro transcribed RNA by producing the in vitro transcribed RNA according to the following steps
i) providing a linear DNA template comprising a template DNA strand encoding the RNA, wherein the template DNA strand comprises a 5′ terminal T nucleotide;
ii) incubating the linear DNA template under conditions to allow (run-off) RNA in vitro transcription;
iii) obtaining the in vitro transcribed RNA comprising a 3′ terminal A nucleotide.

2. Method according to item 1, wherein step i) comprises a step of digestion of a circular DNA template with a restriction endonuclease to generate the linear DNA template comprising a 5′ terminal T nucleotide.

3. Method according to item 2, wherein the circular DNA template comprises a recognition sequence for a restriction endonuclease and a cleavage site for a restriction endonuclease.

4. Method according to item 3, wherein the cleavage site for the restriction endonuclease is located outside of the recognition sequence.

5. Method according to item 1 to 4 wherein the 5′ terminal T nucleotide is a 5′ terminal T overhang.

6. Method according to item 5, wherein the 5′ terminal T overhang comprises at least 3 consecutive T nucleotides.

7. Method according to item 1 to 6, wherein the 5′ terminal T nucleotide is part of a polyT sequence.

8. Method according to item 1 to 7, wherein the linear DNA template comprises a RNA polymerase promotor sequence.

9. Method according to item 2 to 8, wherein the restriction endonuclease is a type II restriction endonuclease.

10. Method according to item 2 to 9, wherein the restriction endonuclease is a type IS restriction endonuclease.

11. Method according to item 10, wherein the type IIS restriction endonuclease is selected from the group consisting of SapI, BSpQI, EciI, BpiI, AarI, AceIII, Acc36I, AloI, BaeI, BbvCI, PpiI and PsrI, BsrDI, BtsI, EarI, BmrI, BsaI, BsmBI, FauI, FaqI, BbsI, BciVUI, BfuAI, Bse3DI, BspMI, BciVI, BseRI, BfuII, BfiII, BmrI, EciI, BtgZI, BpuEI, BsgI, MmeI, CspCI, BaeI, BsaMI, BveI, Mva1269I, FOKL, PctI, Bse3DI, BseMI, Bst6I, Eam11041, Ksp6321, BfiI, Bso31I, BspTNI, Eco31 I, Esp3I, BfuI, Acc36I, AarI, Eco57I, Eco57MI, GsuI, AloI, Hin4I, PpiI, and PsrI or corresponding isoschizomer.

12. Method according to item 10 to 11, wherein the type IIS restriction endonuclease is SapI LguI, PciSI or BSpQI.

13. Method according to item 10 to 12, wherein the type IIS restriction endonuclease is SapI.

14. Method according to any of the preceding items, wherein the in vitro transcription in step ii) leads to the formation of less double stranded RNA side products as compared to an in vitro transcription performed with a linear DNA template that does not comprise a 5′ terminal T nucleotide on the template DNA strand encoding the RNA.

15. Method according to according to any of the preceding items, wherein the in vitro transcription in step ii) leads to the formation of about 10% less double stranded RNA side products as compared to an in vitro transcription performed with a linear DNA template that does not comprise a 5′ terminal T nucleotide on the template DNA strand encoding the RNA.

16. Method according to any one of the preceding items, wherein step ii) comprises incubating the linear DNA template with an RNA polymerase and a nucleotide mixture under conditions to allow (run-off) RNA in vitro transcription.

17. Method according to item 16, wherein the nucleotide mixture is sequence optimized.

18. Method according to item 16 or 17, wherein the nucleotide mixture comprises at least one modified nucleotide and/or at least one nucleotide analogue or nucleotide derivative.

19. Method according to item 18, wherein the at least one modified nucleotide and/or at least one nucleotide analogues is selected from a backbone modified nucleotide, a sugar modified nucleotide and/or a base modified nucleotide, or any combination thereof.

20. Method according to item 18 or 19, wherein the least one modified nucleotide and/or the at least one nucleotide analog is selected from 1-methyladenosine, 2-methyladenosine, N6-methyladenosine, 2′-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl- 2′-O-methyluridine, 5-carboxymethylaminomethyl-2-thiouridine, 5-(isopentenylaminomethyl)uridine, 5-(isopentenylaminomethyl)- 2-thiouridine, or 5-(isopentenylaminomethyl)- 2′-O-methyluridine.

21. Method according to item 16 or 17, wherein the nucleotide mixture is composed of (chemically) non-modified ribonucleoside triphosphates (NTPs) GTP, ATP, CTP and UTP.

22. Method according to item 16 to 22, wherein the nucleotide mixture comprises a cap.

23. Method according to item 23, wherein the cap is a cap0, cap1, cap2, a modified cap0 or a modified cap1, preferably a cap1.

24. Method according to item 1 to 22, wherein the method additionally comprises a step of enzymatic capping after step ii) to generate a cap0 and/or a cap1 structure.

25. Method according to any of the preceding items, wherein the obtained in vitro transcribed RNA comprising a 3′ terminal A nucleotide comprises a 5′-cap structure, preferably a cap1 structure.

26. Method according to any one of the preceding items, wherein about 70%, 75%, 80%, 85%, 90%, 95% of the obtained in vitro transcribed RNA comprising a 3′ terminal A nucleotide comprise a cap1 structure as determined by using a capping detection assay.

27. Method according to any one of the preceding items, wherein the method additionally comprises a step of enzymatic polyadenylation after step ii).

28. Method according to any one of the preceding items, wherein the obtained in vitro transcribed RNA comprising a 3′ terminal A nucleotide comprises at least one coding sequence encoding at least one peptide or protein.

29. Method according to item 29, wherein at least one peptide or protein is or is derived from a therapeutic peptide or protein.

30. Method according to item 30, wherein the therapeutic peptide or protein is or is derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen, a bacterial antigen, a protozoan antigen, an allergen, a tumor antigen, or fragments, variants, or combinations of any of these.

31. Method according to items 29 to 31, wherein the at least one coding sequence is a codon modified coding sequence, wherein the amino acid sequence encoded by the at least one codon modified coding sequence is preferably not being modified compared to the amino acid sequence encoded by the corresponding reference coding sequence.

32. Method according to item 32, wherein the at least one codon modified coding sequence is selected from C increased coding sequence, CAI increased coding sequence, human codon usage adapted coding sequence, G/C content modified coding sequence, and G/C optimized coding sequence, or any combination thereof.

33. Method according to any of the preceding items, wherein the obtained in vitro transcribed RNA comprising a 3′ terminal A nucleotide comprises at least one poly(A) sequence, and/or at least one poly(C) sequence, and/or at least one histone stem-loop sequence/structure.

34. Method according to any of the preceding items, wherein the obtained in vitro transcribed RNA comprising a 3′ terminal A nucleotide comprises at least one heterologous 5′-UTR and/or at least one heterologous 3′-UTR.

35. Method according to item 35, wherein the at least one heterologous 3′-UTR comprises a nucleic acid sequence derived from a 3′-UTR of a gene selected from PSMB3, ALB7, alpha-globin, CASP1, COX6B1, GNAS, NDUFA1 and RPS9, or from a homolog, a fragment or a variant of any one of these genes.

36. Method according to item 35, wherein the at least one heterologous 5′-UTR comprises a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.

37. Method according to item any one of the preceding items, wherein the obtained in vitro transcribed RNA comprising a 3′ terminal A nucleotide is an mRNA.

38. Method according to any of the preceding items, wherein the method comprises a step iv) of purifying the obtained in vitro transcribed RNA comprising a 3′ terminal A nucleotide, preferably to remove double-stranded RNA, non-capped RNA and/or RNA fragments.

39. Method according to item 39, wherein the method comprises a step iv) of purifying the obtained in vitro transcribed RNA comprising a 3′ terminal A nucleotide to remove double-stranded RNA.

40. Method according to item 39 or 40, wherein step iv) comprises at least one step of RP-HPLC and/or at least one step of AEX, and/or at least one step of TFF and/or at least one step of oligo d(T) purification.

41. Method according to item 41, wherein step iv) comprises at least one step of RP-HPLC and at least one step of TFF.

42. Method according to item 42, wherein step iv) comprises at least one step of oligo d(T) purification.

43. Method according to any of the preceding items, wherein the obtained in vitro transcribed RNA comprising a 3′ terminal A nucleotide has an RNA integrity of at least 60%.

44. Method according to any of the preceding items, wherein the obtained in vitro transcribed RNA comprising a 3′ terminal A nucleotide has reduced immunostimulatory properties compared to a corresponding reference in vitro transcribed RNA not comprising a 3′-terminal A nucleotide.

45. Method according to item 45, wherein the immunostimulatory properties are defined as the induction of an innate immune response which is determined by measuring the induction of cytokines.

46. Method according to item 46, wherein the cytokines are selected from the group consisting of IFNalpha (IFNα), TNFalpha (TNFα), IP-10, IFNgamma (IFNγ), IL-6, IL-12, IL-8, MIG, Rantes, MIP-1alpha (MIP1α), MIP-1beta (MIP1β), McP1, or IFNbeta (IFNβ).

47. Method according to item 46 or 47, wherein the induction of cytokines is measured by administration of the obtained in vitro transcribed RNA to cells, a tissue or an organism, preferably hPBMCs, Hela cells or HEK cells.

48. Method according to any of the preceding items, wherein the obtained in vitro transcribed RNA comprising a 3′ terminal A nucleotide is more stable and/or the optionally encoded peptide or protein is more efficiently expressed compared to a corresponding reference in vitro transcribed RNA not comprising a 3′-terminal A nucleotide.

49. Method according to any of the preceding items, wherein the method comprises a further step v) formulating the obtained in vitro transcribed RNA with a cationic compound to obtain an RNA formulation.

50. Method according to item 50, wherein the cationic compound comprises one or more lipids suitable to form liposomes, lipid nanoparticles (LNP), lipoplexes, and/or nanoliposomes.

51. Method according to items 50 and 51, wherein step v) comprises a purification step after formulating the obtained in vitro transcribed RNA.

52. An in vitro transcribed RNA comprising a 3′ terminal A nucleotide having reduced immunostimulatory properties obtainable by the method as defined in any of items 1 to 52.

53. The vitro transcribed RNA comprising a 3′ terminal A nucleotide according to item 53, wherein the innate immune response of a subject and/or cell is reduced upon administration to a subject and/or cell.

54. A pharmaceutical composition comprising an in vitro transcribed RNA comprising a 3′ terminal A nucleotide as defined in items 53 to 54 or a composition obtained by the method as defined in items 1 to 52, optionally comprising one or more pharmaceutically acceptable excipients, carriers, diluents and/or vehicles.

55. Pharmaceutical composition according to item 55, wherein the in vitro transcribed RNA comprising a 3′ terminal A nucleotide is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof.

56. Pharmaceutical composition according to item 55 or 56, wherein at least one in vitro transcribed RNA comprising a 3 terminal A nucleotide is complexed or associated with one or more lipids, thereby forming liposomes, lipid nanoparticles (LNP), lipoplexes, and/or nanoliposomes.

57. Pharmaceutical composition according to item 57, wherein at least one in vitro transcribed RNA comprising a 3′ terminal A nucleotide is complexed with one or more lipids thereby forming lipid nanoparticles (LNP).

58. Pharmaceutical composition according to item 57 and 58, wherein the lipid nanoparticles (LNP) comprise a PEGylated lipid.

59. Pharmaceutical composition according to items 57 to 59, wherein the LNP comprises
(i) at least one cationic lipid;
(ii) at least one neutral lipid;
(iii) at least one steroid or steroid analogue; and
(iv) at least one a PEG-lipid,
wherein (i) to (iv) are in a molar ratio of about 20-60% cationic lipid, 5-25% neutral lipid, 25-55% sterol, and 0.5-15% PEG-lipid.

60. Pharmaceutical composition according to items 55 to 60, wherein the pharmaceutical composition comprises Ringer or Ringer-Lactate solution.

61. Pharmaceutical composition according to items 55 to 61, wherein an administration of the pharmaceutical composition to a cell or subject results in a reduced innate immune response compared to an administration of a corresponding composition that comprises an RNA that does not comprise a 3-terminal A nucleotide.

62. Pharmaceutical composition according to items 62, wherein the subject is a human subject.

63. Pharmaceutical composition according to item 62 or 63, wherein the administration is systemically or locally.

64. Pharmaceutical composition according to item 62 to 64, wherein the administration is transdermally, intradermally, intravenously, intramuscularly, intranorally, intraaterially, intranasally, intrapulmonally, intracranially, intralesionally, intratumorally, intravitreally, subcutaneously or via sublingual, preferably intramuscularly, intranodally, intradermally, intratumorally or intravenously.

65. Pharmaceutical composition according to items 62 to 65, wherein the administration is more than once, for example once or once more than once a day, once or more than once a week, once or more than once a month.

66. Kit or kit of parts comprising the in vitro transcribed RNA comprising a 3′ terminal A nucleotide as defined in items 53 to 54, or pharmaceutical composition as defined in items 55 to 66, optionally comprising a liquid vehicle for solubilizing, and, optionally, technical instructions providing information on administration and/or dosage of the components.

67. An in vitro transcribed RNA comprising a 3-terminal A nucleotide having reduced immunostimulatory properties as defined in items 53 to 54, or a pharmaceutical composition as defined in items 55 to 66, or a kit or kit of parts as defined in item 67, for use as medicament.

68. An in vitro transcribed RNA comprising the 3-terminal A nucleotide having reduced immunostimulatory properties as defined in items 53 to 54, or a pharmaceutical composition as defined in items 55 to 66, or a kit or kit of parts as defined in item 67, for use in the prevention or treatment of cancer, autoimmune diseases, infectious diseases, allergies or protein deficiency disorders.

69. A method of treatment or preventing a disorder, wherein the method comprises applying or administering to a subject in need thereof the in vitro transcribed RNA comprising a 3′-terminal A nucleotide as defined in items 53 to 54, or the pharmaceutical composition as defined in items 55 to 66, or the kit or kit of parts as defined in item 67, preferably wherein applying or administering is performed more than once, for example once or more than once a day, once or more than once a week, once or more than once a month.

70. Method of treatment or preventing a disorder according to item 70, wherein the administration or applying is subcutaneous, intravenous, intramuscular, intra-articular, intra-synovial, intranasal, oral, intrasternal, intrathecal, intrahepatic, intralesional, intracranial, transdermal, intradermal, intrapulmonal, intraperitoneal, intracardial, intraarterial, intraocular, intravitreal, subretinal, intranodal, or intratumoral.

71. Method of treatment according to item 70 or 71, wherein the subject in need is a mammalian subject, preferably a human subject.

72. A method of reducing the induction of an innate immune response induced by an in vitro transcribed RNA upon administration of said RNA to a cell or a subject comprising
(i) obtaining the in vitro transcribed RNA by the method as defined in any of items 1 to 52; and
(ii) administering an effective amount of the in vitro transcribed RNA comprising a 3′ terminal A nucleotide from step (i) having reduced immunostimulatory properties to a cell or a subject.

1. A method of reducing the immunostimulatory properties of an in vitro transcribed RNA by producing the in vitro transcribed RNA according to the following steps
i) providing a linear DNA template comprising a template DNA strand encoding the RNA, wherein the template DNA strand comprises a 5′ terminal T nucleotide;
ii) incubating the linear DNA template under conditions to allow RNA in vitro transcription;
iii) obtaining the in vitro transcribed RNA comprising a 3′ terminal A nucleotide;
iv) purifying the obtained in vitro transcribed RNA comprising a 3′ terminal A nucleotide to remove double-stranded RNA;
wherein the 5′ terminal T nucleotide is a 5′ terminal T overhang and
wherein the 5′ terminal T overhang comprises at least 3 consecutive T nucleotides.

2. The method according to claim 1, wherein the provided linear DNA template leads to reduced double stranded RNA content in the obtained and/or purified in vitro transcribed RNA.

3. The method according to claim 1 or 2, wherein step i) comprises a step of digestion of a circular DNA template with a restriction endonuclease to generate the linear DNA template comprising a 5′ terminal T nucleotide.

4. The method according to claim 3, wherein the circular DNA template comprises a recognition sequence for a restriction endonuclease and a cleavage site for a restriction endonuclease.

5. The method according to claim 4, wherein the cleavage site for the restriction endonuclease is located outside of the recognition sequence.

6. The method according to claims 1 to 5, wherein the 5′ terminal T overhang comprises at least 1, 2, 3, 4, 5 or 6 consecutive T nucleotides.

7. The method according to claims 1 to 6, wherein the 5′ terminal T overhang comprises at least 3 or 4 consecutive T nucleotides, preferably at least 3 consecutive T nucleotides.

8. The method according to claims 1 to 7, wherein the 5′ terminal T overhang comprises at least 3 consecutive T nucleotides, preferably 3 consecutive T nucleotides.

9. The method according to claims 1 to 8, wherein the 5′ terminal T nucleotide is part of a polyT sequence.

10. The method according to claims 1 to 9, wherein the linear DNA template comprises an RNA polymerase promotor sequence.

11. The method according to claims 1 to 10, wherein the linear DNA template comprises a T7 RNA polymerase promotor sequence.

12. The method according to claims 3 to 11, wherein the restriction endonuclease is a type II restriction endonuclease.

13. The method according to claims 3 to 12, wherein the restriction endonuclease is a type IIS restriction endonuclease.

14. The method according to claim 13, wherein the type IIS restriction endonuclease is selected from the group consisting of SapI, BSpQI, EciI, BpiI, AarI, AceIII, Acc36I, AloI, BaeI, BbvCI, PpiI and PsrI, BsrDI, BtsI, EarI, BmrI, BsaI, BsmBI, FauI, FaqI, BbsI, BciVUI, BfuAI, Bse3DI, BspMI, BciVUI, BseRI, BfuII, BfiII, BmrI, EciI, BtgZI, BpuEI, BsgI, MmeI, CspCI, BaeI, BsaMI, BveI, Mva12691, FOKL, PctI, Bse3DI, BseMI, Bst6I, Eam1104I, Ksp632I, BfiI, Bso31I, BspTNI, Eco31I, Esp3I, BfuI, Acc36I, AarI, Eco57I, Eco57MI, GsuI, AloI, Hin4I, PpiI, and PsrI or corresponding isoschizomer.

15. The method according to claim 13 or 14, wherein the type IIS restriction endonuclease is SapI, BbsI, LguI, PciSI or BspQI, or corresponding isoschizomer.

16. The method according to claims 13 to 15, wherein the type IIS restriction endonuclease is SapI, or corresponding isoschizomer.

17. The method according to any of the preceding claims, wherein the in vitro transcription in step ii) leads to the formation of less double stranded RNA side products as compared to an in vitro transcription performed with a linear DNA template that does not comprise a 5′ terminal T nucleotide on the template DNA strand encoding the RNA.

18. The method according to according to any of the preceding claims, wherein the in vitro transcription in step ii) leads to the formation of about 10% less double stranded RNA side products as compared to an in vitro transcription performed with a linear DNA template that does not comprise a 5′ terminal T nucleotide on the template DNA strand encoding the RNA.

19. The method according to any one of the preceding claims, wherein step ii) comprises incubating the linear DNA template with an RNA polymerase and a nucleotide mixture under conditions to allow RNA in vitro transcription, preferably wherein the RNA polymerase is a T7 RNA polymerase

20. The method according to claim 19, wherein the nucleotide mixture is sequence optimized.

21. The method according to claim 19 or 20, wherein the nucleotide mixture comprises at least one modified nucleotide and/or at least one nucleotide analogue or nucleotide derivative.

22. The method according to claim 21, wherein the at least one modified nucleotide and/or at least one nucleotide analogues is selected from a backbone modified nucleotide, a sugar modified nucleotide and/or a base modified nucleotide, or any combination thereof.

23. The method according to claim 21 or 22, wherein the least one modified nucleotide and/or the at least one nucleotide analog is selected from 1-methyladenosine, 2-methyladenosine, N6-methyladenosine, 2-O-methyladenosine, 2-methylthio-N6-methyladenosine, N6-isopentenyladenosine, 2-methylthio-N6-isopentenyladenosine, N6-threonylcarbamoyladenosine, 2-methylthio-N6-threonyl carbamoyladenosine, N6-methyl-N6-threonylcarbamoyladenosine, N6-hydroxynorvalylcarbamoyladenosine, 2-methylthio-N6-hydroxynorvalyl carbamoyladenosine, inosine, 3-methylcytidine, 2′-O-methylcytidine, 2-thiocytidine, N4-acetylcytidine, lysidine, 1-methylguanosine, 7-methylguanosine, 2′-O-methylguanosine, queuosine, epoxyqueuosine, 7-cyano-7-deazaguanosine, 7-aminomethyl-7-deazaguanosine, pseudouridine, dihydrouridine, 5-methyluridine, 2′-O-methyluridine, 2-thiouridine, 4-thiouridine, 5-methyl-2-thiouridine, 3-(3-amino-3-carboxypropyl)uridine′, 5-hydroxyuridine, 5-methoxyuridine, uridine 5-oxyacetic acid, uridine 5-oxyacetic acid methyl ester, 5-aminomethyl-2-thiouridine, 5-methylaminomethyluridine, 5-methylaminomethyl-2-thiouridine, 5-methylaminomethyl-2-selenouridine, 5-carboxymethylaminomethyluridine, 5-carboxymethylaminomethyl- 2′-O-methyluridine, 5-carboxymethylaminomethyl-2-thiouridine, 5-(isopentenylaminomethyl)uridine, 5-(isopentenylaminomethyl)- 2-thiouridine, or 5-(isopentenylaminomethyl)- 2′-O-methyluridine.

24. The method according to claims 21 to 23, wherein at least one modified nucleotide is selected from pseudouridine (y), N1-methylpseudouridine (m14), 5-methylcytosine, and/or 5-methoxyuridine.

25. The method according to claims 21 to 24, wherein at least one modified nucleotide is selected from N1-methylpseudouridine (m1ψ).

26. The method according to claim 19 or 20, wherein the nucleotide mixture is composed of non-modified ribonucleoside triphosphates (NTPs) GTP, ATP, CTP and UTP.

27. The method according to claims 19 to 26, wherein the nucleotide mixture comprises a cap analog.

28. The method according to claim 27, wherein the cap analog is a cap0, cap1, cap2, a modified cap0 or a modified cap1analog, preferably a cap1 analog.

29. The method according to claim 28, wherein the cap1 analog is a cap1 trinucleotide cap analog.

30. The method according to claims 1 to 26, wherein the method additionally comprises a step of enzymatic capping after step ii) to generate a capo and/or a cap1 structure.

31. The method according to any of the preceding claims, wherein the obtained in vitro transcribed RNA comprising a 3′ terminal A nucleotide comprises a 5′-cap structure, preferably a cap1 structure.

32. The method according to any one of the preceding claims, wherein about 70%, 75%, 80%, 85%, 90%, 95% of the obtained in vitro transcribed RNA comprising a 3′ terminal A nucleotide comprise a cap1 structure as determined by using a capping detection assay.

33. The method according to any one of the preceding claims, wherein the method additionally comprises a step of enzymatic polyadenylation after step ii).

34. The method according to any one of the preceding claims, wherein the obtained in vitro transcribed RNA comprising a 3′ terminal A nucleotide comprises at least one coding sequence encoding at least one peptide or protein.

35. The method according to claim 34, wherein at least one peptide or protein is selected or derived from a therapeutic peptide or protein.

36. Method according to claim 35, wherein the therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen, a bacterial antigen, a pathogen antigen, a protozoan antigen, an allergen, a tumor antigen, or fragments, variants, or combinations of any of these.

37. The method according to claim 35 or 36, wherein the therapeutic peptide or protein is or is derived from viral antigen.

38. The method according to claims 34 to 37, wherein the at least one coding sequence is a codon modified coding sequence, wherein the amino acid sequence encoded by the at least one codon modified coding sequence is preferably not being modified compared to the amino acid sequence encoded by the corresponding reference coding sequence.

39. The method according to claim 38, wherein the at least one codon modified coding sequence is selected from C increased coding sequence, CAI increased coding sequence, human codon usage adapted coding sequence, G/C content modified coding sequence, and G/C optimized coding sequence, or any combination thereof.

40. The method according to claim 39, wherein the at least one codon modified coding sequence is selected from G/C optimized coding sequence.

41. The method according to claim 39 or 40, wherein the G/C optimized coding sequence has a GC content of about 50%, 55%, 56%, 57%, 58%, 59%, 60%, 61%, 62%, 63% or 64%.

42. The method according to any of the preceding claims, wherein the obtained in vitro transcribed RNA comprising a 3′ terminal A nucleotide comprises at least one poly(A) sequence, and/or at least one poly(C) sequence, and/or at least one histone stem-loop sequence/structure.

43. The method according to claim 42, wherein the at least one poly(A) sequence comprises about 30, about 60, about 64, about 70, about 100, about 101, about 110 or about 120 adenosine nucleotides.

44. The method according to claim 42 or 43, wherein the at least one poly(A) sequence comprises at least 60, at least 80, at least 100, at least 110 or at least 120 adenosine nucleotides.

45. The method according to claim 42 to 44, wherein the at least one poly(A) sequence comprises about 60 to about 120 adenosine nucleotides.

46. The method according to claim 42 to 45, wherein the at least one poly(A) sequence is interrupted by at least one nucleotide different from an adenosine nucleotide.

47. The method according to any of the preceding claims, wherein the obtained in vitro transcribed RNA comprising a 3′ terminal A nucleotide comprises at least one heterologous 5′-UTR and/or at least one heterologous 3′-UTR.

48. The method according to claim 47, wherein the at least one heterologous 3′-UTR comprises a nucleic acid sequence derived from a 3′-UTR of a gene selected from PSMB3, ALB7, alpha-globin, CASP1, COX6B1, GNAS, NDUFA1, RPS10, human mitochondrial 12S rRNA (mtRNR1), human AES/TLE5 gene, FIG. 4 and RPS9, or from a homolog, a fragment or a variant of any one of these genes.

49. The method according to claim 47 or 48, wherein the at least one heterologous 3′-UTR comprises a nucleic acid sequence derived from a 3′-UTR of a gene selected from alpha globulin or from a homolog, a fragment or a variant of any one of these genes

50. The method according to claim 47 or 48, wherein the at least one heterologous 3′-UTR comprises a nucleic acid sequence derived from a 3′-UTR from PSMB3 or from a homolog, a fragment or a variant of any one of these genes

51. The method according to claim 47 or 48, wherein the at least one heterologous 3′-UTR comprises a nucleic acid sequence derived from a 3′-UTR from human mitochondrial 12S rRNA (mtRNR1) and human AES/TLE5 gene or from a homolog, a fragment or a variant of any one of these genes

52. The method according to claim 47, wherein the at least one heterologous 5′-UTR comprises a nucleic acid sequence derived from a 5′-UTR of a gene selected from HSD17B4, alpha-globulin, RPL32, ASAH1, ATP5A1, MP68, NDUFA4, NOSIP, RPL31, SLC7A3, TUBB4B and UBQLN2, or from a homolog, a fragment or variant of any one of these genes.

53. The method according to claim 52, wherein the at least one heterologous 5′-UTR comprises a nucleic acid sequence derived from a 5′-UTR from HSD17B4, or from a homolog, a fragment or variant of any one of these genes.

54. The method according to claim 52, wherein the at least one heterologous 5′-UTR comprises a nucleic acid sequence derived from a 5′-UTR from alpha-globulin, or from a homolog, a fragment or variant of any one of these genes.

55. The method according to claim 52, wherein the at least one heterologous 5′-UTR comprises a nucleic acid sequence derived from a 5′-UTR from UBQLN2, or from a homolog, a fragment or variant of any one of these genes.

56. The method according to claim 52, wherein the at least one heterologous 5′-UTR comprises a nucleic acid sequence derived from a 5′-UTR from SLC7A3, or from a homolog, a fragment or variant of any one of these genes.

57. The method according to claim any one of the preceding claims, wherein the obtained in vitro transcribed RNA comprising a 3′ terminal A nucleotide is an RNA., preferably an mRNA.

58. The method according to any of the preceding claims, wherein the method comprises a step iv) of purifying the obtained in vitro transcribed RNA comprising a 3′ terminal A nucleotide, to remove double-stranded RNA, non-capped RNA and/or RNA fragments.

59. The method according to according to any of the preceding claims, wherein the method comprises a step iv) of purifying the obtained in vitro transcribed RNA comprising a 3′ terminal A nucleotide to remove double-stranded RNA.

60. The method according to according to any of the preceding claims, wherein step iv) comprises at least one step of RP-HPLC and/or at least one step of AEX, and/or at least one step of TFF and/or at least one step of oligo d(T) purification and/or at least one step of cellulose purification and/or RNAseIII treatment and/or at least one filtration step including a salt treatment and/or at least one precipitation step and/or at least one core-bead flow through chromatography step.

61. The method according to any of the preceding claims, wherein step iv) comprises at least one step of TFF.

62. The method according to any of the preceding claims, wherein step iv) comprises at least one step of RP-HPLC.

63. The method according to any of the preceding claims, wherein step iv) comprises at least one step of oligo d(T) purification.

64. The method according to any of the preceding claims, wherein step iv) comprises at least one step of cellulose purification.

65. The method according to any of the preceding claims, wherein step iv) comprises at least one step of RP-HPLC and at least one step of cellulose purification.

66. The method according to any of the preceding claims, wherein step iv) comprises at least one step of RP-HPLC and at least one step of oligo d(T) purification.

67. The method according to any of the preceding claims, wherein step iv) comprises at least one step of oligo d(T) purification and at least one step of cellulose purification.

68. The method according to any of the preceding claims, wherein step iv) comprises at least one step of RP-HPLC and oligo d(T) purification and at least one step of cellulose purification.

69. The method according to claims 62 to 68, additionally comprising at least one step of TFF.

70. The method according to any of the preceding claims, wherein the obtained and/or purified in vitro transcribed RNA comprising a 3′ terminal A nucleotide has an RNA integrity of at least 60%.

71. The method according to any of the preceding claims, wherein the obtained and/or purified in vitro transcribed RNA comprising a 3′ terminal A nucleotide has reduced immunostimulatory properties compared to a corresponding reference in vitro transcribed RNA not comprising a 3′-terminal A nucleotide.

72. The method according to claim 71, wherein the immunostimulatory properties are defined as the induction of an innate immune response which is determined by measuring the induction of cytokines.

73. The method according to claim 72, wherein the cytokines are selected from the group consisting of IFNalpha (IFNα), TNFalpha (TNFα), IP-10, IFNgamma (IFNγ), IL-6, IL-12, IL-8, MIG, Rantes, MIP-1alpha (MIP1α), MIP-1beta (MIP1β), McP1, or IFNbeta (IFNβ).

74. The method according to claims 72 to 73, wherein the induction of cytokines is measured by administration of the obtained in vitro transcribed RNA to cells, a tissue or an organism, preferably hPBMCs, Hela cells or HEK cells.

75. The method according to claims 72 to 74, wherein the induction of cytokines is measured and quantified by techniques such as bead based cytokine assays, preferably cytometric bead array (CBA), ELISA, FACS, quantitative mass spectrometry and/or western blot.

76. The method according to any of the preceding claims, wherein the obtained and/or purified in vitro transcribed RNA comprising a 3′ terminal A nucleotide is more stable and/or the optionally encoded peptide or protein is more efficiently expressed compared to a corresponding reference in vitro transcribed RNA not comprising a 3′-terminal A nucleotide.

77. The method according to any of the preceding claims, wherein the method comprises a further step v) formulating the obtained in vitro transcribed RNA with a cationic compound to obtain an RNA formulation.

78. The method according to claim 77, wherein the cationic compound comprises one or more lipids suitable to form liposomes, lipid nanoparticles (LNP), lipoplexes, and/or nanoliposomes.

79. The method according to claims 77 or 78, wherein step v) comprises a purification step after formulating the obtained in vitro transcribed RNA.

80. An in vitro transcribed RNA comprising a 3′ terminal A nucleotide having reduced immunostimulatory properties obtainable by the method as defined in any of claims 1 to 79

81. The vitro transcribed RNA comprising a 3′ terminal A nucleotide according to claim 80, wherein the innate immune response of a subject and/or cell is reduced upon administration to a subject and/or cell.

82. A pharmaceutical composition comprising an in vitro transcribed RNA comprising a 3′ terminal A nucleotide as defined in claims 80 to 81 or an RNA formulation obtained by the method as defined in claims 1 to 79, optionally comprising one or more pharmaceutically acceptable excipients, carriers, diluents and/or vehicles.

83. The pharmaceutical composition according to claim 82, wherein the in vitro transcribed RNA comprising a 3′ terminal A nucleotide is complexed or associated with or at least partially complexed or partially associated with one or more cationic or polycationic compound, preferably cationic or polycationic polymer, cationic or polycationic polysaccharide, cationic or polycationic lipid, cationic or polycationic protein, or cationic or polycationic peptide, or any combinations thereof.

84. The pharmaceutical composition according to claim 82 or 83, wherein at least one in vitro transcribed RNA comprising a 3′ terminal A nucleotide is complexed or associated with one or more lipids, thereby forming liposomes, lipid nanoparticles (LNP), lipoplexes, and/or nanoliposomes.

85. The pharmaceutical composition according to claim 84, wherein at least one in vitro transcribed RNA comprising a 3′ terminal A nucleotide is complexed with one or more lipids thereby forming lipid nanoparticles (LNP).

86. The pharmaceutical composition according to claim 84 or 85, wherein the LNPs comprise at least one lipid selected from an aggregation-reducing lipid, a cationic lipid or ionizable lipid, a neutral lipid or phospholipid, or a steroid or steroid analog, or any combinations thereof.

87. The pharmaceutical composition according to claims 84 to 86, wherein the LNPs comprise an aggregation reducing lipid selected from a polymer conjugated lipid, preferably a PEGylated lipid.

88. The pharmaceutical composition according to claim 87, wherein the PEGylated lipid is a PEG-conjugated lipid preferably selected or derived from DMG-PEG 2000, C10-PEG2K, Cer8-PEG2K, or ALC-0159, preferably ALC-0159.

89. The pharmaceutical composition according to claims 84 to 88, wherein the LNPs comprise a cationic lipid selected or derived from ALC-0315, SM-102, SS-33/4PE-15, HEXA-C5DE-PipSS, or compound C26, preferably ALC-0315.

90. The pharmaceutical composition according to claims 84 to 89, wherein the LNPs comprise a neutral lipid selected or derived from DSPC, DHPC, or DphyPE, preferably DSPC.

91. The pharmaceutical composition according to claims 84 to 90, wherein the LNPs comprise a steroid or steroid analog selected or derived from cholesterol, cholesteryl hemisuccinate (CHEMS), preferably cholesterol.

92. The pharmaceutical composition according to claims 84 to 91, wherein the LNP comprises
(i) at least one cationic lipid, preferably selected from a lipid as defined in claim 89;
(ii) at least one neutral lipid, preferably selected from a lipid as defined in claim 90;
(iii) at least one steroid or steroid analogue, preferably selected from a compound of claim 91; and
(iv) at least one a PEG-lipid, preferably selected from a lipid as defined in claim 88;
wherein (i) to (iv) are in a molar ratio of about 20-60% cationic lipid, 5-25% neutral lipid, 25-55% sterol, and 0.5-15% PEG-lipid.

93. The pharmaceutical composition according to claims 82 to 92, wherein the pharmaceutical composition comprises Ringer or Ringer-Lactate solution.

94. The pharmaceutical composition according to claims 82 to 93, wherein an administration of the pharmaceutical composition to a cell or subject results in a reduced innate immune response compared to an administration of a corresponding composition that comprises an RNA that does not comprise a 3′-terminal A nucleotide.

95. Pharmaceutical composition according to claim 94, wherein the subject is a human subject.

96. Pharmaceutical composition according to claim 94 or 95, wherein the administration is systemically or locally.

97. Pharmaceutical composition according to claim 94 to 96, wherein the administration is transdermally, intradermally, intravenously, intramuscularly, intranorally, intraaterially, intranasally, intrapulmonally, intracranially, intralesionally, intratumorally, intravitreally, subcutaneously or via sublingual, preferably intramuscularly, intranodally, intradermally, intratumorally or intravenously, preferably intramuscularly.

98. Pharmaceutical composition according to claims 94 to 97, wherein the administration is more than once, for example once or once more than once a day, once or more than once a week, once or more than once a month.

99. Pharmaceutical composition according to claims 82 to 99, additionally comprising at least one antagonist of at least one RNA sensing pattern recognition receptor, preferably wherein the at least one antagonist of at least one RNA sensing pattern recognition receptor is a single stranded oligonucleotide.

100. A Kit or kit of parts comprising the in vitro transcribed RNA comprising a 3′ terminal A nucleotide as defined in claims 80 to 81, or pharmaceutical composition as defined in claims 82 to 99, optionally comprising a liquid vehicle for solubilizing, and, optionally, technical instructions providing information on administration and/or dosage of the components.

101. An in vitro transcribed RNA comprising a 3-terminal A nucleotide having reduced immunostimulatory properties as defined in claims 80 to 81, or a pharmaceutical composition as defined in claims 82 to 99, or a kit or kit of parts as defined in claim 100, for use as medicament.

102. An in vitro transcribed RNA comprising the 3-terminal A nucleotide having reduced immunostimulatory properties as defined in claims 80 to 81, or a pharmaceutical composition as defined in claims 82 to 99, or a kit or kit of parts as defined in claim 100, for use in the prevention or treatment of cancer, autoimmune diseases, infectious diseases, allergies or protein deficiency disorders.

103. An in vitro transcribed RNA comprising the 3′-terminal A nucleotide having reduced immunostimulatory properties as defined in claims 80 to 81, or a pharmaceutical composition as defined in claims 82 to 99, or a kit or kit of parts as defined in claim 100, for use in the prevention or treatment of infectious diseases.

104. An in vitro transcribed RNA comprising the 3-terminal A nucleotide having reduced immunostimulatory properties as defined in claims 80 to 81, or a pharmaceutical composition as defined in claims 82 to 99, or a kit or kit of parts as defined in claim 100, for use in the prevention of SARS-CoV-2 infections and/or Influenza infections and/or RSV infections.

105. An in vitro transcribed RNA comprising the 3-terminal A nucleotide having reduced immunostimulatory properties as defined in claims 80 to 81, or a pharmaceutical composition as defined in claims 82 to 99, or a kit or kit of parts as defined in claim 100, for use in the prevention or treatment of protein deficiency disorders.

106. A method of treatment or preventing a disorder, wherein the method comprises applying or administering to a subject in need thereof the in vitro transcribed RNA comprising a 3-terminal A nucleotide as defined in claims 80 to 81, or the pharmaceutical composition as defined in claims 82 to 99, or the kit or kit of parts as defined in claim 100, preferably wherein applying or administering is performed more than once, for example once or more than once a day, once or more than once a week, once or more than once a month.

107. Method of treatment or preventing a disorder according to claim 106, wherein the administration or applying is subcutaneous, intravenous, intramuscular, intra-articular, intra-synovial, intranasal, oral, intrasternal, intrathecal, intrahepatic, intralesional, intracranial, transdermal, intradermal, intrapulmonal, intraperitoneal, intracardial, intraarterial, intraocular, intravitreal, subretinal, intranodal, or intratumoral.

108. Method of treatment or preventing a disorder according to claim 106 and 107, wherein the administration or applying is intramuscular.

109. Method of treatment according to claims 106 to 108, wherein the subject in need is a mammalian subject, preferably a human subject.

110. A method of reducing the induction of an innate immune response induced by an in vitro transcribed RNA upon administration of said RNA to a cell or a subject comprising
(i) obtaining the in vitro transcribed RNA by the method as defined in any of claims 1 to 79; and
(ii) administering an effective amount of the in vitro transcribed RNA comprising a 3′ terminal A nucleotide from step (i) having reduced immunostimulatory properties to a cell or a subject.
(iii)

111. The method of reducing the induction of an innate immune response according to claim 110, wherein the obtained in vitro transcribed RNA as defined in (i) induces less reactogenicity in a subject upon administration, compared to a reference in vitro transcribed RNA not comprising the 5-terminal A nucleotide and not being purified as defined in any of claims 1 to 79.

112. The method of reducing the induction of an innate immune response according to claim 111, wherein the induction of less reactogenicity against the in vitro transcribed RNA leads to the possibility to administer a higher dose of the in vitro transcribed RNA compared to a reference in vitro transcribed RNA.

113. A method of inducing a (protective) immune response in a subject, wherein the method comprises applying or administering to a subject in need thereof the in vitro transcribed RNA comprising a 3′-terminal A nucleotide as defined in claims 80 to 81, or the pharmaceutical composition as defined in claims 82 to 99, or the kit or kit of parts as defined in claim 100, preferably wherein applying or administering is performed more than once, for example once or more than once a day, once or more than once a week, once or more than once a month.

114. The method of inducing a (protective) immune response in a subject according to claim 113, wherein the induction of an innate immune response by the in vitro transcribed RNA has been reduced by a method as defined in any of claims 110 to 112.

115. The method of inducing a (protective) immune response in a subject according to claim 113 or 114, wherein a protective immune response against SARS-CoV-2, Influenza virus and/or RSV infections is induced.
